(19) United States
(12) Patent Application Publication
(10) Pub. No.: US 2025/0388905 A1
(43) Pub. Date: Dec. 25, 2025

(54) POLYCISTRONIC miRNA CONSTRUCTS FOR IMMUNE CHECKPOINT INHIBITION

(71) Applicant: Precigen, Inc., Germantown, MD (US)

(72) Inventors: Cheryl BOLINGER, Germantown, MD (US); Rutul SHAH, Germantown, MD (US)

(21) Appl. No.: 18/992,243

(22) PCT Filed: Jul. 10, 2023
(86) PCT No.: PCT/US2023/069857

§ 371 Date: Jan. 8, 2025

Related U.S. Application Data
(60) Provisional application No. 63/368,056, filed on Jul. 10, 2022.

(51) Int. Cl. C12N 15/113; A61K 40/11; A61K 40/31; A61K 40/42; C07K 14/54; C07K 14/705; C07K 14/71; C07K 14/715; C07K 16/28; C07K 16/30; C12N 5/0783

(52) U.S. Cl. CPC C12N 15/1138; A61K 40/11; A61K 40/31; A61K 40/4257; C07K 14/5443; C07K 14/70521; C07K 14/71; C07K 14/7155; C07K 16/2803; C07K 16/3092; C12N 5/0636; C12N 2310/141

(57) ABSTRACT

A ribonucleic acid comprising two non-natural pre-miRNA sequences, wherein each pre-miRNA sequence comprises a guide miRNA that inhibits the expression of an immune checkpoint protein. The pre-miRNA sequences may target a different gene or target a different regions of the same gene. A deoxyribonucleic acid encoding the aforementioned ribonucleic acid. The deoxyribonucleic acid may further encode a protein such as a chimeric antigen receptor, a cytokine, a cell tag, and/or an immune checkpoint inhibitor. A vector comprising the aforementioned ribonucleoic acid or the aforementioned deoxyribonucleic acid. A method for modifying the expression of a gene in a cell, wherein the method comprises introducing the aforementioned ribonucleic acid or the aforementioned deoxyribonucleic acid to the cell. A method for producing a genetically-engineered cell, wherein the method comprises introducing the aforementioned ribonucleic acid or the aforementioned deoxyribonucleic acid to the cell. A genetically-modified cell comprising the aforementioned ribonucleic acid or the aforementioned deoxyribonucleic acid. A composition comprising the aforementioned ribonucleic acid or the aforementioned deoxynbonucleic acid. A kit comprising the aforementioned ribonucleic acid or the aforementioned deoxyribonucleic acid. A method of treating a disease or disorder in a subject, comprising administering the aforementioned ribonucleic acid or the aforementioned deoxyribonucleic acid to the subject. A method of treating a disease or disorder in a subject, comprising administering the aforementioned cell to the subject. The use of the aforementioned ribonucleic acid or the aforementioned deoxyribonucleic acid in the manufacture of a medicament for modifying the expression of a gene. The use of the aforementioned ribonucleic acid or the aforementioned deoxyribonucleic acid in the manufacture of a medicament for the treatment of a disease or disorder in a subject.

### REFERENCE TO SEQUENCE LISTING

The instant application contains a Sequence Listing that has been submitted electronically in XML format and is incorporated by reference in its entirety. The XML copy was created on Jun. 21, 2023, is named 391456_SL.xml and is 501,736 bytes in size.

### BACKGROUND OF THE INVENTION

Immune checkpoint proteins serve to regulate the immune system. Positive immune checkpoint proteins serve to assist T cells in conducting an immune response. Meanwhile, negative immune checkpoint proteins, such as PD-1, TIGIT, CD70, and CTLA-4, serve to downregulate an immune response and thereby prevent T cells from damaging or killing healthy cells. In individuals with cancer, however, such downregulation may also prevent T cells, including T cells modified to contain a chimeric antigen receptor (CAR-T cells), from killing cancerous cells. As such, it is desired to inhibit the activities of such checkpoint proteins.

Immune checkpoint inhibition, which can prevent the switching off of T cells and promote the activity of these cells, has shown promise as an immunotherapy. Examples of checkpoint inhibitor proteins that can be targeted by such therapy include, but are not limited to, PD1, PD-L1, CTLA-4, TIGIT, 4-1BB, PIK3IP1, CD27, CD28, CD40, CD70, CD122, CD137, OX40 (CD134), GITR, ICOS, A2AR, B7-H3 (CD276), B7-H4 (VTCN1), BTLA, IDO, KIR, LAG3, TIM-3, and VISTA. One of the most studied checkpoint inhibition pathway is the PD-1/programmed death ligand 1 (PD-L1) pathway, which plays a vital role in how tumor cells evade immune response. Immunotherapy utilizing PD-1/PD-L1 blocking antibodies has been extensively evaluated in the clinic and has been shown to improve tumor regression across multiple malignancies, especially when administered in conjunction with CAR-T cells.

Checkpoint inhibitor-blocking antibodies, however, have not performed consistently across cancer types. In addition, such antibodies may have limited access to the tumor microenvironment, require repeated administration, and may lose effectiveness over time. Genome editing is an alternate approach and has the advantage of restricting the checkpoint inhibitor blockade to only engineered cells. However, gene editing adds complexity to the manufacturing process, which increases the turnaround time and cost of the cell therapy. There is accordingly a continuing need in the art to obtain new checkpoint inhibitor therapies.

MicroRNAs (miRNAs) are small non-coding RNA molecules that bind mRNA molecules produced from a targeted gene, affecting their translation to proteins. By such action, the miRNA silences the gene.

To increase silencing of a target gene, it is known to encode multiple miRNAs targeting different regions on the same gene within one polycistronic genetic construct that may be delivered to a cell, for example by way of a vector. See Mueller et al., Molecular Therapy, 20:590-600 (2012) (“Mueller”) at FIG. 1. Such an approach has been shown to achieve robust knockdown of the target gene. Id. However, when a construct containing repeating precursor miRNA (pre-miRNA) structures is used, there is a risk of alternate folding of the pre-miRNA stem-loop structures upon transcription. Such may lead to alternatively processed miRNAs with the potential to cause unintended off-target gene silencing, posing a safety risk. In addition, a construct containing repeating pre-miRNA structures may allow for recombination within the vector, leading to the production of an impure population of vectors having sequence variations.

Applicant addresses these risks by designing a polycistronic construct encoding multiple miRNAs but with each pre-miRNA being distinct and non-complementary to each other. In certain embodiments, at least about 10 nucleotides separate the pre-miRNA structures to help ensure appropriate co-transcriptional folding of the RNA. In addition, in certain embodiments, the pre-miRNAs are designed to maintain the predicted stem-loop structure and internal loops based on endogenous human sequences, which is expected to reduce the risk of the RNAi-based toxicity. The pre-miRNAs in these constructs can each target different genes or different regions of the same gene.

The present invention relates to the use of a polycistronic miRNA construct to modify the expression of genes encoding immune checkpoint proteins.

### SUMMARY OF THE DISCLOSURE

The present invention relates in part to a ribonucleic acid comprising two non-natural pre-miRNA sequences, wherein each pre-miRNA sequence comprises a guide miRNA that inhibits the expression of an immune checkpoint protein.

In certain embodiments, the non-natural pre-miRNA sequences have less than about 50% sequence identity with each other.

In certain embodiments, the nucleic acid sequence of at least one non-natural pre-miRNA sequence has at least about 90% sequence identity with that of a naturally-occurring pre-miRNA sequence.

In certain embodiments, the two non-natural pre-miRNA sequences are separated from each other by at least about 10 nucleotides.

In certain embodiments, each non-natural pre-miRNA sequence targets a different gene.

In certain embodiments, each non-natural pre-miRNA sequence targets different regions of the same gene.

In certain embodiments, each non-natural pre-miRNA comprises backbone sequences that are identical to the corresponding backbone segments of a naturally-occurring pre-miRNA.

In certain embodiments, each non-natural pre-miRNA comprises backbone sequences from miR16, miR17, miR19, miR21, miR22, miR26a1, miR29b1, miR30a, miR122, miR126, miR133a1, miR142, miR150, miR155, miR204, miR206, miR214, miR412, miR486, miR494, or miR1915.

In certain embodiments, each non-natural pre-miRNA comprises backbone sequences from miR16, miR17, miR21, miR22, miR26a1, miR142, miR150, miR204, or miR206.

In certain embodiments, each non-natural pre-miRNA comprises backbone sequences from miR16, miR21, miR22, miR204, or miR206.

In certain embodiments, each non-natural pre-miRNA comprises backbone sequences from miR204 or miR206.

In certain embodiments, the non-natural pre-miRNA comprises a mature miRNA sequence that is capable of binding to an mRNA and thereby interfering with the translation thereof and/or prompting its degradation.

In certain embodiments, the non-natural pre-miRNA comprises a mature miRNA sequence that is capable of binding to an mRNA under stringent hybridization conditions.

In certain embodiments, the immune checkpoint protein is CTLA4, CD70, PD-1, PD-L1, TIGIT, TIM3, LAG3, GITR, or PIK3IP1.

In certain embodiments, the immune checkpoint protein is CTLA4, CD70, PD-1, TIGIT, TIM3, LAG3, GITR, or PIK3IP1.

In certain embodiments, the immune checkpoint protein is CD70, PD-1, or TIGIT.

In certain embodiments, the immune checkpoint protein is PD-1.

The present invention also relates in part to a deoxyribonucleic acid encoding the ribonucleic acid of the present invention.

In certain embodiments, the deoxyribonucleic acid further encodes a protein.

In certain embodiments, the protein is a chimeric antigen receptor.

In certain embodiments, the chimeric antigen receptor comprises an antigen-binding domain that binds an antigen that is overexpressed in a cancer.

In certain embodiments, the chimeric antigen receptor comprises an antigen-binding domain that binds CD19, CD33, MUC-16, or ROR-1.

In certain embodiments, the chimeric antigen receptor comprises an antigen-binding domain that binds ROR-1.

In certain embodiments, the protein is a cytokine.

In certain embodiments, the protein comprises IL-15, or a functional fragment or variant thereof, and IL-15Rα, or a functional fragment or variant thereof.

In certain embodiments, the protein is a cell tag.

In certain embodiments, the cell tag comprises a HER1 Domain III, or a functional fragment or variant thereof, and a truncated HER1 Domain IV, or a functional fragment or variant thereof.

In certain embodiments, the cell tag further comprises a CD28 transmembrane domain or a functional fragment or variant thereof.

In certain embodiments, the protein is an immune checkpoint inhibitor.

In certain embodiments, the deoxyribonucleic acid encodes: (a) a chimeric antigen receptor; (b) a protein comprising IL-15, or a functional fragment or variant thereof, and IL-15Rα, or a functional fragment or variant thereof; and (c) a cell tag.

The present invention also relates in part to a vector comprising the ribonucleoic acid of the present invention or the deoxyribonucleic acid of the present invention.

In certain embodiments, the vector is a plasmid, a nanoplasmid, a viral vector, an episomal vector, or a non-viral vector.

In certain embodiments, the vector is a Sleeping Beauty transposon.

In certain embodiments, the vector is a viral vector.

In certain embodiments, the vector is an adenoviral vector.

The present invention also relates in part to a method for modifying the expression of a gene in a cell, wherein the method comprises introducing the ribonucleic acid of the present invention or the deoxyribonucleic acid of the present invention.

The present invention also relates in part to a method for modifying the expression of a gene in a cell, wherein the method comprises transfecting the cell with the ribonucleic acid of the present invention or the deoxyribonucleic acid of the present invention.

In certain embodiments, the method comprises transfecting the cell with the vector of the present invention.

In certain embodiments, the method further comprises transfecting the cell with a vector encoding a transposase.

The present invention also relates in part to a method for producing a genetically-engineered cell, wherein the method comprises introducing the ribonucleic acid of the present invention or the deoxyribonucleic acid of the present invention to the cell.

The present invention also relates in part to a genetically-modified cell comprising the ribonucleic acid of the present invention or the deoxyribonucleic acid of the present invention.

The present invention also relates in part to a genetically-modified cell produced by a method of the present invention.

The present invention also relates in part to a composition comprising the ribonucleic acid of the present invention or the deoxyribonucleic acid of the present invention.

In certain embodiments, the composition is for use in modifying the expression of a gene.

In certain embodiments, the composition is for use in treating a disease or a disorder in a subject.

The present invention also relates in part to a composition comprising the vector of the present invention or the cell of the present invention.

The present invention also relates in part to a kit comprising the ribonucleic acid of the present invention or the deoxynucleic acid of the present invention.

The present invention also relates in part to a kit comprising the cell of the present invention.

The present invention also relates in part to a method of treating a disease or disorder in a subject, comprising administering the ribonucleic acid of the present invention or the deoxynucleic acid of any one of the present invention to the subject.

The present invention also relates in part to a method of treating a disease or disorder in a subject, comprising administering the cell of the present invention or the deoxynucleic acid of any one of the present invention to the subject.

The present invention also relates in part to a use of the ribonucleic acid of the present invention or the deoxyribonucleic acid of the present invention in the manufacture of a medicament for modifying the expression of a gene.

The present invention also relates in part to a use of the ribonucleic acid of the present invention or the deoxyribonucleic acid of the present invention in the manufacture of a medicament for the treatment of a disease or disorder in a subject.

### BRIEF DESCRIPTION OF THE DRAWINGS

FIG. 1A is an exemplary depiction of a vector comprising a combination of one or more checkpoint inhibitor miRNAs with a chimeric receptor (SD=Splice Donor; SA=Splice Acceptor). FIG. 1B is an exemplary depiction of hairpin and loop design of various pri-miRNAs including target miRNA and complementary sequences at various 5′ or 3′ positions. FIG. 1C is exemplary depiction of hairpin and loop design of various pri-miRNAs including its position in a transgene cassette.

FIG. 2 is a graph depicting PD1 relative RNA expression following transfection of various combinations of miRNA constructs in the presence or absence of MUC16-specific CAR. Constructs #1-8 as depicted on the X-axis are as schematically presented in Table 10.

FIGS. 3A, 3B and 3C are graphs depicting normalized absolute transcript counts obtained from gene analysis of >700 genes using a Nanostring human gene panel code set with CD3/CD28 bead-stimulated CD33 CAR-T cells. In FIG. 3A, the Y-axis plots the transcript counts from CAR-T cells containing an intron coding for 2 checkpoint inhibitor miRNAs targeting PD-1 and TIGIT (CD33 CAR-mbIL15-HER1t+miRNA (PD-1+TIGIT), and the X-axis plots the transcripts from CAR-T cells only (not containing any checkpoint inhibitory miRNA). The circles denote the genes of interest. In FIG. 3B, the Y-axis plots the transcript counts from PD-1 miRNA containing CAR-T cells (CD33 CAR-mbIL15-HER1t+miRNA (PD-1+PD-1)) and the X-axis plots the transcripts from CAR-T cells only. FIG. 3C plots the non-targeting miRNA control (CD33 CAR-mbIL15-HER1t+miRNA scrambled*) on the Y-axis and CAR-T cells without a miRNA containing intron on the X-axis. The circles denote the genes of interest and used to depict the on-target specificity of the checkpoint inhibitor miRNA designs. All three graphs were derived from one donor. * Scrambled controls are non-targeting miRNAs.

FIG. 4A-C are graphs depicting the normalized absolute transcript counts obtained from gene analysis of >700 genes using a Nanostring human gene panel code set with CD3/CD28 bead stimulated MUC16-specific CAR-T cells. In FIG. 4A, the Y-axis plots the transcript counts from CAR-T cells containing an intron coding for miRNAs targeting two different sequences within PD-1 and a sequence for TIGIT (MUC16CAR-mbIL15-HER1t (collectively also referred to as “MUC16CAR”)+miRNA (PD-1/PD-1/TIGIT)), and the X-axis plots the transcripts from CAR-T cells without a miRNA-containing intron (MUC16CAR-mbIL15-HER1t). The black circles denote the genes of interest. In FIG. 4B, the X-axis is the same, and the Y-axis plots the transcript counts from dual PD-1 targeting miRNA containing CAR-T cells (MUC16CAR-mbIL15-HER1t+miRNA (PD-1/PD-1)). FIG. 4C plots the non-targeting miRNA control (MUC16CAR-mbIL15-HER1t+miRNA (scrambled)) on the Y-axis and CAR-T cells without a miRNA-containing intron on the X-axis. All three graphs are from one donor.

FIG. 5A is a graph depicting the number of GFP+K562 cells expressing MUC16 over time. The line with black circle filled dots at each time point denotes number GFP+ target cells in wells without CAR-T cells. The line with square open points denotes target cell counts in wells with MUC16 CAR-mbIL15-HER1t CAR-T cells without a miRNA-containing intron, ((“with CAR-T cells”)). The line with grey circle filled points denotes the target cell counts in wells with CAR-T cells containing a synthetic intron with dual PD-1 targeting miRNAs (MUC16 CAR-mbIL15-HER1t+miRNA (PD-1/PD-1) (“with CAR-T+miRNA cells”). Data are from one donor, plotted is the mean+SD of triplicate wells. ***P<0.001 based on a 2-way ANOVA with Dunnett's Multiple Comparison post hoc test.

FIG. 5B is a graph depicting the number of GFP+K562/MUC16+/PD-L1+/CD155+ cells over time. The line with square filled points at each time point denotes number GFP+ target cells only in wells. The line with open circle points denotes target cell counts in wells with MUC16-specific CAR-T cells without a miRNA-containing intron (MUC16 CAR-mbIL15-HER1t (with CAR-T cells). The line with open circle filled points denotes the target cell counts in wells with CAR-T cells containing a synthetic intron with dual PD-1 and a TIGIT targeting miRNAs (MUC16CAR-mbIL15-HER1t+miRNA (PD-1/PD-1/TIGIT) (with CAR-T+miRNA cells)). Data are from one donor, plotted is the mean+SD of triplicate wells.

FIG. 6A-B depicts cytokine expression levels of IFN gamma and GM-CSF in MUC16 CAR-T cells with a combination of one or more checkpoint inhibitor miRNAs following co-culture with tumor target cells (K562/MUC16t). FIG. 6C-D depicts cytokine expression levels of IFN gamma and GM-CSF in MUC16 CAR-T cells with a combination of one or more checkpoint inhibitor miRNAs without co-culture with tumor target cells. Constructs #1-11 as depicted on the X-axis are as schematically presented in Table 11.

FIG. 7 shows the tumor burden in mice treated with MUC16 CAR+mbIL-15+HER1t (shown as “MUC16 CAR”) in combination with various miRNAs.

FIG. 8A demonstrates PD-1 levels in cell populations following gating hCD45/CD3+/HER1t+ expression in the blood of MUC16CAR+mbIL15+HER1t (CAR only) and MUC16CAR+mbIL15+HER1t+miRNA (PD1/PD-1) (CAR+miRNA (PD-1/PD-1)) treated mice. FIG. 8B shows PD-1 levels as measured by median fluorescent intensity (MFI) in CAR and CAR+miRNA (PD-1/PD-1) treated mice.

FIGS. 9A and 9B demonstrates PD-1 and TIGIT MFI levels in cell populations following gating hCD45/CD3+/HER1t+ expression in the blood of various CAR and CAR+miRNA treated mice. Groups #1-9 as depicted on the X-axis are as schematically presented in Table 12.

FIG. 10A demonstrates that the PD1 silencer module produces guide miRNAs and a corresponding decrease in PD1 mRNA expression in UltraCAR-T cells generated from 5 T cell donors. RT-qPCR results of PD1-targeting guide miRNAs are depicted.

FIG. 10B demonstrates that the PD1 silencer module produced guide miRNAs and a corresponding decrease in PD1 mRNA expression in ultraCAR-T cells generated from 5 T cell donors. RT-qPCR results of PD1 mRNA are depicted.

FIG. 11 demonstrates that the PD1 silencing module preferentially produced PD1-targeting guide miRNAs over non-targeting passenger miRNAs.

FIGS. 12A-E demonstrate that guide miRNAs are the predominant small RNA species originating from the PD1 silencer module.

FIG. 13 shows quantification of mature miRNAs mapping to the PD1 silencer module as a percentage of total small RNAseq reads.

FIGS. 14A and B shows differential gene expression in ROR1+PD1 silencer cells compared to ROR1 UltraCAR-T control cells.

FIGS. 15A-D show a comparison of predicted miRNA binding strength to transcript log fold change.

FIG. 16 provides an exemplary scheme for a genetic construct of the present disclosure.

FIG. 17 depicts TIGIT expression in cells relative to that in a control following the administration of anti-TIGIT miRNA.

FIG. 18 depicts TIGIT expression in cells relative to that in a control following the administration of constructs containing either a single anti-TIGIT pre-miRNA or two anti-TIGIT pre-miRNAs.

FIG. 19 depicts CD70 expression in cells relative to that in a control following the administration of constructs containing: a single anti-CD70 pre-miRNA in a 5′ intron; two anti-PD1 pre-miRNAs and one anti-CD70 pre-miRNA in a 5′ intron; two anti-PD1 pre-miRNAs in a 5′ intron and two anti-CD70 pre-miRNAs in a 3′UTR; and two anti-PD1 pre-miRNAs in a 5′ intron and one anti-CD70 pre-miRNA in a 3′UTR.

### DETAILED DESCRIPTION OF THE DISCLOSURE

The following description and examples illustrate embodiments of the present disclosure in detail. It is to be understood that the present disclosure is not limited to the particular embodiments described herein and as such can vary. Those of skill in the art will recognize that there are variations and modifications of the present disclosure, which are encompassed within the scope of the present invention.

Unless defined otherwise, all technical and scientific terms used herein have the same meaning as commonly understood by one of ordinary skill in the art to which the disclosure pertains.

The section headings used herein are for organizational purposes only and are not to be construed as limiting the subject matter described.

Although various features of the disclosure can be described in the context of a single embodiment, the features can also be provided separately or in any suitable combination. Conversely, although the present disclosure can be described herein in the context of separate embodiments for clarity, the present disclosure can also be implemented in a single embodiment.

### I. Definitions

The following definitions supplement those in the art and are directed to the current application and are not to be imputed to any related or unrelated case, e.g., to any commonly owned patent or application. The terminology used herein is for the purpose of describing particular embodiments only, and is not intended to be limiting.

In this application, the use of the singular includes the plural unless specifically stated otherwise. As used in the specification, the singular forms “a,” “an” and “the” include plural referents unless the context clearly dictates otherwise.

In this application, the use of “or” means “and/or” unless stated otherwise. The terms “and/or” and “any combination thereof” and their grammatical equivalents as used herein, can be used interchangeably. These terms can convey that any combination is specifically contemplated. Solely for illustrative purposes, the following phrases “A, B, and/or C” or “A, B, C, or any combination thereof” can mean “A individually; B individually; C individually; A and B; B and C; A and C; and A, B, and C.” The term “or” can be used conjunctively or disjunctively, unless the context specifically refers to a disjunctive use.

Furthermore, use of the term “including” as well as other forms, such as “include,” “includes,” and “included,” is not limiting.

Reference in the specification to “some embodiments,” “an embodiment,” “one embodiment” or “other embodiments” means that a particular feature, structure, or characteristic described in connection with the embodiments is included in at least some embodiments, but not necessarily all embodiments, of the present disclosures.

As used in this specification and the claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the disclosure, and vice versa. Furthermore, compositions of the present disclosure can be used to achieve methods of the present disclosure.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. In another example, the amount “about 10” includes 10 and any amounts from 9 to 11. In yet another example, the term “about” in relation to a reference numerical value can also include a range of values plus or minus 10%, 9%, 8%, 7%, 6%, 5%, 4%, 3%, 2%, or 1% from that value. Alternatively, particularly with respect to biological systems or processes, the term “about” can mean within an order of magnitude, preferably within 5-fold, and more preferably within 2-fold, of a value. Where particular values are described in the application and claims, unless otherwise stated the term “about” meaning within an acceptable error range for the particular value should be assumed.

A “therapeutically-effective amount” or “therapeutically-effective dose” refers to an amount or dose effective, for periods of time necessary, to achieve a desired therapeutic result. The amount can vary according to factors such as the disease state, age, sex, and weight of the individual, and the ability of the inventive nucleic acid sequences to elicit a desired response in the individual.

“Polynucleotide” or “oligonucleotide” refers to a polymeric form of nucleotides or nucleic acids of any length, either ribonucleotides or deoxyribonucleotides. This term refers only to the primary structure of the molecule. Thus, this term includes double and single stranded deoxyribonucleic acid (DNA), triplex DNA, as well as double and single stranded ribonucleic acid (RNA). It also includes modified, for example, by methylation and/or by capping, and unmodified forms of the polynucleotide. The term is also meant to include molecules that include non-naturally occurring or synthetic nucleotides as well as nucleotide analogs.

Unless otherwise stated, nucleic acid sequences in the text of this specification are given, when read from left to right, in the 5′ to 3′ direction.

The terms “transfection,” “transformation,” “nucleofection,” or “transduction” as used herein refer to the introduction of one or more exogenous polynucleotides into a host cell or organism by using physical, chemical, and/or electrical methods. The nucleic acid sequences and vectors disclosed herein can be introduced into a cell or organism by any such methods, including, for example, by electroporation, calcium phosphate co-precipitation, strontium phosphate DNA co-precipitation, liposome mediated-transfection, DEAE dextran mediated-transfection, polycationic mediated-transfection, tungsten particle-facilitated microparticle bombardment, viral, and/or non-viral mediated transfection. In some cases, the method of introducing nucleic acids into the cell or organism involves the use of viral, retroviral, lentiviral, or transposon, or transposable element-mediated (e.g., Sleeping Beauty) vectors.

“Polypeptide,” “peptide,” and their grammatical equivalents as used herein refer to a polymer of amino acid residues. The polypeptide can optionally include glycosylation or other modifications typical for a given protein in a given cellular environment. Polypeptides and proteins disclosed herein (including functional fragments and functional variants thereof) can comprise synthetic amino acids in place of one or more naturally-occurring amino acids. Such synthetic amino acids are known in the art, and include, for example, aminocyclohexane carboxylic acid, norleucine, α-amino n-decanoic acid, homoserine, S-acetylaminomethyl-cysteine, trans-3- and trans-4-hydroxyproline, 4-aminophenylalanine, 4-nitrophenylalanine, 4-chlorophenylalanine, 4-carboxyphenylalanine, β-phenylserine β-hydroxyphenylalanine, phenylglycine, α-naphthylalanine, cyclohexylalanine, cyclohexylglycine, indoline-2-carboxylic acid, 1,2,3,4-tetrahydroisoquinoline-3-carboxylic acid, aminomalonic acid, aminomalonic acid monoamide, N′-benzyl-N′-methyl-lysine, N′,N′-dibenzyl-lysine, 6-hydroxylysine, ornithine, α-aminocyclopentane carboxylic acid, α-aminocyclohexane carboxylic acid, α-aminocycloheptane carboxylic acid, α-(2-amino-2-norbornane)-carboxylic acid, α,γ-diaminobutyric acid, α,β-diaminopropionic acid, homophenylalanine, and α-tert-butylglycine. The present disclosure further contemplates that expression of polypeptides or proteins described herein in an engineered cell can be associated with post-translational modifications of one or more amino acids of the polypeptide or protein. Non-limiting examples of post-translational modifications include phosphorylation, acylation including acetylation and formylation, glycosylation (including N-linked and O-linked), amidation, hydroxylation, alkylation including methylation and ethylation, ubiquitylation, addition of pyrrolidone carboxylic acid, formation of disulfide bridges, sulfation, myristoylation, palmitoylation, isoprenylation, farnesylation, geranylation, glypiation, lipoylation and iodination.

The term “conservative amino acid substitution” or “conservative mutation” refers to the replacement of one amino acid by another amino acid with a common property. A functional way to define common properties between individual amino acids is to analyze the normalized frequencies of amino acid changes between corresponding proteins of homologous organisms (Schulz, G. E. and Schirmer, R. H., Principles of Protein Structure, Springer-Verlag, New York (1979)). According to such analyses, groups of amino acids can be defined where amino acids within a group exchange preferentially with each other, and therefore resemble each other most in their impact on the overall protein structure (Schulz, G. E. and Schirmer, R. H., supra). Examples of conservative mutations include amino acid substitutions of amino acids within the sub-groups below, for example, lysine for arginine and vice versa such that a positive charge can be maintained; glutamic acid for aspartic acid and vice versa such that a negative charge can be maintained; serine for threonine such that a free-OH can be maintained; and glutamine for asparagine such that a free —NH₂ can be maintained. Exemplary conservative amino acid substitutions are shown in the following chart:

| Type of Amino Acid | Substitutable Amino Acids |
|---|---|
| Hydrophilic | Ala, Pro, Gly, Glu, Asp, Gln, Asn, Ser, Thr |
| Sulphydryl | Cys |
| Aliphatic | Val, Ile, Leu, Met |
| Basic | Lys, Arg, His |
| Aromatic | Phe, Tyr, Trp |

An amino acid sequence that differs from a reference amino acid sequence by only conservative amino acid substitutions will be referred to herein as a “conservatively-substituted variant” of the reference sequence.

In some embodiments, the functional variants can comprise the amino acid sequence of the reference protein with at least one non-conservative amino acid substitution. The term “non-conservative mutations” involve amino acid substitutions between different groups, for example, lysine for tryptophan, or phenylalanine for serine, etc. In this case, it is preferable for the non-conservative amino acid substitution to not interfere with, or inhibit the biological activity of, the functional variant. The non-conservative amino acid substitution can enhance the biological activity of the functional variant, such that the biological activity of the functional variant is increased as compared to the homologous parent protein. Amino acid substitutability is discussed in more detail, for example, in L. Y. Yampolsky and A. Stoltzfus, “The Exchangeability of Amino acids in Proteins,” Genetics 2005 August; 170(4):1459-1472.

The terms “identical” and its grammatical equivalents as used herein or “sequence identity” in the context of two nucleic acid sequences or amino acid sequences of polypeptides refer to the residues in the two sequences which are the same when aligned for maximum correspondence over a specified comparison window. A “comparison window,” as used herein, refers to a segment of at least about 20 contiguous positions, usually about 50 to about 200, more usually about 100 to about 150 in which a sequence can be compared to a reference sequence of the same number of contiguous positions after the two sequences are aligned optimally. Methods of alignment of sequences for comparison are well-known in the art. Optimal alignment of sequences for comparison can be conducted by the local homology algorithm of Smith and Waterman, Adv. Appl. Math., 2:482 (1981); by the alignment algorithm of Needleman and Wunsch, J. Mol. Biol., 48:443 (1970); by the search for similarity method of Pearson and Lipman, Proc. Nat. Acad. Sci U.S.A., 85:2444 (1988); by computerized implementations of these algorithms (including, but not limited to CLUSTAL in the PC/Gene program by Intelligentics, Mountain View Calif., GAP, BESTFIT, BLAST, FASTA, and TFASTA in the Wisconsin Genetics Software Package, Genetics Computer Group (GCG), 575 Science Dr., Madison, Wis., U.S.A.); the CLUSTAL program is well described by Higgins and Sharp, Gene, 73:237-244 (1988) and Higgins and Sharp, CABIOS, 5:151-153 (1989); Corpet et al., Nucleic Acids Res., 16:10881-10890 (1988); Huang et al., Computer Applications in the Biosciences, 8:155-165 (1992); and Pearson et al., Methods in Molecular Biology, 24:307-331 (1994). Alignment is also often performed by inspection and manual alignment. In one class of embodiments, the polypeptides herein are at least 80%, 85%, 90%, 98% 99% or 100% identical to a reference polypeptide (i.e., the full length thereof), or a fragment thereof, e.g., as measured by BLASTP (or CLUSTAL, or any other available alignment software) using default parameters. Similarly, nucleic acids can also be described with reference to a starting nucleic acid, e.g., they can be 50%, 60%, 70%, 75%, 80%, 85%, 90%, 98%, 99% or 100% identical to a reference nucleic acid (i.e., the full length thereof) or a fragment thereof, e.g., as measured by BLASTN (or CLUSTAL, or any other available alignment software) using default parameters. When one molecule is said to have certain percentage of sequence identity with a larger molecule, it means that when the two molecules are optimally aligned, the percentage of residues in the smaller molecule finds a match residue in the larger molecule in accordance with the order by which the two molecules are optimally aligned.

For purposes of this specification and the claims, it is understood that the phrase “having at least 50% sequence identity with” a reference sequence, or referencing any range therein (e.g., “at least 80% sequence identity with”) encompasses the reference sequence itself. Thus, for example, a claim reciting “a nucleic acid having at least 80% sequence identity with SEQ ID NO: 0” encompasses SEQ ID NO: 0 itself.

The term “substantially identical” and its grammatical equivalents as applied to nucleic acid or amino acid sequences mean that a nucleic acid or amino acid sequence comprises a sequence that has at least 95% sequence identity with a reference sequence using the programs described above, e.g., BLAST, using standard parameters.

“Homology” is generally inferred from sequence identity between two or more nucleic acids or proteins (or sequences thereof). The precise percentage of identity between sequences that is useful in establishing homology varies with the nucleic acid and protein at issue, but as little as 25% sequence identity is routinely used to establish homology. Higher levels of sequence identity, e.g., 30%, 40%, 50%, 60%, 70%, 80%, 90%, 95%, or 99% or more can also be used to establish homology. Methods for determining sequence identity percentages (e.g., BLASTP and BLASTN using default parameters) are described herein and are generally available. Nucleic acids and/or nucleic acid sequences are “homologous” when they are derived, naturally or artificially, from a common ancestral nucleic acid or nucleic acid sequence. Proteins and/or protein sequences are “homologous” when their encoding DNAs are derived, naturally or artificially, from a common ancestral nucleic acid or nucleic acid sequence. The homologous molecules can be termed “homologs.” For example, any naturally occurring proteins can be modified by any available mutagenesis method. When expressed, this mutagenized nucleic acid encodes a polypeptide that is homologous to the protein encoded by the original nucleic acid.

Also contemplated and included herein are nucleic acid molecules that hybridize to the disclosed sequences. Hybridization conditions may be mild, moderate, or stringent, as is warranted.

Appropriate stringency conditions which promote DNA hybridization, for example, 6× sodium chloride/sodium citrate (SSC) at about 45° C., followed by a wash of 2×SSC at 50° C., are known or can be found in Current Protocols in Molecular Biology, John Wiley & Sons, N.Y. (1989), 6.3.1-6.3.6. “Stringent hybridization conditions” are those that include a salt concentration of 1.0 M NaCl in 50% formamide, at a temperature of 37° C. for 4 to 12 hours, followed by a wash in 0.1×SSC at 60-65° C.

As will be appreciated by the skilled practitioner, slight changes in nucleic acid sequence do not necessarily alter the amino acid sequence of the encoded polypeptide. This disclosure embraces the degeneracy of codon usage as would be understood by one of ordinary skill in the art. For example, as known in the art, different codons will code for the same amino acid as illustrated in the following chart.

| Amino Acid | Codons |
|---|---|
| Ala/A | GCT, GCC, GCA, GCG |
| Arg/R | CGT, CGC, CGA, CGG, AGA, AGG |
| Asn/N | AAT, AAC |
| Asp/D | GAT, GAC |
| Cys/C | TGT, UGC |
| Gln/Q | CAA, CAG |
| Glu/E | GAA, GAG |
| Gly/G | GGT, GGC, GGA, GGG |
| His/H | CAT, CAC |
| Ile/I | ATT, ATC, ATA |
| Leu/L | TTA, TTG, CTT, CTC, CTA, CTG |
| Lys/K | AAA, AAG |
| Met/M | ATG |
| Phe/F | TTT, TTC |
| Pro/P | CCT, CCC, CCA, CCG |
| Ser/S | TCT, TCC, TCA, TCG, AGT, AGC |
| Thr/T | ACT, ACC, ACA, ACG |
| Trp/W | TGG |
| Tyr/Y | TAT, TAC |
| Val/V | GTT, GTC, GTA, GTG |
| START | ATG |
| STOP | TAG, TGA, TAA |

As used herein, the phrase “codon degenerate variant” when used with reference to a nucleic acid sequence means a nucleic acid sequence that differs from the referenced sequence, but that encodes a polypeptide having the same amino acid sequence as that encoded by the referenced sequence.

Additionally, it will be appreciated by persons skilled in the art that partial sequences often work as effectively as full-length versions. The ways in which the nucleotide sequence can be varied or shortened are well known to persons skilled in the art, as are ways of testing the suitability or effectiveness of the altered genes. In certain embodiments, suitability and/or effectiveness of the altered gene may easily be tested by, for example, conventional gas chromatography. All such variations of the genes are therefore included as part of the present disclosure.

The term “isolated” and its grammatical equivalents as used herein refer to the removal of a nucleic acid from its natural environment. It is to be understood, however, that nucleic acids and proteins can be formulated with diluents or adjuvants and still for practical purposes be isolated.

The term “purified” and its grammatical equivalents as used herein refer to a molecule or composition, whether removed from nature (including genomic DNA and mRNA) or synthesized (including cDNA) and/or amplified under laboratory conditions, that has been increased in purity, wherein “purity” is a relative term, not “absolute purity.” For example, nucleic acids typically are mixed with an acceptable carrier or diluent when used for introduction into cells. The term “substantially purified” and its grammatical equivalents as used herein refer to a nucleic acid sequence, polypeptide, protein or other compound that is essentially free, i.e., is more than about 50% free of, more than about 70% free of, more than about 90% free of, the polynucleotides, proteins, polypeptides and other molecules that the nucleic acid, polypeptide, protein or other compound is naturally associated with.

“T cell” or “T lymphocyte” as used herein is a type of lymphocyte that plays a central role in cell-mediated immunity. They can be distinguished from other lymphocytes, such as B cells and natural killer cells (NK cells), by the presence of a T-cell receptor (TCR) on the cell surface.

“Transposon,” “transposable element” or “TE” refers to a DNA sequence that can change its position within the genome, sometimes creating or reversing mutations and altering the cell's genome size. Transposition often results in duplication of the transposon. Class I transposons are copied in two stages: first, they are transcribed from DNA to RNA, and the RNA produced is then reverse transcribed to DNA. This copied DNA is then inserted at a new position into the genome. The reverse transcription step is catalyzed by a reverse transcriptase, which can be encoded by the transposon itself. The characteristics of retrotransposons are similar to retroviruses, such as HIV. The cut-and-paste transposition mechanism of class II transposons does not involve an RNA intermediate. The transpositions are catalyzed by several transposase enzymes. Some transposases non-specifically bind to any target site in DNA, whereas others bind to specific DNA sequence targets. The transposase makes a staggered cut at the target site resulting in single-strand 5′ or 3′ DNA overhangs (sticky ends). This step cuts out the DNA transposon, which is then ligated into a new target site; this process involves activity of a DNA polymerase that fills in gaps and of a DNA ligase that closes the sugar-phosphate backbone. This results in duplication of the target site. The insertion sites of DNA transposons can be identified by short direct repeats which can be created by the staggered cut in the target DNA and filling in by DNA polymerase, followed by a series of inverted repeats important for the transposon excision by transposase. Cut-and-paste transposons can be duplicated if their transposition takes place during S phase of the cell cycle when a donor site has already been replicated, but a target site has not yet been replicated. Transposition can be classified as either “autonomous” or “non-autonomous” in both Class I and Class II transposons. Autonomous transposons can move by themselves while non-autonomous transposons require the presence of another transposon to move. This is often because non-autonomous transposons lack transposase (for class II) or reverse transcriptase (for class I).

“Transposase” refers an enzyme that binds to the end of a transposon and catalyzes the movement of the transposon to another part of the genome by a cut and paste mechanism or a replicative transposition mechanism. In some embodiments, the transposase's catalytic activity can be utilized to move gene(s) from a vector to the genome.

An “expression vector” or “vector” is any genetic element, e.g., a plasmid, a mini-circle, a nanoplasmid, chromosome, virus, transposon, behaving either as an autonomous unit of polynucleotide replication within a cell. (i.e. capable of replication under its own control) or being rendered capable of replication by insertion into a host cell chromosome, having attached to it another polynucleotide segment, so as to bring about the replication and/or expression of the attached segment. Suitable vectors include, but are not limited to, plasmids, transposons, bacteriophages and cosmids. Vectors can contain polynucleotide sequences that are necessary to effect ligation or insertion of the vector into a desired host cell and to effect the expression of the attached segment. Such sequences differ depending on the host organism; they include promoter sequences to effect transcription, enhancer sequences to increase transcription, ribosomal binding site sequences and transcription and translation termination sequences. Alternatively, expression vectors can be capable of directly expressing nucleic acid sequence products encoded therein without ligation or integration of the vector into host cell DNA sequences. In some embodiments, the vector is an “episomal expression vector” or “episome,” which is able to replicate in a host cell, and persists as an extrachromosomal segment of DNA within the host cell in the presence of appropriate selective pressure (see, e.g., Conese et al., Gene Therapy, 11:1735-1742 (2004)). Representative commercially-available episomal expression vectors include, but are not limited to, episomal plasmids that utilize Epstein Barr Nuclear Antigen 1 (EBNA1) and the Epstein Barr Virus (EBV) origin of replication (oriP). The vectors pREP4, pCEP4, pREP7, and pcDNA3.1 from Invitrogen (Carlsbad, Calif.) and pBK-CMV from Stratagene (La Jolla, Calif.) represent non-limiting examples of an episomal vector that uses T-antigen and the SV40 origin of replication in lieu of EBNA1 and oriP. A vector also can comprise a selectable marker gene. In certain embodiments where nano plasmids are utilized, strains such as R6K that utilizes an antisense RNA selection marker (e.g. sucrose tolerance) can be used.

The term “selectable marker gene” refers to a nucleic acid sequence that allows cells expressing the nucleic acid sequence to be specifically selected for or against, in the presence of a corresponding selective agent. Suitable selectable marker genes are known in the art and described in, e.g., International Patent Application Publications WO 1992/08796 and WO 1994/28143; Wigler et al., Proc. Natl. Acad. Sci. USA, 77: 3567 (1980); O'Hare et al., Proc. Natl. Acad. Sci. USA, 78: 1527 (1981); Mulligan & Berg, Proc. Natl. Acad. Sci. USA, 78: 2072 (1981); Colberre-Garapin et al., J. Mol. Biol., 150:1 (1981); Santerre et al., Gene, 30: 147 (1984); Kent et al., Science, 237: 901-903 (1987); Wigler et al., Cell, 11: 223 (1977); Szybalska & Szybalski, Proc. Natl. Acad. Sci. USA, 48: 2026 (1962); Lowy et al., Cell, 22: 817 (1980); and U.S. Pat. Nos. 5,122,464 and 5,770,359.

The term “coding sequence” refers to a segment of a polynucleotide that encodes for protein or polypeptide. The region or sequence is bounded nearer the 5′ end by a start codon and nearer the 3′ end with a stop codon. Coding sequences can also be referred to as open reading frames.

The term “operably linked” refers to refers to the physical and/or functional linkage of a DNA segment to another DNA segment in such a way as to allow the segments to function in their intended manners. A DNA sequence encoding a gene product is operably linked to a regulatory sequence when it is linked to the regulatory sequence, such as, for example, promoters, enhancers and/or silencers, in a manner, that allows modulation of transcription of the DNA sequence, directly or indirectly. For example, a DNA sequence is operably linked to a promoter when it is ligated to the promoter downstream with respect to the transcription initiation site of the promoter and in the correct reading frame with respect to the transcription initiation site so as to allow transcription elongation to proceed through the DNA sequence. An enhancer or silencer is operably linked to a DNA sequence coding for a gene product when it is ligated to the DNA sequence in such a manner as to, respectively, increase or decrease the transcription of the DNA sequence. Enhancers and silencers can be located upstream or downstream of or embedded within the coding regions of the DNA sequence. A DNA for a signal sequence is operably linked to DNA coding for a polypeptide if the signal sequence is expressed as a pre-protein that participates in the secretion of the polypeptide. Linkage of DNA sequences to regulatory sequences is typically accomplished by ligation at suitable restriction sites or via adapters or linkers inserted in the sequence using restriction endonucleases known to one of skill in the art.

The terms “induce,” “induction” and their grammatical equivalents as used herein refer to an increase in nucleic acid sequence transcription, promoter activity and/or expression brought about by a transcriptional regulator, relative to some basal level of transcription.

The term “transcriptional regulator” refers to a biochemical element that acts to prevent or inhibit the transcription of a promoter-driven DNA sequence under certain environmental conditions (e.g., a repressor or nuclear inhibitory protein), or to permit or stimulate the transcription of the promoter-driven DNA sequence under certain environmental conditions (e.g., an inducer or an enhancer).

The term “enhancer,” as used herein, refers to a DNA sequence that increases transcription of, for example, a nucleic acid sequence to which it is operably linked. Enhancers can be located many kilobases away from the coding region of the nucleic acid sequence and can mediate the binding of regulatory factors, patterns of DNA methylation, or changes in DNA structure. A large number of enhancers from a variety of different sources are well known in the art and are available as or within cloned polynucleotides (from, e.g., depositories such as the ATCC as well as other commercial or individual sources). A number of polynucleotides comprising promoters (such as the commonly-used CMV promoter) also comprise enhancer sequences. Enhancers can be located upstream or downstream of coding sequences or within coding sequences. The term “Ig enhancers” refers to enhancer elements derived from enhancer regions mapped within the immunoglobulin (Ig) locus (such enhancers include for example, the heavy chain (mu) 5′ enhancers, light chain (kappa) 5′ enhancers, kappa and mu intronic enhancers, and 3′ enhancers (see generally Paul W. E. (ed), Fundamental Immunology, 3rd Edition, Raven Press, New York (1993), pages 353-363; and U.S. Pat. No. 5,885,827).

The term “promoter” refers to a region of a polynucleotide that initiates transcription of a coding sequence. Promoters are located near the transcription start sites of genes, on the same strand and upstream on the DNA (towards the 5′ region of the sense strand). Some promoters are constitutive as they are active in all circumstances in the cell, while others are regulated becoming active in response to specific stimuli, e.g., an inducible promoter. The term “promoter activity” and its grammatical equivalents as used herein refer to the extent of expression of nucleotide sequence that is operably linked to the promoter whose activity is being measured. Promoter activity can be measured directly by determining the amount of RNA transcript produced, for example by Northern blot analysis or indirectly by determining the amount of product coded for by the linked nucleic acid sequence, such as a reporter nucleic acid sequence linked to the promoter.

“Inducible promoter” refers to a promoter, that is induced into activity by the presence or absence of transcriptional regulators, e.g., biotic or abiotic factors. Inducible promoters are useful because the expression of genes operably linked to them can be turned on or off at certain stages of development of an organism or in a particular tissue. Non-limiting examples of inducible promoters include alcohol-regulated promoters, tetracycline-regulated promoters, steroid-regulated promoters, metal-regulated promoters, pathogenesis-regulated promoters, temperature-regulated promoters and light-regulated promoters. The inducible promoter can be part of a gene switch or genetic switch.

“T cell” or “T lymphocyte” as used herein is a type of lymphocyte that plays a central role in cell-mediated immunity. They can be distinguished from other lymphocytes, such as B cells and natural killer cells (NK cells), by the presence of a T-cell receptor (TCR) on the cell surface.

As used herein, the phrase “functional fragment” when used with reference to a polypeptide refers to a fragment of such polypeptide that possesses the primary function of the referenced polypeptide. For example, a functional fragment of a polypeptide that serves as a transmembrane domain is a fragment of that polypeptide that also serves as a transmembrane domain. In certain embodiments, the functional fragment of a polypeptide is shorter than the referenced polypeptide by at most 30, 25, 20, 15, 10, 9, 8, 7, 6, 5, 4, 3, 2, or 1 amino acid residues at the N- and/or C-terminus. When used with reference to a nucleic acid, the phrase “functional fragment” refers to a fragment of the referenced nucleic acid that encodes a polypeptide having the same primary function as the polypeptide encoded by the referenced nucleic acid.

As used herein, the phrase “functional variant” when used with reference to a polypeptide refers to a polypeptide that differs from the referenced polypeptide but possesses the primary function of the referenced polypeptide. For example, a functional variant of a polypeptide that serves as a transmembrane domain is a fragment of that polypeptide that also serves as a transmembrane domain. In certain embodiments, the functional variant has at least 80%, 85%, 90%, 95%, 96%, 97%, 98%, or 99% sequence identity with the referenced amino acid sequence and/or is a conservatively-substituted variant of the referenced sequence. When used with reference to a nucleic acid, the phrase “functional variant” refers to a nucleic acid that differs from the referenced nucleic acid but encodes a polypeptide having the same primary function as the polypeptide encoded by the referenced nucleic acid. In certain embodiments, the functional variant has at least 80%, 85%, 90%, 95%, 96%, 97%, 98%, or 99% sequence identity with the referenced nucleic acid sequence, hybridizes under stringent hybridization conditions with the complement of the referenced nucleic acid sequence, or is a codon degenerate variant of the nucleic acid sequence.

The term “antibody,” also known as immunoglobulin (Ig), as used herein can refer to a monoclonal or polyclonal antibody. The term “monoclonal antibodies,” as used herein, refers to antibodies that are produced by a single clone of B-cells and bind to the same epitope. In contrast, “polyclonal antibodies” refer to a population of antibodies that is produced by different B-cells and bind to different epitopes of the same antigen. The antibodies can be from any animal origin. An antibody can be IgG (including IgG1, IgG2, IgG3, and IgG4), IgA (including IgA1 and IgA2), IgD, IgE, or IgM, and IgY. In some embodiments, the antibody can a single-chain whole antibody. An antibody typically consists of four polypeptides: two identical copies of a heavy (H) chain polypeptide and two identical copies of a light (L) chain polypeptide. Each of the heavy chains contains one N-terminal variable (V_{H}) region and three C-terminal constant (CH1, CH2 and CH3) regions, and each light chain contains one N-terminal variable (V_{L}) region and one C-terminal constant (C_{L}) region. The variable regions of each pair of light and heavy chains form the antigen-binding site of an antibody. The V_{H} and V_{L} regions have a similar general structure, with each region comprising four framework regions, whose sequences are relatively conserved. The framework regions are connected by three complementarity determining regions (CDRs). The three CDRs, known as CDR1, CDR2, and CDR3, form the “hypervariable region” of an antibody, which is responsible for antigen binding. These particular regions have been described by Kabat et al., J. Biol. Chem. 252, 6609-6616 (1977) and Kabat et al., Sequences of protein of immunological interest. (1991), by Chothia et al., J. Mol. Biol. 196:901-917 (1987), and by MacCallum et al., J. Mol. Biol. 262:732-745 (1996), where the definitions include overlapping or subsets of amino acid residues when compared against each other. Preferably, the term “CDR” is a CDR as defined by Kabat, based on sequence comparisons. CDRH1, CDRH2 and CDRH3 denote the heavy chain CDRs, and CDRL1, CDRL2 and CDRL3 denote the light chain CDRs.

The terms “fragment of an antibody,” “antibody fragment,” “fragment of an antibody,” “antigen-binding portion” and their grammatical equivalents are used interchangeably herein to mean one or more fragments or portions of an antibody that retain the ability to specifically bind to an antigen (see, generally, Holliger et al., Nat. Biotech., 23(9):1126-1129 (2005). The antibody fragment desirably comprises, for example, one or more CDRs, the variable region (or portions thereof), the constant region (or portions thereof), or combinations thereof. Non-limiting examples of antibody fragments include (1) a Fab fragment, which is a monovalent fragment consisting of the V_{L}, V_{H}, C_{L}, and CH1 domains; (2) a F(ab′)2 fragment, which is a bivalent fragment comprising two Fab fragments linked by a disulfide bridge at the stalk region; (3) a Fv fragment consisting of the V_{L} and V_{H} domains of a single arm of an antibody; (4) a single chain Fv (scFv), which is a monovalent molecule consisting of the two domains of the Fv fragment (i.e., V_{L} and V_{H}) joined by a linker that enables the two domains to be synthesized as a single polypeptide chain (see, e.g., Bird et al., Science, 242: 423-426 (1988); Huston et al., Proc. Natl. Acad. Sci. USA, 85: 5879-5883 (1988); and Osbourn et al., Nat. Biotechnol., 16: 778 (1998)) and (5) a diabody, which is a dimer of polypeptide chains, wherein each polypeptide chain comprises a V_{H} connected to a V_{L} by a peptide linker that is too short to allow pairing between the V_{H} and V_{L} on the same polypeptide chain, thereby driving the pairing between the complementary domains on different V_{H}-V_{L} polypeptide chains to generate a dimeric molecule having two functional antigen-binding sites. Antibody fragments are known in the art and are described in more detail in, e.g., U.S. Pat. No. 8,603,950.

The terms “antigen recognition moiety,” “antigen recognition domain,” “antigen-binding domain,” and “antigen binding region” refer to a molecule or portion of a molecule that specifically binds to an antigen. In one embodiment, the antigen recognition moiety is an antibody, antibody like molecule or fragment thereof.

The term “proliferative disease” refers to a unifying concept in which excessive proliferation of cells and/or turnover of cellular matrix contributes significantly to the pathogenesis of the disease, including cancer. In some embodiments, the proliferative disease is cancer.

“Patient” or “subject” refers to a mammalian subject diagnosed with or suspected of having or developing a proliferative disorder such as cancer. In some embodiments, the term “patient” refers to a mammalian subject with a higher than average likelihood of developing a proliferative disorder such as cancer. Exemplary patients can be humans, apes, dogs, pigs, cattle, cats, horses, goats, sheep, rodents and other mammalians that can benefit from the therapies disclosed herein. Exemplary human patients can be male and/or female. “Patient in need thereof” or “subject in need thereof” means a patient diagnosed with or suspected of having a disease or disorder, for instance, but not restricted to cancer.

“Administering” refers to herein as providing one or more compositions described herein to a patient or a subject. By way of example and not limitation, composition administration, e.g., injection, can be performed by intravenous (i.v.) injection, sub-cutaneous (s.c.) injection, intradermal (i.d.) injection, intraperitoneal (i.p.) injection, or intramuscular (i.m.) injection. One or more such routes can be employed. Parenteral administration can be, for example, by bolus injection or by gradual perfusion over time. Alternatively, or concurrently, administration can be by the oral route. Additionally, administration can also be by surgical deposition of a bolus or pellet of cells, or positioning of a medical device.

As used herein, the terms “treatment,” “treating,” and their grammatical equivalents refer to obtaining a desired pharmacologic and/or physiologic effect. In some embodiments, the effect is therapeutic, i.e., the effect partially or completely cures a disease and/or adverse symptom attributable to the disease. In some embodiments, the term “treating” can include “preventing” a disease or a condition.

As used herein, a “treatment interval” refers to a treatment cycle, for example, a course of administration of a therapeutic agent that can be repeated, e.g., on a regular schedule. In some embodiments, a dosage regimen can have one or more periods of no administration of the therapeutic agent in between treatment intervals.

The terms “administered in combination,” “co-administration,” “co-administering,” and “co-providing” as used herein, mean that two (or more) different treatments are delivered to the subject during the course of the subject's affliction with the disorder, e.g., the two or more treatments are delivered after the subject has been diagnosed with the disorder and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery.” In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

In some embodiments, the first treatment and second treatment can be administered simultaneously (e.g., at the same time), in the same or in separate compositions, or sequentially. Sequential administration refers to administration of one treatment before (e.g., immediately before, less than 5, 10, 15, 30, 45, 60 minutes; 1, 2, 3, 4, 6, 8, 10, 12, 16, 20, 24, 48, 72, 96 or more hours; 4, 5, 6, 7, 8, 9 or more days; 1, 2, 3, 4, 5, 6, 7, 8 or more weeks before) administration of an additional, e.g., secondary, treatment. The order of administration of the first and secondary treatment can also be reversed.

The terms “therapeutically effective amount,” therapeutic amount,” “immunologically effective amount,” “anti-tumor effective amount,” “tumor-inhibiting effective amount,” and their grammatical equivalents refer to an amount effective, at dosages and for periods of time necessary, to achieve a desired therapeutic result. The therapeutically effective amount can vary according to factors such as the disease state, age, sex, and weight of the individual, and the ability of a composition described herein to elicit a desired response in one or more subjects. The precise amount of the compositions of the present disclosure to be administered can be determined by a physician with consideration of individual differences in age, weight, tumor size, extent of infection or metastasis, and condition of the patient (subject).

Alternatively, the pharmacologic and/or physiologic effect of administration of one or more compositions described herein to a patient or a subject of can be “prophylactic,” i.e., the effect completely or partially prevents a disease or symptom thereof. A “prophylactically effective amount” refers to an amount effective, at dosages and for periods of time necessary, to achieve a desired prophylactic result (e.g., prevention of disease onset).

As used herein, the term “immune checkpoint protein” refers to a molecule that transmits a suppressive signal or has an immunosuppressive function. Examples of such immune checkpoint proteins include, but are not limited to, CTLA-4, PD-1, PD-L1 (programmed cell death-ligand 1), PD-L2 (programmed cell death-ligand 2), LAG-3 (Lymphocyte activation gene 3), TIM3 (T cell immunoglobulin and mucin-3), BTLA (B and T lymphocyte attenuator), B7H3, B7H4, CD160, CD39, CD70, CD73, A2aR (adenosine A2a receptor), KIR (killer inhibitory receptor), VISTA (V-domain Ig-containing suppressor of T cell activation), IDO1 (Indoleamine 2,3-dioxygenase), Arginase I, TIGIT (T cell immunoglobulin and ITIM domain), CD70, CD115, and the like (see, Nature Reviews Cancer, 12, p. 252-264, 2012 and Cancer Cell, 27, p. 450-461, 2015).

As used herein, terms used in the identification of biological moieties may include, or may not include, a dash “-” within the term. The presence or absence of a dash does not change the intended meaning or identification of the biological moiety. By way of illustration only, and without limitation to these biological moieties, each of the following paired terms (shown with/without a dash) indicate and identify the same biological entities: CCR-4/CCR4, CD-3/CD3, CD-4/CD4, CD-33/CD33, EGFR-2/EGFR2, FLT-1/FLT1, HER-1/HER1, HER-1t/HER1t, IL-12/IL12, IL-15/IL15, IL-15Rα/IL15Rα MUC-1/MUC1, MUC-16/MUC16, ROR-1/ROR1, ROR-1R/ROR1R, TGF-Beta/TGFBeta, VEGF-1/VEGF1, VEGF-R2/VEGFR2.”

### II. miRNA(s)

As used herein, the terms “miR,” “mir” and “miRNA” are used to refer to microRNA, a class of small non-coding RNA molecules that are capable of affecting the expression of a gene (the “target gene”) by modulating the translation of messenger RNA transcribed therefrom (either increasing or decreasing the gene's expression) and/or destabilizing such messenger RNA.

The term “primary miRNA,” abbreviated “pri-miRNA,” refers to an miRNA containing at least one RNA hairpin. The RNA hairpin(s) are cleaved from the pri-miRNA in the cell nucleus to form one or more precursor miRNAs (“pre-miRNAs”). This pre-miRNA is exported into the cytoplasm where the stem loop structure is cleaved to produce a double-stranded miRNA comprising a miRNA-5p strand from the former 5′ arm of the hairpin loop and a miRNA-3p strand from the former 3′ arm of the hairpin loop. The Argonaute protein then binds the double-stranded miRNA and one of the strands (either the miRNA-5p sequence or the miRNA-3p sequence) is released. The remaining bound strand becomes the “guide strand” whereas the released strand is known as the “passenger strand” and preferably degrades. The guide strand then goes on to interact with the messenger RNA derived from the target gene, thus affecting its translation.

Both the miRNA-5p and miRNA-3p strand sequences will be referred to herein as “mature miRNA” sequences. The remaining portions of a pri-miRNA or pre-miRNA (the portion thereof 5′ to the miRNA-5p sequence, the portion thereof 3′ to the miRNA-3p sequence, and the stem loop sequence in between the miRNA-5p and miRNA-3p sequences) will be collectively referred to as miRNA backbone sequences. The term “5′ backbone sequence” will be used herein to refer to the backbone sequence that, in a pri- or pre-miRNA, is 5′ of the miRNA-5p sequence. The term “3′ backbone sequence” will be used herein to refer to the backbone sequence that, in a pri- or pre-miRNA, is 3′ of the miRNA-3p sequence. The term “loop sequence” refers to the backbone sequence that, in a pri- or pre-miRNA, is between the miRNA-5p and miRNA-3p sequences.

The term “miRNA,” unless otherwise indicated, refers generically to the mature, primary, and precursor forms of a particular microRNA and functional fragments and variants thereof.

The miRNAs can be non-naturally occurring. The terms “non-naturally occurring,” “non-natural,” “synthetic,” and “artificial,” as used to describe miRNA(s) herein, are used interchangeably and refer to an miRNA having a sequence that does not occur in nature.

The present invention relates in part to a ribonucleic acid comprising two non-natural pre-miRNA sequences, wherein each pre-miRNA sequence comprises a guide miRNA that inhibits the expression of an immune checkpoint protein. In certain embodiments, the RNA comprises more than two such non-natural pre-miRNA sequences, for example three, four, five, six, seven, eight, nine, ten, or more such sequences. It is understood that each guide miRNA may target the same or different gene. In embodiments wherein two or more guide miRNAs target the same gene, such guide miRNAs may target the same or different regions of such gene.

In certain embodiments, each non-natural pre-miRNA sequence in the ribonucleic acid forms a stem-loop secondary structure that is distinct and non-complementary from that formed by a different non-natural pre-miRNA sequence in the ribonucleic acid. In certain embodiments, the non-natural pre-miRNA sequences have less than about 95%, less than about 90%, less than about 85%, less than about 80%, less than about 75%, less than about 70%, less than about 65%, less than about 60%, less than about 55%, less than about 55%, or less than about 50% sequence identity with each other.

In certain embodiments, the secondary structure of each non-natural pre-miRNA is sufficiently similar to that of a naturally-occurring pre-miRNA sequence so as to reduce or prevent cellular RNAi-based anti-pathogen toxicity. In certain such embodiments, the nucleic acid sequence of a non-natural pre-miRNA has at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99% sequence identity with that of a naturally-occurring pre-miRNA and/or can hybridize under stringent hybridization conditions with a naturally-occurring pre-miRNA.

In certain embodiments, the secondary structure of each pri-miRNA containing a non-natural pre-miRNA (hereinafter, a “non-natural pri-miRNA”) is sufficiently similar to that of a naturally-occurring pri-miRNA sequence so as to reduce or prevent cellular RNAi-based anti-pathogen toxicity. In certain such embodiments, the nucleic acid sequence of a non-natural pri-miRNA has at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99% sequence identity with that of a naturally-occurring pri-miRNA and/or can hybridize under stringent hybridization conditions with a naturally-occurring pri-miRNA.

The non-natural pre-miRNA of the present invention may be produced from a naturally-occurring pre-miRNA by removing the native mature miRNA sequences and replacing them with non-native mature miRNA sequences wherein one of the sequences is capable of serving as a guide miRNA targeting a gene of interest.

In certain embodiments, each non-natural pre-miRNA comprises backbone sequences derived from a naturally-occurring pre-miRNA, for example from that present in mouse, rat, or human. In certain embodiments, the backbone sequences (the 3′ backbone sequence, the 5′ backbone sequence, and the loop sequence) of the non-natural pre-miRNA have at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99% sequence identity with the corresponding backbone sequences of a naturally-occurring pre-miRNA and/or can hybridize under stringent hybridization conditions with such corresponding backbone segments. In certain embodiments, the backbone segments of the non-natural pre-miRNA sequences are identical to the corresponding backbone segments of a naturally-occurring pre-miRNA. In certain embodiments, the naturally-occurring pre-miRNA is miR16, miR17, miR19, miR21, miR22, miR26a1, miR29b1, miR30a, miR122, miR126, miR133a1, miR142, miR150, miR155, miR204, miR206, miR214, miR412, miR486, miR494, or miR1915. In certain embodiments, the naturally-occurring pre-miRNA is miR16, miR17, miR21, miR22, miR26a1, miR142, miR150, miR204, or miR206. In certain embodiments, the naturally-occurring pre-miRNA is miR16, miR21, miR22, miR204, or miR206. In certain embodiments, the naturally-occurring pre-miRNA is miR204 or miR206.

In certain embodiments, each non-natural pri-miRNA comprises backbone sequences derived from a naturally-occurring pri-miRNA, for example from that present in mouse, rat, or human. In certain embodiments, the backbone sequences (the 3′ backbone sequence, the 5′ backbone sequence, and the loop sequence) of the non-natural pri-miRNA have at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99% sequence identity with the corresponding backbone sequences of a naturally-occurring pri-miRNA and/or can hybridize under stringent hybridization conditions with such corresponding backbone segments. In certain embodiments, the backbone segments of the non-natural pri-miRNA sequences are identical to the corresponding backbone segments of a naturally-occurring pri-miRNA. In certain embodiments, the naturally-occurring pri-miRNA is miR16, miR17, miR19, miR21, miR22, miR26a1, miR29b1, miR30a, miR122, miR126, miR133a1, miR142, miR150, miR155, miR204, miR206, miR214, miR412, miR486, miR494, or miR1915. In certain embodiments, the naturally-occurring pre-miRNA is miR16, miR17, miR21, miR22, miR26a1, miR142, miR150, miR204, or miR206. In certain embodiments, the naturally-occurring pre-miRNA is miR16, miR21, miR22, miR204, or miR206. In certain embodiments, the naturally-occurring pre-miRNA is miR204 or miR206.

While the miRNA-5p and miRNA-3p sequences hybridize with each other, they are not necessarily exactly complementary. In the design of a non-naturally occurring miRNA, compensatory mutations can be made in the miRNA-5p and/or miRNA-3p sequences so as to maintain the RNA folding and free energy of the native miRNA. In certain embodiments, the sequence encoding the miRNA-3p sequence has at least 50%, 60%, 70%, 80%, 85%, 90%, 95%, 96%, 97%, 98%, or 99% sequence identity with the complement to the sequence encoding the miRNA-5p sequence or is capable of hybridizing under stringent hybridization conditions with the sequence encoding the miRNA-5p sequence.

In certain embodiments, the two non-natural pre-miRNA sequences are separated from each other by at least about 1, at least about 2, at last about 3, at least about 4, at least about 5, at least about 6, at least about 7, at least about 8, at least about 9, at least about 10, at least about 15, at least about 20, at least about 25, at least about 30, at least about 35, at least about 40, at least about 45, at least about 50, at least about 60, at least about 70, at least about 80, at least about 90, at least about 100, at least about 110, at least about 120, at least about 130, at least about 140, at least about 150, at least about 160, at least about 170, at least about 180, at least about 190, at least about 200, at least about 210, at least about 220, at least about 230, at least about 240, or at least about 250 nucleotides. In certain embodiments, the two non-natural pre-miRNA sequences are separated from each other by about 5 to 250 nucleotides, about 10 to 250 nucleotides, about 10 to 200 nucleotides, about 10 to 150 nucleotides, about 10 to 100 nucleotides, about 10 to 50 nucleotides, about 10 to 40 nucleotides, about 10 to 30 nucleotides, about 10 to 20 nucleotides, about 16 to 250 nucleotides, about 16 to 200 nucleotides, about 16 to 150 nucleotides, about 16 to 100 nucleotides, about 16 to 50 nucleotides, about 16 to 40 nucleotides, about 16 to 30 nucleotides, about 16 to 20 nucleotides, about 20 to 200 nucleotides, about 20 to 150 nucleotides, about 20 to 100 nucleotides, about 20 to 50 nucleotides, about 20 to 45 nucleotides, about 20 to 40 nucleotides, about 20 to 35 nucleotides, about 20 to 30 nucleotides, about 20 to 25 nucleotides, about 30 to 200 nucleotides, about 30 to 150 nucleotides, about 30 to 100 nucleotides, about 30 to 50 nucleotides, or about 30 to 40 nucleotides. In certain embodiments, the two non-natural pre-miRNA sequences are separated from each other by at least about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 2010, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, or 250 nucleotides.

In certain embodiments, two non-natural pri-miRNA sequences adjoin each other with the 3′ nucleotide of one pri-miRNA being directly bonded to the 5′ nucleotide of another pri-miRNA. In such embodiments, the nucleotides separating the respective non-natural pre-miRNAs contained in each pri-miRNA form part of the pri-miRNA sequences.

In certain embodiments, the non-natural pre-miRNA comprises a mature miRNA sequence that is capable of binding to an mRNA and thereby interfering with the translation thereof and/or prompting its degradation. The mRNA may be produced from the expression of a target gene.

In certain embodiments, the target gene encodes an immune checkpoint protein. Thus, the pre-miRNA sequences inhibit the expression of an immune checkpoint protein by targeting the gene expressing the same. In certain such embodiments, the immune checkpoint protein is PD-1, PD-L1, CTLA4, TIGIT, 4-1BB, PIK3IP1, CD27, CD28, CD40, CD70, CD122, CD137, OX40 (CD134), GITR, ICOS, A2AR, B7-H3 (CD276), B7-H4 (VTCN1), BTLA, IDO, KIR, LAG3, TIM3, or VISTA. In certain such embodiments, the immune checkpoint protein is CTLA4, CD70, PD-1, PD-L1, TIGIT, TIM3, LAG3, GITR, or PIK3IP1. In certain embodiments, immune checkpoint protein is CTLA4, CD70, PD-1, TIGIT, TIM3, LAG3, GITR, or PIK3IP1. In certain embodiments, immune checkpoint protein is CD70, PD-1, or TIGIT. In certain embodiments, immune checkpoint protein is PD-1.

In certain embodiments, each non-natural pre-miRNA targets a different gene. In certain different regions of the same gene.

In certain embodiments, the ribonucleic acid comprises: (a) a pre-miRNA comprising backbone sequences from miR204 and a guide miRNA that targets PD-1; and (b) a pre-miRNA comprising backbone sequences from miR206 and a guide miRNA that targets PD-1.

In certain embodiments, the ribonucleic acid comprises: (a) a pri-miRNA comprising backbone sequences from miR204 and a guide miRNA that targets PD-1; and (b) a pri-miRNA comprising backbone sequences from miR206 and a guide miRNA that targets PD-1.

In certain embodiments, the ribonucleic acid comprises: (a) a pre-miRNA comprising backbone sequences from miR16 and a guide miRNA that targets PD-1; and (b) a pre-miRNA comprising backbone sequences from miR22 and a guide miRNA that targets PD-1.

In certain embodiments, the ribonucleic acid comprises: (a) a pri-miRNA comprising backbone sequences from miR16 and a guide miRNA that targets PD-1; and (b) a pri-miRNA comprising backbone sequences from miR22 and a guide miRNA that targets PD-1.

In certain embodiments, the ribonucleic acid comprises: (a) a pre-miRNA comprising backbone sequences from miR204 and a guide miRNA that targets TIGIT; and (b) a pre-miRNA comprising backbone sequences from miR206 and a guide miRNA that targets TIGIT.

In certain embodiments, the ribonucleic acid comprises: (a) a pri-miRNA comprising backbone sequences from miR204 and a guide miRNA that targets TIGIT; and (b) a pri-miRNA comprising backbone sequences from miR206 and a guide miRNA that targets TIGIT.

In certain embodiments, the ribonucleic acid comprises: (a) a pre-miRNA comprising backbone sequences from miR16 and a guide miRNA that targets TIGIT; and (b) a pre-miRNA comprising backbone sequences from miR21 and a guide miRNA that targets TIGIT.

In certain embodiments, the ribonucleic acid comprises: (a) a pri-miRNA comprising backbone sequences from miR16 and a guide miRNA that targets TIGIT; and (b) a pri-miRNA comprising backbone sequences from miR21 and a guide miRNA that targets TIGIT.

In certain embodiments, the ribonucleic acid comprises: (a) a pre-miRNA comprising backbone sequences from miR16 and a guide miRNA that targets TIGIT; and (b) a pre-miRNA comprising backbone sequences from miR22 and a guide miRNA that targets TIGIT.

In certain embodiments, the ribonucleic acid comprises: (a) a pri-miRNA comprising backbone sequences from miR16 and a guide miRNA that targets TIGIT; and (b) a pri-miRNA comprising backbone sequences from miR22 and a guide miRNA that targets TIGIT.

In certain embodiments, the ribonucleic acid comprises: (a) a pre-miRNA comprising backbone sequences from miR22 and a guide miRNA that targets TIGIT; and (b) a pre-miRNA comprising backbone sequences from miR204 and a guide miRNA that targets TIGIT.

In certain embodiments, the ribonucleic acid comprises: (a) a pri-miRNA comprising backbone sequences from miR22 and a guide miRNA that targets TIGIT; and (b) a pri-miRNA comprising backbone sequences from miR204 and a guide miRNA that targets TIGIT.

In certain embodiments, the ribonucleic acid comprises: (a) a pre-miRNA comprising backbone sequences from miR22 and a guide miRNA that targets TIGIT; and (b) a pre-miRNA comprising backbone sequences from miR142 and a guide miRNA that targets TIGIT.

In certain embodiments, the ribonucleic acid comprises: (a) a pri-miRNA comprising backbone sequences from miR22 and a guide miRNA that targets TIGIT; and (b) a pri-miRNA comprising backbone sequences from miR142 and a guide miRNA that targets TIGIT.

In certain embodiments, the ribonucleic acid comprises: (a) a pre-miRNA comprising backbone sequences from miR22 and a guide miRNA that targets TIGIT; and (b) a pre-miRNA comprising backbone sequences from miR206 and a guide miRNA that targets TIGIT.

In certain embodiments, the ribonucleic acid comprises: (a) a pri-miRNA comprising backbone sequences from miR22 and a guide miRNA that targets TIGIT; and (b) a pri-miRNA comprising backbone sequences from miR206 and a guide miRNA that targets TIGIT.

In certain embodiments, the ribonucleic acid comprises: (a) a pre-miRNA comprising backbone sequences from miR22 and a guide miRNA that targets TIGIT; and (b) a pre-miRNA comprising backbone sequences from miR21 and a guide miRNA that targets TIGIT.

In certain embodiments, the ribonucleic acid comprises: (a) a pri-miRNA comprising backbone sequences from miR22 and a guide miRNA that targets TIGIT; and (b) a pri-miRNA comprising backbone sequences from miR21 and a guide miRNA that targets TIGIT.

In certain embodiments, the ribonucleic acid comprises: (a) a pre-miRNA comprising backbone sequences from miR204 and a guide miRNA that targets TIGIT; and (b) a pre-miRNA comprising backbone sequences from miR21 and a guide miRNA that targets TIGIT.

In certain embodiments, the ribonucleic acid comprises: (a) a pri-miRNA comprising backbone sequences from miR204 and a guide miRNA that targets TIGIT; and (b) a pri-miRNA comprising backbone sequences from miR21 and a guide miRNA that targets TIGIT.

In certain embodiments, the ribonucleic acid comprises: (a) a pre-miRNA comprising backbone sequences from miR142 and a guide miRNA that targets TIGIT; and (b) a pre-miRNA comprising backbone sequences from miR206 and a guide miRNA that targets TIGIT.

In certain embodiments, the ribonucleic acid comprises: (a) a pri-miRNA comprising backbone sequences from miR142 and a guide miRNA that targets TIGIT; and (b) a pri-miRNA comprising backbone sequences from miR206 and a guide miRNA that targets TIGIT.

In certain embodiments, the ribonucleic acid comprises: (a) a pre-miRNA comprising backbone sequences from miR142 and a guide miRNA that targets TIGIT; and (b) a pre-miRNA comprising backbone sequences from miR204 and a guide miRNA that targets TIGIT.

In certain embodiments, the ribonucleic acid comprises: (a) a pri-miRNA comprising backbone sequences from miR142 and a guide miRNA that targets TIGIT; and (b) a pri-miRNA comprising backbone sequences from miR204 and a guide miRNA that targets TIGIT.

In certain embodiments, the ribonucleic acid comprises: (a) a pre-miRNA comprising backbone sequences from miR142 and a guide miRNA that targets TIGIT; and (b) a pre-miRNA comprising backbone sequences from miR21 and a guide miRNA that targets TIGIT.

In certain embodiments, the ribonucleic acid comprises: (a) a pri-miRNA comprising backbone sequences from miR142 and a guide miRNA that targets TIGIT; and (b) a pri-miRNA comprising backbone sequences from miR21 and a guide miRNA that targets TIGIT.

In certain embodiments, the ribonucleic acid comprises: (a) a pre-miRNA comprising backbone sequences from miR16 and a guide miRNA that targets TIGIT; and (b) a pre-miRNA comprising backbone sequences from miR206 and a guide miRNA that targets TIGIT.

In certain embodiments, the ribonucleic acid comprises: (a) a pri-miRNA comprising backbone sequences from miR16 and a guide miRNA that targets TIGIT; and (b) a pri-miRNA comprising backbone sequences from miR206 and a guide miRNA that targets TIGIT.

In certain embodiments, the ribonucleic acid comprises: (a) a pre-miRNA comprising backbone sequences from miR16 and a guide miRNA that targets TIGIT; and (b) a pre-miRNA comprising backbone sequences from miR204 and a guide miRNA that targets TIGIT.

In certain embodiments, the ribonucleic acid comprises: (a) a pri-miRNA comprising backbone sequences from miR16 and a guide miRNA that targets TIGIT; and (b) a pri-miRNA comprising backbone sequences from miR204 and a guide miRNA that targets TIGIT.

In certain embodiments, the ribonucleic acid comprises: (a) a pre-miRNA comprising backbone sequences from miR204 and a guide miRNA that targets CD70; and (b) a pre-miRNA comprising backbone sequences from miR206 and a guide miRNA that targets CD70.

In certain embodiments, the ribonucleic acid comprises: (a) a pri-miRNA comprising backbone sequences from miR204 and a guide miRNA that targets CD70; and (b) a pri-miRNA comprising backbone sequences from miR206 and a guide miRNA that targets CD70.

In certain embodiments, the ribonucleic acid comprises: (a) a pre-miRNA comprising backbone sequences from miR16 and a guide miRNA that targets CD70; and (b) a pre-miRNA comprising backbone sequences from miR22 and a guide miRNA that targets CD70.

In certain embodiments, the ribonucleic acid comprises: (a) a pri-miRNA comprising backbone sequences from miR16 and a guide miRNA that targets CD70; and (b) a pri-miRNA comprising backbone sequences from miR22 and a guide miRNA that targets CD70.

In certain embodiments, the ribonucleic acid comprises: (a) a pre-miRNA comprising backbone sequences from miR26a1 and a guide miRNA that targets CD70; and (b) a pre-miRNA comprising backbone sequences from miR206 and a guide miRNA that targets CD70.

In certain embodiments, the ribonucleic acid comprises: (a) a pri-miRNA comprising backbone sequences from miR26a1 and a guide miRNA that targets CD70; and (b) a pri-miRNA comprising backbone sequences from miR206 and a guide miRNA that targets CD70.

In certain embodiments, the ribonucleic acid comprises: (a) a pre-miRNA comprising backbone sequences from miR26a1 and a guide miRNA that targets CD70; and (b) a pre-miRNA comprising backbone sequences from miR16 and a guide miRNA that targets CD70.

In certain embodiments, the ribonucleic acid comprises: (a) a pri-miRNA comprising backbone sequences from miR26a1 and a guide miRNA that targets CD70; and (b) a pri-miRNA comprising backbone sequences from miR16 and a guide miRNA that targets CD70.

In certain embodiments, the ribonucleic acid comprises: (a) a pre-miRNA comprising backbone sequences from miR204 and a guide miRNA that targets CD70; and (b) a pre-miRNA comprising backbone sequences from miR26a1 and a guide miRNA that targets CD70.

In certain embodiments, the ribonucleic acid comprises: (a) a pri-miRNA comprising backbone sequences from miR204 and a guide miRNA that targets CD70; and (b) a pri-miRNA comprising backbone sequences from miR26a1 and a guide miRNA that targets CD70.

In certain embodiments, the ribonucleic acid comprises: (a) a pre-miRNA comprising backbone sequences from miR204 and a guide miRNA that targets CD70; and (b) a pre-miRNA comprising backbone sequences from miR16 and a guide miRNA that targets CD70.

In certain embodiments, the ribonucleic acid comprises: (a) a pri-miRNA comprising backbone sequences from miR204 and a guide miRNA that targets CD70; and (b) a pri-miRNA comprising backbone sequences from miR16 and a guide miRNA that targets CD70.

In certain embodiments, the ribonucleic acid comprises: (a) a pre-miRNA comprising backbone sequences from miR150 and a guide miRNA that targets TIGIT; and (b) a pre-miRNA comprising backbone sequences from miR206 and a guide miRNA that targets PD1.

In certain embodiments, the ribonucleic acid comprises: (a) a pri-miRNA comprising backbone sequences from miR150 and a guide miRNA that targets TIGIT; and (b) a pie-miRNA comprising backbone sequences from miR206 and a guide miRNA that targets PD1.

In certain embodiments, the ribonucleic acid comprises: (a) a pre-miRNA comprising backbone sequences from miR204 and a guide miRNA that targets PD1; (b) a pre-miRNA comprising backbone sequences from miR206 and a guide miRNA that targets PD1; and (c) a pre-miRNA comprising backbone sequences from miR17 and a guide miRNA that targets TIGIT.

In certain embodiments, the ribonucleic acid comprises: (a) a pri-miRNA comprising backbone sequences from miR204 and a guide miRNA that targets PD1; (b) a pri-miRNA comprising backbone sequences from miR206 and a guide miRNA that targets PD1; and (c) a pri-miRNA comprising backbone sequences from miR17 and a guide miRNA that targets TIGIT.

In certain embodiments, the ribonucleic acid comprises: (a) a pre-miRNA comprising backbone sequences from miR17 and a guide miRNA that targets TIGIT; and (b) a pre-miRNA comprising backbone sequences from miR206 and a guide miRNA that targets PD1.

In certain embodiments, the ribonucleic acid comprises: (a) a pri-miRNA comprising backbone sequences from miR17 and a guide miRNA that targets TIGIT; and (b) a pri-miRNA comprising backbone sequences from miR206 and a guide miRNA that targets PD1.

In certain embodiments, the ribonucleic acid comprises: (a) a pre-miRNA comprising backbone sequences from miR204 and a guide miRNA that targets PD1; (b) a pre-miRNA comprising backbone sequences from miR206 and a guide miRNA that targets PD1; and (c) a pre-miRNA comprising backbone sequences from miR150 and a guide miRNA that targets TIGIT.

In certain embodiments, the ribonucleic acid comprises: (a) a pri-miRNA comprising backbone sequences from miR204 and a guide miRNA that targets PD1; (b) a pri-miRNA comprising backbone sequences from miR206 and a guide miRNA that targets PD1; and (c) a pri-miRNA comprising backbone sequences from miR150 and a guide miRNA that targets TIGIT.

In certain embodiments, the ribonucleic acid comprises: (a) a pre-miRNA comprising backbone sequences from miR204 and a guide miRNA that targets PD1; (b) a pre-miRNA comprising backbone sequences from miR206 and a guide miRNA that targets PD1; and (c) a pre-miRNA comprising backbone sequences from miR16 and a guide miRNA that targets CD70.

In certain embodiments, the ribonucleic acid comprises: (a) a pri-miRNA comprising backbone sequences from miR204 and a guide miRNA that targets PD1; (b) a pri-miRNA comprising backbone sequences from miR206 and a guide miRNA that targets PD1; and (c) a pri-miRNA comprising backbone sequences from miR16 and a guide miRNA that targets CD70.

In certain embodiments, the ribonucleic acid comprises: (a) a pre-miRNA comprising backbone sequences from miR204 and a guide miRNA that targets PD1; (b) a pre-miRNA comprising backbone sequences from miR206 and a guide miRNA that targets PD1; and (c) a pre-miRNA comprising backbone sequences from miR22 and a guide miRNA that targets CD70.

In certain embodiments, the ribonucleic acid comprises: (a) a pri-miRNA comprising backbone sequences from miR204 and a guide miRNA that targets PD1; (b) a pri-miRNA comprising backbone sequences from miR206 and a guide miRNA that targets PD1; and (c) a pri-miRNA comprising backbone sequences from miR22 and a guide miRNA that targets CD70.

In certain embodiments, the ribonucleic acid comprises: (a) a pre-miRNA comprising backbone sequences from miR204 and a guide miRNA that targets PD1; (b) a pre-miRNA comprising backbone sequences from miR206 and a guide miRNA that targets PD1; and (c) a pre-miRNA comprising backbone sequences from miR22 and a guide miRNA that targets CD70.

In certain embodiments, the ribonucleic acid comprises: (a) a pri-miRNA comprising backbone sequences from miR204 and a guide miRNA that targets PD1; (b) a pri-miRNA comprising backbone sequences from miR206 and a guide miRNA that targets PD1; and (c) a pri-miRNA comprising backbone sequences from miR22 and a guide miRNA that targets CD70.

The present invention also relates in part to a deoxyribonucleic acid encoding any of the aforementioned ribonucleic acids.

Examples of deoxyribonucleic acid sequences that encode backbone sequences that may be used in the practice of the present invention include, but are not limited, to those listed in Table 1 below. The symbols of “X” and “Y” in Table 1 indicate nucleic acid sequences encoding, respectively, the guide miRNA (which may be either miRNA-5p or miRNA-3p) and the passenger miRNA (which may be either miRNA-5p or miRNA-3p), whereas the symbol of “n” indicates the number of nucleotides in such sequences, for example 16-30, preferably 18-25. In some embodiments, n can be 6, 7, 8, 9, 10, 11, 12, 13, 14, 15, 16, 17, 18, 19, 20, 21, 22, 23, 24, 25, 26, 27, 28, 29, 30, 31, 32, 33, 34 or 35 nucleotides. In certain embodiments, the deoxyribonucleic acids encoding backbone sequences are those that hybridize under stringent hybridization conditions with the complement of any one of the sequences listed in Table 1.

TABLE 1
Deoxyribonucleic acid sequences
encoding miRNA backbone sequences

| Pri-miRNA from | |
|---|---|
| which backbone | Nucleic acid sequences encoding |
| sequences are | 5′ Backbone Sequence-Xₙ-Stem Loop |
| derived | Sequence-Yₙ-3′ Backbone Sequence |
| miR204 | SEQ ID NO: 1-Xₙ-SEQ ID NO: 2-Yₙ- |
| | SEQ ID NO: 3 |
| miR206 | SEQ ID NO: 4-Yₙ-SEQ ID NO: 5-Xₙ- |
| | SEQ ID NO: 6 |
| miR17 | SEQ ID NO: 7-Xₙ-SEQ ID NO: 8-Yₙ- |
| | SEQ ID NO: 9 |
| miR150 | SEQ ID NO: 10-Xₙ-SEQ ID NO: 11-Yₙ- |
| | SEQ ID NO: 12 |
| miR150 | SEQ ID NO: 13-Yₙ-SEQ ID NO: 14-Xₙ- |
| | SEQ ID NO: 15 |
| miR16 | SEQ ID NO: 16-Xₙ-SEQ ID NO: 17-Yₙ- |
| | SEQ ID NO: 18 |
| miR30a | SEQ ID NO: 19-Xₙ(or Yₙ)-SEQ ID |
| | NO: 20-Yₙ(or Xₙ)-SEQ ID NO: 21 |
| miR126 | SEQ ID NO: 22-Yₙ-SEQ ID NO: 23-Xₙ- |
| | SEQ ID NO: 24 |
| miR122 | SEQ ID NO: 25-Xₙ-SEQ ID NO: 26-Yₙ- |
| | SEQ ID NO: 27 |
| miR214 | SEQ ID NO: 28-Yₙ-SEQ ID NO: 29-Xₙ- |
| | SEQ ID NO: 30 |
| miR214 | SEQ ID NO: 31-Xₙ-SEQ ID NO: 32-Yₙ- |
| | SEQ ID NO: 33 |
| miR29b1 | SEQ ID NO: 34-Yₙ-SEQ ID NO: 35-Xₙ- |
| | SEQ ID NO: 36 |
| miR29b1 | SEQ ID NO: 37-Xₙ-SEQ ID NO: 38-Yₙ- |
| | SEQ ID NO: 39 |
| miR133a1 | SEQ ID NO: 40-Yₙ-SEQ ID NO: 41-Xₙ- |
| | SEQ ID NO: 42 |
| miR26a1 | SEQ ID NO: 43-Xₙ-SEQ ID NO: 44-Yₙ- |
| | SEQ ID NO: 45 |
| miR412 | SEQ ID NO: 46-Xₙ-SEQ ID NO: 47-Yₙ- |
| | SEQ ID NO: 48 |
| miR19 | SEQ ID NO: 49-Xₙ-SEQ ID NO: 50-Yₙ- |
| | SEQ ID NO: 51 |
| miR21 | SEQ ID NO: 52-Xₙ-SEQ ID NO: 53-Yₙ- |
| | SEQ ID NO: 54 |
| miR142 | SEQ ID NO: 55-Xₙ-SEQ ID NO: 56-Yₙ- |
| | SEQ ID NO: 57 |
| miR494 | SEQ ID NO: 58-Xₙ-SEQ ID NO: 59-Yₙ- |
| | SEQ ID NO: 60 |
| miR1915 | SEQ ID NO: 61-Xₙ-SEQ ID NO: 62-Yₙ- |
| | SEQ ID NO: 63 |
| miR22 | SEQ ID NO: 338-Yₙ-SEQ ID |
| | NO: 339-Xₙ-SEQ ID NO: 340 |
| miR26a1 | SEQ ID NO: 341-Xₙ-SEQ ID |
| | NO: 342-Yₙ-SEQ ID NO: 343 |
| miR486 | SEQ ID NO: 344-Xₙ-SEQ ID |
| | NO: 345-Yₙ-SEQ ID NO: 346 |

In any of the foregoing embodiments, the sequence encoding the pre-miRNA comprises:
SEQ ID NO: 1, SEQ ID NO: 2, and SEQ ID NO: 3, respectively;
SEQ ID NO: 4, SEQ ID NO: 5, and SEQ ID NO: 6, respectively;
SEQ ID NO: 7, SEQ ID NO: 8, and SEQ ID NO: 9, respectively;
SEQ ID NO: 10, SEQ ID NO: 11, and SEQ ID NO: 12, respectively;
SEQ ID NO: 13, SEQ ID NO: 14, and SEQ ID NO: 15, respectively;
SEQ ID NO: 16, SEQ ID NO: 17, and SEQ ID NO: 18, respectively;
SEQ ID NO: 19, SEQ ID NO: 20, and SEQ ID NO: 21, respectively;
SEQ ID NO: 22, SEQ ID NO: 23, and SEQ ID NO: 24, respectively;
SEQ ID NO: 25, SEQ ID NO: 26, and SEQ ID NO: 27, respectively;
SEQ ID NO: 28, SEQ ID NO: 29, and SEQ ID NO: 30, respectively;
SEQ ID NO: 31, SEQ ID NO: 32, and SEQ ID NO: 33, respectively;
SEQ ID NO: 34, SEQ ID NO: 35, and SEQ ID NO: 36, respectively;
SEQ ID NO: 37, SEQ ID NO: 38, and SEQ ID NO: 39, respectively;
SEQ ID NO: 40, SEQ ID NO: 41, and SEQ ID NO: 42, respectively;
SEQ ID NO: 43, SEQ ID NO: 44, and SEQ ID NO: 45, respectively;
SEQ ID NO: 46, SEQ ID NO: 47, and SEQ ID NO: 48, respectively;
SEQ ID NO: 49, SEQ ID NO: 50, and SEQ ID NO: 51, respectively;
SEQ ID NO: 52, SEQ ID NO: 53, and SEQ ID NO: 54, respectively;
SEQ ID NO: 55, SEQ ID NO: 56, and SEQ ID NO: 57, respectively;
SEQ ID NO: 58, SEQ ID NO: 59, and SEQ ID NO: 60, respectively;
SEQ ID NO: 61, SEQ ID NO: 62, and SEQ ID NO: 63, respectively;
SEQ ID NO: 338, SEQ ID NO: 339, and SEQ ID NO: 340, respectively;
SEQ ID NO: 341, SEQ ID NO: 342, and SEQ ID NO: 343, respectively; or
SEQ ID NO: 344, SEQ ID NO: 345, and SEQ ID NO: 346, respectively;
or sequences having at least 80%, 85%, 90%, 95%, 96%, 97%, 98%, or 99% sequence identity with any of the foregoing sequences, or that are capable of hybridizing under stringent hybridization conditions to the complements of such sequences.

Non-limiting examples of nucleic acid sequences encoding the guide miRNA targeting genes encoding such checkpoint inhibitors are listed in Table 2. Table 2 also lists the sequences encoding the passenger strand. As previously discussed, the guide and passenger strand are not necessarily complementary. It is contemplated that the passenger strand may also serve to target the messenger RNA associated with the target gene. It is also contemplated that sequences that sequences that hybridize under stringent hybridization conditions with the complements of the sequences listed in Table 2 may also be used. The mature miRNA sequences used may be combined with a specific pri-miRNA backbone. Table 2 also lists backbones that can be combined with the mature guide and passenger miRNAs listed therein.

TABLE 2
Deoxyribonucleic acid sequences
encoding mature miRNA sequences

| miRNA | | DNA encoding | DNA encoding |
|---|---|---|---|
| Target | Backbone | guide miRNA | passenger miRNA |
| CTLA4 | miR-204 | (SEQ ID NO: 64) | (SEQ ID NO: 65) |
| CTLA4 | miR-26a | (SEQ ID NO: 66) | (SEQ ID NO: 67) |
| CTLA4 | miR-30a | (SEQ ID NO: 68) | (SEQ ID NO: 69) |
| CTLA4 | miR-206 | (SEQ ID NO: 70) | (SEQ ID NO: 71) |
| PD1 | miR-204 | (SEQ ID NO: 72) | (SEQ ID NO: 73) |
| PD1 | miR-204 | (SEQ ID NO: 704) | |
| PD1 | miR-204 | (SEQ ID NO: 705) | |
| PD1 | miR-204 | | (SEQ ID NO: 706) |
| PD1 | miR-204 | | (SEQ ID NO: 707) |
| PD1 | miR-204 | | (SEQ ID NO: 708) |
| PD1 | miR-206 | (SEQ ID NO: 74) | (SEQ ID NO: 75) |
| PD1 | miR-206 | (SEQ ID NO: 709) | |
| PD1 | miR-206 | (SEQ ID NO: 710) | |
| PD1 | miR-206 | | (SEQ ID NO: 711) |
| PD1 | miR-206 | | (SEQ ID NO: 712) |
| PD1 | miR-206 | | (SEQ ID NO: 713) |
| PD1 | miR-30a | (SEQ ID NO: 76) | (SEQ ID NO: 77) |
| PD1 | miR-412 | (SEQ ID NO: 78) | (SEQ ID NO: 79) |
| PD1 | miR122 | (SEQ ID NO: 80) | (SEQ ID NO: 81) |
| PD1 | miR-17 | (SEQ ID NO: 82) | (SEQ ID NO: 83) |
| PD1 | miR-150 | (SEQ ID NO: 74) | (SEQ ID NO: 85) |
| PD1 | miR-486 | (SEQ ID NO: 76) | (SEQ ID NO: 87) |
| TIGIT | miR-17 | (SEQ ID NO: 88) | (SEQ ID NO: 89) |
| TIGIT | miR-150 | (SEQ ID NO: 90) | (SEQ ID NO: 91) |
| TIGIT | miR-204 | (SEQ ID NO: 92) | (SEQ ID NO: 93) |
| TIGIT | miR29b1 | (SEQ ID NO: 94) | (SEQ ID NO: 95) |
| TIGIT | miR214 | (SEQ ID NO: 96) | (SEQ ID NO: 97) |
| TIGIT | miR-206 | (SEQ ID NO: 98) | (SEQ ID NO: 99) |
| TIGIT | miR-204 | (SEQ ID NO: 100) | (SEQ ID NO: 101) |
| TIGIT | miR-22 | (SEQ ID NO: 102) | (SEQ ID NO: 103) |
| TIGIT | miR-16 | (SEQ ID NO: 104) | (SEQ ID NO: 105) |
| TIGIT | miR-21 | (SEQ ID NO: 106) | (SEQ ID NO: 107) |
| TIGIT | miR-494 | (SEQ ID NO: 108) | (SEQ ID NO: 109) |
| TIGIT | miR-142 | (SEQ ID NO: 110) | (SEQ ID NO: 111) |
| TIGIT | miR-19 | (SEQ ID NO: 112) | (SEQ ID NO: 113) |
| TIGIT | miR-1915 | (SEQ ID NO: 114) | (SEQ ID NO: 115) |
| TIGIT | miR-206 | (SEQ ID NO: 116) | (SEQ ID NO: 117) |
| TIGIT | miR-204 | (SEQ ID NO: 118) | (SEQ ID NO: 119) |
| TIGIT | miR-22 | (SEQ ID NO: 120) | (SEQ ID NO: 121) |
| TIGIT | miR-142 | (SEQ ID NO: 122) | (SEQ ID NO: 123) |
| TIGIT | miR-16 | (SEQ ID NO: 124) | (SEQ ID NO: 125) |
| TIGIT | miR-206 | (SEQ ID NO: 126) | (SEQ ID NO: 127) |
| TIGIT | miR-204 | (SEQ ID NO: 128) | (SEQ ID NO: 129) |
| TIGIT | miR-21 | (SEQ ID NO: 130) | (SEQ ID NO: 131) |
| TIGIT | miR-494 | (SEQ ID NO: 132) | (SEQ ID NO: 133) |
| TIGIT | miR-19 | (SEQ ID NO: 134) | (SEQ ID NO: 135) |
| TIGIT | miR-1915 | (SEQ ID NO: 136) | (SEQ ID NO: 137) |
| TIGIT | miR-204 | (SEQ ID NO: 138) | (SEQ ID NO: 139) |
| TIGIT | miR-206 | (SEQ ID NO: 140) | (SEQ ID NO: 141) |
| TIGIT | miR-21 | (SEQ ID NO: 142) | (SEQ ID NO: 143) |
| TIGIT | miR-22 | (SEQ ID NO: 144) | (SEQ ID NO: 145) |
| TIM3 | miR204 | (SEQ ID NO: 146) | (SEQ ID NO: 147) |
| TIM3 | miR206 | (SEQ ID NO: 148) | (SEQ ID NO: 149) |
| TIM3 | miR17 | (SEQ ID NO: 150) | (SEQ ID NO: 151) |
| TIM3 | miR126 | (SEQ ID NO: 152) | (SEQ ID NO: 153) |
| TIM3 | miR122 | (SEQ ID NO: 154) | (SEQ ID NO: 155) |
| TIM3 | miR214 | (SEQ ID NO: 156) | (SEQ ID NO: 157) |
| LAG3 | miR-30a | (SEQ ID NO: 158) | (SEQ ID NO: 159) |
| LAG3 | miR122 | (SEQ ID NO: 160) | (SEQ ID NO: 161) |
| GITR | miR206 | (SEQ ID NO: 162) | (SEQ ID NO: 163) |
| GITR | miR29b1 | (SEQ ID NO: 164) | (SEQ ID NO: 165) |
| PIK3IP1 | miR206 | (SEQ ID NO: 166) | (SEQ ID NO: 167) |
| PIK3IP1 | miR126 | (SEQ ID NO: 168) | (SEQ ID NO: 169) |
| PIK3IP1 | miR30a | (SEQ ID NO: 170) | (SEQ ID NO: 171) |
| CD70 | miR206 | (SEQ ID NO: 293) | (SEQ ID NO: 294) |
| CD70 | miR204 | (SEQ ID NO: 295) | (SEQ ID NO: 296) |
| CD70 | miR22 | (SEQ ID NO: 297) | (SEQ ID NO: 298) |
| CD70 | miR16 | (SEQ ID NO: 299) | (SEQ ID NO: 300) |
| CD70 | miR21 | (SEQ ID NO: 301) | (SEQ ID NO: 302) |
| CD70 | miR206 | (SEQ ID NO: 303) | (SEQ ID NO: 304) |
| CD70 | miR204 | (SEQ ID NO: 305) | (SEQ ID NO: 306) |
| CD70 | miR19 | (SEQ ID NO: 307) | (SEQ ID NO: 308) |
| CD70 | miR494 | (SEQ ID NO: 309) | (SEQ ID NO: 310) |
| CD70 | miR150 | (SEQ ID NO: 311) | (SEQ ID NO: 312) |
| CD70 | miR206 | (SEQ ID NO: 313) | (SEQ ID NO: 314) |
| CD70 | miR204 | (SEQ ID NO: 315) | (SEQ ID NO: 316) |
| CD70 | miR22 | (SEQ ID NO: 317) | (SEQ ID NO: 318) |
| CD70 | miR142 | (SEQ ID NO: 319) | (SEQ ID NO: 320) |
| CD70 | miR26a1 | (SEQ ID NO: 321) | (SEQ ID NO: 322) |

In certain embodiments, the present invention relates to a deoxyribonucleic acid comprising a nucleic acid sequence having at least 80% sequence identity with any one of SEQ ID NOs: 64-83, 85, 87-171, 293-322, and 704-713 or that is capable of hybridizing under stringent hybridization conditions to the complement of any one of SEQ ID NOs: 64-83, 85, 87-171, 293-322, and 704-713. In certain such embodiments, the present invention relates to a deoxyribonucleic acid comprising a nucleic acid sequence having at least 80%, 85%, 90%, 95%, 96%, 97%, 98%, or 99% sequence identity with any one of SEQ ID NOs: 64, 66, 68, 70, 72, 74, 76, 78, 80, 82, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 293, 295, 297, 299, 301, 303, 305, 307, 309, 311, 313, 315, 317, 319, 321, 704, 705, 709, and 710 or that is capable of hybridizing under stringent hybridization conditions to the complement of any one of SEQ ID NOs: 64, 66, 68, 70, 72, 74, 76, 78, 80, 82, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 293, 295, 297, 299, 301, 303, 305, 307, 309, 311, 313, 315, 317, 319, 321, 704, 705, 709, and 710.

In certain embodiments, the sequence encoding the guide miRNA sequence has at least 80%, 85%, 90%, 95%, 96%, 97%, 98%, or 99% sequence identity with any one of SEQ ID NOs: 64, 66, 68, 70, 72, 74, 76, 78, 80, 82, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 293, 295, 297, 299, 301, 303, 305, 307, 309, 311, 313, 315, 317, 319, 321, 704, 705, 709, and 710; or is capable of hybridizing under stringent hybridization conditions to the complement of any one of such sequences.

In certain embodiments, the sequence encoding the passenger miRNA sequence has at least 80%, 85%, 90%, 95%, 96%, 97%, 98%, or 99% sequence identity to any one of SEQ ID NOs: 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 294, 296, 298, 300, 302, 304, 306, 308, 310, 312, 314, 316, 318, 320, 322, 706-708, and 711-713; or is capable of hybridizing under stringent hybridization conditions to the complement of any one of such sequences.

In certain embodiments, the deoxyribonucleic acid encodes a pre-miRNA that targets CTLA. In certain such embodiments, the present invention relates to a polynucleotide comprising a nucleic acid sequence having at least 80%, 85%, 90%, 95%, 96%, 97%, 98%, or 99% sequence identity with any one of SEQ ID NOs: 65-71 or that is capable of hybridizing under stringent hybridization conditions to the complement of any one of SEQ ID NOs: 65-71. In certain embodiments, the present invention relates to a deoxyribonucleic acid comprising a nucleic acid sequence having at least 80%, 85%, 90%, 95%, 96%, 97%, 98%, or 99% sequence identity with any one of SEQ ID NOs: 64, 66, 68, and 70 or that is capable of hybridizing under stringent hybridization conditions to the complement of any one of SEQ ID NOs: 64, 66, 68, and 70.

In certain embodiments, the deoxyribonucleic acid encodes a pre-miRNA that targets PD-1. In certain such embodiments, the present invention relates to a polynucleotide comprising a nucleic acid sequence having at least 80%, 85%, 90%, 95%, 96%, 97%, 98%, or 99% sequence identity with any one of SEQ ID NOs: 72-83, 85, 87, and 704-713 or that is capable of hybridizing under stringent hybridization conditions to the complement of any one of SEQ ID NOs: 72-83, 85, 87, and 704-713. In certain embodiments, the present invention relates to a deoxyribonucleic acid comprising a nucleic acid sequence having at least 80%, 85%, 90%, 95%, 96%, 97%, 98%, or 99% sequence identity with any one of SEQ ID NOs: 72, 74, 76, 78, 80, 82, 704, 705, 709, and 710 or that is capable of hybridizing under stringent hybridization conditions to the complement of any one of SEQ ID NOs: 72, 74, 76, 78, 80, 82, 704, 705, 709, and 710.

In certain embodiments, the deoxyribonucleic acid encodes a pre-miRNA that targets TIGIT. In certain such embodiments, the present invention relates to a polynucleotide comprising a nucleic acid sequence having at least 80%, 85%, 90%, 95%, 96%, 97%, 98%, or 99% sequence identity with any one of SEQ ID NOs: 88-145 or that is capable of hybridizing under stringent hybridization conditions to the complement of any one of SEQ ID NOs: 88-145. In certain embodiments, the present invention relates to a deoxyribonucleic acid comprising a nucleic acid sequence having at least 80%, 85%, 90%, 95%, 96%, 97%, 98%, or 99% sequence identity with any one of SEQ ID NOs: 88, 90, 92, 94, 96, 98, 100, 102, 104, 106, 108, 110, 112, 114, 116, 118, 120, 122, 124, 126, 128, 130, 132, 134, 136, and 138 or that is capable of hybridizing under stringent hybridization conditions to the complement of any one of SEQ ID NOs: 64, 66, 68, and 70 SEQ ID NOs: 88, 90, 92, 94, 96, 98, 100, 102, 104, 106, 108, 110, 112, 114, 116, 118, 120, 122, 124, 126, 128, 130, 132, 134, 136, and 138.

In certain embodiments, the deoxyribonucleic acid encodes a pre-miRNA that targets TIM3. In certain such embodiments, the present invention relates to a polynucleotide comprising a nucleic acid sequence having at least 80%, 85%, 90%, 95%, 96%, 97%, 98%, or 99% sequence identity with any one of SEQ ID NOs: 146-157 or that is capable of hybridizing under stringent hybridization conditions to the complement of any one of SEQ ID NOs: 146-157. In certain embodiments, the present invention relates to a deoxyribonucleic acid comprising a nucleic acid sequence having at least 80%, 85%, 90%, 95%, 96%, 97%, 98%, or 99% sequence identity with any one of SEQ ID NOs: 146, 148, 150, 152, 154, and 156 or that is capable of hybridizing under stringent hybridization conditions to the complement of any one of SEQ ID NOs: 146, 148, 150, 152, 154, and 156.

In certain embodiments, the deoxyribonucleic acid encodes a pre-miRNA that targets LAG3. In certain such embodiments, the present invention relates to a polynucleotide comprising a nucleic acid sequence having at least 80%, 85%, 90%, 95%, 96%, 97%, 98%, or 99% sequence identity with any one of SEQ ID NOs: 158-161 or that is capable of hybridizing under stringent hybridization conditions to the complement of any one of SEQ ID NOs: 158-161. In certain embodiments, the present invention relates to a deoxyribonucleic acid comprising a nucleic acid sequence having at least 80%, 85%, 90%, 95%, 96%, 97%, 98%, or 99% sequence identity with any one of SEQ ID NOs: 158 and 160 or that is capable of hybridizing under stringent hybridization conditions to the complement of any one of SEQ ID NOs: 158 and 160.

In certain embodiments, the deoxyribonucleic acid encodes a pre-miRNA that targets GITR. In certain such embodiments, the present invention relates to a polynucleotide comprising a nucleic acid sequence having at least 80%, 85%, 90%, 95%, 96%, 97%, 98%, or 99% sequence identity with any one of SEQ ID NOs: 162-165 or that is capable of hybridizing under stringent hybridization conditions to the complement of any one of SEQ ID NOs: 162-165. In certain embodiments, the present invention relates to a deoxyribonucleic acid comprising a nucleic acid sequence having at least 80%, 85%, 90%, 95%, 96%, 97%, 98%, or 99% sequence identity with any one of SEQ ID NOs: 162 and 164 or that is capable of hybridizing under stringent hybridization conditions to the complement of any one of SEQ ID NOs: 162 and 164.

In certain embodiments, the deoxyribonucleic acid encodes a pre-miRNA that targets PIK3IP1. In certain such embodiments, the present invention relates to a polynucleotide comprising a nucleic acid sequence having at least 80%, 85%, 90%, 95%, 96%, 97%, 98%, or 99% sequence identity with any one of SEQ ID NOs: 166-171 or that is capable of hybridizing under stringent hybridization conditions to the complement of any one of SEQ ID NOs: 166-171. In certain embodiments, the present invention relates to a deoxyribonucleic acid comprising a nucleic acid sequence having at least 80%, 85%, 90%, 95%, 96%, 97%, 98%, or 99% sequence identity with any one of SEQ ID NOs: 166, 168, and 170 or that is capable of hybridizing under stringent hybridization conditions to the complement of any one of SEQ ID NOs: 166, 168, and 170.

In certain embodiments, the deoxyribonucleic acid encodes a pre-miRNA that targets CD70. In certain such embodiments, the present invention relates to a polynucleotide comprising a nucleic acid sequence having at least 80%, 85%, 90%, 95%, 96%, 97%, 98%, or 99% sequence identity with any one of SEQ ID NOs: 293-322 or that is capable of hybridizing under stringent hybridization conditions to the complement of any one of SEQ ID NOs: 293-322. In certain embodiments, the present invention relates to a deoxyribonucleic acid comprising a nucleic acid sequence having at least 80%, 85%, 90%, 95%, 96%, 97%, 98%, or 99% sequence identity with any one of SEQ ID NOs: 293, 295, 297, 299, 301, 303, 305, 307, 309, 311, 313, 315, 317, 319, and 321 or that is capable of hybridizing under stringent hybridization conditions to the complement of any one of SEQ ID NOs: 293, 295, 297, 299, 301, 303, 305, 307, 309, 311, 313, 315, 317, 319, and 321.

In certain embodiments, the present invention relates to a deoxyribonucleic acid wherein each sequence encoding a pre-miRNA comprises:
a) a sequence encoding a 5′ miRNA backbone sequence;
b) a sequence encoding a guide miRNA sequence;
c) a sequence encoding a stem loop sequence;
d) a sequence encoding a passenger miRNA sequence; and
e) a sequence encoding a 3′ backbone sequence.

In certain embodiments, the sequence encoding the pre-miRNA comprises:
a) a guide miRNA sequence having at least 80%, 85%, 90%, 95%, 96%, 97%, 98%, or 99% sequence identity with any one of SEQ ID NOs: 64, 66, 68, 70, 72, 74, 76, 78, 80, 82, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 704, 705, 709, and 710, or that is capable of hybridizing under stringent hybridization conditions to the complement of any one of such sequences; and
b) a passenger sequence having at least 80%, 85%, 90%, 95%, 96%, 97%, 98%, or 99% sequence identity to, respectively, any one of SEQ ID NOs: 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 294, 296, 298, 300, 302, 304, 306, 308, 310, 312, 314, 316, 318, 320, 322, 706-708, and 711-713, or that is capable of hybridizing under stringent hybridization conditions to the complement of any one of such sequences.

Deoxyribonucleic acids encoding exemplary non-natural pre-miRNA sequences targeting specific checkpoint inhibitors are described in Table 3. In certain embodiments, the deoxyribonucleic acid may comprise a sequence that is capable of hybridizing under stringent hybridization conditions with the complement of any one of the sequences listed in Table 3.

TABLE 3
Deoxyribonucleic acid sequences encoding
non-natural miRNA sequences

| | Backbone | DNA Sequence | DNA Sequence |
|---|---|---|---|
| miRNA | sequences | Encoding pri- | Encoding pre- |
| Target | derived from | miRNA Sequence | miRNA Sequence |
| CTLA4 | miR204 | (SEQ ID NO: 178) | (SEQ ID NO: 347) |
| PD1 | miR204 | (SEQ ID NO: 179) | (SEQ ID NO: 348) |
| PD1 | miR206 | (SEQ ID NO: 180) | (SEQ ID NO: 349) |
| TIGIT | miR17 | (SEQ ID NO: 181) | (SEQ ID NO: 350) |
| TIGIT | miR150 | (SEQ ID NO: 182) | (SEQ ID NO: 351) |
| TIGIT | miR204 | (SEQ ID NO: 183) | (SEQ ID NO: 352) |
| TIGIT | miR-206 | (SEQ ID NO: 184) | (SEQ ID NO: 353) |
| TIGIT | miR-204 | (SEQ ID NO: 185) | (SEQ ID NO: 354) |
| TIGIT | miR-22 | (SEQ ID NO: 186) | (SEQ ID NO: 355) |
| TIGIT | miR-16 | (SEQ ID NO: 187) | (SEQ ID NO: 356) |
| TIGIT | miR-16 | (SEQ ID NO: 188) | (SEQ ID NO: 357) |
| TIGIT | miR-21 | (SEQ ID NO: 189) | (SEQ ID NO: 358) |
| TIGIT | miR-494 | (SEQ ID NO: 190) | (SEQ ID NO: 359) |
| TIGIT | miR-142 | (SEQ ID NO: 191) | (SEQ ID NO: 360) |
| TIGIT | miR-19 | (SEQ ID NO: 192) | (SEQ ID NO: 361) |
| TIGIT | miR-1915 | (SEQ ID NO: 193) | (SEQ ID NO: 362) |
| TIGIT | miR-206 | (SEQ ID NO: 194) | (SEQ ID NO: 363) |
| TIGIT | miR-204 | (SEQ ID NO: 195) | (SEQ ID NO: 364) |
| TIGIT | miR-22 | (SEQ ID NO: 196) | (SEQ ID NO: 365) |
| TIGIT | miR-142 | (SEQ ID NO: 197) | (SEQ ID NO: 366) |
| TIGIT | miR-16 | (SEQ ID NO: 198) | (SEQ ID NO: 367) |
| TIGIT | miR-206 | (SEQ ID NO: 199) | (SEQ ID NO: 368) |
| TIGIT | miR-204 | (SEQ ID NO: 200) | (SEQ ID NO: 369) |
| TIGIT | miR-21 | (SEQ ID NO: 201) | (SEQ ID NO: 370) |
| TIGIT | miR-494 | (SEQ ID NO: 202) | (SEQ ID NO: 371) |
| TIGIT | miR-19 | (SEQ ID NO: 203) | (SEQ ID NO: 372) |
| TIGIT | miR-1915 | (SEQ ID NO: 204) | (SEQ ID NO: 373) |
| TIGIT | miR-204 | (SEQ ID NO: 205) | (SEQ ID NO: 374) |
| TIGIT | miR-206 | (SEQ ID NO: 206) | (SEQ ID NO: 375) |
| TIGIT | miR-21 | (SEQ ID NO: 207) | (SEQ ID NO: 376) |
| TIGIT | miR-22 | (SEQ ID NO: 208) | (SEQ ID NO: 377) |
| TIM3 | miR204 | (SEQ ID NO: 209) | (SEQ ID NO: 378) |
| TIM3 | miR150 | (SEQ ID NO: 210) | (SEQ ID NO: 379) |
| TIM3 | miR30a | (SEQ ID NO: 211) | (SEQ ID NO: 380) |
| TIM3 | miR206 | (SEQ ID NO: 212) | (SEQ ID NO: 381) |
| TIM3 | miR16 | (SEQ ID NO: 213) | (SEQ ID NO: 382) |
| TIM3 | miR17 | (SEQ ID NO: 214) | (SEQ ID NO: 383) |
| TIM3 | miR126 | (SEQ ID NO: 215) | (SEQ ID NO: 384) |
| TIM3 | miR122 | (SEQ ID NO: 216) | (SEQ ID NO: 385) |
| TIM3 | miR214 | (SEQ ID NO: 217) | (SEQ ID NO: 386) |
| TIM3 | miR29b1 | (SEQ ID NO: 218) | (SEQ ID NO: 387) |
| TIM3 | miR204 | (SEQ ID NO: 219) | (SEQ ID NO: 388) |
| TIM3 | miR133a1 | (SEQ ID NO: 220) | (SEQ ID NO: 389) |
| LAG3 | miR30a | (SEQ ID NO: 221) | (SEQ ID NO: 390) |
| LAG3 | miR206 | (SEQ ID NO: 222) | (SEQ ID NO: 391) |
| LAG3 | miR204 | (SEQ ID NO: 223) | (SEQ ID NO: 392) |
| LAG3 | miR150 | (SEQ ID NO: 224) | (SEQ ID NO: 393) |
| LAG3 | miR17 | (SEQ ID NO: 225) | (SEQ ID NO: 394) |
| LAG3 | miR122 | (SEQ ID NO: 226) | (SEQ ID NO: 395) |
| LAG3 | miR126 | (SEQ ID NO: 227) | (SEQ ID NO: 396) |
| GITR | miR30a | (SEQ ID NO: 228) | (SEQ ID NO: 397) |
| GITR | miR206 | (SEQ ID NO: 229) | (SEQ ID NO: 398) |
| GITR | miR17 | (SEQ ID NO: 230) | (SEQ ID NO: 399) |
| GITR | miR122 | (SEQ ID NO: 231) | (SEQ ID NO: 400) |
| GITR | miR150 | (SEQ ID NO: 232) | (SEQ ID NO: 401) |
| GITR | miR29 | (SEQ ID NO: 233) | (SEQ ID NO: 402) |
| GITR | miR181a1 | (SEQ ID NO: 234) | (SEQ ID NO: 403) |
| TIGIT | miR206 | (SEQ ID NO: 235) | (SEQ ID NO: 404) |
| TIGIT | miR30a | (SEQ ID NO: 236) | (SEQ ID NO: 405) |
| TIGIT | miR133a1 | (SEQ ID NO: 237) | (SEQ ID NO: 406) |
| TIGIT | miR122 | (SEQ ID NO: 238) | (SEQ ID NO: 407) |
| TIGIT | miR29b1 | (SEQ ID NO: 239) | (SEQ ID NO: 408) |
| TIGIT | miR214 | (SEQ ID NO: 240) | (SEQ ID NO: 409) |
| PD1 | miR30a | (SEQ ID NO: 241) | (SEQ ID NO: 410) |
| PD1 | miR412 | (SEQ ID NO: 242) | (SEQ ID NO: 411) |
| PD1 | miR17 | (SEQ ID NO: 243) | (SEQ ID NO: 412) |
| PD1 | miR122 | (SEQ ID NO: 244) | (SEQ ID NO: 413) |
| PD1 | miR150 | (SEQ ID NO: 245) | (SEQ ID NO: 414) |
| PD1 | miR486 | (SEQ ID NO: 246) | (SEQ ID NO: 415) |
| PD1 | miR206 | (SEQ ID NO: 247) | (SEQ ID NO: 416) |
| PD1 | miR122 | (SEQ ID NO: 248) | (SEQ ID NO: 417) |
| PD1 | miR30a | (SEQ ID NO: 249) | (SEQ ID NO: 418) |
| CTLA4 | miR206 | (SEQ ID NO: 250) | (SEQ ID NO: 419) |
| CTLA4 | miR26a1 | (SEQ ID NO: 251) | (SEQ ID NO: 420) |
| CTLA4 | miR30a | (SEQ ID NO: 252) | (SEQ ID NO: 421) |
| PIK3IP1 | miR206 | (SEQ ID NO: 253) | (SEQ ID NO: 422) |
| PIK3IP1 | miR126 | (SEQ ID NO: 254) | (SEQ ID NO: 423) |
| PIK3IP1 | miR30a | (SEQ ID NO: 255) | (SEQ ID NO: 424) |
| TCRa3′UTR | miR150 | (SEQ ID NO: 256) | (SEQ ID NO: 425) |
| TCRa3′UTR | miR204 | (SEQ ID NO: 257) | (SEQ ID NO: 426) |
| TCRa3′UTR | miR206 | (SEQ ID NO: 258) | (SEQ ID NO: 427) |
| TCRa3′UTR | miR26a1 | (SEQ ID NO: 259) | (SEQ ID NO: 428) |
| TCRa3′UTR | miR150 | (SEQ ID NO: 260) | (SEQ ID NO: 429) |
| TCRa3′UTR | miR16 | (SEQ ID NO: 261) | (SEQ ID NO: 430) |
| TCRa3′UTR | miR206 | (SEQ ID NO: 262) | (SEQ ID NO: 431) |
| TCRa3′UTR | miR26a1 | (SEQ ID NO: 263) | (SEQ ID NO: 432) |
| CD70 | miR206 | (SEQ ID NO: 323) | (SEQ ID NO: 433) |
| CD70 | miR204 | (SEQ ID NO: 324) | (SEQ ID NO: 434) |
| CD70 | miR22 | (SEQ ID NO: 325) | (SEQ ID NO: 435) |
| CD70 | miR16 | (SEQ ID NO: 326) | (SEQ ID NO: 436) |
| CD70 | miR21 | (SEQ ID NO: 327) | (SEQ ID NO: 437) |
| CD70 | miR206 | (SEQ ID NO: 328) | (SEQ ID NO: 438) |
| CD70 | miR204 | (SEQ ID NO: 329) | (SEQ ID NO: 439) |
| CD70 | miR19 | (SEQ ID NO: 330) | (SEQ ID NO: 440) |
| CD70 | miR494 | (SEQ ID NO: 331) | (SEQ ID NO: 441) |
| CD70 | miR150 | (SEQ ID NO: 332) | (SEQ ID NO: 442) |
| CD70 | miR206 | (SEQ ID NO: 333) | (SEQ ID NO: 443) |
| CD70 | miR204 | (SEQ ID NO: 334) | (SEQ ID NO: 444) |
| CD70 | miR22 | (SEQ ID NO: 335) | (SEQ ID NO: 445) |
| CD70 | miR142 | (SEQ ID NO: 336) | (SEQ ID NO: 446) |
| CD70 | miR26a1 | (SEQ ID NO: 337) | (SEQ ID NO: 447) |

In certain embodiments, the present invention relates to a deoxyribonucleic acid comprising a sequence having at least 80%, 85%, 90%, 95%, 96%, 97%, 98%, or 99% sequence identity with any one of SEQ ID NOs: 347-447 or that is capable of hybridizing under stringent hybridization conditions to the complement of any one of SEQ ID NOs: 347-447.

In certain embodiments, the present invention relates to a deoxyribonucleic acid comprising a sequence having at least 80%, 85%, 90%, 95%, 96%, 97%, 98%, or 99% sequence identity with any one of SEQ ID NOs: 178-263 and 323-337 or that is capable of hybridizing under stringent hybridization conditions to the complement of any one of SEQ ID NOs: 178-263 and 323-337.

In certain embodiments, a miRNA targets CTLA4. In certain such embodiments, the present invention relates to a deoxyribonucleic acid comprising a sequence having at least 80%, 85%, 90%, 95%, 96%, 97%, 98%, or 99% sequence identity with any one of SEQ ID NOs: 347, 419, 420, and 421 or that is capable of hybridizing under stringent hybridization conditions to the complement of any one of SEQ ID NOs: 347, 419, 420, and 421. In certain such embodiments, the present invention relates to a deoxyribonucleic acid comprising a sequence having at least 80%, 85%, 90%, 95%, 96%, 97%, 98%, or 99% sequence identity with any one of SEQ ID NOs: 178 and 250-252 or that is capable of hybridizing under stringent hybridization conditions to the complement of any one of SEQ ID NOs: 178 and 250-252.

In certain embodiments, a pre-miRNA targets PD-1. In certain such embodiments, the present invention relates to a deoxyribonucleic acid comprising a sequence having at least 80%, 85%, 90%, 95%, 96%, 97%, 98%, or 99% sequence identity with any one of SEQ ID NOs: 348, 349, and 410-418 or that is capable of hybridizing under stringent hybridization conditions to the complement of any one of SEQ ID NOs: 348, 349, and 410-418. In certain such embodiments, the present invention relates to a deoxyribonucleic acid comprising a sequence having at least 80%, 85%, 90%, 95%, 96%, 97%, 98%, or 99% sequence identity with any one of SEQ ID NOs: 179, 180, and 241-249 or that is capable of hybridizing under stringent hybridization conditions to the complement of any one of SEQ ID NOs: 179, 180, and 241-249. In certain embodiments, the present invention relates to a deoxyribonucleic acid comprising a sequence having at least 80%, 85%, 90%, 95%, 96%, 97%, 98%, or 99% sequence identity with any one of SEQ ID NO: 348 or 349 or that is capable of hybridizing under stringent hybridization conditions to the complement of any one of SEQ ID NOs: 348 or 349. In certain embodiments, the present invention relates to a deoxyribonucleic acid comprising a sequence having at least 80%, 85%, 90%, 95%, 96%, 97%, 98%, or 99% sequence identity with any one of SEQ ID NO: 179 or 180 or that is capable of hybridizing under stringent hybridization conditions to the complement of any one of SEQ ID NOs: 179 or 180.

In certain embodiments, a pre-miRNA targets TIGIT. In certain such embodiments, the present invention relates to a deoxyribonucleic acid comprising a sequence having at least 80%, 85%, 90%, 95%, 96%, 97%, 98%, or 99% sequence identity with any one of SEQ ID NOs: 350-377 and 404-409 or that is capable of hybridizing under stringent hybridization conditions to the complement of any one of SEQ ID NOs: 350-377 and 404-409. In certain such embodiments, the present invention relates to a deoxyribonucleic acid comprising a sequence having at least 80%, 85%, 90%, 95%, 96%, 97%, 98%, or 99% sequence identity with any one of SEQ ID NOs: 181-208 and 235-240 or that is capable of hybridizing under stringent hybridization conditions to the complement of any one of SEQ ID NOs: 181-208 and 235-240.

In certain embodiments, a pre-miRNA targets TIM3. In certain such embodiments, the present invention relates to a deoxyribonucleic acid comprising a sequence having at least 80%, 85%, 90%, 95%, 96%, 97%, 98%, or 99% sequence identity with any one of SEQ ID NOs: 378-389 or that is capable of hybridizing under stringent hybridization conditions to the complement of any one of SEQ ID NOs: 378-389. In certain such embodiments, the present invention relates to a deoxyribonucleic acid comprising a sequence having at least 80%, 85%, 90%, 95%, 96%, 97%, 98%, or 99% sequence identity with any one of SEQ ID NOs: 209-220 or that is capable of hybridizing under stringent hybridization conditions to the complement of any one of SEQ ID NOs: 209-220.

In certain embodiments, a pre-miRNA targets LAG3. In certain such embodiments, the present invention relates to a deoxyribonucleic acid comprising a sequence having at least 80%, 85%, 90%, 95%, 96%, 97%, 98%, or 99% sequence identity with any one of SEQ ID NOs: 390-396 or that is capable of hybridizing under stringent hybridization conditions to the complement of any one of SEQ ID NOs: 390-396. In certain such embodiments, the present invention relates to a deoxyribonucleic acid comprising a sequence having at least 80%, 85%, 90%, 95%, 96%, 97%, 98%, or 99% sequence identity with any one of SEQ ID NOs: 221-227 or that is capable of hybridizing under stringent hybridization conditions to the complement of any one of SEQ ID NOs: 221-227.

In certain embodiments, a pre-miRNA targets GITR. In certain such embodiments, the present invention relates to a deoxyribonucleic acid comprising a sequence having at least 80%, 85%, 90%, 95%, 96%, 97%, 98%, or 99% sequence identity with any one of SEQ ID NOs: 397-403 or that is capable of hybridizing under stringent hybridization conditions to the complement of any one of SEQ ID NOs: 397-403. In certain such embodiments, the present invention relates to a deoxyribonucleic acid comprising a sequence having at least 80%, 85%, 90%, 95%, 96%, 97%, 98%, or 99% sequence identity with any one of SEQ ID NOs: 228-234 or that is capable of hybridizing under stringent hybridization conditions to the complement of any one of SEQ ID NOs: 228-234.

In certain embodiments, a pre-miRNA targets PIK3IP1. In certain such embodiments, the present invention relates to a deoxyribonucleic acid comprising a sequence having at least 80%, 85%, 90%, 95%, 96%, 97%, 98%, or 99% sequence identity with any one of SEQ ID NOs: 422-424 or that is capable of hybridizing under stringent hybridization conditions to the complement of any one of SEQ ID NOs: 422-424. In certain such embodiments, the present invention relates to a deoxyribonucleic acid comprising a sequence having at least 80%, 85%, 90%, 95%, 96%, 97%, 98%, or 99% sequence identity with any one of SEQ ID NOs: 253-255 or that is capable of hybridizing under stringent hybridization conditions to the complement of any one of SEQ ID NOs: 253-255.

In certain embodiments, a pre-miRNA targets CD70. In certain such embodiments, the present invention relates to a deoxyribonucleic acid comprising a sequence having at least 80%, 85%, 90%, 95%, 96%, 97%, 98%, or 99% sequence identity with any one of SEQ ID NOs: 433-447 or that is capable of hybridizing under stringent hybridization conditions to the complement of any one of SEQ ID NOs: 433-447. In certain such embodiments, the present invention relates to a deoxyribonucleic acid comprising a sequence having at least 80%, 85%, 90%, 95%, 96%, 97%, 98%, or 99% sequence identity with any one of SEQ ID NOs: 323-337 or that is capable of hybridizing under stringent hybridization conditions to the complement of any one of SEQ ID NOs: 323-337.

In embodiments of the present invention, the two or more pre-miRNAs encoded by the deoxyribonucleic acid may each contain guide miRNA sequences that target the same target gene or the various guide miRNAs may target different genes. In addition, each pre-miRNA design of that of the pri-miRNA containing them may be based on a different native miRNA backbone to reduce the likelihood of misfolding of one miRNA with another. Table 4 provides examples of deoxyribonucleic acid sequences encoding two or more pri-miRNAs.

TABLE 4
Deoxyribonucleic acid sequences
comprising two or more pri-miRNAs

| | Backbone sequences | |
|---|---|---|
| miRNA Target | derived from | DNA Sequence |
| PD1 + PD1 | miR204 + miR206 | (SEQ ID NO: 267) |
| PD1 + TIGIT | miR204 + miR150 | (SEQ ID NO: 268) |
| PD1 + TIGIT | miR206 + miR150 | (SEQ ID NO: 269) |
| TIGIT + PD1 | miR17 + miR204 | (SEQ ID NO: 270) |
| TIGIT + PD1 | miR17 + miR206 | (SEQ ID NO: 271) |
| TIGIT + PD1 | miR204 + miR206 | (SEQ ID NO: 272) |
| TIGIT + PD1 | miR150 + miR204 | (SEQ ID NO: 273) |
| TIGIT + PD1 | miR150 + miR206 | (SEQ ID NO: 274) |
| TIGIT + PD1 + | miR17 + miR204 + miR206 | (SEQ ID NO: 275) |
| PD1 | | |
| TIGIT + PD1 + | miR17 + miR204 + miR206 | (SEQ ID NO: 276) |
| PD1 | extra spacing 1 | |
| TIGIT + PD1 + | miR17 + miR204 + miR206 | (SEQ ID NO: 277) |
| PD1 | extra spacing 2 | |
| PD1 + PD1 + | miR204 + miR206 + miR17 | (SEQ ID NO: 278) |
| TIGIT | | |
| PD1 + PD1 + | miR204 + miR206 + miR17 | (SEQ ID NO: 279) |
| TIGIT | extra spacing 1 | |
| PD1 + PD1 + | miR204 + miR206 + miR17 | (SEQ ID NO: 280) |
| TIGIT | extra spacing 2 | |
| PD1 + CTLA4 | miR204 + miR26a1 | (SEQ ID NO: 281) |
| PD1 + PD1 | miR204 + miR206 | (SEQ ID NO: 282) |
| PD1 + CTLA4 | miR206 + miR26a1 | (SEQ ID NO: 283) |
| PD1 + CTLA4 | miR206 + miR204 | (SEQ ID NO: 284) |
| TIGIT + CTLA4 | miR17 + miR26a1 | (SEQ ID NO: 285) |
| TIGIT + CTLA4 | miR17 + miR204 | (SEQ ID NO: 286) |
| TIGIT + PD1 | miR17 + miR204 | (SEQ ID NO: 287) |
| TIGIT + PD1 | miR17 + 206 | (SEQ ID NO: 288) |
| TIGIT + CTLA4 | miR204 + miR26a1 | (SEQ ID NO: 289) |
| TIGIT + PD1 | miR204 + miR206 | (SEQ ID NO: 290) |
| PD1 + PD1 + | miR204 + miR206 + miR16 | (SEQ ID NO: 448) |
| CD70 | | |
| PD1 + CD70 + | miR204 + miR206 + miR22 | (SEQ ID NO: 449) |
| CD70 | | |
| PD1 + CD70 | miR16 + miR204 + miR206 | (SEQ ID NO: 450) |
| CD70 + PD1 + | miR22 + miR204 + miR206 | (SEQ ID NO: 451) |
| PD1 | | |
| CD70 + CD70 | miR204 + miR206 | (SEQ ID NO: 452) |
| CD70 + CD70 | miR26a1 + miR206 | (SEQ ID NO: 453) |
| CD70 + CD70 | miR26a1 + miR16 | (SEQ ID NO: 454) |
| CD70 + CD70 | miR204 + miR26a1 | (SEQ ID NO: 455) |
| CD70 + CD70 | miR16 + miR26a1 | (SEQ ID NO: 456) |
| CD70 + CD70 | miR16 + miR22 | (SEQ ID NO: 457) |
| CD70 + CD70 | miR22 + miR16 | (SEQ ID NO: 458) |
| CD70 + CD70 | miR204 + miR16 | (SEQ ID NO: 459) |
| CD70 + CD70 | miR204 + miR16 | (SEQ ID NO: 460) |

In certain such embodiments, the present invention relates to a deoxyribonucleic acid comprising a nucleic acid sequence having at least 80%, 85%, 90%, 95%, 96%, 97%, 98%, or 99% sequence identity with any one of SEQ ID NOs: 267-290 and 448-460 or that is capable of hybridizing under stringent hybridization conditions to the complement of any one of SEQ ID NOs: 267-290 and 448-460.

In certain such embodiments, the deoxyribonucleic acid encodes two pre-miRNAs that target PD-1. In certain embodiments, the present invention relates to a deoxyribonucleic acid comprising a sequence having at least 80%, 85%, 90%, 95%, 96%, 97%, 98%, or 99% sequence identity with any one of SEQ ID NOs: 267 and 282 or that is capable of hybridizing under stringent hybridization conditions to the complement of any one of SEQ ID NOs: 267 and 282.

In certain such embodiments, the deoxyribonucleic acid encodes a pre-miRNA that targets PD-1 and a pre-miRNA that targets TIGIT. In certain embodiments, the present invention relates to a deoxyribonucleic acid comprising a sequence having at least 80%, 85%, 90%, 95%, 96%, 97%, 98%, or 99% sequence identity with any one of SEQ ID NOs: 269-274, 287, 288, and 290 or that is capable of hybridizing under stringent hybridization conditions to the complement of any one of SEQ ID NOs: 269-274, 287, 288, and 290.

In certain such embodiments, the deoxyribonucleic acid encodes two pre-miRNAs that target PD-1 and a pre-miRNA that targets TIGIT. In certain embodiments, the present invention relates to a deoxyribonucleic acid comprising a sequence having at least 80%, 85%, 90%, 95%, 96%, 97%, 98%, or 99% sequence identity with any one of SEQ ID NOs: 275-280 or that is capable of hybridizing under stringent hybridization conditions to the complement of any one of SEQ ID NOs: 275-280.

In certain such embodiments, the deoxyribonucleic acid encodes a pre-miRNA that targets PD-1 and a pre-miRNA that targets CTLA4. In certain embodiments, the present invention relates to a deoxyribonucleic acid comprising a sequence having at least 80%, 85%, 90%, 95%, 96%, 97%, 98%, or 99% sequence identity with any one of SEQ ID NOs: 281, 283, and 284 or that is capable of hybridizing under stringent hybridization conditions to the complement of any one of SEQ ID NOs: 281, 283, and 284.

In certain such embodiments, the deoxyribonucleic acid encodes a pre-miRNA that targets TIGIT and a pre-miRNA that targets CTLA4. In certain embodiments, the present invention relates to a deoxyribonucleic acid comprising a sequence having at least 80%, 85%, 90%, 95%, 96%, 97%, 98%, or 99% sequence identity with any one of SEQ ID NOs: 285, 286, and 289 or that is capable of hybridizing under stringent hybridization conditions to the complement of any one of SEQ ID NOs: 285, 286, and 289.

In certain such embodiments, the deoxyribonucleic acid encodes two pre-miRNAs that target PD1 and a pre-miRNA that targets CD70. In certain embodiments, the present invention relates to a deoxyribonucleic acid comprising a sequence having at least 80%, 85%, 90%, 95%, 96%, 97%, 98%, or 99% sequence identity with SEQ ID NO: 448 or 451 or that is capable of hybridizing under stringent hybridization conditions to the complement of SEQ ID NO: 448 or 451.

In certain such embodiments, the deoxyribonucleic acid encodes a pre-miRNAs that targets PD1 and two pre-miRNAs that target CD70. In certain embodiments, the present invention relates to a deoxyribonucleic acid comprising a sequence having at least 80%, 85%, 90%, 95%, 96%, 97%, 98%, or 99% sequence identity with SEQ ID NO: 449 or that is capable of hybridizing under stringent hybridization conditions to the complement of SEQ ID NO: 449.

In certain such embodiments, the deoxyribonucleic acid encodes a pre-miRNAs that targets PD1 and a pre-miRNA that target CD70. In certain embodiments, the present invention relates to a deoxyribonucleic acid comprising a sequence having at least 80%, 85%, 90%, 95%, 96%, 97%, 98%, or 99% sequence identity with SEQ ID NO: 450 or that is capable of hybridizing under stringent hybridization conditions to the complement of SEQ ID NO: 450.

In certain such embodiments, the deoxyribonucleic acid encodes two pre-miRNAs that each CD70. In certain embodiments, the present invention relates to a deoxyribonucleic acid comprising a sequence having at least 80%, 85%, 90%, 95%, 96%, 97%, 98%, or 99% sequence identity with any one of SEQ ID NOs: 452-460 or that is capable of hybridizing under stringent hybridization conditions to the complement of any one of SEQ ID NOs: 452-460.

### III. Protein of Interest

In certain embodiments, the deoxyribonucleic acid encoding the pre-miRNAs is contained in the same genetic construct as that comprising one or more genes encoding protein(s) of interest (e.g., a chimeric antigen receptor, a cytokine, a cell tag, or a checkpoint inhibitor).

### A. Chimeric Receptors

In any of the foregoing embodiments, a deoxyribonucleic acid of the present disclosure can further encode a chimeric receptor, such as a chimeric antigen receptor (CAR), or a chimeric T cell receptor (TCR). Thus, a deoxyribonucleic acid of the present disclosure can encode miRNAs and a chimeric receptor, such as a CAR or TCR.

In certain embodiments, a deoxyribonucleic acid of the present invention encodes a CAR and is introduced into a T cell, thereby generating a chimeric antigen receptor T cell (a CAR-T cell).

A CAR is an engineered receptor that grafts an exogenous specificity onto an immune effector cell. In some instances, a CAR comprises an extracellular domain (ectodomain) that comprises an antigen-binding domain, a transmembrane domain, and an intracellular (endodomain) domain. The intracellular domain comprises an intracellular signaling domain. In certain embodiments, the extracellular domain further comprises a region of amino acids (i.e., a spacer) between the antigen-binding domain and the transmembrane domain.

An antigen-binding domain can comprise complementary determining regions of a monoclonal antibody and/or antigen binding fragments thereof. A complementarity determining region (CDR) is a short amino acid sequence found in the variable domains of antigen receptor (e.g., immunoglobulin and T-cell receptor) proteins that bind an antigen and therefore provides the receptor with its specificity for that particular antigen. Each polypeptide chain of an antigen receptor can contain three CDRs (CDR1, CDR2, and CDR3).

In certain embodiments, the antigen-binding domain comprises an antibody, or functional fragment or variant thereof, that binds to a target antigen. The functional fragment or variant may comprise the variable domain of the heavy chain of an antibody (V_{H}) and/or the variable domain of the light chain of an antibody (V_{L}), or functional fragments or variants thereof. In certain embodiments, the antigen-binding domain comprises a Fv, Fab, Fab₂, Fab′, F(ab′)₂, or F(ab′)₃ fragment of an antibody. In certain embodiments, the antigen-binding domain comprises a scFv, sc(Fv)₂, a dsFv, a diabody, a minibody, a nanobody, or binding fragments thereof. In certain embodiments, the antigen-binding domain further comprises an Fc fragment of an antibody, for example it may comprise an scFv linked with an Fc fragment.

In some embodiments, the CAR targets an antigen that is overexpressed in cancer cells, in autoimmune cells, or in cells that are infected by a virus, bacteria or parasite. Pathogens that may be targeted include, without limitation, Plasmodium, trypanosome, Aspergillus, Candida, Hepatitis A, Hepatitis B, Hepatitis C, HSV, HPV, RSV, EBV, CMV, JC virus, BK virus, or Ebola pathogens. Autoimmune diseases can include graft-versus-host disease, rheumatoid arthritis, lupus, celiac disease, Crohn's disease, Sjogren Syndrome, polymyalgia rheumatic, multiple sclerosis, neuromyelitis optica, ankylosing spondylitis, Type 1 diabetes, alopecia areata, vasculitis, temporal arteritis, bullous pemphigoid, psoriasis, pemphigus vulgaris, and autoimmune uveitis.

The pathogen recognized by a CAR may be essentially any kind of pathogen, but in some embodiments, the pathogen is a fungus, bacteria, or virus. Exemplary viral pathogens include those of the families of Adenoviridae, Epstein-Barr virus (EBV), Cytomegalovirus (CMV), Respiratory Syncytial Virus (RSV), JC virus, BK virus, HPV, HSV, HHV family of viruses, Hepatitis family of viruses, Picornaviridae, Herpesviridae, Hepadnaviridae, Flaviviridae, Retroviridae, Orthomyxoviridae, Paramyxoviridae, Papovaviridae, Polyomavirus, Rhabdoviridae, and Togaviridae. Exemplary pathogenic viruses cause smallpox, influenza, mumps, measles, chickenpox, ebola, and rubella. Exemplary pathogenic fungi include Candida, Aspergillus, Cryptococcus, Histoplasma, Pneumocystis, and Stachybotrys. Exemplary pathogenic bacteria include Streptococcus, Pseudomonas, Shigella, Campylobacter, Staphylococcus, Helicobacter, E. coli, Rickettsia, Bacillus, Bordetella, Chlamydia, Spirochetes, and Salmonella. In some embodiments, the pathogen receptor Dectin-1 may be used to generate a CAR that recognizes the carbohydrate structure on the cell wall of fungi such as Aspergillus. In another embodiment, CARs can be made based on an antibody recognizing viral determinants (e.g., the glycoproteins from CMV and Ebola) to interrupt viral infections and pathology.

In certain embodiments, the CAR comprises an antigen-binding domain that binds an antigen that is overexpressed in a cancer.

In some embodiments, a CAR comprises an antigen-binding domain that binds to an epitope on B7H4, BCMA, BTLA, CAIX, CA125, CCR4, CD3, CD4, CD5, CD7, CD16, CD19, CD20, CD22, CD24, CD25, CD28, CD30, CD33, CD38, CD40, CD44, CD44v6, CD44v7/v8, CD47, CD52, CD56, CD70, CD79b, CD80, CD81, CD86, CD123, CD133, CD137, CD138, CD151, CD171, CD174, CD276, CEA, CEACAM6, CLL-1, c-MET, CS1, CSPG4, CTLA-4, DLL3, EDB-F, EGFR, EGFR2, EGFRVIII, EGP-2, EGP-40, EphA2, FAP, FLT1, FLT4, Folate-binding Protein, Folate Receptor, Folate receptor α, α-Folate receptor, Frizzled, GD2, GD3, GHR, GHRHR, GITR, GPC3, Gp100, gp130, HBV antigens, HER1, HER2, HER3, HER4, HER1/HER3, h5T4, HPV antigens, HVEM, IGFIR, IgKAppa, IL-1-RAP, IL-2R, IL6R, IL-11Rα, IL-13R-a2, KDR, KRASG12V, LewisA, LewisY, L1-CAM, LIFRP, LRP5, LTPR, MAGE-A, MAGE-A1, MAGE-A10, MAGE-A3, MAGEA3/A6, MAGE-A4, MAGE-A6, MART-1, MCAM, mesothelin, PSCA, Mucins such as MUC1, MUC-4 or MUC16, NGFR, NKG2D, Notch-1-4, NY-ESO-1, O-acetylGD2, O-acetylGD3, OX40, P53, PD1, PDE10A, PD-L1, PD-L2, PMSA, PRAME, PSCA, PSMA, PTCH1, RANK, Robol, ROR1, ROR1R, ROR2, TACI, TAG-72, TCRa, TCRp, TGF, TGFBeta, TGFBeta-II, TGFBR1, TGFBR2, Titin, TLR7, TLR9, TNFR1, TNFR2, TNFRSF4, TRBC1, TWEAK-R, VEGF, VEGF-R2, or WT-1.

In some embodiments, a CAR described herein comprises an antigen-binding domain that binds to an epitope on CD19, CD33, MUC1, MUC16, ROR1, HLA-A2, myelin oligodendrocyte glycoprotein (MOG), factor VIII (FVIII), MAdCAM1, SDF1, and/or collagen type II.

In some embodiments, a CAR described herein comprises an antigen-binding domain that binds to an epitope on CD19, CD33, MUC1, MUC16, and/or ROR1.

In some embodiments, the CAR comprises an antigen-binding domain that binds to an epitope on CD19. Examples of CARs that bind an epitope on CD19 are known to those skilled in the art and are described, for example in International Application Publication Nos. WO 2016/033570; WO 2015/123642; and WO2015/187528

In some embodiments, the CAR comprises an antigen-binding domain that binds to an epitope on CD33. Examples of CARs that bind an epitope on CD33 are known to those skilled in the art and are described, for example in International Application Publication No. WO 2017/214333.

In some embodiments, the CAR comprises an antigen-binding domain that binds to an epitope on MUC1. Examples of CARs that bind an epitope on MUC1 are known to those skilled in the art and are described.

In some embodiments, the CAR comprises an antigen-binding domain that binds to an epitope on MUC16. Examples of CARs that bind an epitope on MUC16 are known to those skilled in the art and are described, for example in International Application Publication No. WO 2019/236577.

In some embodiments, the CAR comprises an antigen-binding domain that binds to an epitope on ROR1. Examples of CARs that bind an epitope on ROR1 are known to those skilled in the art and are described, for example in International Application Publication No. WO 2020/014366.

Antigen binding can be assessed by flow cytometry or a cell based assay or any other equivalent assay. Cell based assays may utilize a cell type expressing antigen of interest on the surface to assess antigen-binding. An antigen or a fragment thereof expressed as a soluble protein can be utilized to assess antigen-binding using flow cytometry or similar assay. Improvements in antigen-binding may be indirectly assessed by functional measurement of antigen-binding domain or a chimeric receptor. For example, improved antigen-binding of a chimeric receptor or a CAR, as described herein, can be measured by increased specific cytotoxicity against target cells expressing the antigen.

Cell surface expression level of a polypeptide of the present disclosure can be assessed, for example, using a flow cytometry based assay. Improved expression of an antigen-binding polypeptide can be measured as percentage of analyzed cells expressing said antigen-binding polypeptide or alternatively as average density of said antigen-binding polypeptide on the surface of a cell. Additional suitable methods that can be used for assessing cell surface expression of the antigen-binding polypeptides described herein include western blotting or any other equivalent assay.

### B. Cytokine

In any of the foregoing embodiments, a deoxyribonucleic acid of the present disclosure can further encode a cytokine. Thus, a deoxyribonucleic acid of the present disclosure can encode miRNAs and a cytokine.

In certain embodiments, the deoxyribonucleic acid also encodes a CAR and is introduced into a T cell, thereby generating a CAR-T cell. In certain other embodiments, a deoxyribonucleic acid of the present invention encoding an miRNA and a cytokine is introduced into a CAR-T cell.

In some cases, the cytokine comprises at least one chemokine, interferon, interleukin, lymphokine, tumor necrosis factor, or variant or combination thereof. In certain embodiments, the cytokine is an interferon, GM-CSF, G-CSF, M-CSF, LT-beta, TNF-alpha, growth factors, hGH, and/or a ligand of human Toll-like receptors TLR1, TLR2, TLR3, TLR4, TLR5, TLR6, TLR7, TLR8, TLR9, TLR10, IFN-alpha, IFN-beta, or IFN-gamma.

In certain embodiments, the cytokine is an interleukin. In some cases the interleukin is IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-35, or a functional variant or fragment thereof.

In certain embodiments, the cytokine may be IL-12, or a functional fragment or variant thereof. In some embodiments, the IL-12 is a single chain IL-12 (scIL-12), protease sensitive IL-12, destabilized IL-12, membrane bound IL-12, intercalated IL-12. In some instances, the IL-12 variants are as described in International Application Publications Nos. WO2015/095249, WO2016/048903, WO2017/062953.

In certain embodiments, the cytokine may be IL-15, or a functional fragment or variant thereof. In certain embodiments, the IL-15, or functional fragment or variant thereof, is membrane-bound. Such may occur when IL-15, or a functional fragment or variant thereof, is bound to membrane-bound IL-15Rα, or a functional fragment or variant thereof. Thus, certain embodiments of the present invention may involve a fusion protein comprising IL-15, or a functional fragment or variant thereof, and IL-15Rα, or a functional fragment or variant thereof. In certain embodiments, the IL-15, or functional fragment or variant thereof, is linked to the IL-15Rα, or functional fragment thereof by way of a linker. Examples of fusion proteins comprising IL-15, or a functional fragment or variant thereof, bound to membrane-bound IL-15Rα, or a functional fragment or variant thereof, are known to those skilled in the art and are described, for example in International Application Publication No. WO 2014/186469.

In certain embodiments, the cytokine is linked to a signal peptide. Any signal for use in eukaryotic cells, including those described above for use with the CARs may be linked to the cytokine. In certain embodiments, the cytokine is linked to an IgE signal peptide.

### C. Cell Tag

In any of the foregoing embodiments, a deoxyribonucleic acid of the present disclosure can further encode a cell tag. Thus, a deoxyribonucleic acid of the present disclosure can encode miRNAs and a cell tag.

In certain embodiments, the deoxyribonucleic acid also encodes a CAR and is introduced into a T cell, thereby generating a CAR-T cell. In certain other embodiments, a deoxyribonucleic acid of the present invention encoding an miRNA and a cell tag is introduced into a CAR-T cell. In certain such embodiments, the deoxyribonucleic acid also encodes a cytokine. In certain embodiments, the deoxyribonucleic acid of the present invention encodes: (a) a CAR; (b) a protein comprising IL-15, or a functional fragment or variant thereof, and IL-15Rα, or a functional fragment or variant thereof; and (c) a cell tag.

In certain embodiments, the cell tag is used as a kill switch, selection marker, a biomarker, or a combination thereof.

In certain embodiments, the cell tag is capable of being bound by a predetermined binding partner. In certain such embodiments wherein the deoxyribonucleic acid encoding the cell tag is introduced into a cell and the cell is introduced to a subject, the administration of the predetermined binding partner to the subject allows for depletion of the cells. For example, the administration of cetuximab or any antibody that recognizes HER1 allows for the elimination of cells expressing a cell tag comprising truncated non-immunogenic HER1.

In certain such embodiments, the cell tag is non-immunogenic. This may be accomplished, for example, wherein the cell tag comprises a polypeptide that is truncated so that it is non-immunogenic. For example, the truncation of the HER1 sequence eliminates the potential for EGF ligand binding, homo- and hetero-dimerization of EGFR, and/or EGFR-mediated signaling while keeping cetuximab-binding ability intact (Ferguson, K., 2008. A structure-based view of Epidermal Growth Factor Receptor regulation. Annu Rev Biophys, Volume 37, pp. 353-373).

In certain embodiments, the cell tag comprises at least one of a truncated non-immunogenic HER1 polypeptide, a truncated non-immunogenic LNGFR polypeptide, a truncated non-immunogenic CD20 polypeptide, or a truncated non-immunogenic CD52 polypeptide, or a functional fragment or variant thereof.

In certain embodiments, the cell tag comprises a HER1 Domain III, or a functional fragment or variant thereof, and a truncated HER1 Domain IV, or a functional fragment or variant thereof. Examples of such cell tags are known to those skilled in the art and are described, for example in International Application Publication No. WO 2014/186469.

In certain embodiments, the cell tag comprises a truncated non-immunogenic CD20, or CD20t−1, or a functional fragment or variant thereof.

In certain embodiments, the cell tag further comprises a transmembrane domain. The transmembrane domain can be derived from either a natural or a synthetic source. Where the source is natural, the domain can, for example, be derived from any membrane-bound or transmembrane protein. Suitable transmembrane domains can include the transmembrane domain(s) of alpha, beta or zeta chain of the T-cell receptor; or a transmembrane domain from CD28, CD3 epsilon, CD3ζ, CD45, CD4, CD5, CD8, CD9, CD16, CD22, CD33, CD37, CD64, CD80, CD86, CD134, CD137 or CD154, or a functional fragment or variant thereof. In certain embodiments, the cell tag further comprises a CD28 transmembrane domain or a functional fragment or variant thereof. Alternatively, the transmembrane domain can be synthetic, and can comprise hydrophobic residues such as leucine and valine. In some embodiments, a triplet of phenylalanine, tryptophan and valine is found at one or both termini of a synthetic transmembrane domain.

In certain embodiments, the cell tag comprises a truncated HER1, or functional fragment or variant thereof, and a transmembrane domain, or a functional fragment or variant thereof.

In certain embodiments, the cell tag is linked with a signal peptide. The signal peptide can be any signal peptide suitable for use in a eukaryotic cell including those described with respect to CARs herein. In certain embodiments, the signal peptide is a Igϰ signal peptide, or a functional fragment or variant thereof.

### D. Immune Checkpoint Inhibitors

In any of the foregoing embodiments, a deoxyribonucleic acid of the present disclosure can further encode an immune checkpoint inhibitor. Thus, a deoxyribonucleic acid of the present disclosure can encode miRNAs and an immune checkpoint inhibitor. The use of such a deoxyribonucleic acid thus provides two modes of action for reducing the activity of immune checkpoints.

In certain embodiments, the immune checkpoint inhibitor inhibits the activity of an immune checkpoint protein such as PD1, PD-L1, CTLA-4, TIGIT, 4-1BB, PIK3IP1, CD27, CD28, CD40, CD70, CD122, CD137, OX40 (CD134), GITR, ICOS, A2AR, B7-H3 (CD276), B7-H4 (VTCN1), BTLA, IDO, KIR, LAG3, TIM-3, or VISTA.

In certain embodiments, the immune checkpoint inhibitor is an antibody or a functional fragment or variant thereof.

In some embodiments, the immune checkpoint inhibitor is: an anti-PD1 antibody such as cemiplimab, pembrolizumab, nivolumab, torpalimab, sintilimab, LY3434172, JTX-4014, 609A, Sym021, LZM009, budigalimab, IB, SCT-I10A, SG001, AMP-224, AMG 404, AK112, CS1003, MEDI0680, RO7121661, F520, sasanlimab, BI 754091, cetrelimab, HerinCAR-PD-1, HX008, zimberelimab, retifanlimab, balstilimab, pidilizumab, teripalimab, CBT-501, BAT1306, tislelizumab, AK105, spartalizumab, prolgolimab, serplulimab, dostarlimab, camrelizumab, IBI319, KY1043, STI-1110, CA05100948, Nb97, ENUM 388D4, hAb-10D3, ANB030, MCLA-134, and hAb21; an anti-CTLA-4 antibody such as ipilimumab (YERVOY) and tremelimumab; an anti-PD-L1 antibody such as BMS935559 (MDX-1105), atezolizumab, avelumab, or durvalumab; an anti-CD28 antibody; an anti-TIGIT antibody; an anti-LAG3 antibody such as BMS-986016 and LAG525; an anti-TIM3 antibody; an anti-GITR antibody; an anti-4-1BB antibody such as PF-05082566; or an anti-OX-40 antibody such as MEDI6469, MEDI0562, and MOXR0916.

### IV. Genetic Construct

As previously discussed, in certain embodiments, the deoxyribonucleic acid encoding the pre-miRNAs is contained in the same genetic construct as that comprising one or more genes encoding protein(s) of interest (e.g., a chimeric antigen receptor, a cytokine, or a cell tag).

In certain such embodiments, such a genetic construct includes a nucleic acid sequence encoding a 5′ untranslated region (5′UTR) directly upstream of a gene encoding a protein of interest and the pre-miRNA sequences are included in the 5′UTR. In certain embodiments, such a genetic construct includes a nucleic acid sequence encoding a 3′ untranslated region (3′UTR) directly downstream of a gene encoding a protein of interest and the pre-miRNA sequences are included in the 3′UTR. In certain embodiments, such a genetic construct includes nucleic acid sequences encoding both a 5′UTR and a 3′UTR with each such region containing at least one pre-miRNA sequence (for example, each UTR can include one pre-miRNA, the 5′UTR can include one pre-miRNA and the 3′UTR can include 2 pre-miRNAs, the 5′UTR can include two pre-miRNAs and the 3′UTR can include one pre-miRNA, both UTRs can include two pre-miRNAs, etc.).

In embodiments wherein the sequence encoding the pri-miRNA is included in the sequence corresponding to the 5′UTR, the transcribed RNA can include additional sequences such as splice donor, branchpoint and/or acceptor site sequences. The inclusion of splice donor, branchpoint, and acceptor sites is important for splicing of the miRNAs from the transcribed RNA. Without splicing, the highly structured miRNA sequence is likely to impede ribosome scanning to the translation initiation sequence relating to the gene of interest. Examples of sequences encoding such splice donor/acceptor sites include SEQ ID NOs: 291 and 292, sequences having at least 80%, 85%, 90%, 95%, 96%, 97%, 98%, or 99% sequence identity with such sequences and sequences that are capable of hybridizing with the complement of such sequences under stringent hybridization conditions.

Thus, in certain embodiments, the deoxyribonucleic acids of the present invention further comprises: a) a nucleic acid sequence having at least 80% sequence identity with SEQ ID NO: 291 or that is capable of hybridizing under stringent hybridization conditions to the complement of SEQ ID NO: 291; and b) a nucleic acid sequence having at least 80% sequence identity with SEQ ID NO: 292 or that is capable of hybridizing under stringent hybridization conditions to the complement of SEQ ID NO: 292.

In certain embodiments, the portion of the deoxyribonucleic acid encoding a pre-miRNA is contained in a section corresponding to an intron within the gene encoding a protein of interest. In certain such embodiments, at least one pre-miRNA is contained in an intron located within the 5′UTR (hereinafter referred to as the “5′UTR intron”).

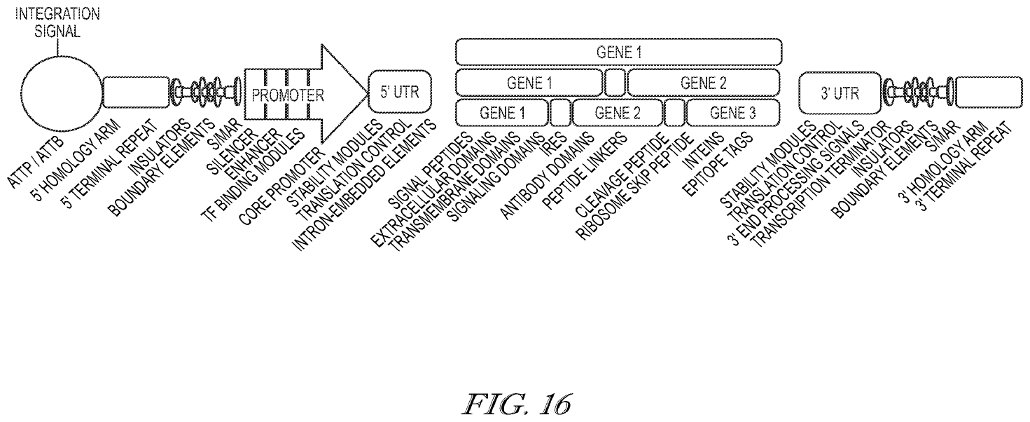

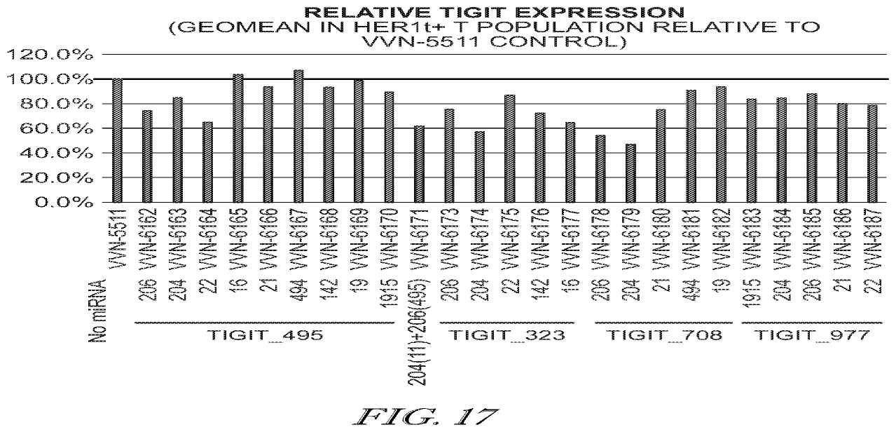

An exemplary polynucleotide which can be used as template for the expression of various genes and other regulatory elements in cells is depicted in FIG. 16. It should be understood that various elements can be included or omitted in the polynucleotide and that different options are shown for various exemplary sites in the polynucleotide.

As shown in FIG. 16, the polynucleotide can include an integration signal for attP/attB phage integration of the polynucleotide into a bacterial genome. The polynucleotide can further include a 5′ homology arm or 5′ terminal repeat and a 3′ homology arm or 3′ terminal repeat. The polynucleotide can further include insulators, boundary elements and S/MAR positioned 3′ adjacent to the 5′ homology arm or 5′ terminal repeat and 5′ adjacent to the 3′ homology arm or 3′ terminal repeat. Between the insulators, boundary elements, or S/MAR, the polynucleotide can include, from 5′ to 3′, a promoter which can include a silencer, enhancer, TF binding modules and a core promoter; a 5′ untranslated region which can include stability modules, translation control elements, and intron-embedded elements such as miRNA encoding sequences; one or more genes which can include signal peptides, extracellular domains, transmembrane domains, signaling domains, antibody domains, peptide linkers, inteins and epitope tags; and a 3′ untranslated region that can include stability modules, translation control, 3′ end processing signals and a transcription terminator.

As previously discussed, the miRNA may be encoded in the same genetic construct with additional proteins of interest (e.g., a CAR, a cytokine, and/or a cell tag). An advantage of having two or more of such components expressed using one genetic construct is stoichiometric expression of such components.

As understood by those skilled in the art, genes encoding the polypeptides of interest may be linked by way of linkers. Any suitable linker known to link genes may be used in the practice of the present invention, Examples, of such linkers include those encoding an internal ribosome entry site (IRES), cleavable peptides, and ribosomal skipping peptides. Examples cleavable peptides encoded by such linkers include Furinlink, fmdv, and 2A linkers (e.g., P2A, GSG-P2A, FP2A, T2A, and Furin-T2A), or functional fragments or variants thereof.

The polynucleotide of the invention can be present in the construct in operable linkage with a promoter. Appropriate promoters can be selected based on the host cell and effect sought. Suitable promoters include constitutive and inducible promoters. The promoters can be tissue specific, such promoters being well known in the art.

Examples of constitutive promoters for use in the present invention include, but are not limited to, immediate early cytomegalovirus (CMV) promoter; human elongation growth factor 1 alpha 1 (hEF1A1); simian virus 40 (SV40) early promoter; mouse mammary tumor virus (MMTV); human immunodeficiency virus (HIV) long terminal repeat (LTR) promoter; MoMuLV promoter; avian leukemia virus promoter; Epstein-Barr virus immediate early promoter; Rous sarcoma virus promoter; and human gene promoters such as, but not limited to, the actin promoter, the myosin promoter, the hemoglobin promoter, and the creatine kinase promoter; and functional fragments and variants thereof.

In contrast to constitutive promoters, the use of an inducible promoter provides a molecular switch capable of turning on the expression of the polynucleotide sequence which it is operatively linked when such expression is desired, or turning off the expression when expression is not desired. Examples of inducible promoters include, but are not limited to, a metallothionine promoter, a glucocorticoid promoter, a progesterone promoter, and a tetracycline promoter. In one aspect, the inducible promoter can be a gene switch ligand inducible promoter. In some cases, an inducible promoter can be a small molecule ligand-inducible two polypeptide ecdysone receptor-based gene switch, such as a RHEOSWITCH® gene switch.

The present invention relates in part to a deoxyribonucleic acid comprising a genetic construct as described above.

### V. Vectors and Delivery Systems

The ribonucleic acid and/or the deoxyribonucleic acid of the present invention can be delivered to cells on a long oligonucleotide, which is then inserted into a specific genome location. In certain embodiments, the ribonucleic acid and/or the deoxyribonucleic acid of the present invention can be integrated into a cell's genome through gene editing systems that utilize CRISPR, TALEN or Zinc-Finger nucleases.

The polynucleotide of the present invention can be delivered to a target cell by any suitable delivery system, including non-viral and viral delivery systems. The present invention thus also relates in part to a vector comprising the ribonucleic acid or the deoxyribonucleic acid of the present invention.

Ay vector known in the art for use in delivering ribonucleic acids or deoxyribonucleic acids may be used in the practice of the present invention. In certain embodiments, the vector is a plasmid, a mini-circle DNA, a nanoplasmid, a viral vector, an episomal vector, or a non-viral vector. Examples of viral vectors for use in the present invention include lentiviral vectors and retroviral vectors. Examples of non-viral vectors for use in the present invention include Sleeping Beauty transposons. In certain embodiments, the vector may include sequences for serine recombinase mediated integration (e.g., for an aatP or attB site). Where the vector is a plasmid, mini-circle DNA, or a nanoplasmid, the plasmid, mini-circle DNA or nanoplasmid can further include a bacterial origin of replication, for example one from a ColE1 plasmid.

An example of a non-viral vector for use in delivering a deoxyribonucleic acid or ribonucleic acid of the present invention is a lipid formulation. Any lipid formulation known in the art for delivering such nucleic acids may be used in the practice of the present invention. In certain embodiments, the nucleic acid can be associated with a lipid. For example, the nucleic acid may encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid.

Another example of a non-viral vector is a transposon. Any transposon known in the art for delivering a deoxyribonucleic acid or a ribonucleic acid may be used in the practice of the present invention. When a transposon is used to deliver a nucleic acid, a transposase or a nucleic acid encoding the same is typically also delivered to the cell. A transposase is an enzyme that binds to a transposon and catalyzes its integration into the genome of a cell. In certain embodiments, the vector is a Sleeping Beauty transposon. When used, a Sleeping Beauty transposase, or a functional fragment or variant thereof, or a nucleic acid encoding the same is also delivered to the cell. Examples of such transposases include, but are not limited to, SB10, SB11, SB100x, and SB110 transposases. Sleeping Beauty transposon systems are known in the art and are described for example, in U.S. Pat. Nos. 6,489,458 and 8,227,432.

Any viral vector known in the art for delivering deoxyribonucleic acids or ribonucleic acids may be used in the practice of the present invention. Examples of such vectors include, but are not limited to, adenoviral vectors (e.g., the adenovirus-based Per.C6 system available from Crucell, Inc. (Leiden, The Netherlands)), adeno-associated virus based vectors, lentivirus-based vectors (e.g., the lentiviral-based pLPI from Life Technologies (Carlsbad, Calif.)), retroviral vectors (e.g., the pFB-ERV plus pCFB-EGSH), and herpes virus-based vectors.

In an embodiment, the viral vector is an adenoviral vector.

In an embodiment, the viral vector is a lentiviral vector. Vectors derived from retroviruses such as the lentivirus are suitable tools to achieve long-term gene transfer since they allow long-term, stable integration of a transgene and its propagation in daughter cells. Lentiviral vectors have added advantage over vectors derived from onco-retroviruses such as murine leukemia viruses in that they can transduce non-proliferating cells, such as hepatocytes. They also have the added advantage of low immunogenicity.

In order to assess the expression of one or more miRNA(s) and a CAR described herein or portions thereof, the expression vector to be introduced into a cell can also contain either a selectable marker gene or a reporter gene or both to facilitate identification and selection of expressing cells from the population of cells sought to be transfected or infected through viral vectors or non-viral vectors. In other aspects, the selectable marker can be carried on a separate piece of DNA and used in a co-transfection procedure. Both selectable markers and reporter genes can be flanked with appropriate regulatory sequences to enable expression in the host cells. Useful selectable markers include, for example, antibiotic-resistance genes, such as neomycin resistance gene (neo) and ampicillin resistance gene and the like. In some embodiments, a truncated epidermal growth factor receptor (HER1t or HER1t−1) tag can be used as a selectable marker gene.

Reporter genes can be used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes include genes encoding luciferase, beta-galactosidase, chloramphenicol acetyl transferase, secreted alkaline phosphatase, or the green fluorescent protein gene (e.g., Ui-Tei et al., FEBS Letters 479:79-82 (2000)). Suitable expression systems are well known and can be prepared using known techniques or obtained commercially. In general, the construct with the minimal 5′ flanking region showing the highest level of expression of reporter gene is identified as the promoter. Such promoter regions can be linked to a reporter gene and used to evaluate agents for the ability to modulate promoter-driven transcription.

In certain embodiments, the vector comprises: (a) a nucleic acid sequence encoding a ribonucleic acid comprising at least one pri-miRNA; (b) a nucleic acid sequence encoding a CAR; (c) a nucleic acid encoding a cytokine; and (d) a nucleic acid encoding a cell tag. The miRNA, CAR, cytokine, and cell tag encoded in a single construct allows for manufacturing consistency.

In come embodiments, the ribonucleic acid comprises two pri-miRNAs. In certain embodiments, both miRNAs target PD-1. In certain embodiments, the ribonucleic acid comprises: (a) a pri-miRNA comprising backbone sequences from miR204 and a guide miRNA that targets PD-1; and (b) a pri-miRNA comprising backbone sequences from miR206 and a guide miRNA that targets PD-1.

In some embodiments, the CAR comprises an antigen-binding domain as described elsewhere in herein, for example, an antigen binding domain that binds to an epitope on CD19, CD33, MUC1, MUC16, ROR1, HLA-A2, myelin oligodendrocyte glycoprotein (MOG), factor VIII (FVIII), MAdCAM1, SDF1, and/or collagen type II. In certain embodiments, the CAR comprises an antigen-binding domain that binds to an epitope on CD19, CD33, MUC1, MUC16, and/or ROR1. In certain such embodiments, the CAR comprises an antigen-binding domain that binds to an epitope on ROR1.

In some embodiments, the cytokine is IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-35, or a functional variant or fragment thereof. In certain embodiments, the cytokine is IL-15, or a functional fragment or variant thereof. In certain embodiments, the IL-15, or functional fragment or variant thereof, is membrane-bound. In certain embodiments, the vector encodes a fusion protein comprising IL-15, or a functional fragment or variant thereof, and IL-15Rα, or a functional fragment or variant thereof.

In some embodiments, the cell tag comprises a HER1 Domain III, or a functional fragment or variant thereof, and a truncated HER1 Domain IV, or a functional fragment or variant thereof. In certain embodiments, the cell tag further comprises a CD28 transmembrane domain or a functional fragment or variant thereof.

In certain embodiments, the nucleic acid encoding the CAR and the nucleic acid encoding the cytokine are linked by a nucleic acid encoding a linker, for example, a furin-T2A linker. In certain embodiments, the nucleic acid encoding the nucleic acid encoding the cytokine and the nucleic acid encoding the cell tag are linked by a nucleic acid encoding a linker, for example, a T2A linker.

In some embodiments, the vector further encodes a splice donor site and a splice acceptor site. In certain embodiments, the splice donor side comprises SEQ ID NO: 291 and the splice acceptor site comprises SEQ ID NO: 292.

In some embodiments, the vector comprises: (a) a nucleic acid sequence encoding: (i) a pri-miRNA comprising backbone sequences from miR204 and a guide miRNA that targets PD-1; and (ii) a pri-miRNA comprising backbone sequences from miR206 and a guide miRNA that targets PD-1; (b) a nucleic acid sequence encoding a CAR comprising an antigen-binding domain that binds to an epitope on ROR; (c) a nucleic acid encoding a fusion protein comprising IL-15, or a functional fragment or variant thereof, and IL-15Rα, or a functional fragment or variant thereof; and (d) a nucleic acid encoding a cell tag comprising: (i) a HER1 Domain III, or a functional fragment or variant thereof; (ii) a truncated HER1 Domain IV, or a functional fragment or variant thereof; and (iii) a CD28 transmembrane domain or a functional fragment or variant thereof. In certain such embodiments, the nucleic acid encoding the CAR and the nucleic acid encoding the cytokine are linked by a nucleic acid encoding a furin-T2A linker, and the nucleic acid encoding the nucleic acid encoding the cytokine and the nucleic acid encoding the cell tag are linked by a nucleic acid encoding a T2A linker. In certain such embodiments, the vector is in the form of a splice donor Sleeping Beauty transposon. In some embodiments, the vector further encodes a splice donor side comprising SEQ ID NO: 291 and a splice acceptor site comprising SEQ ID NO: 292.

### VI. Methods for Introducing the miRNA into Cells

The present invention relates in part to a method for modifying the expression of a gene in a cell, wherein the method comprises introducing the ribonucleic acid of the present invention or the deoxyribonucleic acid of the present invention to the cell. The present invention also relates in part to the use of a ribonucleic acid of the present invention or the deoxyribonucleic acid of the present invention in the manufacture of a medicament for modifying the expression of a gene.

The present invention also relates in part to a method for producing a genetically-engineered cell, wherein the method comprises introducing the ribonucleic acid of the present invention or the deoxyribonucleic acid of the present invention to a cell.

In certain embodiments of the above methods, the method comprises transfecting a cell with the ribonucleic acid of the present invention or the deoxyribonucleic acid of the present invention. In certain embodiments, transfection involves electroporation.

In certain embodiments, the deoxyribonucleic acid may comprise a transposon, for example a Sleeping Beauty transposon. In embodiments wherein a Sleeping Beauty transposon is used, a Sleeping Beauty transposase or a functional fragment or variant thereof, or a nucleic acid encoding the same, may be introduced to the cell. In certain embodiments wherein the cell is transfected with a transposon, the method further comprises transfecting the cell with a vector encoding a transposase.

In certain embodiments of the above methods, the cell is transduced with the ribonucleic acid of the present invention or the deoxyribonucleic acid of the present invention. The cells may be transduced with a viral vector comprising such ribonucleic acid or deoxyribonucleic acid.

Methods of introducing and expressing genes into a cell are known in the art. In the context of an expression vector, the vector can be readily introduced into a host cell, e.g., mammalian, bacterial, yeast, or insect cell by any method known in the art. For example, the vector can be transferred into a cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are known in the art. See, for example, Sambrook et al. (Molecular Cloning: A Laboratory Manual, Cold Spring Harbor Laboratory, New York (2001)). In some embodiments, a method for the introduction of a polynucleotide into a host cell is calcium phosphate transfection or polyethylenimine (PEI) Transfection. In some embodiments, a method for introduction of a polynucleotide into a host cell is electroporation.

Chemical methods for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

As a biological method, the ribonucleic acid or the deoxyribonucleic acid of the present invention may, for example, be introduced to the cell using viral-based delivery systems. Representative viral expression vectors include, but are not limited to, the adenovirus-based vectors (e.g., the adenovirus-based Per.C6 system available from Crucell, Inc. (Leiden, The Netherlands)), adeno-associated virus based vectors, lentivirus-based vectors (e.g., the lentiviral-based pLPI from Life Technologies (Carlsbad, Calif.)), retroviral vectors (e.g., the pFB-ERV plus pCFB-EGSH), and herpes virus-based vectors. In an embodiment, the viral vector is a lentivirus vector. Vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity. In general, and in embodiments, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

Regardless of the method used to introduce exogenous nucleic acids into a host cell, in order to confirm the presence of the recombinant DNA sequence in the host cell, a variety of assays can be performed. Such assays include, for example, “molecular biological” assays well known to those of skill in the art, such as Southern and Northern blotting, RT-PCR and PCR and “biochemical” assays, such as detecting the presence or absence of a particular peptide, e.g., by immunological means (ELISAs and Western blots).

### VII. Genetically-Modified Cells

The present invention relates in part to a genetically-modified cell comprising the ribonucleic acid or the deoxyribonucleic acid of the present invention. The cell may be produced by any method known in the art for introducing such ribonucleic acids or the deoxyribonucleic acids into a cell. In certain embodiments, the cell is produced using a method as described herein.

In certain embodiments, the cell is a modified immune effector cell. In certain embodiments, the modified immune effector cell is a modified T cell, a natural killer (NK) cell, or a macrophage. In certain embodiments, the modified T cell is a modified cytotoxic T cell, for example a T cell that destroys virus-infected cells and/or tumor cells.

In certain embodiments, an immune effector cell is obtained from a subject, for example by isolation from umbilical cord blood, peripheral blood, human embryonic stem cells, iPSCs, bone marrow, lymph node tissue, thymus tissue, tissue from a site of infection, ascites, pleural effusion, spleen tissue, and tumors. Such cells may then be modified, for example by the methods described herein, to contain the ribonucleic acid or the deoxyribonucleic acid of the present invention. The present invention thus contemplates that, in methods such as those described herein for producing a genetically-engineered cell, there may be an initial step of obtaining a cell from a subject.

Following modification, for example by transfection or transduction, the cells can be immediately infused into the subject or can be cryo-preserved. In certain embodiments, the cells are incubated for less than 10 days, less than 9 days, less than 8 days, less than 7 days, less than 6 days, less than 5 days, less than 4 days, less than 3 days, less than 2 days, less than 1 day, or less than 12 hours following transfection or transduction before being delivered to (e.g. by infusion into) the subject. In certain embodiments, the cells are manufactured to allow for delivery to the subject within 10 days, less than 9 days, less than 8 days, less than 7 days, less than 6 days, less than 5 days, less than 4 days, less than 3 days, less than 2 days, less than 1 day, or less than 12 hours following transfection or transduction. In certain embodiments, the cells do not undergo propagation, activation, incubation, or culturing prior to delivery (e.g., by infusion) to the subject.

In certain aspects, following transfection or transduction, the cells can be preserved in a cytokine bath that can include IL-2 and/or IL-21 until ready for infusion. In certain aspects, following modification, the cells can be propagated for days, weeks, or months ex vivo as a bulk population within about 1, 2, 3, 4, 5 days or more following gene transfer into cells. In a further aspect, following modification, the modified cells are cloned and a clone demonstrating presence of a single integrated or episomally maintained expression cassette or plasmid, and expression of the miRNA and/or protein of interest is expanded ex vivo.

Recombinant T cells can be expanded by stimulation with IL-2, or other cytokines that bind the common gamma-chain (e.g., IL-7, IL-12, IL-15, IL-21, and others). Recombinant T cells can also be expanded by stimulation with artificial antigen presenting cells (aAPCs) or with an antibody, such as OKT3, which cross links CD3 on the T cell surface.

### VIII. Kits and Compositions

The present invention relates in part to a kit or composition comprising the ribonucleic acid or the deoxyribonucleic acid of the present invention. In certain embodiments, the kit or composition comprises the vector of the present invention. In certain embodiments, the kit or composition comprises the genetically-modified cell of the present invention.

The present invention also relates in part to a kit or composition as described above for use in modifying the expression of a gene. The present invention further relates in part to a kit or composition as described above for use in treating a disease or disorder in a subject or for use in the production of a medicament for treating a disease or disorder in a subject.

In certain embodiments, the kit or composition comprises a transposase.

In certain embodiments, the kit or composition comprises gene switch components, such as components of the RHEOSWITCH® gene switch components.

In certain embodiments, the composition further comprises a carrier, a diluents, and/or an excipient. Any carrier, diluent, or excipient known in the art for use with a nucleic acid, vector, or cell is contemplated for use in the practice of the present invention. For example, compositions of the present invention may comprise: buffers such as neutral buffered saline, phosphate buffered saline and the like; carbohydrates such as glucose, mannose, sucrose, dextrans, or mannitol; proteins; polypeptides or amino acids such as glycine; antioxidants; chelating agents such as EDTA or glutathione; adjuvants (e.g., aluminum hydroxide); and preservatives.

In certain embodiments, the kit comprises a carrier, package, label, or container. Suitable containers include, for example, bottles, vials, syringes, and test tubes.

### IX. Methods of Treatment

The present invention also relates to a method of treating a disease or disorder in a subject comprising administering a ribonucleic acid, a deoxyribonucleic acid, a vector, a cell, or a composition of the present invention to the subject. Administration may be in a therapeutically effective amount. In certain embodiments, the subject is a mammal, for example a human.

The present invention also relates to the use of a ribonucleic acid, a deoxyribonucleic acid, a vector, a cell, or a composition of the present invention in the manufacture of a medicament for the treatment of a disease or disorder in a subject.

In certain embodiments, the disease or disorder is a disease or disorder for which the reduction or silencing of the expression of an immune checkpoint protein would provide a benefit. In certain such embodiments, the disease or disorder is a cancer, an autoimmune disorder, or caused by an infection by a virus, bacteria, or parasite.

In some embodiments, the disease is cancer. The cancer may be a hematological or solid tumor. In some cases, the cancer is metastatic. Examples of cancers that may be treated include, but are not limited to, human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's sarcoma, Ewing's tumor, leiomyosarcoma, rhabdomyosarcoma, colon carcinoma, colorectal cancer, pancreatic cancer, breast cancer, beast adenocarcinomas e.g. triple negative breast cancer, ovarian cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, liver cancer, hepatocellular carcinoma (HCC), choriocarcinoma, seminoma, embryonal carcinoma, Wilms' tumor, cervical cancer, bone cancer, brain tumor, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute myeloid leukemia, acute lymphocytic leukemia, mantle cell lymphoma, acute lymphoblastic leukemia, and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); diffuse large B-cell lymphoma; and polycythemia vera, lymphoma (Hodgkin's disease and non-Hodgkin's disease), multiple myeloma, Waldenstrom's macroglobulinemia, and heavy chain disease.

In some embodiments, the disease is an autoimmune disease. Examples of such autoimmune diseases include, but are not limited to, graft-versus-host disease, rheumatoid arthritis, lupus, celiac disease, Crohn's disease, Sjogren Syndrome, polymyalgia rheumatic, multiple sclerosis, neuromyelitis optica, ankylosing spondylitis, Type 1 diabetes, alopecia areata, vasculitis, temporal arteritis, bullous pemphigoid, psoriasis, pemphigus vulgaris, and autoimmune uveitis.

In some embodiments, the disease is a disease caused by an infection by a virus, bacteria, or parasite. Examples of such disease include, but are not limited to those caused by Plasmodium, trypanosome, Aspergillus, Candida, Hepatitis A, Hepatitis B, Hepatitis C, HSV, HPV, RSV, EBV, CMV, JC virus, BK virus, and Ebola pathogens.

In some embodiments, the disease or disorder is associated with the overexpression of an antigen. In certain embodiments, the antigen is CD19, CD33, ROR1, MUC1, or MUC16.

In some embodiments, the disease is associated with the overexpression of MUC16. In certain such embodiments, the disease is ovarian cancer, breast cancer, pancreatic cancer, endometrial cancer, or lung cancer.

In some embodiments, the disease is associated with the overexpression of CD33. In certain such embodiments, the disease is acute myeloid leukemia (AML) or a myelodysplastic syndrome (MDS).

In some embodiments, the disease is associated with the overexpression of ROR1. In certain such embodiments, the disease involves a hematological tumor, for example chronic lymphocytic leukemia (CLL), mantle cell lymphoma (MCL), acute lymphoblastic leukemia (ALL), and diffuse large B-cell lymphoma (DLBCL). In certain such embodiments, the disease involves a solid tumor, for example breast adenocarcinomas encompassing triple negative breast cancer (TNBC), pancreatic cancer, ovarian cancer, and lung adenocarcinoma.

In certain embodiments, the method involves the administration of a nucleic acid, vector, cell, or composition described herein. Such methods may be carried out in any manner known in the art, including by aerosol inhalation, injection, ingestion, transfusion, implantation or transplantation. The nucleic acid, vector, cell, or composition described herein can be administered to a patient subcutaneously, intradermally, intratumorally, intranodally, intramedullary, intramuscularly, by intravenous (i.v.) injection, or intraperitoneally.

In certain embodiments, the method involves the administration of a genetically-modified cell to the subject. The cell may be the cell of the present invention as described herein. Such a method may comprise obtaining a sample of cells from the subject, modifying cells of the sample with the ribonucleic acid or deoxyribonucleic acid of the present invention, and administering the modified cells to the subject, for example by infusion.

In certain embodiments, the modified immune cells are directly administered to a particular site on a body, for example targeted to a cancer via regional delivery directly to the tumor tissue. For example, in ovarian cancer, modified immune effector cells can be delivered intraperitoneally (IP) to the abdomen or peritoneal cavity. Such IP delivery can be performed via a port or pre-existing port placed for delivery of chemotherapy drugs. Other methods of regional delivery of modified cells can include catheter infusion into resection cavity, ultrasound guided intratumoral injection, hepatic artery infusion or intrapleural delivery.

In some embodiments, the subject is subjected to lymphodepletion before the step of administering the modified cells to the subject. As used herein, “lymphodepletion” involves methods that reduce the number of lymphocytes in a subject, for example by administration of a lymphodepletion agent. Examples of lymphodepletion include nonmyeloablative lymphodepleting chemotherapy, myeloablative lymphodepleting chemotherapy. Lymphodepletion can also be attained by partial body or whole body fractioned radiation therapy. A lymphodepletion agent can be a chemical compound or composition capable of decreasing the number of functional lymphocytes in a mammal when administered to the mammal. Such agents and dosages are known, and can be selected by a treating physician depending on the subject to be treated. Examples of lymphodepletion agents include, but are not limited to, fludarabine, cyclophosphamide, cladribine, denileukin diftitox, or combinations thereof. In some embodiments, the subject is not subjected to lymphodepletion before the step of administering the modified immune effector cells to the subject.

In some embodiments, patients or subjects are not lymphodepleted prior to blood being withdrawn to produce the autologous modified immune effector cells.

In some embodiments, the modified immune effector cells are autologous to the subject. In some embodiments, the modified immune effector cells are allogeneic to the subject.

The dosage of the above treatments to be administered to a patient will vary with the precise nature of the condition being treated and the recipient of the treatment. The scaling of dosages for human administration can be performed according to art-accepted practices. The appropriate dose can be adjusted accordingly for an adult or a pediatric patient.

In some cases, an effective amount of the modified cells for administration comprises about 10⁴ to about 10⁹ modified cells/kg, about 10⁴ to about 10⁵ modified cells/kg, about 10⁵ to about 10⁶ modified cells/kg, about 10⁶ to about 10⁷ modified effector cells/kg, >10⁴ but ≤10⁵ modified cells/kg, >10⁵ but ≤10⁶ modified effector cells/kg, or >10⁶ but ≤10⁷ modified effector cells/kg.

Alternatively, a typical amount of modified cells administered to a mammal (e.g., a human) can be, for example, in the range of one hundred, one thousand, ten thousand, one million to 100 billion cells; however, amounts below or above this exemplary range are within the scope of the invention. For example, the dose of such cells can be about 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), or about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells, or a range defined by any two of the foregoing values).

It is to be noted that dosage values may vary with the type and severity of the condition to be alleviated. It is to be further understood that, for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

Therapeutic or prophylactic efficacy can be monitored by periodic assessment of treated patients. For repeated administrations over several days or longer, depending on the condition, the treatment is repeated until a desired suppression of disease symptoms occurs. However, other dosage regimens can be useful and are within the scope of the invention. The desired dosage can be delivered by a single bolus administration of the composition, by multiple bolus administrations of the composition, or by continuous infusion administration of the composition.

In some embodiments, an amount of modified cells is administered to a subject in need thereof and the amount is determined based on the efficacy and the potential of inducing a cytokine-associated toxicity.

In some embodiments, the compositions described herein can be administered as a combination therapy with an additional therapeutic agent. Examples of such agents include biologic agents and small molecules.

### EXAMPLES

These Examples are provided for illustrative purposes only and not to limit the scope of the claims provided herein. The following table includes abbreviations and special terms that apply to the Examples only. These abbreviations and special terms are not otherwise limiting, and neither replace nor narrow the broader definitions set forth above, which shall continue to apply to the claims.

TABLE 5
Abbreviations and Special Terms for Use in the Examples

| Abbreviation of Special Term | Explanation |
|---|---|
| Artificial miRNA | A non-naturally occurring miRNA designed to target a |
| | specific gene |
| CAR-T | Chimeric Antigen Receptor T Cell |
| Guide miRNA | The mature miRNA processed from the pre-miRNA that is |
| | complementary to an intended target gene |
| LFC | Log2 fold change |
| Mature miRNA | The fully processed 21-23nt miRNA |
| mbIL15 | A fusion protein comprising IL-15 and IL-15Rα, the protein |
| | comprising the amino acid sequence of SEQ ID NO: 525 |
| miRNA | microRNA, a small RNA species generally 21-23nt in length |
| | that is complementary to a target mRNA transcript and |
| | reduces expression of the target transcript through either |
| | RNA degradation or inhibition of translation |
| Passenger miRNA | A mature miRNA that may be processed from the side |
| | opposite of the guide miRNA in the RNA stem-loop |
| PD1 Silencer Module | A genetic module encoded within the VVN-5351 transgene |
| | cassette designed to reduce expression of PD1 within |
| | ROR1 + PD1 silencer cells. The module consists of a splice |
| | unit containing a non-coding RNA that is processed by |
| | internal cellular machinery into 2 unique microRNAs with |
| | homology to PD1 mRNA. |
| Pre-miRNA | Precursor miRNA, which is the intermediate miRNA species |
| | that has undergone the first processing step to remove the |
| | RNA stem-loop structure from the primary mRNA transcript. |
| | Pre-miRNA undergoes a second processing step in which the |
| | mature miRNA is cleaved and loaded into the RNA-induced |
| | silencing complex (RISC). |
| ROR1 + PD1 silencer cells | ROR1 CAR-T cells expressing mbIL15 and HER1t with anti- |
| | PD-1 miRNAs |
| Pri-miRNA/pri-miRNA scaffold | The primary transcript encoding RNA stem-loop structures |
| | that are processed by the endogenous cellular machinery into |
| | precursor and then mature miRNA. The artificial pri-miRNA |
| | sequence and structures are based on endogenous human pri- |
| | miRNAs, herein referred to as a scaffold, with the natural |
| | guide and passenger strands replaced with a guide miRNA to |
| | target PD1 and a passenger miRNA to maintain the predicted |
| | folded RNA structure. |
| UTR | Untranslated region |
| VVN-5351 | A DNA transposon plasmid encoding the ROR1 UltraCAR-T |
| | transgene cassette plus a dual PD1-targeting miRNA in the |
| | 5′UTR encoded within a synthetic splice unit. |
| VVN-5355 | A DNA transposon plasmid encoding the ROR1 UltraCAR-T |
| | transgene cassette. |

### Example 1. PD1 Module Design

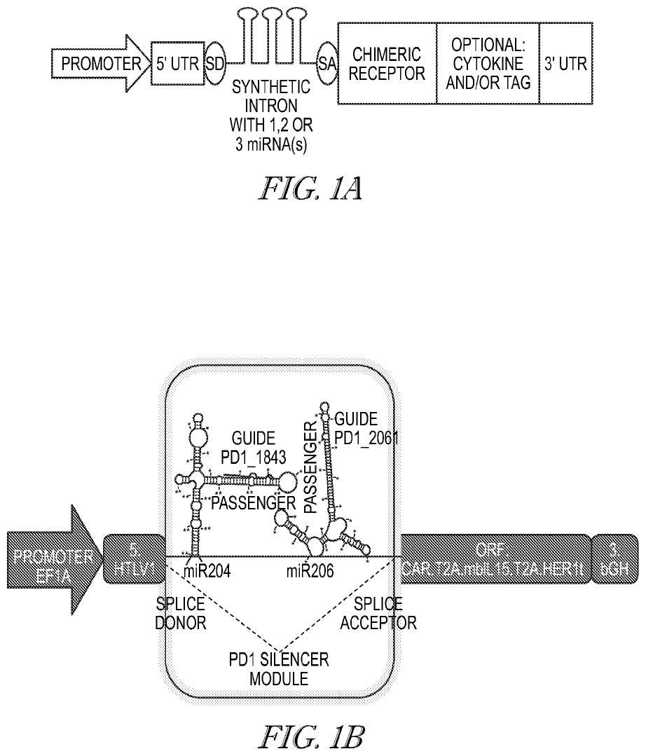

The PD1-silencing module of miRNA-expressing ROR1 UltraCAR-T cells encodes two artificial miRNAs designed to specifically reduce expression of PD-1 mRNA within UltraCAR-T cells while avoiding off-target silencing of other endogenous transcripts. The two artificial miRNAs of miRNA-expressing ROR1 UltraCAR-T cells are encoded within a dual primary miRNA (pri-miRNA) sequence placed within a 5′ UTR splice unit of the UltraCAR-T transgene cassette (FIG. 1B). The dual pri-miRNA forms stem-loop structures that are recognized and processed by cellular complexes to generate two unique 21-24 nucleotide mature guide miRNAs that are homologous to specific sequences within the PD1 target transcript. Interaction of the guide miRNAs with the PD1 target sequence is expected to trigger the silencing of PD1 by induction of RNA degradation or translational inhibition (Guo et al, 2010).

The guide miRNAs encoded within miRNA-expressing ROR1 UltraCAR-T cells were designed to be highly specific for the PD1 target transcript NM_005018.3 by implementation of an internally-designed computational workflow using a combination of twenty-one ranking parameters based on three validated rules-based siRNA prediction algorithms (Amarzguioui and Prydz, 2004; Reynolds et al, 2004; Ui-Tei et al, 2004). Multi-level specificity profiling was performed against the human reference exome (RefSeq) and the activated T-cell transcriptome (Zhao et al, 2014) to ensure that the mature miRNAs are highly specific for the PD1 target gene. To further reduce risk of off-target silencing by the non-PD1 targeting passenger strand miRNAs, pri-miRNA scaffolds were selected that produce a high ratio of guide:passenger miRNA (Miniarikova et al, 2016). The PD1-targeting guide miRNA PD1_1843 was incorporated into a pri-miRNA scaffold based on human miRNA hsa-miR-204 (accession #MI0000284) while the PD1_2061 guide miRNA was incorporated into the hsa-miR-206 (accession #MI0000490) scaffold. Mutations were created on the passenger strand side of each pri-miRNA to assure the specific miRNA structure was maintained and that thermodynamic stability was not substantially altered. RNA structures were predicted using CLC Main Workbench software. The PD1 silencing module contains the miR204 PD1_1843 pri-miRNA placed directly upstream of miR206 PD1_2061 pri-miRNA within a synthetic splice unit in the 5′ UTR of the CAR-T transgene expression cassette. The splice units were ordered as gBlock from IDT and can be cloned into Sleeping Beauty CAR vectors and can be cut using ClaI/NheI for cloning into the 5′UTR of any other Sleeping Beauty CAR vector.

### Example 2. Reduction of PD1 Transcript Expression by miRNAs

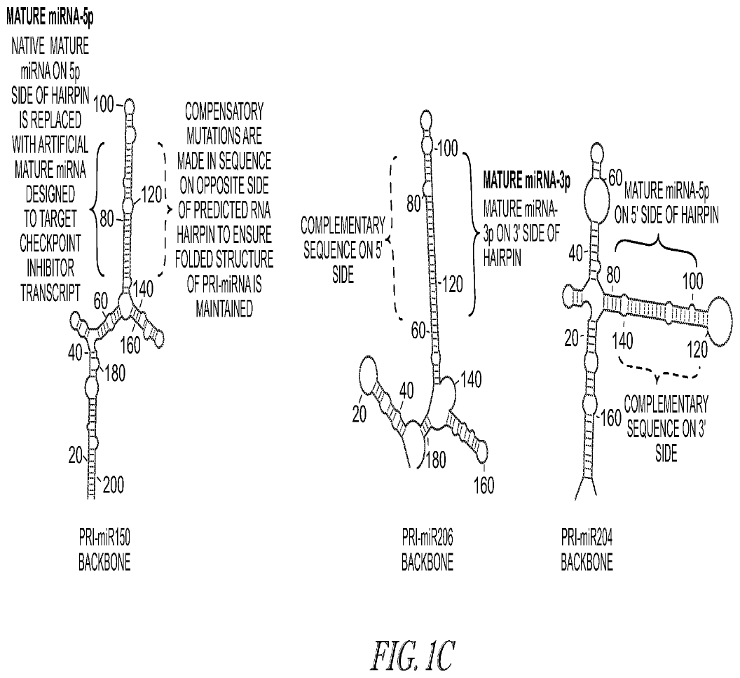

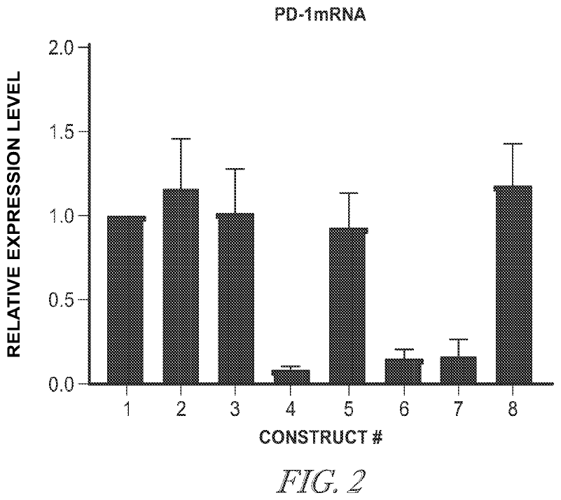

Primary human T cells were transfected with the constructs listed in Table 6 and expanded in vitro with the use of AaPC cells expressing cognate antigen that were grown in large batches and then frozen into aliquots. The generated CAR-T cells were then further activated using anti-CD3/anti-CD28 beads at a bead:T cell ratio of 1:1 and 1×10⁶ T cells/mL for 48 hrs prior to harvesting the cells for RNA isolation. RNA isolation was performed according to manufacturer's recommended protocol (Qiagen) then subjected to RT-qPCR analysis using the SuperScript VILO Master Mix with ezDNase (Invitrogen) and TaqMan FAST Advanced master mix for qPCR to evaluate PD-1 expression levels using specific primer/probes (Human PD1: Hs00169472_m1; Human TIGIT: Hs00545087_m1; and Human ACTb: Hs99999903_m1). All samples were also normalized to beta-actin expression levels. The relative expression values are based upon ΔΔCT method by normalizing to construct #1 (MUC16 CAR-T cells only). Results are shown in FIG. 2. Shown is the mean±SD from 3 donors.

TABLE 6
Description of constructs 1-8 as utilized in FIG.
2 sequences of miRNA are as described in Table 4)

| Con- | | |
|---|---|---|
| struct | | |
| # | miRNA | Effector Genes |
| 1 | N/A | MUC16 CAR-mbIL15-HER1t |
| 2 | Scrambled 1 (SEQ ID NO: 586) | MUC16 CAR-mbIL15-HER1t |
| 3 | Scrambled 2 (SEQ ID NO: 582) | MUC16 CAR-mbIL15-HER1t |
| 4 | miR204 PD1 + miR206 PD1 | MUC16 CAR-mbIL15-HER1t |
| 5 | miR17 TIGIT | MUC16 CAR-mbIL15-HER1t |
| 6 | miR150 TIGIT + miR206 PD1 | MUC16 CAR-mbIL15-HER1t |
| 7 | miR204 PD1 + miR206 PD1 + | MUC16 CAR-mbIL15-HER1t |
| | miR17 TIGIT | |
| 8 | N/A | MUC16 CAR + HER1t |

The data demonstrates the specificity of PD-1 checkpoint inhibitor miRNA targeting the intended sequence. CAR constructs that expressed the scrambled miRNA (Constructs 2 and 3) along with the CAR construct that did not express mbIL-15 (construct #8) did not show any decrease in PD-1 expression whereas CAR constructs that contain PD-1 miRNA (construct 4) or a combination of PD-1 and TIGIT miRNA (constructs 6 and 7) did show a decrease in PD-1 expression. On the other hand, the CAR construct containing only a TIGIT miRNA sequence (construct 5) did not show any decrease in PD-1 mRNA expression levels further demonstrating the specificity in targeting.

### Example 3. Downregulation of Targeted mRNA

Primary human T cells were transfected with a vector encoding CD33-specific CAR or a vector encoding MUC16-specific CAR. The vectors comprise a synthetic intron containing miRNA sequences that target PD-1, PD-1 and/or TIGIT or a non-targeting scrambled control miRNA. T cell cultures were in vitro expanded using antigen presenting cells with cognate tumor antigen that were K562 cells modified to express either CD33 or MUC16 plus other co-stimulatory molecules (based on “Clone 1”) and at a ratio of 1:1 (AaPC:T cell). The generated CAR+ cells were then stimulated with anti-CD3/anti-CD28 beads (1:1 bead:T cell ratio) in the absence of cytokines for 48 hrs. RNA was isolated using Qiagen kits (AllPrep Universal DNA/RNA/miRNA kit #80224) as per manufacturer's protocol and utilized for the Human PanCancer Immune gene set panel Kit from Nanostring. Briefly, the RNA was hybridized with capture and reporter probe sets and samples processed then hybridized to the slide using the nCounter Prep Station and transcript counts were generated by the nCounter Digital Analyzer according to the manufacturer's protocol. Data validation, QC and normalization were performed by nSolver software (Nanostring Technologies).

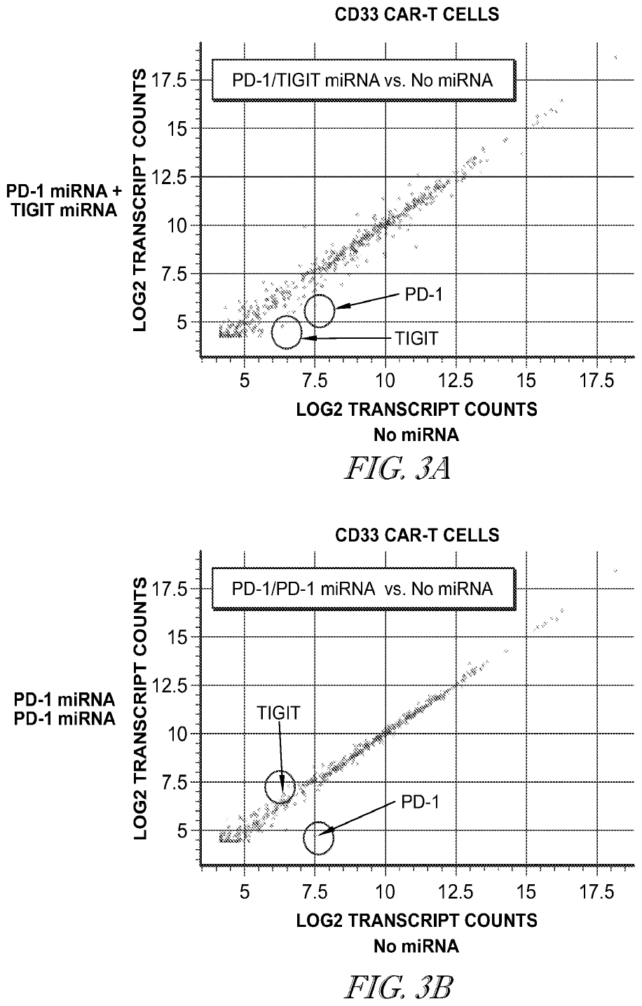

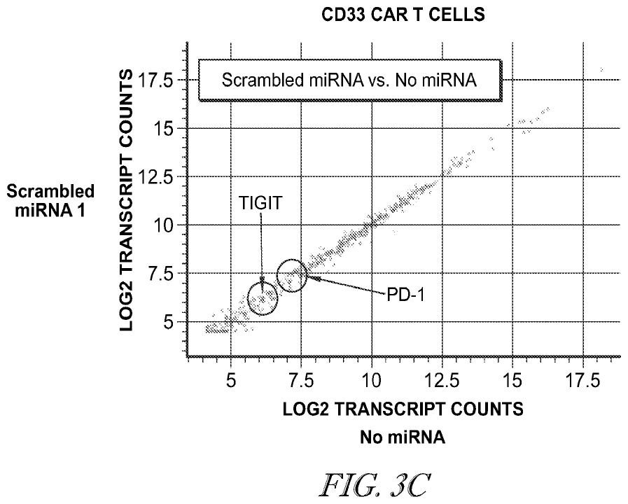

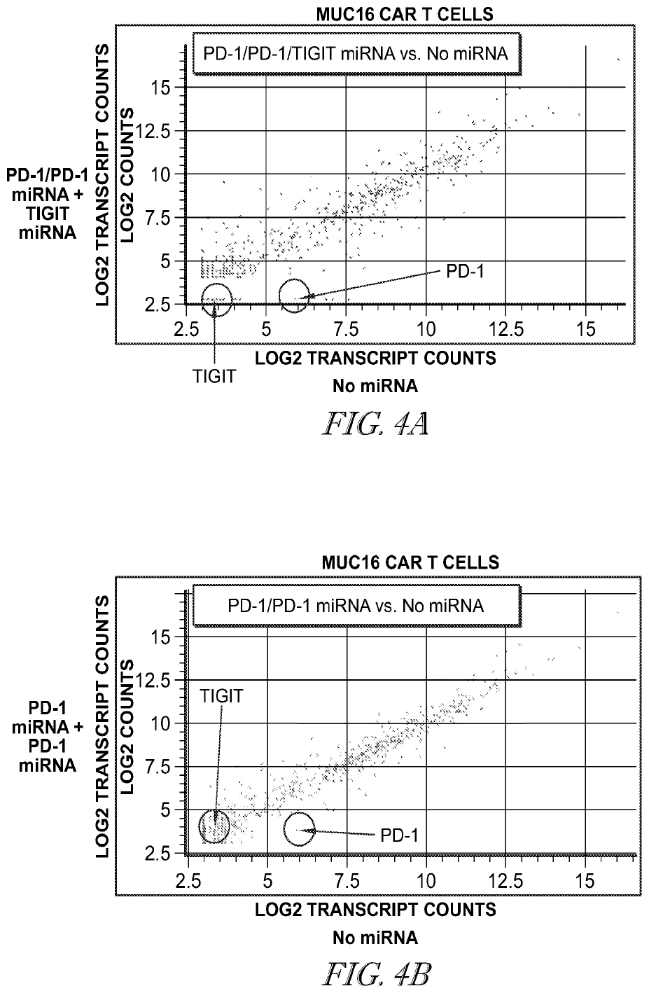

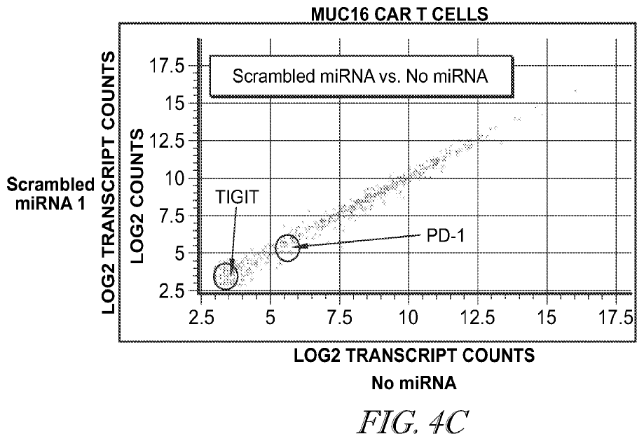

The distribution of the transcript count data on the graph would be a line (from lower left to top right corner) with the slope of 1 if the samples were identical. Data points falling off this line represent variations in transcript counts between the two samples. Counts that were found below the main line represent reduced expression whereas counts above the main line represent increased expression to the sample being compared. The transcript targeted by the miRNA, either PD-1 or TIGIT expressed in the CAR-T cells, specifically showed lower expression of the transcripts of their respective targeted genes compared to the CAR-T cells without the miRNA. See FIG. 3A-B. In addition, to further demonstrate the specificity of targeting, the CAR-T cells with the scrambled control miRNA had no alterations to the transcript levels of either PD-1 or TIGIT, as the data distribution in this graph remained close to the diagonal indicating very little variation in transcript levels. See FIG. 3C. Similar results were observed in T cells transfected with the MUC16 CAR vector comprising a synthetic intron containing miRNA sequences that target PD-1, PD-1 and/or TIGIT (see FIG. 4A-C).

### Example 4. Enhancement of Tumor Cell Cytotoxicity Effect of miRNA MUC16 CAR-T Cells

Primary human T cells were transfected with a vector encoding MUC16-specific CAR and miRNA sequences that target two sequences within the PD-1 transcript, or with a vector that encodes the MUC16-specific CAR but not an miRNA. CAR-T cells used in this assay were in vitro expanded, normalized for CAR expression then seeded in triplicates with GFP+K562 cells expressing MUC16 (“tumor cells”) in 96 well plates at the 3:1 E:T ratios. The plate was loaded into the IncuCyteS3 instrument and 4 images per well were taken every 2 hr for 7 days. The IncuCyte Software was used to analyze the data and normalize the GFP+ cell counts/image to the 0 hr time point.

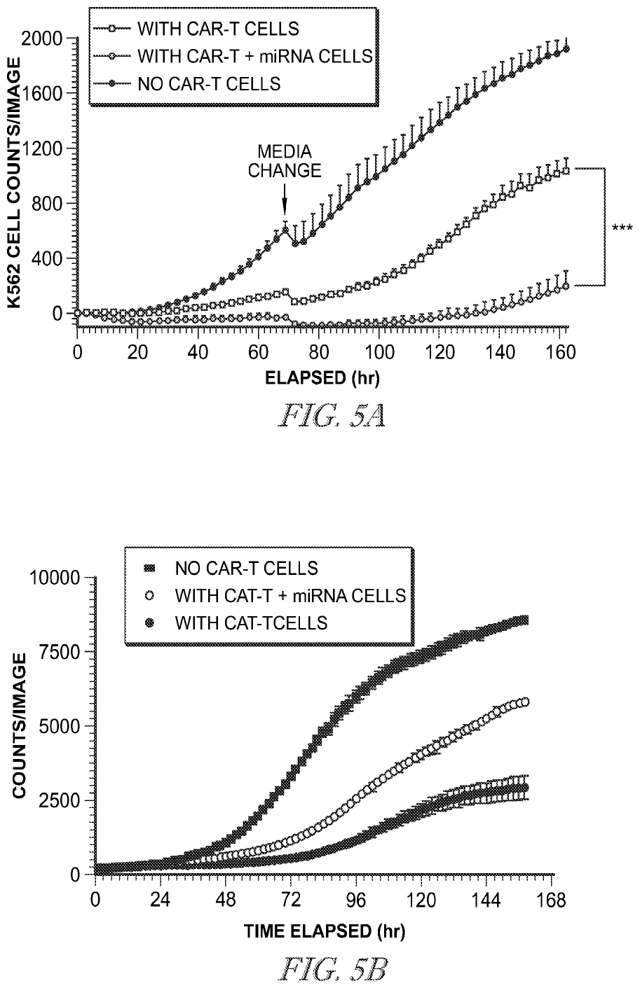

The outgrowth assay determines the rate of target cell growth in the presence or absence of CAR-T cells over the course of 7 days in culture. The lower target cell count in cultures containing CAR-T cells indicate the CAR-T cytolytic activity. FIG. 5A depicts the difference between the tumor target cells only (black circle, filled) and the cells expressing the MUC16-specific CAR only (open square), demonstrating the killing capacity of the MUC16 CAR-T cells. The cells further expressing miRNA targeting PD-1 (grey circle filled) further demonstrates improved cytolytic activity, based upon the sustained low GFP+ counts over the time course evaluation, compared to the cells expressing MUC16 specific CAR only (open square). This data demonstrates the enhanced cytolytic activity of CAR-T cells containing PD-1 targeting miRNA.

A similar experiment was conducted utilizing GFP+K562 cells expressing MUC16, PD-L1, and CD155. As shown in FIG. 5B, the difference between the tumor target cells only (square, filled) and cells expressing the CAR only (circle, open), demonstrates the killing capacity of the MUC16 CAR-T cells. The CAR-T cells incorporating miRNAs targeting both PD-1 and TIGIT (circle, filled) further demonstrates improved cytolytic activity, based upon the sustained low GFP+ counts over the time course evaluation, compared to the MUC16 CAR-T cells only (circle, filled). This data demonstrates the enhanced cytolytic activity of CAR-T cells containing 3 targeting miRNAs (2 for PD-1 and 1 for TIGIT) in a single construct.

### Example 5. Improvement of Cytokine Expression

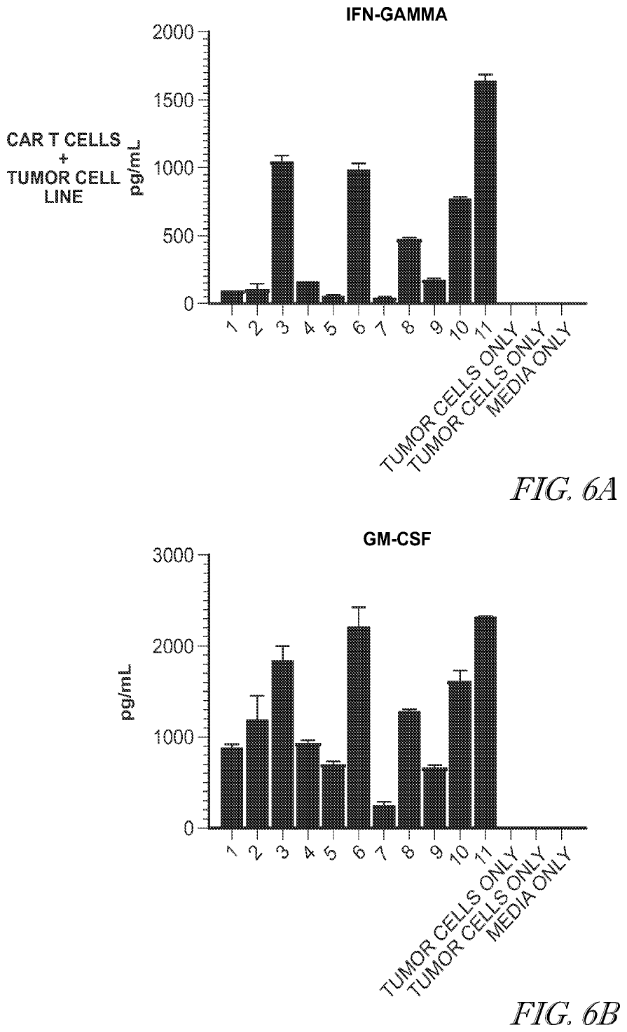

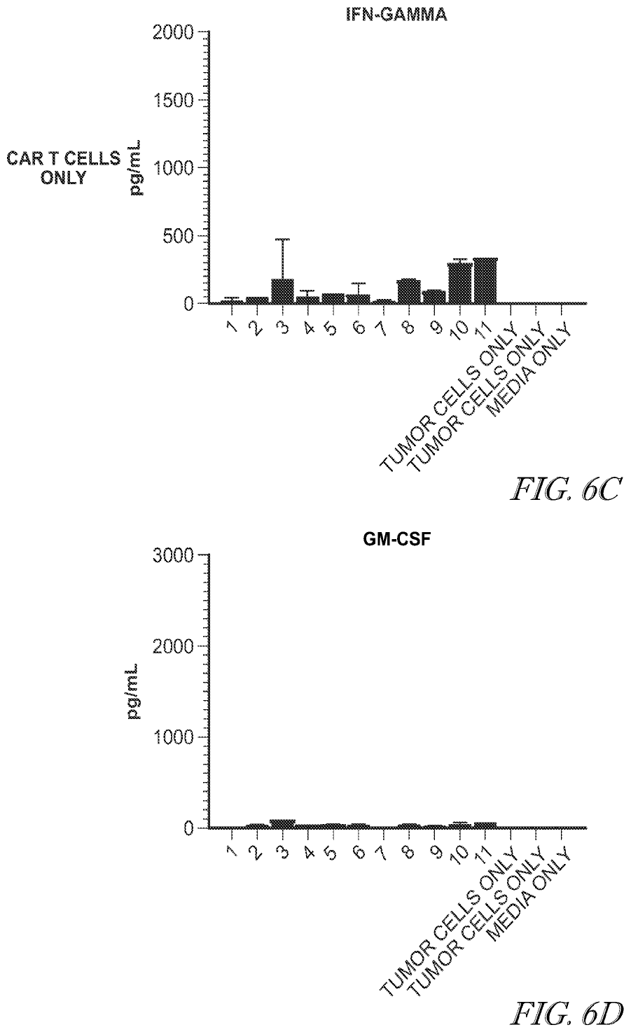

Primary human T cells were transfected with vectors encoding MUC16-specific CAR but not miRNA targeting sequences, and vectors encoding MUC16-specific CAR and single or combinations of miRNA targeting sequences of PD-1 and TIGIT (see Table 7). In vitro expanded CAR-T cells were in vitro expanded, normalized for CAR expression then seeded in triplicates in 96 well plates with K562 tumor target cells expressing truncated MUC16 9MUC16t) at an effector to target ratio of 1:1 or the CAR-T cells were cultured in media alone. Culture supernatants were collected after co-culturing for 3 days. Culture supernatants were collected and interferon-gamma (IFNγ) and granulocyte/macrophage-colony stimulating factor (GM-CSF) was assessed by multiplex cytokine analysis (Luminex) according to manufacturer's protocol. Shown in FIG. 6 is the mean±SD from duplicate wells.

For the CAR-T cell constructs cultured in media only had basal levels of IFNγ and GM-CSF detected. No cytokines were observed from the use of target cells or media only. The supernatants following co-culture of MUC16 CAR-T cells only (Vector 1) with the tumor cells provided a baseline value for expression levels of IFNγ and GM-CSF. With the inclusion of checkpoint inhibitor miRNA into the CAR constructs, improved cytokine expression may be observed, particularly through inhibition via the PD-1 pathway. In the constructs that contained dual PD-1 (Construct #3) or the combination of a single PD-1 and TIGIT miRNA (Construct #6) or dual PD-1 and a TIGIT miRNA (Constructs #10 and 11) provided higher levels of cytokine expression.

TABLE 7
Description of Constructs #1-11 in FIGS. 6A-D
(sequences of miRNA are as described in Table 4)

| Con- | | |
|---|---|---|
| structs | miRNA | Effector genes |
| 1 | N/A | MUC16 CAR-mbIL15-HER1t |
| 2 | Scrambled control 1 | MUC16 CAR-mbIL15-HER1t |
| 3 | miR204 PD1 + miR206 PD1 | MUC16 CAR-mbIL15-HER1t |
| 4 | miR17 TIGIT | MUC16 CAR-mbIL15-HER1t |
| 5 | miR17 TIGIT + miR206 PD1 | MUC16 CAR-mbIL15-HER1t |
| 6 | miR150 TIGIT + miR206 PD1 | MUC16 CAR-mbIL15-HER1t |
| 7 | miR17 TIGIT + miR204 PD1 + | MUC16 CAR-mbIL15-HER1t |
| | miR206 PD1 | |
| 8 | miR17 TIGIT + miR204 PD1 + | MUC16 CAR-mbIL15-HER1t |
| | miR206 PD1extended v1 | |
| 9 | miR17 TIGIT + miR204 PD1 + | MUC16 CAR-mbIL15-HER1t |
| | miR206 PD1extended v2 | |
| 10 | miR204 PD1 + miR206 PD1 + | MUC16 CAR-mbIL15-HER1t |
| | miR17 TIGIT | |
| 11 | miR204 PD1 + miR206 PD1 + | MUC16 CAR-mbIL15-HER1t |
| | miR17 TIGITextended v1 | |

### Example 6. Tumor Burden in Treated Mice

Non-obese diabetic/severe combined immunodeficiency (NOD/SCID) gamma mice (NSG) mice were intraperitoneally implanted with fLUC-GFP+SK-OV-3 tumor cells expressing MUCt on Day 0. Tumor burden was monitored in these mice throughout the study using an in vivo imaging system (IVIS) by luminescence with an IVIS Spectrum instrument (Perkin Elmer). IVIS data was analyzed using the Living Image Software (Version 4.1) based upon a defined region of interest to obtain total flux values (photons/sec). Prior to administration of the CAR-T cells, mice were randomized based upon tumor burden and body weight into the different groups then administered the test articles on Day 6 All CARs tested expressed the MUC16-specific CAR along with mbIL15 and HER1t and are referred to as MUC16 CAR. All test articles were normalized to a 0.5×10⁶ CAR-T cells/mouse and administered intraperitoneally. IVIS imaging was performed twice/week to monitor overall tumor burden in the mice. Data shown is the mean±SEM from n=4-8 mice/group.

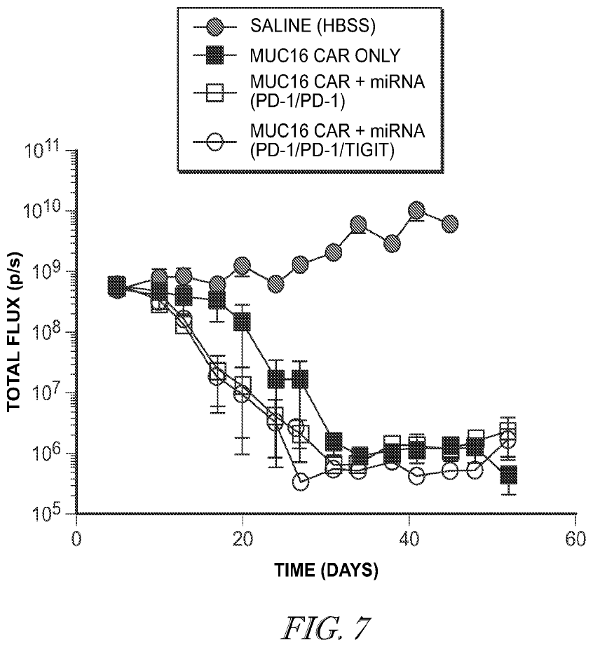

As shown in FIG. 7, mice given saline only (gray-filled circles), had continuous tumor growth as evidenced by the increasing total flux levels observed throughout the course of the study. Eventually, these mice succumbed to the tumor burden and were euthanized. Mice given the MUC16 CAR only (black-filled squares) were able to control tumor burden. CAR-T cells expressing the checkpoint inhibitor miRNA to PD-1 and TIGIT within the constructs (open squares and circles) were found to maintain anti-tumor activity, based upon the decrease in tumor flux values to background levels. In addition, the CAR-T cells expressing the checkpoint inhibitor miRNA to PD-1 and TIGIT showed a faster time frame and the rate of tumor burden decrease compared to the MUC16 CAR only construct.

### Example 7. In Vivo Phenotyping Experiment

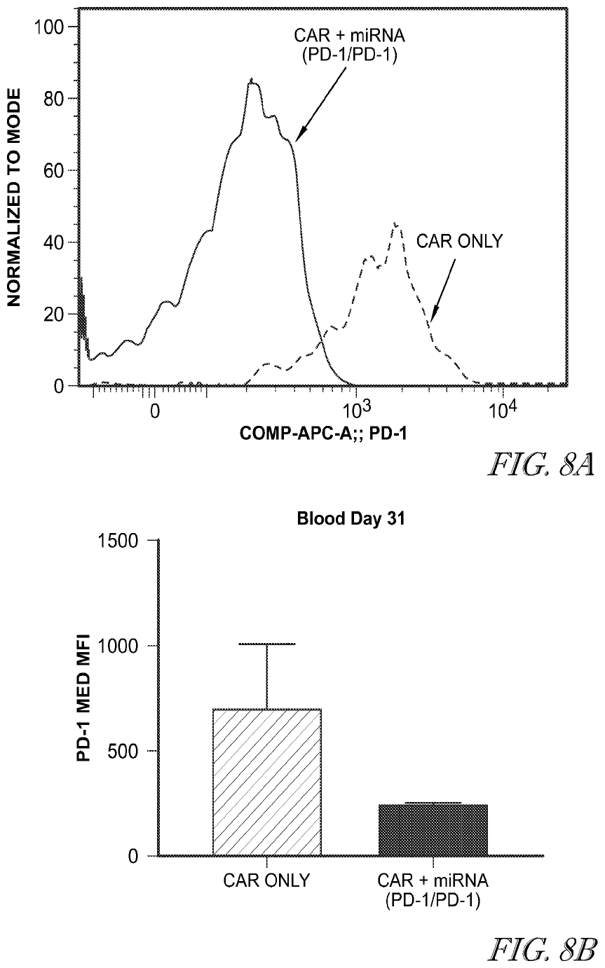

SKOV-3/MUC16 tumor bearing mice were administered CAR-T cells (expressing MUC16-specific CAR, mbIL15 and HER1t) of either CAR only or CAR with PD-1/PD-1 miRNA on Study Day 6. Whole blood from mice were taken on Study Day 31 for phenotypic evaluation by flow cytometry of the administered CAR-T cells. Briefly, cocktails of fluorescently conjugated antibodies were used to stain the whole blood samples then concurrently fixed along with red blood cells lysis using a one-step Fix/Lyse buffer. The fixed samples were read on the flow cytometer (BD LSRFortessaX-20) instrument. CAR-T cells were identified based upon gating of hCD45/CD3+/HER1t+ expression. In, FIG. 8A, the sample of CAR only (dotted line) shows high PD-1 expression, whereas the CAR with PD-1/PD-1 miRNA (solid line) shows a significant decrease in the level of PD-1 expression detected. To further quantify the reduced expression of PD-1 detected on the CAR+miRNA (PD-1/PD-1) group, the median fluorescent intensity (MFI) was examined (FIG. 8B). The mean MFI of PD1 expression in mice given CAR-T cells (stripe bar) only was ˜709 whereas the mean MFI of the CAR-T cells with a PD-1/PD-1 miRNA (solid bar) was reduced down to ˜236. The mean±SEM from 5-8 mice is shown.

### Example 8. Specific PD-1 and TIGIT Downregulation

SKOV-3 tumor bearing mice were administered CAR-T cells (expressing MUC16-specific CAR (“MUC16 CAR”), mbIL15 and HER1t) of either CAR only or CAR with different checkpoint miRNA inhibitors (PD-1 and TIGIT) on Study Day 6. See Table 8.

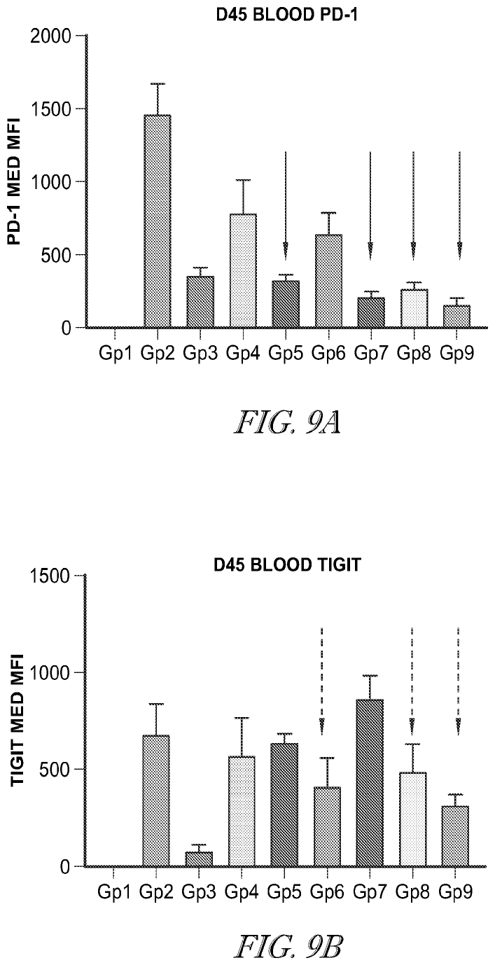

TABLE 8
Description of Groups #1-9 in FIGS. 9A-B

| Group | CAR-T Cells |
|---|---|
| 1 | Saline control |
| 2 | MUC16 CAR/mbIL15/HER1t |
| 3 | MUC16 CAR/mbIL15/HER1t + anti-PD1 |
| 4 | MUC16 CAR/mbIL15/HER1t + scrambled miRNA 2 |
| 5 | MUC16 CAR/mbIL15/HER1t + miR206 PD1 |
| 6 | MUC16 CAR/mbIL15/HER1t + miR17 TIGIT |
| 7 | MUC16 CAR/mbIL15/HER1t + miR204 PD1/miR206 PD1 |
| 8 | MUC16 CAR/mbIL15/HER1t + miR150 TIGIT/miR206 PD-1 |
| 9 | MUC16 CAR/mbIL15/HER1t + miR204 PD1/miR206 PD1/ |
| | miR150 TIGIT |

Whole blood from mice were taken on Study Day 45 (D45) for phenotypic evaluation by flow cytometry of the administered CAR-T cells. Briefly, cocktails of fluorescently conjugated antibodies, which included specific antibodies for human PD-1 and human TIGIT, were used to stain the whole blood samples then fixed using a one step Fix/Lyse buffer. The fixed samples were read on the flow cytometer (BD LSRFortessaX-20) instrument. CAR-T cells were identified in the mouse based upon gating of hCD45/CD3+/HER1t+ expression. To further evaluate the specificity of miRNA used in the CAR vector for the checkpoint inhibitor, the median fluorescent intensity (MFI) for expression of PD-1 and TIGIT was analyzed (FIGS. 9A and B). The MFI for PD-1 is shown on the left and the MFI for TIGIT is shown on the right for a quantitative assessment of the expression levels for the same set of vectors. As shown in FIG. 9A, reduced expression of PD-1 was seen on the CAR-T cells for the groups indicated with the down arrows (solid line for PD-1), which are constructs with a PD-1 miRNA (single, double and in combination with another miRNA checkpoint inhibitor), when compared to the CAR vector only. On the right side (downward dashed arrows) highlights cell populations with a reduced expression of TIGIT expression seen on the CAR-T cells. The downregulated expression of TIGIT corresponded to the samples that contained a miRNA for TIGIT (either as a single or in combination with other checkpoint miRNA inhibitors). The mean±SEM from 5-8 mice is shown.

### Example 9. Expression of PD1-Targeting miRNAs in ROR1-Targeted CAR-T Cells

Briefly, miRNA-expressing ROR1 UltraCAR-T cells or control ROR1 UltraCAR-T cells were generated from T cells from five donors. PanT cells from five healthy donors were transfected with an indicated transposon vector (VVN-5355 or VVN-5351) plus SB11 transposase vector, and expanded by weekly stimulations for 4 weeks (˜35 days before adding beads) with ROR1 antigen presenting cells. Following a rest period of 7-8 days, UltraCAR-T cells were activated with CD3/CD28 dynabeads (1:1 bead:T cell ratio, T cells at 1×10⁶ cells/mL) for 48 hours prior to RNA harvest, 7-8 days after the last AaPC stimulation. Expression of PD1-targeting guide miRNAs and the impact on PD1 mRNA expression were verified by RT-qPCR. Small RNAseq, which is an established method to identify the predicted and alternate miRNA sequences that may arise from a pri-miRNA (Borel et al, 2018; Miniarikova et al, 2016), was performed to compare expression levels of the PD1-targeting guide miRNAs to additional small RNA species, such as passenger miRNA, that may be generated from the PD1 silencer module. Small RNAseq was also used to assess potential changes on global endogenous miRNA expression (Mueller et al, 2012). RNAseq analysis was performed to evaluate global transcript expression and to identify changes in molecular pathway signaling or off-target gene silencing attributed to the PD1 silencer. In silico miRNA target prediction was performed using the miRanda algorithm to identify most likely targets of miRNAs generated from the PD1 silencer module. Expression of the predicted target genes was evaluated by RNAseq. Details of each method is included in the sections below.

To characterize expression of miRNAs encoded by the PD1 silencer and impact on PD1 transcript levels, RT-qPCR and small RNAseq were performed. To characterize changes in specific genes or cellular pathways, RNAseq was performed.

Nucleic acids were purified from cell pellets using Qiagen's AllPrep DNA/RNA/miRNA Universal kit (Cat #80224) following the manufacturers protocol. Total RNA was eluted in 50 uL nuclease-free water and concentration measured on a Nanodrop™ 2000 spectrophotometer.

To quantify the expression of PD1 guide and passenger miRNAs, total RNA was used as input for cDNA synthesis using Qiagen's miRCURY LNA RT Kit (#339340). Per the manufacturer's protocol, cDNA was diluted 1:60 with nuclease-free water and 3 μL of the diluted cDNA was used as input for qPCR using miRCURY LNA SYBR Green PCR Kit (Qiagen #339345) with custom miRCURY LNA primers specific to the two PD1 guide miRNAs. An endogenous miRNA, hsa-let−7a-5p, was quantified as a reference small RNA to allow for input normalization (Qiagen product #339306 with custom #YP00205727). Samples were run in a 384 well format on a QuantStudio 6 Flex instrument. Relative quantification (dCT) calculations were performed in Microsoft Excel and data graphed in GraphPad Prism 9. Calculations for dCT were performed on each technical replicate as follows:

$$dCT = CT ( guide miRNA ) - CT ( hsa - let - 7 a )$$

$$ddCT = dCT ( miRNACART replicate ) - dCT ( average of VVN - 5355 technical replicates )$$

$$Fold Change = 2 \hat{} - ddCT$$

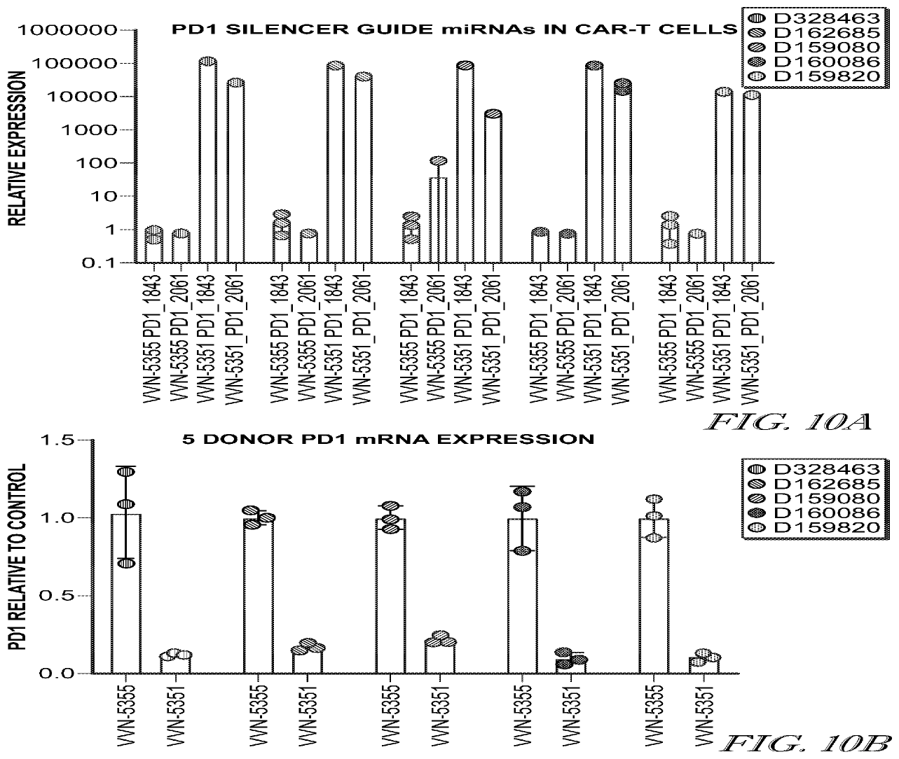

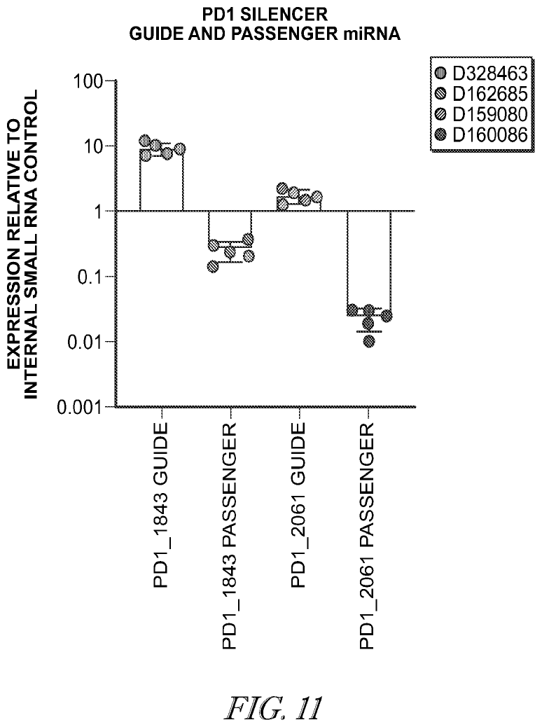

The VVN-5355 ROR1 UltraCAR-T control sample serves as the reference control sample for comparison to miRNA expressing ROR1 UltraCAR-T cells within each donor set. Average fold change and standard deviation of technical replicates was calculated and reported in FIG. 10A.

To quantify and compare the production of guide and passenger miRNAs originating from the PD1 silencing module, RT-qPCR was performed as described above, except this second experiment included primer assays to detect the passenger miRNAs as well as an additional endogenous reference small RNA, RNU1A1. Expression calculations were performed to compare expression of each guide or passenger strand mature miRNA relative to the average of the endogenous control small RNAs as follows:

$$\begin{matrix}dCT = CT ( mature miRNA ) - CT ( average of hsa - let - 7 a and RNU 1 A 1 ) \\ Fold change = 2 \hat{} - dCT\end{matrix}$$

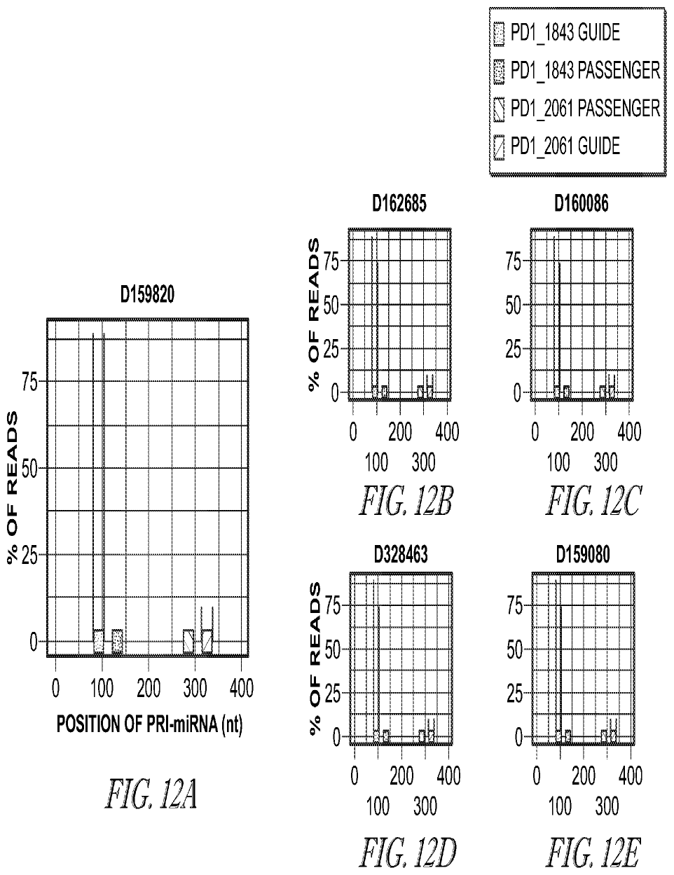

Average fold change was calculated from three technical replicates. These values are plotted in FIG. 11 with mean and standard deviation shown for the donor sample sets tested.

To quantify the expression of endogenous PD1 mRNA, cDNA synthesis was performed using a final RNA concentration of 5 ng/μL using Invitrogen's SuperScript IV VILO Master Mix with ezDNase enzyme kit (#11766050). Multiplex Taqman qPCR was performed using Invitrogen's TaqMan MastAdvanced Master Mix (#4444963) with 1 microliter of cDNA and Invitrogen (ThermoFisher Scientific) Taqman assays. Human PD1 Taqman assay (Invitrogen #Hs00169472_m1) was FAM labeled and the internal normalizer gene, ACTb (Invitrogen #Hs99999903_m1), which was VIC-labelled. Samples were run in a 384 well format on a QuantStudio 6 Flex instrument. Relative quantification (dCT) calculations were performed in Microsoft Excel as described above and data graphed in GraphPad Prism 9.

TABLE 9
Test and Control Articles

| Sample Name | Description | Designation |
|---|---|---|
| VVN-5355 | CAR-T cells generated using Sleeping Beauty transposon | Control |
| | plasmid VVN-5355, which contains a transgene cassette | |
| | to express ROR1-specific CAR + mbIL-15 + HER1t. | |
| VVN-5351 | CAR-T cells generated using Sleeping Beauty transposon | Test Article |
| | plasmid VVN-5351, which contains a transgene cassette | |
| | to express ROR1-specific CAR + mbIL-15 + HER1t plus | |
| | a PD1 silencer module. | |

The PD1 Silencer Module is designed to produce two mature guide miRNAs that bind to the PD1 transcript to silence PD1 expression. Expression of the two PD1-targeting guide miRNAs, referred to as PD1_1843 and PD1_2061, was confirmed in miRNA expressing ROR1 UltraCAR-T cells generated from multiple donors (FIG. 10A). A corresponding reduction in PD1 mRNA expression was verified in the miRNA-expressing ROR1 UltraCAR-T cells from all donors tested (FIG. 10B). This result demonstrated that the PD1 Silencer module produces the intended guide miRNAs and functions as designed.

To reduce the risk of silencing genes other than PD1, the PD1 silencer module was designed using pri-miRNA scaffolds that preferentially produce PD1-targeting guide miRNA over the non-targeting passenger miRNA. Both guide and passenger mature miRNAs were quantified from miRNA expressing ROR1 UltraCAR-T cells by RT-qPCR, which verified PD1 targeting guide miRNAs as the predominant species compared to non-targeting passenger strand miRNA (FIG. 4). The strong processing preference for the PD1 targeted guide miRNA was confirmed by small RNAseq, with 99.7% of reads mapping to the PD1 Silencer Module matching the intended PD1 targeting guide miRNAs (FIGS. 12A-E). Furthermore, the start and stop position of the miRNAs was as expected, with miRNAs of 21-23 nucleotides detected that had the same 5′ end and variable length at the 3′ end (FIGS. 12A-E). The extremely low incidence of passenger strand miRNAs and lack of unexpected small RNAs generated by aberrant RNA processing substantially reduced the risk for off-target gene silencing.

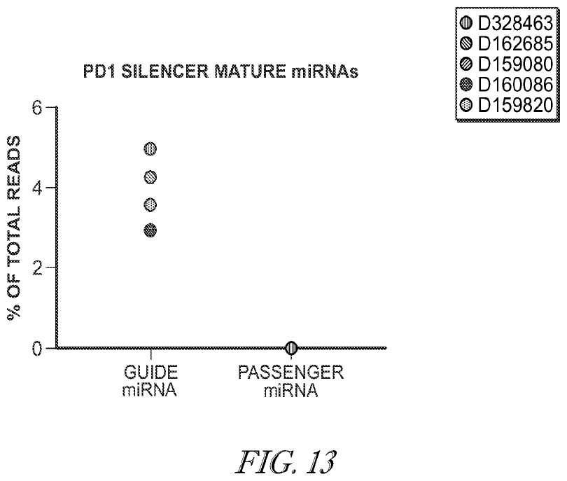

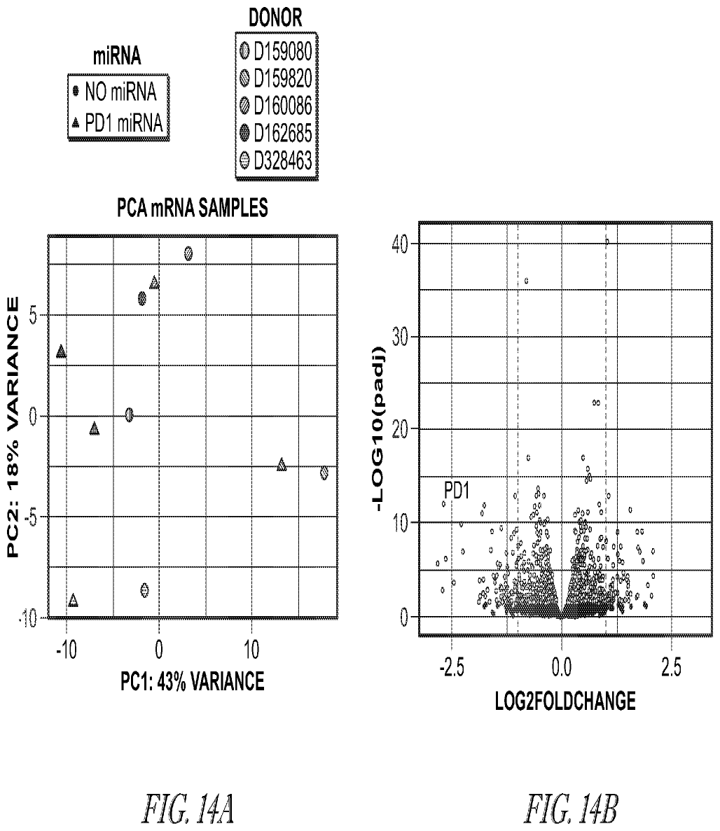

To ensure that expression of the PD1 Silencer module does not overwhelm the internal cellular RNAi machinery, endogenous miRNA counts were compared from miRNA expressing ROR1 UltraCAR-T cells vs control ROR1 UltraCAR-T cells. Examination of the top twenty expressed endogenous miRNAs demonstrated no statistically significant changes in expression across the samples (Table 11). Furthermore, the mature miRNAs generated from the PD1 silencer module accounted for approximately 4% of all quantified small RNAs (FIG. 13). The data indicated that expression of miRNAs from the PD1 silencer module did not saturate the cellular RNAi machinery and had no detectable impact on global endogenous miRNA expression.

TABLE 10
Small RNAseq Comparison of
Guide and Passenger miRNA Counts

| Mature miRNA_Length | Normalized | % |
|---|---|---|
| (Sequence) | Mean | Total |
| Guide PD1_204_21 nt | 486,432.3 | 88.2% |
| (TTCAGGAATGGGTTCCAAGGA; | | |
| SEQ ID NO: 704) | | |
| Guide PD1_204_22 nt | 12,763.5 | |
| (TTCAGGAATGGGTTCCAAGGAT; | | |
| SEQ ID NO: 72) | | |
| Guide PD1_204_23 nt | 59,759.4 | |
| (TTCAGGAATGGGTTCCAAGGATG; | | |
| SEQ ID NO: 705) | | |
| Passenger PD1_204_21 nt | 380.6 | 0.1% |
| (TCCTGGAAGCTATTCCTGACG; | | |
| SEQ ID NO: 706) | | |
| Passenger PD1_204_22 nt | 20.2 | |
| (TCCTGGAAGCTATTCCTGACGT; | | |
| SEQ ID NO: 707) | | |
| Passenger PD1_204_23 nt | 54.7 | |
| (TCCTGGAAGCTATTCCTGACGTT; | | |
| SEQ ID NO: 708) | | |
| Guide PD1_206_21 nt | 3,752.7 | 11.5% |
| (TATAATATAATAGAACCACAG; | | |
| SEQ ID NO: 709) | | |
| Guide PD1_206_22 nt | 49,181.3 | |
| (TATAATATAATAGAACCACAGG; | | |
| SEQ ID NO: 74) | | |
| Guide PD1_206_23 nt | 19,695.3 | |
| (TATAATATAATAGAACCACAGGA; | | |
| SEQ ID NO: 710) | | |
| Passenger PD1_206_21 nt | 1,581.0 | 0.3% |
| (TGTGGTTCTGTTATATCCATA; | | |
| SEQ ID NO: 711) | | |
| Passenger PD1_206_22 nt | 3.1 | |
| (TGTGGTTCTGTTATATCCATAT; | | |
| SEQ ID NO: 712) | | |
| Passenger PD1_206_23 nt | 13.2 | |
| (TGTGGTTCTGTTATATCCATATA; | | |
| SEQ ID NO: 713) | | |

TABLE 11
Top 20 Expressed Endogenous Mature
miRNAs Detected by Small RNAseq

| | | Log2 Fold | P-value |
|---|---|---|---|
| Endogenous miRNA | Base Mean | Change | Adjusted |
| hsa-miR-181a-5p | 796180.25 | 0.11 | 0.7948 |
| hsa-miR-191-5p | 421094.34 | −0.19 | 0.5622 |
| hsa-miR-21-5p | 386727.78 | 0.03 | 0.9411 |
| hsa-miR-92a-3p | 368149.34 | 0.26 | 0.2298 |
| hsa-miR-142-5p | 345451.62 | 0.08 | 0.8549 |
| hsa-miR-146b-5p | 286127.09 | 0.24 | 0.5471 |
| hsa-miR-16-5p | 261883.66 | 0.00 | 0.9954 |
| hsa-miR-146a-5p | 229059.23 | −0.27 | 0.4569 |
| hsa-let-7f-5p | 188491.61 | 0.16 | 0.6018 |
| hsa-miR-22-3p | 123453.58 | −0.01 | 0.9899 |
| hsa-miR-26a-5p | 110714.77 | 0.19 | 0.5166 |
| hsa-let-7a-5p | 104253.31 | 0.00 | 0.9921 |
| hsa-miR-155-5p | 72533.08 | −0.29 | 0.2298 |
| hsa-miR-181b-5p | 67527.35 | 0.12 | 0.7687 |
| hsa-let-7i-5p | 64631.72 | 0.30 | 0.2116 |
| hsa-miR-21-3p | 52519.27 | 0.25 | 0.5358 |
| hsa-miR-186-5p | 52479.56 | −0.20 | 0.4189 |
| hsa-miR-25-3p | 50411.16 | 0.02 | 0.9696 |
| hsa-miR-27a-3p | 47911.68 | −0.10 | 0.7818 |
| hsa-let-7g-5p | 47159.42 | 0.01 | 0.9811 |

### Example 10: PD1 Silencer Module Specifically Reduces Expression of PD-1

An in silico miRNA target prediction algorithm, miRanda (Betel et al, 2008; Betel et al, 2010), was used to predict the most likely target transcripts for the guide and passenger miRNAs generated from the PD1 silencer module. The algorithm assigned a score for each potential miRNA target gene, with higher scores indicating higher likelihood of silencing by the input miRNA sequence. A summary of the top ten hits for each mature miRNA generated from the PD1 silencing module is listed in Table 12. PD1 is the only gene with perfect homology to any of the guide and passenger miRNAs and has the highest predicted miRanda score of any potential target gene. Expression of each predicted target gene was characterized from the RNAseq differential expression data set. PD1 was the most downregulated of all predicted target genes, with a log 2 fold change (LFC) of −2.63 (˜84% PD1 reduction in miRNA expressing ROR1 UltraCAR-T cells compared to control ROR1 CAR-T cells) and a highly significant adjusted p-value. The expression of other predicted target genes was unchanged; those genes with adjusted p-values below 0.05 had LFC in the range of −0.33 to 0.22, which is an expression decrease or increase of <20%. One exception is a weakly predicted target gene of PD1_2061 guide miRNA, HDAC9, which has a LFC of −1.51. HDAC9 is a transcriptional repressor that is mechanistically linked to PD1 expression through BCL6 (Xie et al, 2017; Gil et al, 2016). It is likely that HDAC9 is not directly targeted by PD1_2061 guide miRNA, and that the reduction in HDAC9 is an indirect effect of reduced PD1 expression.

TABLE 12
In silico Predicted miRNA Target Genes

| | Target | miRanda | free | % | Base | log2 | | P | Adjusted |
|---|---|---|---|---|---|---|---|---|---|
| miRNA | Gene | Score | energy | Identity | Mean | FoldCha | lfcSE | value | P Value |
| PD1_1843 | PD1 | 200 | −41.9 | 100.0 | 84 | −2.63 | 0.293 | 2.51E−19 | 2.64E−16 |
| miR204 | MIER3 | 184 | −32.1 | 84.2 | 1073 | 0.01 | 0.063 | 0.9104 | 0.9688 |
| Guide | SLFN12L | 182 | −34.2 | 89.5 | 1404 | −0.22 | 0.068 | 0.0015 | 0.0134 |
| miRNA | FMO4 | 180 | −28.5 | 84.2 | 15 | −0.18 | 0.461 | 0.6975 | NA |
| | GIGYF1 | 180 | −33.5 | 84.2 | 2749 | −0.07 | 0.046 | 0.1123 | 0.3211 |
| | FAM83G | 180 | −31.5 | 84.2 | 171 | 0.33 | 0.144 | 0.0214 | 0.1026 |
| | PPRC1 | 180 | −31.2 | 84.2 | 2679 | −0.18 | 0.045 | 0.0001 | 0.0011 |
| | PRMT9 | 180 | −29.1 | 84.2 | 335 | 0.09 | 0.100 | 0.3704 | 0.6432 |
| | MGAT4B | 179 | −29.3 | 88.9 | 4480 | −0.21 | 0.063 | 0.0007 | 0.0076 |
| | BRWD1 | 179 | −25.0 | 88.9 | 1816 | 0.05 | 0.052 | 0.3581 | 0.6328 |
| PD1_1843 | DOCK9 | 179 | −28.5 | 83.3 | 1239 | −0.12 | 0.077 | 0.1240 | 0.3428 |
| miR204 | NFYA | 179 | −27.5 | 83.3 | 1615 | −0.02 | 0.052 | 0.7446 | 0.8911 |
| Passenger | IFIT5 | 178 | −30.4 | 89.5 | 379 | −0.11 | 0.102 | 0.2629 | 0.5351 |
| miRNA | OTUB2 | 178 | −33.7 | 84.2 | 248 | −0.32 | 0.131 | 0.0154 | 0.0805 |
| | UBIAD1 | 177 | −32.1 | 88.9 | 1064 | −0.07 | 0.066 | 0.3085 | 0.5859 |
| | MYO15A | 176 | −29.5 | 93.3 | 11 | −0.25 | 0.510 | 0.6263 | NA |
| | ADRB2 | 175 | −27.1 | 83.3 | 62 | 0.61 | 0.264 | 0.0206 | 0.0997 |
| | HIC2 | 175 | −28.5 | 83.3 | 312 | −0.01 | 0.102 | 0.8962 | 0.9621 |
| | NECTIN3 | 175 | −33.5 | 72.2 | 662 | 0.05 | 0.081 | 0.5587 | 0.7864 |
| | TBC1D10C | 175 | −27.3 | 77.8 | 7562 | 0.03 | 0.041 | 0.4409 | 0.7056 |
| PD1_2061 | PD1 | 200 | −34.3 | 100.0 | 84 | −2.63 | 0.293 | 2.51E−19 | 2.64E−16 |
| miR206 | LCOR | 180 | −16.7 | 84.2 | 1321 | −0.19 | 0.064 | 0.0029 | 0.0230 |
| Guide | STARD4 | 179 | −16.7 | 88.9 | 6871 | −0.18 | 0.055 | 0.0013 | 0.0120 |
| miRNA | UQCRC2 | 179 | −19.1 | 81.0 | 4759 | 0.03 | 0.043 | 0.4641 | 0.7216 |
| | EXD2 | 178 | −17.5 | 88.2 | 58 | 0.18 | 0.265 | 0.5055 | 0.7511 |
| | NOL8 | 177 | −18.6 | 88.9 | 2190 | −0.08 | 0.050 | 0.1007 | 0.2996 |
| | ATP23 | 176 | −18.6 | 84.2 | 318 | 0.22 | 0.100 | 0.0265 | 0.1201 |
| | ELAPOR2 | 176 | −20.6 | 84.2 | 70 | −0.08 | 0.211 | 0.7139 | 0.8793 |
| | HDAC9 | 176 | −19.5 | 84.2 | 73 | −1.51 | 0.280 | 6.77E−08 | 3.53E−06 |
| | WDR47 | 176 | −19.6 | 79.0 | 625 | 0.08 | 0.089 | 0.3892 | 0.6617 |
| PD1_2061 | COG2 | 172 | −18.9 | 93.3 | 1244 | 0.06 | 0.060 | 0.3399 | 0.6170 |
| miR206 | NRG4 | 171 | −25.6 | 77.8 | | | | | |
| Passenger | AGBL3 | 170 | −19.2 | 79.0 | 49 | 0.01 | 0.250 | 0.9811 | 0.9934 |
| miRNA | PDK3 | 170 | −20.9 | 100.0 | 605 | 0.18 | 0.081 | 0.0256 | 0.1169 |
| | PDXK | 170 | −21.7 | 82.4 | 1786 | 0.22 | 0.058 | 0.0002 | 0.0022 |
| | N4BP2 | 169 | −25.1 | 77.8 | 2834 | −0.33 | 0.051 | 1.86E−10 | 1.95E−08 |
| | ZNF271P | 169 | −23.8 | 83.3 | 777 | 0.03 | 0.074 | 0.7197 | 0.8812 |
| | NFKB1 | 168 | −18.8 | 88.2 | 7889 | −0.11 | 0.039 | 0.0036 | 0.0269 |
| | XYLT1 | 168 | −26.9 | 82.4 | 5281 | 0.08 | 0.051 | 0.1100 | 0.3177 |
| | CNOT6 | 167 | −20.1 | 72.2 | 2030 | −0.24 | 0.052 | 0.0000 | 0.0001 |

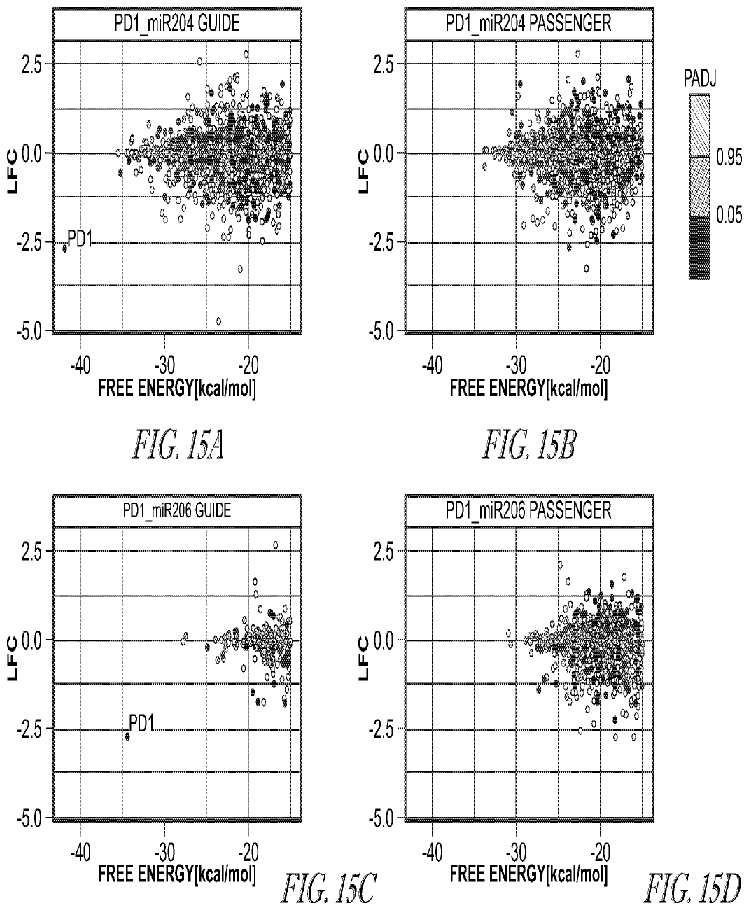

It is expected that changes in PD1 expression will impact expression of other genes in downstream pathways. As expected, analysis of RNAseq data confirmed the differential expression of several genes in miRNA expressing ROR1 UltraCAR-T cells compared to the control ROR1 UltraCAR-T cells (FIGS. 14A and B, Table 13). To elucidate direct versus indirect changes in gene expression, the differential expression (LFC) in miRNA expressing ROR1 UltraCAR-T cells compared to control ROR1 CAR-T cells was plotted against the predicted binding potential (predicted free energy) of the PD1 miRNAs for genes, which were in silico predicted as potential PD1 miRNA targets (FIGS. 15A-D). Genes with a highly negative free energy and a statistically significant reduction in expression are likely to be directly targeted by miRNA, while downregulated genes with a weak free energy are likely to be indirectly impacted by the miRNAs. PD1 is clearly separated from all other genes in the plots with a strong reduction in expression and high miRNA binding potential, which suggests a strong and preferential direct targeting of PD1, but not other genes, by the PD1 silencer miRNAs.

TABLE 13
Top 10 Down- and Up-Regulated Genes in ROR1 + PD1
Silencer Cells Relative to Control ROR1 UltraCAR-T Cells

| | | base | Log2Fold | | | | |
|---|---|---|---|---|---|---|---|
| Ensembl_ID | Symbol | Mean | Change | lfcSE | stat | pvalue | padj |
| ENSG00000188389 | PDCD1 | 84.2 | −2.6 | 0.3 | −9.0 | 2.51E−19 | 2.64E−16 |
| ENSG00000172020 | GAP43 | 71.6 | −2.2 | 0.4 | −5.7 | 1.37E−08 | 9.21E−07 |
| ENSG00000171951 | SCG2 | 110.5 | −2.2 | 0.4 | −6.1 | 1.13E−09 | 9.89E−08 |
| ENSG00000105974 | CAV1 | 172.3 | −2.1 | 0.5 | −4.2 | 2.93E−05 | 5.78E−04 |
| ENSG00000136193 | SCRN1 | 37.4 | −1.9 | 0.5 | −3.8 | 0.000136 | 1.97E−03 |
| ENSG00000130287 | NCAN | 206.1 | −1.7 | 0.4 | −4.4 | 1.23E−05 | 2.84E−04 |
| ENSG00000156052 | GNAQ | 145.0 | −1.7 | 0.2 | −7.8 | 6.88E−15 | 2.18E−12 |
| ENSG00000135898 | GPR55 | 84.5 | −1.6 | 0.3 | −5.7 | 1.47E−08 | 9.48E−07 |
| ENSG00000152495 | CAMK4 | 856.5 | −1.6 | 0.2 | −7.5 | 9.08E−14 | 2.21E−11 |
| ENSG00000102271 | KLHLA | 184.3 | −1.6 | 0.3 | −4.7 | 3.00E−06 | 8.90E−05 |
| ENSG00000165025 | SYK | 120.2 | 1.6 | 0.4 | 4.0 | 6.65E−05 | 1.10E−03 |
| ENSG00000276597 | TRBV11-3 | 123.1 | 1.6 | 0.6 | 2.7 | 0.006913 | 4.44E−02 |
| ENSG00000082781 | ITGB5 | 329.1 | 1.7 | 0.3 | 5.7 | 1.45E−08 | 9.44E−07 |
| ENSG00000244242 | IFITM10 | 555.8 | 1.7 | 0.2 | 7.6 | 4.18E−14 | 1.10E−11 |
| ENSG00000143842 | SOX13 | 116.9 | 1.7 | 0.2 | 7.6 | 3.87E−14 | 1.06E−11 |
| ENSG00000180549 | FUT7 | 318.7 | 1.7 | 0.4 | 4.3 | 1.71E−05 | 3.71E−04 |
| ENSG00000137474 | MYO7A | 1057.3 | 1.8 | 0.2 | 8.2 | 3.56E−16 | 1.62E−13 |
| ENSG00000177508 | IRX3 | 317.9 | 1.8 | 0.5 | 3.3 | 0.001046 | 1.03E−02 |
| ENSG00000136689 | IL1RN | 242.1 | 1.9 | 0.5 | 4.0 | 5.65E−05 | 9.70E−04 |
| ENSG00000189013 | KIR2DL4 | 800.0 | 1.9 | 0.3 | 6.4 | 1.51E−10 | 1.62E−08 |

### Example 11: Dual-miRNA Designs for Reducing Expression of TIGIT

A screen of various non-natural pri-miRNAs comprising guide miRNAs that target TIGIT was conducted.

Jurkat cells overexpressing TIGIT were transfected with: expression vectors encoding a CAR, membrane-bound IL15, and truncated HER1 with nucleic acid encoding pri-miRNA targeting TIGIT located in the section corresponding to a 5′ UTR intron; or a control vector expressing the CAR, membrane-bound IL15, and truncated HER1 but not the pri-miRNA.

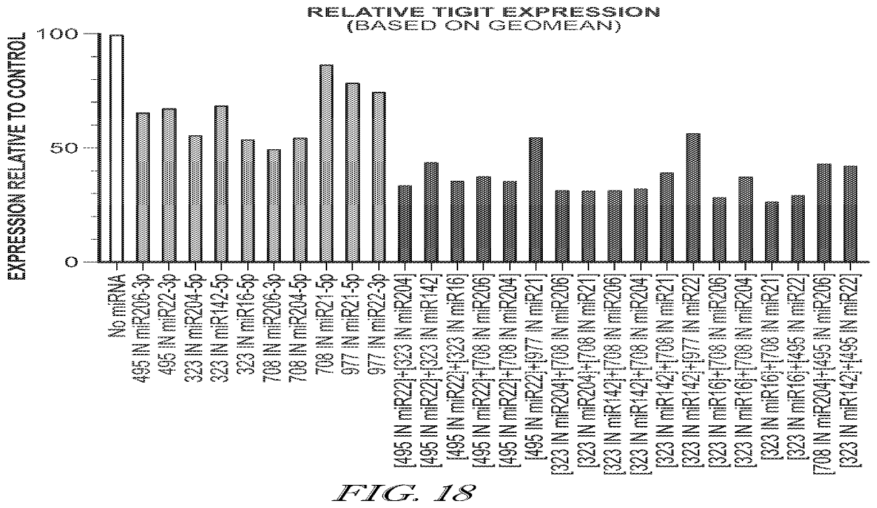

Two days post-transfection, flow cytometry was performed to quantify TIGIT expression (geometric mean fluorescence). The results are depicted in Table 14 and in FIG. 17.

TABLE 14

| | | | | Relative |
|---|---|---|---|---|
| | | Guide | | TIGIT |
| Backbone | Guide | miRNA | SEQ | Expression |
| Derived From | miRNA | Position | ID NO: | (%) |
| miR16 | TIGIT_495 | 5p | 104 | 103.6% |
| miR19 | TIGIT_495 | 3p | 112 | 99.2% |
| miR21 | TIGIT_495 | 5p | 106 | 94.1% |
| miR22 | TIGIT_495 | 3p | 102 | 65.0% |
| miR142 | TIGIT_495 | 5p | 110 | 93.7% |
| miR204 | TIGIT_495 | 5p | 100 | 85.2% |
| miR206 | TIGIT_495 | 3p | 98 | 74.7% |
| miR494 | TIGIT_495 | 3p | 108 | 107.5% |
| miR1915 | TIGIT_495 | 5p | 114 | 90.0% |
| miR204 + | TIGIT_11 + | 5p, 3p | | 62.1% |
| miR206 | TIGIT_495 | (respectively) | | |
| | (respectively) | | | |
| miR16 | TIGIT_323 | 5p | 124 | 65.3% |
| miR22 | TIGIT_323 | 3p | 120 | 87.8% |
| miR142 | TIGIT_323 | 5p | 122 | 73.2% |
| miR204 | TIGIT_323 | 5p | 118 | 57.5% |
| miR206 | TIGIT_323 | 3p | 116 | 76.1% |
| miR19 | TIGIT_708 | 3p | 134 | 94.6% |
| miR21 | TIGIT_708 | 5p | 130 | 76.6% |
| miR204 | TIGIT_708 | 5p | 128 | 47.1% |
| miR206 | TIGIT_708 | 3p | 126 | 54.0% |
| miR494 | TIGIT_708 | 3p | 132 | 92.1% |
| miR21 | TIGIT_977 | 5p | 142 | 80.3% |
| miR22 | TIGIT_977 | 3p | 144 | 79.0% |
| miR204 | TIGIT_977 | 5p | 138 | 85.0% |
| miR206 | TIGIT_977 | 3p | 140 | 88.8% |
| miR1915 | TIGIT_977 | 5p | 136 | 84.3% |

Following the above initial screen, an additional screen using the same protocol was conducted except here the expression vectors tested contained either one TIGIT-targeting pri-miRNA or two TIGIT-targeting pri-miRNAs. As with the previous screen, each test expression vector encoded a CAR, membrane-bound IL15, and truncated HER1 with the nucleic acid encoding the pri-miRNA(s) located in the section corresponding to a 5′ UTR intron. The control vector expressed a CAR, membrane-bound IL15, and truncated HER1 but not the pri-miRNA. The results are depicted in Table 15 and in FIG. 18.

TABLE 15

| | TIGIT |
|---|---|
| | Expression |
| | (%) relative |
| Pri-miRNA construct | to control |
| TIGIT_495 (SEQ ID NO: 98) in backbone derived from | 66% |
| miR206 as a miRNA-3p sequence | |
| TIGIT_495 (SEQ ID NO: 102) in backbone derived from | 68% |
| miR22 as a miRNA-3p sequence | |
| TIGIT_323 (SEQ ID NO: 118) in backbone derived from | 56% |
| miR204 as a miRNA-5p sequence | |
| TIGIT_323 (SEQ ID NO: 122) in backbone derived from | 69% |
| miR142 as a miRNA-5p sequence | |
| TIGIT_323 (SEQ ID NO: 124) in backbone derived from | 54% |
| miR16 as a miRNA-5p sequence | |
| TIGIT_708 (SEQ ID NO: 126) in backbone derived from | 50% |
| miR206 as a miRNA-3p sequence | |
| TIGIT_708 (SEQ ID NO: 128) in backbone derived from | 55% |
| miR204 as a miRNA-5p sequence | |
| TIGIT_708 (SEQ ID NO: 130) in backbone derived from | 87% |
| miR21 as a miRNA-5p sequence | |
| TIGIT_977 (SEQ ID NO: 142) in backbone derived from | 79% |
| miR21 as a miRNA-5p sequence | |
| TIGIT_977 (SEQ ID NO: 144) in backbone derived from | 75% |
| miR22 as a miRNA-3p sequence | |
| TIGIT_495 (SEQ ID NO: 102) in backbone derived from | 34% |
| miR22 as a miRNA-3p sequence + TIGIT_323 (SEQ ID | |
| NO: 118) in backbone derived from miR204 as a miRNA- | |
| 5p sequence | |
| TIGIT_495 (SEQ ID NO: 102) in backbone derived from | 44% |
| miR22 as a miRNA-3p sequence + TIGIT_323 (SEQ ID | |
| NO: 122) in backbone derived from miR142 as a miRNA- | |
| 5p sequence | |
| TIGIT_495 (SEQ ID NO: 102) in backbone derived from | 36% |
| miR22 as a miRNA-3p sequence + TIGIT_323 (SEQ ID | |
| NO: 124) in backbone derived from miR16 as a miRNA-5p | |
| sequence | |
| TIGIT_495 (SEQ ID NO: 102) in backbone derived from | 38% |
| miR22 as a miRNA-3p sequence + TIGIT_708 (SEQ ID | |
| NO: 126) in backbone derived from miR206 as a miRNA- | |
| 3p sequence | |
| TIGIT_495 (SEQ ID NO: 102) in backbone derived from | 36% |
| miR22 as a miRNA-3p sequence + TIGIT_708 (SEQ ID | |
| NO: 128) in backbone derived from miR204 as a miRNA- | |
| 5p sequence | |
| TIGIT_495 (SEQ ID NO: 102) in backbone derived from | 55% |
| miR22 as a miRNA-3p sequence + TIGIT_977 (SEQ ID | |
| NO: 142) in backbone derived from miR21 as a miRNA-5p | |
| sequence | |
| TIGIT_323 (SEQ ID NO: 118) in backbone derived from | 32% |
| miR204 as a miRNA-5p sequence + TIGIT_708 (SEQ ID | |
| NO: 126) in backbone derived from miR206 as a miRNA- | |
| 3p sequence | |
| TIGIT_323 (SEQ ID NO: 118) in backbone derived from | 32% |
| miR204 as a miRNA-5p sequence + TIGIT_708 (SEQ ID | |
| NO: 130) in backbone derived from miR21 as a miRNA-5p | |
| sequence | |
| TIGIT_323 (SEQ ID NO: 122) in backbone derived from | 32% |
| miR142 as a miRNA-5p sequence + TIGIT_708 (SEQ ID | |
| NO: 126) in backbone derived from miR206 as a miRNA- | |
| 3p sequence | |
| TIGIT_323 (SEQ ID NO: 122) in backbone derived from | 33% |
| miR142 as a miRNA-5p sequence + TIGIT_708 (SEQ ID | |
| NO: 128) in backbone derived from miR204 as a miRNA- | |
| 5p sequence | |
| TIGIT_323 (SEQ ID NO: 122) in backbone derived from | 40% |
| miR142 as a miRNA-5p sequence + TIGIT_708 (SEQ ID | |
| NO: 130) in backbone derived from miR21 as a miRNA-5p | |
| sequence | |
| TIGIT_323 (SEQ ID NO: 122) in backbone derived from | 57% |
| miR142 as a miRNA-5p sequence + TIGIT_977 (SEQ ID | |
| NO: 144) in backbone derived from miR22 as a miRNA-3p | |
| sequence | |
| TIGIT_323 (SEQ ID NO: 124) in backbone derived from | 29% |
| miR16 as a miRNA-5p sequence + TIGIT_708 (SEQ ID | |
| NO: 126) in backbone derived from miR206 as a miRNA- | |
| 3p sequence | |
| TIGIT_323 (SEQ ID NO: 124) in backbone derived from | 38% |
| miR16 as a miRNA-5p sequence + TIGIT_708 (SEQ ID | |
| NO: 128) in backbone derived from miR204 as a miRNA- | |
| 5p sequence | |
| TIGIT_323 (SEQ ID NO: 124) in backbone derived from | 27% |
| miR16 as a miRNA-5p sequence + TIGIT_708 (SEQ ID | |
| NO: 130) in backbone derived from miR21 as a miRNA-5p | |
| sequence | |
| TIGIT_323 (SEQ ID NO: 124) in backbone derived from | 30% |
| miR16 as a miRNA-5p sequence + TIGIT_495 (SEQ ID | |
| NO: 102) in backbone derived from miR22 as a miRNA-3p | |
| sequence | |
| TIGIT_708 (SEQ ID NO: 128) in backbone derived from | 44% |
| miR204 as a miRNA-5p sequence + TIGIT_495 (SEQ ID | |
| NO: 98) in backbone derived from miR206 as a miRNA-3p | |
| sequence | |
| TIGIT_323 (SEQ ID NO: 122) in backbone derived from | 43% |
| miR142 as a miRNA-5p sequence + TIGIT_495 (SEQ ID | |
| NO: 102) in backbone derived from miR22 as a miRNA-3p | |
| sequence | |

### Example 12: miRNA Designs for Reducing Expression of CD70

A screen of various constructs encoding non-natural pri-miRNAs comprising guide miRNAs that target CD70 was conducted.

Jurkat JRFTCR cells were transfected with: expression vectors encoding anti-MUC16 (4A5) CAR, membrane-bound IL-15, and truncated HER1 (HER1t). The expression vectors further encode: (i) a single CD70-targeting pri-miRNA located in a 5′UTR intron; (ii) two PD1-targeting pri-miRNAs and a single CD70-targeting pri-miRNA located in the 5′UTR intron; (iii) two PD1-targeting pri-miRNAs located in the 5′UTR intron and two CD70-targeting pri-miRNAs located in the 3′UTR; or (iv) two PD1-targeting pri-miRNAs located in the 5′UTR intron and a single CD70-targeting pri-miRNAs located in the 3′UTR. In addition, certain cells were transfected with control vectors expressing: (i) anti-MUC16 (4A5) CAR, membrane-bound IL-15, and truncated HER1 but no pri-miRNAs; or (ii) anti-MUC16 (4A5) CAR, membrane-bound IL-15, and truncated HER1 and two PD1-targeting pre-miRNAs located in the 5′UTR intron.

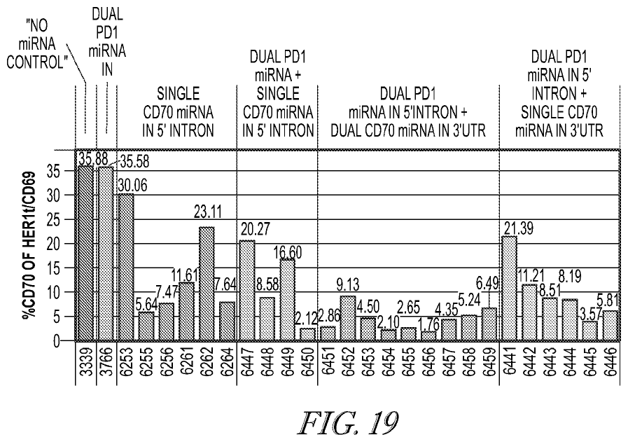

The cells were PMA/ion activated 1 day post-transfection to stimulate expression of CD70 and flow cytometry was performed 2 days post-transfection to quantify CD70 expression (% CD70-positive cells in the activated transfected population as well as geometric mean fluorescence). The results are depicted in Table 16 and in FIG. 19.

TABLE 16

| | | SEQ ID NO | |
|---|---|---|---|
| | | for single or | % CD70+ of |
| | | dual pri-miRNA | transfected |
| Construct | Location of pre-miRNA | targeting CD70 | activated cells |
| 3339 | No miRNA | | 35.88 |
| (Control) | | | |
| 3766 | Two PD1-targeting pre-miRNAs in 5′UTR | | 35.58 |
| (Control) | intron | | |
| 6253 | Single CD70-targeting pre-miRNA in | 326 | 30.06 |
| | 5′UTR intron | | |
| 6255 | Single CD70-targeting pre-miRNA in | 328 | 5.64 |
| | 5′UTR intron | | |
| 6256 | Single CD70-targeting pre-miRNA in | 329 | 7.47 |
| | 5′UTR intron | | |
| 6261 | Single CD70-targeting pre-miRNA in | 334 | 11.61 |
| | 5′UTR intron | | |
| 6262 | Single CD70-targeting pre-miRNA in | 335 | 23.11 |
| | 5′UTR intron | | |
| 6264 | Single CD70-targeting pre-miRNA in | 337 | 7.64 |
| | 5′UTR intron | | |
| 6447 | Two PD1-targeting pre-miRNAs followed | 448 | 20.27 |
| | by a single CD70-targeting pre-miRNA in 5′ | | |
| | to 3′ order in 5′UTR intron | | |
| 6448 | Two PD1-targeting pre-miRNAs followed | 449 | 8.58 |
| | by a single CD70-targeting pre-miRNA in 5′ | | |
| | to 3′ order in 5′UTR intron | | |
| 6449 | Single CD70-targeting pre-miRNA followed | 450 | 16.60 |
| | by two PD1-targeting pre-miRNAs in 5′ to | | |
| | 3′ order in 5′UTR intron | | |
| 6450 | Single CD70-targeting pre-miRNA followed | 451 | 2.12 |
| | by two PD1-targeting pre-miRNAs in 5′ to | | |
| | 3′ order in 5′UTR intron | | |
| 6451 | Two PD1-targeting pre-miRNAs in the | 452 | 2.86 |
| | 5′UTR intron and two CD70-targeting pre- | | |
| | miRNAs in the 3′UTR | | |
| 6452 | Two PD1-targeting pre-miRNAs in the | 453 | 9.13 |
| | 5′UTR intron and two CD70-targeting pre- | | |
| | miRNAs in the 3′UTR | | |
| 6453 | Two PD1-targeting pre-miRNAs in the | 454 | 4.50 |
| | 5′UTR intron and two CD70-targeting pre- | | |
| | miRNAs in the 3′UTR | | |
| 6454 | Two PD1-targeting pre-miRNAs in the | 455 | 2.10 |
| | 5′UTR intron and two CD70-targeting pre- | | |
| | miRNAs in the 3′UTR | | |
| 6455 | Two PD1-targeting pre-miRNAs in the | 456 | 2.65 |
| | 5′UTR intron and two CD70-targeting pre- | | |
| | miRNAs in the 3′UTR | | |
| 6456 | Two PD1-targeting pre-miRNAs in the | 457 | 1.76 |
| | 5′UTR intron and two CD70-targeting pre- | | |
| | miRNAs in the 3′UTR | | |
| 6457 | Two PD1-targeting pre-miRNAs in the | 458 | 4.35 |
| | 5′UTR intron and two CD70-targeting pre- | | |
| | miRNAs in the 3′UTR | | |
| 6458 | Two PD1-targeting pre-miRNAs in the | 459 | 5.24 |
| | 5′UTR intron and two CD70-targeting pre- | | |
| | miRNAs in the 3′UTR | | |
| 6459 | Two PD1-targeting pre-miRNAs in the | 460 | 6.49 |
| | 5′UTR intron and two CD70-targeting pre- | | |
| | miRNAs in the 3′UTR | | |
| 6441 | Two PD1-targeting pre-miRNAs in the | 326 | 21.39 |
| | 5′UTR intron and a single CD70-targeting | | |
| | pre-miRNA in the 3′UTR | | |
| 6442 | Two PD1-targeting pre-miRNAs in the | 335 | 11.21 |
| | 5′UTR intron and a single CD70-targeting | | |
| | pre-miRNA in the 3′UTR | | |
| 6443 | Two PD1-targeting pre-miRNAs in the | 337 | 8.51 |
| | 5′UTR intron and a single CD70-targeting | | |
| | pre-miRNA in the 3′UTR | | |
| 6444 | Two PD1-targeting pre-miRNAs in the | 334 | 8.19 |
| | 5′UTR intron and a single CD70-targeting | | |
| | pre-miRNA in the 3′UTR | | |
| 6445 | Two PD1-targeting pre-miRNAs in the | 328 | 3.57 |
| | 5′UTR intron and a single CD70-targeting | | |
| | pre-miRNA in the 3′UTR | | |
| 6446 | Two PD1-targeting pre-miRNAs in the | 329 | 5.81 |
| | 5′UTR intron and a single CD70-targeting | | |
| | pre-miRNA in the 3′UTR | | |

The sequence for the dual pri-miRNA targeting PD-1 for all constructs above was SEQ ID NO: 267.

### Example 13

As shown in the above examples, a dual miR204+miR206 combination has been demonstrated to provide robust gene silencing of all target human genes tested to date (PD1, TIGIT, CD70). Furthermore, small RNAseq analysis has confirmed that ˜99.9% of small RNAs produced from the dual miR204+miR206 combination map to the predicted guide or passenger mature miRNA sequences (˜93% guide, 7% passenger), confirming the appropriate folding and processing of the dual pri-miRNA designs.

Nucleic Acid Sequences Encoding Backbone pri-miRNA Sequences

| Synthetic | DNA encoding 5′ | DNA encoding | DNA encoding 3′ |
|---|---|---|---|
| miRNA | backbone sequence | stem loop | backbone sequence |
| miR204 | AGGAGGGTGGGGG | GAGAATATATGAAG | GTTCAATTGTCATCA |
| | TGGAGGCAAGCAGA | G | CTGGCATCTTTTTTG |
| | GGACTTCCTGATCGC | (SEQ ID NO: 2) | ATCATTGCACCATCA |
| | GTACCCATGGCTACA | | TCAAATGCATTGGGA |
| | GTCTTTCTTCATGTG | | TAACCATGAC |
| | ACTCGTGGAC | | (SEQ ID NO: 3) |
| | (SEQ ID NO: 1) | | |
| miR206 | GATGCTACAAGTGG | TATGGATTACTTTGC | TTTCGGCAAGTGCCT |
| | CCCACTTCTGAGATG | TA | CCTCGCTGGCCCCAG |
| | CGGGCTGCTTCTGG | (SEQ ID NO: 5) | GGTACCACCCGGAG |
| | ATGACACTGCTTCCC | | CACAGGTTTGGTGAC |
| | GAGG | | CTT |
| | (SEQ ID NO: 4) | | (SEQ ID NO: 6) |
| miR17 | TTAGCAGGAAAAAA | TGATATGTGCAT | GCATTATGGTGACA |
| | GAGAACATCACCTTG | (SEQ ID NO: 8) | GCTGCCTCGGGAAG |
| | TAAAACTGAAGATTG | | CCAAGTTGGGCTTTA |
| | TGACCAGTCAGAATA | | AAGTGCAGGGCCTG |
| | ATGT | | CTGATGT |
| | (SEQ ID NO: 7) | | (SEQ ID NO: 9) |
| miR150 | AGGGACTGGGCCCA | CTGGGCTCAG | CAGGGACCTGGGGA |
| | CGGGGAGGCAGCGT | (SEQ ID NO: 11) | CCCCGGCACCGGCA |
| | CCCCGAGGCAGCAG | | GGCCCCAAGGGGTG |
| | CGGCAGCGGCGGCT | | AGGTGAGCGGGCAT |
| | CCTCTCCCCATGGCC | | TGGGACCTCCCCTCC |
| | CTG | | CTGTACTC |
| | (SEQ ID NO: 10) | | (SEQ ID NO: 12) |
| miR150 | AGGGACTGGGCCCA | TGCTGGGCTCAGACC | GGACCTGGGGACCC |
| | CGGGGAGGCAGCGT | (SEQ ID NO: 14) | CGGCACCGGCAGGC |
| | CCCCGAGGCAGCAG | | CCCAAGGGGTGAGG |
| | CGGCAGCGGCGGCT | | TGAGCGGGCATTGG |
| | CCTCTCCCCATGGCC | | GACCTCCCCTCCCTG |
| | (SEQ ID NO: 13) | | TACTC |
| | | | (SEQ ID NO: 15) |
| miR16 | CTTCTGAAGAAAATA | TTAAGATTCTAAAAT | AAGTAAGGTTGACCA |
| | TATTTCTTTTTATTCA | TATCT | TACTCTACAGTTGTG |
| | TAGCTCTTATGATAG | (SEQ ID NO: 17) | TTTTAATGTATATTA |
| | CAATGTCAGCAGTGC | | ATGTTACTAATGTGT |
| | CT | | TTT |
| | (SEQ ID NO: 16) | | (SEQ ID NO: 18) |
| miR30a | CAGGTTAACCCAACA | CTGTGAAGCCACAGA | TGCCTACTGCCTCGG |
| | GAAGGCTAAAGAAG | TGGG | ACTTCAAGGGGCTAC |
| | GTATATTGCTGTTGA | (SEQ ID NO: 20) | TTTAGGAGCAATTAT |
| | CAGTGAGCGAC | | CTTGTTTA |
| | (SEQ ID NO: 19) | | (SEQ ID NO: 21) |
| miR126 | CCGGGGTCCTGTCTG | CTGTGACACTTCAAA | CCGTCCACGGCACCG |
| | CATCCAGCGCAGCAT | C | CATCGAAAACGCCGC |
| | TCTGGAAGACGCCAC | (SEQ ID NO: 23) | TGAGACCTCAGCCTT |
| | GCCTCCGCTGGCGAC | | GACCTCCCTCAGCGT |
| | GG | | GG |
| | (SEQ ID NO: 22) | | (SEQ ID NO: 24) |
| miR122 | CAATGGTGGAATGT | TGTCTAAACTAT | TAGCTACTGCTAGGC |
| | GGAGGTGAAGTTAA | (SEQ ID NO: 26) | AATCCTTCCCTCGAT |
| | CACCTTCGTGGCTAC | | AAATGTCTTGGCATC |
| | AGAGTTTCCTTAGCA | | GTTTGCTTTGAGCAA |
| | GAGCTG | | GAAGGTTCATCTGAT |
| | (SEQ ID NO: 25) | | ATCAGTCTTCTCAAT |
| | | | CT |
| | | | (SEQ ID NO: 27) |
| miR214 | ACAGGCTGATTGTAT | GCAGAACATCCGCTC | CACATGACAACCCAG |
| | CTGTCTATGAGCAAA | ACCTGT | CCTGAATGACAACCA |
| | GGAAACCTGAAGGA | (SEQ ID NO: 29) | GCCATTGAAAGAAA |
| | ACCAAGGGCCTGGC | | GCAGCCCTCACACCA |
| | TGGACAGAGTTGTC | | TAGCATCTA |
| | ATGTG | | (SEQ ID NO: 30) |
| | (SEQ ID NO: 28) | | |
| miR214 | ACAGGCTGATTGTAT | AGAACATCCGCTCAC | CACATGACAACCCAG |
| | CTGTCTATGAGCAAA | CT | CCTGAATGACAACCA |
| | GGAAACCTGAAGGA | (SEQ ID NO: 32) | GCCATTGAAAGAAA |
| | ACCAAGGGCCTGGC | | GCAGCCCTCACACCA |
| | TGGACAGAGTTGTC | | TAGCATCTA |
| | ATGTGTC | | (SEQ ID NO: 33) |
| | (SEQ ID NO: 31) | | |
| miR29b1 | GGGTTTATTGTAAGA | GATTTAAATAGTGAT | CTTGGGGGAGACCA |
| | GAGCATTATGAAGA | TGTC | GCTGCGCTGCACTAC |
| | AAAAAATAGATCATA | (SEQ ID NO: 35) | CAACAGCAAAAGAA |
| | AAGCTTCTTCAGG | | GTGAATGGGACAGC |
| | (SEQ ID NO: 34) | | T |
| | | | (SEQ ID NO: 36) |
| miR29b1 | GGGTTTATTGTAAGA | TTTAAATAGTGATTG | GTTCTTGGGGGAGA |
| | GAGCATTATGAAGA | (SEQ ID NO: 38) | CCAGCTGCGCTGCAC |
| | AAAAAATAGATCATA | | TACCAACAGCAAAAG |
| | AAGCTTCTTCAGGAA | | AAGTGAATGGGACA |
| | (SEQ ID NO: 37) | | GCT |
| | | | (SEQ ID NO: 39) |
| miR133a1 | TTTACCAATGAAAAG | TCGCCTCTTCAATGG | TAGCTATGCATTGAT |
| | CATTTAACTGTTTTG | A | TACTACGGGACAACC |
| | GATTCCAAACTAGCA | (SEQ ID NO: 41) | AACGTTTTCATTTGT |
| | GCACTACAATGCTTT | | GAATATCAATTACTT |
| | GCTA | | GCCA |
| | (SEQ ID NO: 40) | | (SEQ ID NO: 42) |
| miR26a1 | TGAAGCCACAGGAG | GTGCAGGTCCCAAT | CGGGGACGCGGGCC |
| | CCAAGAGCAGGAGG | G | TGGACGCCGGCATCC |
| | ACCAAGGCCCTGGC | (SEQ ID NO: 44) | GGGCTCAGGACCCC |
| | GAAGGCCGTGGCCT | | CCTCTCTGCCAGAGG |
| | CG | | C |
| | (SEQ ID NO: 43) | | (SEQ ID NO: 45) |
| miR412 | GTCTTGGAGGCTGG | GTTTCT | GCCGTCCGTATCCGC |
| | GGCACCTCGGGGAA | (SEQ ID NO: 47) | TGCAGCCTGTGGGG |
| | GGACGCCGGCATCA | | CCTGCGGGCCGGGG |
| | GCACCATTCTGGGGT | | AGCCGATCGCGCTTC |
| | ACGGGGATGGA | | AGCTCAGCGCCT |
| | (SEQ ID NO: 46) | | (SEQ ID NO: 48) |
| miR-19 | CCAATAATTCAAGCC | TACAAGAAGAATGTA | TGGTGGCCTGCTATT |
| | AAGCAAGTATATAG | GT | TCCTTCAAATGAATG |
| | GTGTTTTAATAGTTT | (SEQ ID NO: 50) | ATTTTTACTAATTTT |
| | TTGTTTGCAGTCCTC | | GTGTACTTTTATTGT |
| | TG | | G |
| | (SEQ ID NO: 49) | | (SEQ ID NO: 51) |
| miR-21 | TGTCTGCTTGTTTTG | CTGTTGAATCTCATG | TCTGACATTTTGGTA |
| | CCTACCATCGTGACA | G | TCTTTCATCTGACCA |
| | TCTCCATGGCTGTAC | (SEQ ID NO: 53) | TCCATATCCAATGTT |
| | CACCTTGTCGGG | | CTCATTTAAACA |
| | (SEQ ID NO: 52) | | (SEQ ID NO: 54) |
| miR-142 | GGAGTCAGGAGGCC | AACAGCACTGGAGG | GATGAGTGTACTGT |
| | TGGGCAGCCTGAAG | G | GGGCTTCGGAGATC |
| | AGTACACGCCGACG | (SEQ ID NO: 56) | ACGCCACTGCTGCCG |
| | GACAGACAGACAGT | | CCCGCTGCCCGCCAC |
| | GCAGTCACC | | CATCTTC |
| | (SEQ ID NO: 55) | | (SEQ ID NO: 57) |
| miR-494 | CAGTTCTGTTTTGAT | TCTTTATTTATGA | TTTTTTAGTATCAAA |
| | TTTTTTTGTTTGTTTT | (SEQ ID NO: 59) | TCCCACCCTGGAGGC |
| | TTGATCAGTGCTAAT | | ACTTCCTGTTCCTGA |
| | CTTCGATACTCGAAG | | TGCAGCCTTCAGGG |
| | GA | | AGG |
| | (SEQ ID NO: 58) | | (SEQ ID NO: 60) |
| miR-1915 | CGGACCACGGTGTCC | GTGCACCCGTG | GCGGCCCTAGCGAC |
| | CCTTCTCTCCAGCTG | (SEQ ID NO: 62) | CTGCGGCGGCGCCG |
| | GGGGTCTCGGGTCC | | GGAAAGCCCTGCCTC |
| | TGGCGCTGAGAGGC | | TGCAGCGGGTCCCA |
| | CGC | | GGGGTC |
| | (SEQ ID NO: 61) | | (SEQ ID NO: 63) |
| miR-22 | CATTTTCCCTCCCTTT | TATGTCCTGACCCAG | TGCCCTCTGCCCCTG |
| | CCCTTAGGAGCCTGT | CTA | GCTTCGAGGAGGAG |
| | TCCTCTCACGCCCTC | (SEQ ID NO: 339) | GAGGAGCTGCTTTCC |
| | ACCTGGCTGAGCCGC | | CCATCATCTGGAAGG |
| | A | | TG |
| | (SEQ ID NO: 338) | | (SEQ ID NO: 340) |
| miR-26a1 | ATGAAGCCACAGGA | GTGCAGGTCCCAAT | CGGGGACGCGGGCC |
| | GCCAAGAGCAGGAG | G | TGGACGCCGGCATCC |
| | GACCAAGGCCCTGG | (SEQ ID NO: 342) | GGGCTCAGGACCCC |
| | CGAAGGCCGTGGCC | | CCTCTCTGCCAGAGG |
| | TCG | | C |
| | (SEQ ID NO: 341) | | (SEQ ID NO: 343) |
| miR-486 | TGTGGTGCTGGGGG | CCCTTCATGCTGC | ACTCGGGGTGGGAG |
| | CTTCAGCGGCCGGCT | (SEQ ID NO: 345) | TCAGCAGGAGGTGA |
| | CTGATCTCCATCCTC | | GGGGGCATGGTGGC |
| | CCTGGGGCA | | CCCAGTGCAGC |
| | (SEQ ID NO: 344) | | (SEQ ID NO: 346) |

Nucleic acid sequences encoding mature miRNA sequences

| miRNA | | DNA encoding | DNA encoding |
|---|---|---|---|
| Target | Backbone | guide miRNA | passenger miRNA |
| CTLA4 | miR-204 | AATATAGTCTTCTCCCTCGCTT | AAGCAGGGACAGGACT |
| | | (SEQ ID NO: 64) | ATATT |
| | | | (SEQ ID NO: 65) |
| CTLA4 | miR-26a | AATATAGTCTTCTCCCTCGCTT | GAGCGAGGGATAGGAC |
| | | (SEQ ID NO: 66) | TATACT |
| | | | (SEQ ID NO: 67) |
| CTLA4 | miR-30a | AATATAGTCTTCTCCCTCGCTG | CAGTGAGGGAAGACTA |
| | | (SEQ ID NO: 68) | TGTT |
| | | | (SEQ ID NO: 69) |
| CTLA4 | miR-206 | AATATAGTCTTCTCCCTCGCTG | CAGCGAGGGAGAAGAT |
| | | (SEQ ID NO: 70) | TACCATT |
| | | | (SEQ ID NO: 71) |
| PD1 | miR-204 | TTCAGGAATGGGTTCCAAGGAT | ATCCTGGAAGCTATTCC |
| | | (SEQ ID NO: 72) | TGAC |
| | | | (SEQ ID NO: 73) |
| PD1 | miR-206 | TATAATATAATAGAACCACAGG | CCTGTGGTTCTGTTATA |
| | | (SEQ ID NO: 74) | TCCATA |
| | | | (SEQ ID NO: 75) |
| PD1 | miR-30a | TTCAGGAATGGGTTCCAAGGAG | CTCCTTGGACCATTCCT |
| | | (SEQ ID NO: 76) | GAA |
| | | | (SEQ ID NO: 77) |
| PD1 | miR-412 | TTCAGGAATGGGTTCCAAGGAATT | AATGTCCTGAAGCCATT |
| | | (SEQ ID NO: 78) | CATGGA |
| | | | (SEQ ID NO: 79) |
| PD1 | miR122 | TTCAGGAATGGGTTCCAAGGAG | CTCCTTGGAACACATTC |
| | | (SEQ ID NO: 80) | CTACA |
| | | | (SEQ ID NO: 81) |
| PD1 | miR-17 | TTCAGGAATGGGTTCCAAGGAAG | CTTCTTGGTACACGTTC |
| | | (SEQ ID NO: 82) | CTGCTCA |
| | | | (SEQ ID NO: 83) |
| PD1 | miR-150 | TATAATATAATAGAACCACAGG | ACGTGTGGTTTATCCTG |
| | | (SEQ ID NO: 74) | TTGTA |
| | | | (SEQ ID NO: 85) |
| PD1 | miR-486 | TATAATATAATAGAACCACAGG | CCAGCTTGTGGTTCTAT |
| | | (SEQ ID NO: 74) | TATGTTATA |
| | | | (SEQ ID NO: 87) |
| PD1 | miR-204 | TTCAGGAATGGGTTCCAAGGA | TCCTGGAAGCTATTCCT |
| | | (SEQ ID NO: 704) | GACG |
| | | | (SEQ ID NO: 706) |
| PD1 | miR-204 | TTCAGGAATGGGTTCCAAGGATG | TCCTGGAAGCTATTCCT |
| | | (SEQ ID NO: 705) | GACGTT |
| | | | (SEQ ID NO: 708) |
| PD1 | miR-22 | | TCCTGGAAGCTATTCCT |
| | | | GACGT |
| | | | (SEQ ID NO: 707) |
| PD1 | miR-206 | TATAATATAATAGAACCACAG | TGTGGTTCTGTTATATC |
| | | (SEQ ID NO: 709) | CATA |
| | | | (SEQ ID NO: 711) |
| PD1 | miR-206 | TATAATATAATAGAACCACAGGA | |
| | | SEQ ID NO: 710) | |
| PD1 | miR-206 | | TGTGGTTCTGTTATATC |
| | | | CATAT |
| | | | (SEQ ID NO: 712) |
| PD1 | miR-206 | | TGTGGTTCTGTTATATC |
| | | | CATATA |
| | | | (SEQ ID NO: 713) |
| TIGIT | miR-17 | AGATCCACGTTACTCACCCTAG | CTAGGGTGTGTCATGT |
| | | (SEQ ID NO: 88) | GGATGAA |
| | | | (SEQ ID NO: 89) |
| TIGIT | miR-150 | AGATCCACGTTACTCACCCGTG | ACCCGGGTGATAATAT |
| | | (SEQ ID NO: 90) | GGATCT |
| | | | (SEQ ID NO: 91) |
| TIGIT | miR-204 | AGATCCACGTTACTCACCCCCT | AGGGGTGAGAAGCGT |
| | | (SEQ ID NO: 92) | GGATCC |
| | | | (SEQ ID NO: 93) |
| TIGIT | miR29b1 | AGATCCACGTTACTCACCCTTA | TAGGTGAGTTACGTGG |
| | | (SEQ ID NO: 94) | ATCTGTT |
| | | | (SEQ ID NO: 95) |
| TIGIT | miR214 | AGATCCACGTTACTCACCCTGC | GTAGGGTGAGACTCGT |
| | | (SEQ ID NO: 96) | GGATCT |
| | | | (SEQ ID NO: 97) |
| TIGIT | miR-206 | ACCACGATGACTGCTGTGCAGA | TCTGCGTAGCAGTCATC |
| | | (SEQ ID NO: 98) | GCCGGT |
| | | | (SEQ ID NO: 99) |
| TIGIT | miR-204 | ACCACGATGACTGCTGTGCAGA | TCTGATGGCCGTCATCG |
| | | (SEQ ID NO: 100) | TGGC |
| | | | (SEQ ID NO: 101) |
| TIGIT | miR-22 | ACCACGATGACTGCTGTGCAGA | TCTGTACAGCAGCATCG |
| | | (SEQ ID NO: 102) | ATGGT |
| | | | (SEQ ID NO: 103) |
| TIGIT | miR-16 | ACCACGATGACTGCTGTGCAGA | TGCGCAGCTGTTCATCG |
| | | (SEQ ID NO: 104) | TGGT |
| | | | (SEQ ID NO: 105) |
| TIGIT | miR-21 | ACCACGATGACTGCTGTGCAGA | CTGCGAAGCAGCATCG |
| | | (SEQ ID NO: 106) | TGGC |
| | | | (SEQ ID NO: 107) |
| TIGIT | miR-494 | ACCACGATGACTGCTGTGCAGA | TCTGCAACGCAGTCTCG |
| | | (SEQ ID NO: 108) | CTGG |
| | | | SEQ ID NO: 109) |
| TIGIT | miR-142 | ACCACGATGACTGCTGTGCAGA | GATGCACAGCAATCATC |
| | | (SEQ ID NO: 110) | GTGGT |
| | | | (SEQ ID NO: 111) |
| TIGIT | miR-19 | ACCACGATGACTGCTGTGCAGAT | ATCTGCACAGCAGTTCG |
| | | (SEQ ID NO: 112) | TGG |
| | | | (SEQ ID NO: 113) |
| TIGIT | miR-1915 | ACCACGATGACTGCTGTGCAGA | TCTGCCAATAGCTGTAA |
| | | (SEQ ID NO: 114) | TCGTGG |
| | | | (SEQ ID NO: 115) |
| TIGIT | miR-206 | TATCGTTCACGGTCAGCGACTG | CAGTCGTTGACTGTGG |
| | | (SEQ ID NO: 116) | ACTTATA |
| | | | (SEQ ID NO: 117) |
| TIGIT | miR-204 | TATCGTTCACGGTCAGCGACTG | CAGTGCTGAACGTGAA |
| | | (SEQ ID NO: 118) | CGATC |
| | | | (SEQ ID NO: 119) |
| TIGIT | miR-22 | TATCGTTCACGGTCAGCGACTG | TAGTCGCTGACTTGAAC |
| | | (SEQ ID NO: 120) | AGATA |
| | | | (SEQ ID NO: 121) |
| TIGIT | miR-142 | TATCGTTCACGGTCAGCGACT | TGTCGCTGACAGTGAA |
| | | (SEQ ID NO: 122) | CGATA |
| | | | (SEQ ID NO: 123) |
| TIGIT | miR-16 | TATCGTTCACGGTCAGCGACTG | GTCGCTGAGCGCTGAA |
| | | (SEQ ID NO: 124) | CGATG |
| | | | (SEQ ID NO: 125) |
| TIGIT | miR-206 | TAACTCAGGACATTGAAGTAGT | ACTACTTCAATGTCCTG |
| | | (SEQ ID NO: 126) | ACCTTA |
| | | | (SEQ ID NO: 127) |
| TIGIT | miR-204 | TAACTCAGGACATTGAAGTAGT | ACTATTCAGAGTCCTGA |
| | | (SEQ ID NO: 128) | GTTC |
| | | | (SEQ ID NO: 129) |
| TIGIT | miR-21 | TAACTCAGGACATTGAAGTAGT | CTACTCCAATGCCTGAG |
| | | (SEQ ID NO: 130) | TTG |
| | | | (SEQ ID NO: 131) |
| TIGIT | miR-494 | TAACTCAGGACATTGAAGTAGT | ACTACTGTAATGTCTGA |
| | | (SEQ ID NO: 132) | CGTT |
| | | | (SEQ ID NO: 133) |
| TIGIT | miR-19 | TAACTCAGGACATTGAAGTAGTC | GACTACTTCAATGTTGA |
| | | (SEQ ID NO: 134) | GTT |
| | | | (SEQ ID NO: 135) |
| TIGIT | miR-1915 | AGATCCACGTTACTCACCCTAG | CTAGGCAGTGAGCAAA |
| | | (SEQ ID NO: 136) | GTGGATC |
| | | | (SEQ ID NO: 137) |
| TIGIT | miR-204 | AGATCCACGTTACTCACCCTAG | CTAGGTGAGAAACGTG |
| | | (SEQ ID NO: 138) | GATCG |
| | | | (SEQ ID NO: 139) |
| TIGIT | miR-206 | AGATCCACGTTACTCACCCTAG | CTAGGGTGAGTAACGT |
| | | (SEQ ID NO: 140) | GGCCTCT |
| | | | (SEQ ID NO: 141) |
| TIGIT | miR-21 | AGATCCACGTTACTCACCCTAG | TAGGGCGAGTACGTGG |
| | | (SEQ ID NO: 142) | ATCG |
| | | | (SEQ ID NO: 143) |
| TIGIT | miR-22 | AGATCCACGTTACTCACCCTAG | CTAGGGTGAGTACGTG |
| | | (SEQ ID NO: 144) | GCATCT |
| | | | (SEQ ID NO: 145) |
| TIM3 | miR204 | CATTATGCCTGGGATTTGGATC | GATCAGATCGCAGGCA |
| | | (SEQ ID NO: 146) | TAATT |
| | | | (SEQ ID NO: 147) |
| TIM3 | miR206 | CATTATGCCTGGGATTTGGATC | GATCCGGATCCTAGGT |
| | | (SEQ ID NO: 148) | ATCCATG |
| | | | (SEQ ID NO: 149) |
| TIM3 | miR17 | CATTATGCCTGGGATTTGGATCG | CGGCCAGAACCAAGGC |
| | | (SEQ ID NO: 150) | ATAAGTA |
| | | | (SEQ ID NO: 151) |
| TIM3 | miR126 | CATTATGCCTGGGATTTGGATC | ACTCCAAATCCCGTGCA |
| | | (SEQ ID NO: 152) | TAATCG |
| | | | (SEQ ID NO: 153) |
| TIM3 | miR122 | CATTATGCCTGGGATTTGGATC | GATTCGAATCCAAGGC |
| | | (SEQ ID NO: 154) | ATATGG |
| | | | (SEQ ID NO: 155) |
| TIM3 | miR214 | TAATTCACATCCCTTTCATCAG | ATGATGAAAGGACAGT |
| | | (SEQ ID NO: 156) | GAATTA |
| | | | (SEQ ID NO: 157) |
| LAG3 | miR-30a | TAGTCGTTGGGTAAAGTCGCCA | TGGTGACTTCTCAACGA |
| | | (SEQ ID NO: 158) | TTA |
| | | | (SEQ ID NO: 159) |
| LAG3 | miR122 | GTTGCTTTCCGCTAAGTGGTGA | TCACCACTTAGAGGAAA |
| | | (SEQ ID NO: 160) | GCCTC |
| | | | (SEQ ID NO: 161) |
| GITR | miR206 | TTTGCAGTGGCCTTCGTGGCCC | GGGCTATGAAGGCCAT |
| | | (SEQ ID NO: 162) | TGAGAAA |
| | | | (SEQ ID NO: 163) |
| GITR | miR29b1 | AGCCTCCCGTCCTAAGACCCCAC | GTGGGTCTTAGGTCGG |
| | | (SEQ ID NO: 164) | GAGCTGCT |
| | | | (SEQ ID NO: 165) |
| PIK3IP1 | miR206 | TGAACGACCAGTGTTTAACCGG | CCGGTTGAACATTGGT |
| | | (SEQ ID NO: 166) | TGCCTCA |
| | | | (SEQ ID NO: 167) |
| PIK3IP1 | miR126 | TTCTCCTTGGAGTTCATCCGCG | ATGAACTTAGAGGAGA |
| | | (SEQ ID NO: 168) | CG |
| | | | (SEQ ID NO: 169) |
| PIK3IP1 | miR30a | TTCTCCTTGGAGTTCATCCGCG | CGCGGATGATCCAAGG |
| | | (SEQ ID NO: 170) | AGGA |
| | | | (SEQ ID NO: 171) |
| CD70 | miR206 | TGTGCGAAGCGCTGGATGCACA | TGTGCGTCCAGCGCTTC |
| | | (SEQ ID NO: 293) | GAGACA |
| | | | (SEQ ID NO: 294) |
| CD70 | miR204 | TGTGCGAAGCGCTGGATGCACA | TGTGGTCCACCGCTTCG |
| | | (SEQ ID NO: 295) | CACC |
| | | | (SEQ ID NO: 296) |
| CD70 | miR22 | TGTGCGAAGCGCTGGATGCACA | TGTGCATCCAGCCTTCG |
| | | (SEQ ID NO: 297) | ACACA |
| | | | (SEQ ID NO: 298) |
| CD70 | miR16 | TGTGCGAAGCGCTGGATGCACA | TGTGCGAAGCGCTGGA |
| | | (SEQ ID NO: 299) | TGCACA |
| | | | (SEQ ID NO: 300) |
| CD70 | miR21 | TGTGCGAAGCGCTGGATGCACA | TGTGCGAAGCGCTGGA |
| | | (SEQ ID NO: 301) | TGCACA |
| | | | (SEQ ID NO: 302) |
| CD70 | miR206 | TCCTGTGTGATTCAGCTGCAGC | GCTGCAGCTGAATCAC |
| | | (SEQ ID NO: 303) | ACTCGGA |
| | | | (SEQ ID NO: 304) |
| CD70 | miR204 | TCCTGTGTGATTCAGCTGCAGC | TCCTGTGTGATTCAGCT |
| | | (SEQ ID NO: 305) | GCAGC |
| | | | (SEQ ID NO: 306) |
| CD70 | miR19 | TCCTGTGTGATTCAGCTGCAGCT | AGCTGCAGCTGAATCA |
| | | (SEQ ID NO: 307) | CAGG |
| | | | (SEQ ID NO: 308) |
| CD70 | miR494 | TCCTGTGTGATTCAGCTGCAGC | GCTGCATATGAATCCAC |
| | | (SEQ ID NO: 309) | CAGG |
| | | | (SEQ ID NO: 310) |
| CD70 | miR150 | TCCTGTGTGATTCAGCTGCAGC | TCCTGTGTGATTCAGCT |
| | | (SEQ ID NO: 311) | GCAGC |
| | | | (SEQ ID NO: 312) |
| CD70 | miR206 | ATGTGTACCATGTAGATGCCAT | ATGGCATCTACATGGTA |
| | | (SEQ ID NO: 313) | CTTCAT |
| | | | (SEQ ID NO: 314) |
| CD70 | miR204 | ATGTGTACCATGTAGATGCCAT | ATGGATCTAGATGGTA |
| | | (SEQ ID NO: 315) | CACAC |
| | | | (SEQ ID NO: 316) |
| CD70 | miR22 | ATGTGTACCATGTAGATGCCAT | ATGGCATCTACAGGTAC |
| | | (SEQ ID NO: 317) | AACAT |
| | | | (SEQ ID NO: 318) |
| CD70 | miR142 | ATGTGTACCATGTAGATGCCA | ATGTGTACCATGTAGAT |
| | | (SEQ ID NO: 319) | GCCA |
| | | | (SEQ ID NO: 320) |
| CD70 | miR26a1 | ATGTGTACCATGTAGATGCCAT | ATGTGTACCATGTAGAT |
| | | (SEQ ID NO: 321) | GCCAT |
| | | | (SEQ ID NO: 322) |

Nucleic acid sequences encoding
non-naturally occurring miRNA sequences

| | Encoded | DNA Sequence | DNA Sequence |
|---|---|---|---|
| miRNA | pri- | Encoding | Encoding |
| Target | miRNA(s) | pri-miRNA | pre-miRNA |
| CTLA4 | miR204 | AGGAGGGTGGGGGTGG | GGCTACAGTCTTTC |
| | | AGGCAAGCAGAGGACTT | TTCATGTGACTCGT |
| | | CCTGATCGCGTACCCAT | GGACAATATAGTCT |
| | | GGCTACAGTCTTTCTTCA | TCTCCCTCGCTTGA |
| | | TGTGACTCGTGGACAAT | GAATATATGAAGG |
| | | ATAGTCTTCTCCCTCGCT | AAGCAGGGACAGG |
| | | TGAGAATATATGAAGGA | A |
| | | AGCAGGGACAGGACTAT | (SEQ ID NO: 347) |
| | | ATTGTTCAATTGTCATCA | |
| | | CTGGCATCTTTTTTGATC | |
| | | ATTGCACCATCATCAAAT | |
| | | GCATTGGGATAACCATG | |
| | | AC | |
| | | (SEQ ID NO: 178) | |
| PD1 | miR204 | AGGAGGGTGGGGGTGG | GGCTACAGTCTTTC |
| | | AGGCAAGCAGAGGACTT | TTCATGTGACTCGT |
| | | CCTGATCGCGTACCCAT | GGACTTCAGGAAT |
| | | GGCTACAGTCTTTCTTCA | GGGTTCCAAGGAT |
| | | TGTGACTCGTGGACTTC | GAGAATATATGAA |
| | | AGGAATGGGTTCCAAG | GGATCCTGGAAGC |
| | | GATGAGAATATATGAAG | TAT |
| | | GATCCTGGAAGCTATTC | (SEQ ID NO: 348) |
| | | CTGACGTTCAATTGTCA | |
| | | TCACTGGCATCTTTTTTG | |
| | | ATCATTGCACCATCATCA | |
| | | AATGCATTGGGATAACC | |
| | | ATGAC | |
| | | (SEQ ID NO: 179) | |
| PD1 | miR206 | GATGCTACAAGTGGCCC | TGCTTCCCGAGGCC |
| | | ACTTCTGAGATGCGGGC | TGTGGTTCTGTTAT |
| | | TGCTTCTGGATGACACT | ATCCATATATGGAT |
| | | GCTTCCCGAGGCCTGTG | TACTTTGCTATATA |
| | | GTTCTGTTATATCCATAT | ATATAATAGAACCA |
| | | ATGGATTACTTTGCTAT | CAGGTTTCGGCAA |
| | | ATAATATAATAGAACCA | G |
| | | CAGGTTTCGGCAAGTGC | (SEQ ID NO: 349) |
| | | CTCCTCGCTGGCCCCAG | |
| | | GGTACCACCCGGAGCAC | |
| | | AGGTTTGGTGACCTT | |
| | | (SEQ ID NO: 180) | |
| TIGIT | miR17 | TTAGCAGGAAAAAAGAG | GTCAGAATAATGTA |
| | | AACATCACCTTGTAAAAC | GATCCACGTTACTC |
| | | TGAAGATTGTGACCAGT | ACCCTAGTGATATG |
| | | CAGAATAATGTAGATCC | TGCATCTAGGGTGT |
| | | ACGTTACTCACCCTAGT | GTCATGTGGATGA |
| | | GATATGTGCATCTAGGG | AGCATTATGGTGAC |
| | | TGTGTCATGTGGATGAA | (SEQ ID NO: 350) |
| | | GCATTATGGTGACAGCT | |
| | | GCCTCGGGAAGCCAAGT | |
| | | TGGGCTTTAAAGTGCAG | |
| | | GGCCTGCTGATGT | |
| | | (SEQ ID NO: 181) | |
| TIGIT | miR150 | AGGGACTGGGCCCACG | CTCCCCATGGCCCT |
| | | GAGGCAGCGTCCCCGA | GAGATCCACGTTAC |
| | | GGCAGCAGCGGCAGCG | TCACCCGTGCTGGG |
| | | GCGGCTCCTCTCCCCAT | CTCAGACCCGGGT |
| | | GGCCCTGAGATCCACGT | GATAATATGGATCT |
| | | TACTCACCCGTGCTGGG | CAGGGACCTGGGG |
| | | CTCAGACCCGGGTGATA | AC |
| | | ATATGGATCTCAGGGAC | (SEQ ID NO: 351) |
| | | CTGGGGACCCCGGCACC | |
| | | GGCAGGCCCCAAGGGG | |
| | | TGAGGTGAGCGGGCAT | |
| | | TGGGACCTCCCCTCCCT | |
| | | GTACTC | |
| | | (SEQ ID NO: 182) | |
| TIGIT | miR204 | AGGAGGGTGGGGGTGG | GGCTACAGTCTTTC |
| | | AGGCAAGCAGAGGACTT | TTCATGTGACTCGT |
| | | CCTGATCGCGTACCCAT | GGACAGATCCACGT |
| | | GGCTACAGTCTTTCTTCA | TACTCACCCCCTGA |
| | | TGTGACTCGTGGACAGA | GAATATATGAAGG |
| | | TCCACGTTACTCACCCCC | AGGGGTGAGAAGC |
| | | TGAGAATATATGAAGGA | G |
| | | GGGGTGAGAAGCGTGG | (SEQ ID NO: 352) |
| | | ATCCGTTCAATTGTCATC | |
| | | ACTGGCATCTTTTTTGAT | |
| | | CATTGCACCATCATCAAA | |
| | | TGCATTGGGATAACCAT | |
| | | GAC | |
| | | (SEQ ID NO: 183) | |
| TIGIT | miR-206 | GATGCTACAAGTGGCCC | TGCTTCCCGAGGTC |
| | | ACTTCTGAGATGCGGGC | TGCGTAGCAGTCAT |
| | | TGCTTCTGGATGACACT | CGCCGGTTATGGAT |
| | | GCTTCCCGAGGTCTGCG | TACTTTGCTAACCA |
| | | TAGCAGTCATCGCCGGT | CGATGACTGCTGTG |
| | | TATGGATTACTTTGCTA | CAGATTTCGGCAA |
| | | ACCACGATGACTGCTGT | (SEQ ID NO: 353) |
| | | GCAGATTTCGGCAAGTG | |
| | | CCTCCTCGCTGGCCCCA | |
| | | GGGTACCACCCGGAGCA | |
| | | CAGGTTTGGTGACCTT | |
| | | (SEQ ID NO: 184) | |
| TIGIT | miR-204 | AGGAGGGTGGGGGTGG | GGCTACAGTCTTTC |
| | | AGGCAAGCAGAGGACTT | TTCATGTGACTCGT |
| | | CCTGATCGCGTACCCAT | GGACACCACGATG |
| | | GGCTACAGTCTTTCTTCA | ACTGCTGTGCAGA |
| | | TGTGACTCGTGGACACC | GAGAATATATGAA |
| | | ACGATGACTGCTGTGCA | GGTCTGATGGCCG |
| | | GAGAGAATATATGAAG | TCA |
| | | GTCTGATGGCCGTCATC | (SEQ ID NO: 354) |
| | | GTGGCGTTCAATTGTCA | |
| | | TCACTGGCATCTTTTTTG | |
| | | ATCATTGCACCATCATCA | |
| | | AATGCATTGGGATAACC | |
| | | ATGAC | |
| | | (SEQ ID NO: 185) | |
| TIGIT | miR-22 | CATTTTCCCTCCCTTTCC | GGCTGAGCCGCAT |
| | | CTTAGGAGCCTGTTCCT | CTGTACAGCAGCAT |
| | | CTCACGCCCTCACCTGG | CGATGGTTATGTCC |
| | | CTGAGCCGCATCTGTAC | TGACCCAGCTAACC |
| | | AGCAGCATCGATGGTTA | ACGATGACTGCTGT |
| | | TGTCCTGACCCAGCTAA | GCAGATGCCCTCTG |
| | | CCACGATGACTGCTGTG | (SEQ ID NO: 355) |
| | | CAGATGCCCTCTGCCCCT | |
| | | GGCTTCGAGGAGGAGG | |
| | | AGGAGCTGCTTTCCCCA | |
| | | TCATCTGGAAGGTG | |
| | | (SEQ ID NO: 186) | |
| TIGIT | miR-16 | cttcTGAAGAAAATATATT | GTCAGCAGTGCCTA |
| | | TCTTTTTATTCATAGCTC | CCACGATGACTGCT |
| | | TTATGATAGCAATGTCA | GTGCAGATTAAGAT |
| | | GCAGTGCCTACCACGAT | TCTAAAATTATCTT |
| | | GACTGCTGTGCAGATTA | GCGCAGCTGTTCAT |
| | | AGATTCTAAAATTATCTT | CGTGGTAGTAAGG |
| | | GCGCAGCTGTTCATCGT | (SEQ ID NO: 356) |
| | | GGTAGTAAGGTTGACCA | |
| | | TACTCTACAGTTGTGTTT | |
| | | TAATGTATATTAATGTTA | |
| | | CTAATGTGTTTT | |
| | | (SEQ ID NO: 187) | |
| TIGIT | miR-16 | cttcTGAAGAAAATATATT | GTCAGCAGTGCCTA |
| | | TCTTTTTATTCATAGCTC | CCACGATGACTGCT |
| | | TTATGATAGCAATGTCA | GTGCAGATTAAGAT |
| | | GCAGTGCCTACCACGAT | TCTAAAATTATCTT |
| | | GACTGCTGTGCAGATTA | GCGCAGCTGTTCAT |
| | | AGATTCTAAAATTATCTT | CGTGGTAAGTAAG |
| | | GCGCAGCTGTTCATCGT | (SEQ ID NO: 357) |
| | | GGTAAGTAAGGTTGACC | |
| | | ATACTCTACAGTTGTGTT | |
| | | TTAATGTATATTAATGTT | |
| | | ACTAATGTGTTTT | |
| | | (SEQ ID NO: 188) | |
| TIGIT | miR-21 | TGTCTGCTTGTTTTGCCT | TGTCGGGACCACG |
| | | ACCATCGTGACATCTCCA | ATGACTGCTGTGCA |
| | | TGGCTGTACCACCTTGT | GACTGTTGAATCTC |
| | | CGGGACCACGATGACTG | ATGGCTGCGAAGC |
| | | CTGTGCAGACTGTTGAA | AGCATCGTGGCTCT |
| | | TCTCATGGCTGCGAAGC | GACA |
| | | AGCATCGTGGCTCTGAC | (SEQ ID NO: 358) |
| | | ATTTTGGTATCTTTCATC | |
| | | TGACCATCCATATCCAAT | |
| | | GTTCTCATTTAAACA | |
| | | (SEQ ID NO: 189) | |
| TIGIT | miR-494 | CAGTTCTGTTTTGATTTT | GATACTCGAAGGAT |
| | | TTTTGTTTGTTTTTTGAT | CTGCAACGCAGTCT |
| | | CAGTGCTAATCTTCGAT | CGCTGGTCTTTATT |
| | | ACTCGAAGGATCTGCAA | TATGAACCACGATG |
| | | CGCAGTCTCGCTGGTCT | ACTGCTGTGCAGAT |
| | | TTATTTATGAACCACGAT | TTTTTAGTATC |
| | | GACTGCTGTGCAGATTT | (SEQ ID NO: 359) |
| | | TTTAGTATCAAATCCCAC | |
| | | CCTGGAGGCACTTCCTG | |
| | | TTCCTGATGCAGCCTTCA | |
| | | GGGAGG | |
| | | (SEQ ID NO: 190) | |
| TIGIT | miR-142 | GGAGTCAGGAGGCCTG | GACAGTGCAGTCAC |
| | | GGCAGCCTGAAGAGTAC | CACCACGATGACTG |
| | | ACGCCGACGGACAGACA | CTGTGCAGAACAGC |
| | | GACAGTGCAGTCACCAC | ACTGGAGGGATGC |
| | | CACGATGACTGCTGTGC | ACAGCAATCATCGT |
| | | AGAACAGCACTGGAGG | GGTGATGAGTGTA |
| | | GATGCACAGCAATCATC | (SEQ ID NO: 360) |
| | | GTGGTGATGAGTGTACT | |
| | | GTGGGCTTCGGAGATCA | |
| | | CGCCACTGCTGCCGCCC | |
| | | GCTGCCCGCCACCATCTT | |
| | | C | |
| | | (SEQ ID NO: 191) | |
| TIGIT | miR-19 | CCAATAATTCAAGCCAA | GCAGTCCTCTGATC |
| | | GCAAGTATATAGGTGTT | TGCACAGCAGTTCG |
| | | TTAATAGTTTTTGTTTGC | TGGTACAAGAAGA |
| | | AGTCCTCTGATCTGCAC | ATGTAGTACCACGA |
| | | AGCAGTTCGTGGTACAA | TGACTGCTGTGCAG |
| | | GAAGAATGTAGTACCAC | ATTGGTGGCCTGC |
| | | GATGACTGCTGTGCAGA | (SEQ ID NO: 361) |
| | | TTGGTGGCCTGCTATTT | |
| | | CCTTCAAATGAATGATTT | |
| | | TTACTAATTTTGTGTACT | |
| | | TTTATTGTG | |
| | | (SEQ ID NO: 192) | |
| TIGIT | miR-1915 | CGGACCACGGTGTCCCC | TGAGAGGCCGCAC |
| | | TTCTCTCCAGCTGGGGG | CACGATGACTGCTG |
| | | TCTCGGGTCCTGGCGCT | TGCAGAGTGCACCC |
| | | GAGAGGCCGCACCACGA | GTGTCTGCCAATAG |
| | | TGACTGCTGTGCAGAGT | CTGTAATCGTGGGC |
| | | GCACCCGTGTCTGCCAA | GGCCCTAGCGA |
| | | TAGCTGTAATCGTGGGC | (SEQ ID NO: 362) |
| | | GGCCCTAGCGACCTGCG | |
| | | GCGGCGCCGGGAAAGC | |
| | | CCTGCCTCTGCAGCGGG | |
| | | TCCCAGGGGTC | |
| | | (SEQ ID NO: 193) | |
| TIGIT | miR-206 | GATGCTACAAGTGGCCC | TGCTTCCCGAGGCA |
| | | ACTTCTGAGATGCGGGC | GTCGTTGACTGTG |
| | | TGCTTCTGGATGACACT | GACTTATATATGGA |
| | | GCTTCCCGAGGCAGTCG | TTACTTTGCTATAT |
| | | TTGACTGTGGACTTATA | CGTTCACGGTCAGC |
| | | TATGGATTACTTTGCTAT | GACTGTTTCGGCAA |
| | | ATCGTTCACGGTCAGCG | (SEQ ID NO: 363) |
| | | ACTGTTTCGGCAAGTGC | |
| | | CTCCTCGCTGGCCCCAG | |
| | | GGTACCACCCGGAGCAC | |
| | | AGGTTTGGTGACCTT | |
| | | (SEQ ID NO: 194) | |
| TIGIT | miR-204 | AGGAGGGTGGGGGTGG | GGCTACAGTCTTTC |
| | | AGGCAAGCAGAGGACTT | TTCATGTGACTCGT |
| | | CCTGATCGCGTACCCAT | GGACTATCGTTCAC |
| | | GGCTACAGTCTTTCTTCA | GGTCAGCGACTGG |
| | | TGTGACTCGTGGACTAT | AGAATATATGAAG |
| | | CGTTCACGGTCAGCGAC | GCAGTGCTGAACG |
| | | TGGAGAATATATGAAGG | (SEQ ID NO: 364) |
| | | CAGTGCTGAACGTGAAC | |
| | | GATCGTTCAATTGTCAT | |
| | | CACTGGCATCTTTTTTGA | |
| | | TCATTGCACCATCATCAA | |
| | | ATGCATTGGGATAACCA | |
| | | TGAC | |
| | | (SEQ ID NO: 195) | |
| TIGIT | miR-22 | CATTTTCCCTCCCTTTCC | GGCTGAGCCGCAT |
| | | CTTAGGAGCCTGTTCCT | AGTCGCTGACTTGA |
| | | CTCACGCCCTCACCTGG | ACAGATATATGTCC |
| | | CTGAGCCGCATAGTCGC | TGACCCAGCTATAT |
| | | TGACTTGAACAGATATA | CGTTCACGGTCAGC |
| | | TGTCCTGACCCAGCTAT | GACTGTGCCCTCTG |
| | | ATCGTTCACGGTCAGCG | (SEQ ID NO: 365) |
| | | ACTGTGCCCTCTGCCCCT | |
| | | GGCTTCGAGGAGGAGG | |
| | | AGGAGCTGCTTTCCCCA | |
| | | TCATCTGGAAGGTG | |
| | | (SEQ ID NO: 196) | |
| TIGIT | miR-142 | GGAGTCAGGAGGCCTG | GACAGTGCAGTCAC |
| | | GGCAGCCTGAAGAGTAC | CTATCGTTCACGGT |
| | | ACGCCGACGGACAGACA | CAGCGACTAACAGC |
| | | GACAGTGCAGTCACCTA | ACTGGAGGGTGTC |
| | | TCGTTCACGGTCAGCGA | GCTGACAGTGAAC |
| | | CTAACAGCACTGGAGGG | GATAGATGAGTGT |
| | | TGTCGCTGACAGTGAAC | A |
| | | GATAGATGAGTGTACTG | (SEQ ID NO: 366) |
| | | TGGGCTTCGGAGATCAC | |
| | | GCCACTGCTGCCGCCCG | |
| | | CTGCCCGCCACCATCTTC | |
| | | (SEQ ID NO: 197) | |
| TIGIT | miR-16 | cttcTGAAGAAAATATATT | GTCAGCAGTGCCTT |
| | | TCTTTTTATTCATAGCTC | ATCGTTCACGGTCA |
| | | TTATGATAGCAATGTCA | GCGACTGTTAAGAT |
| | | GCAGTGCCTTATCGTTC | TCTAAAATTATCTG |
| | | ACGGTCAGCGACTGTTA | TCGCTGAGCGCTGA |
| | | AGATTCTAAAATTATCT | ACGATGAAGTAAG |
| | | GTCGCTGAGCGCTGAAC | (SEQ ID NO: 367) |
| | | GATGAAGTAAGGTTGAC | |
| | | CATACTCTACAGTTGTG | |
| | | TTTTAATGTATATTAATG | |
| | | TTACTAATGTGTTTT | |
| | | (SEQ ID NO: 198) | |
| TIGIT | miR-206 | GATGCTACAAGTGGCCC | TGCTTCCCGAGGAC |
| | | ACTTCTGAGATGCGGGC | TACTTCAATGTCCT |
| | | TGCTTCTGGATGACACT | GACCTTATATGGAT |
| | | GCTTCCCGAGGACTACT | TACTTTGCTATAAC |
| | | TCAATGTCCTGACCTTAT | TCAGGACATTGAAG |
| | | ATGGATTACTTTGCTAT | TAGTTTTCGGCAAG |
| | | AACTCAGGACATTGAAG | (SEQ ID NO: 368) |
| | | TAGTTTTCGGCAAGTGC | |
| | | CTCCTCGCTGGCCCCAG | |
| | | GGTACCACCCGGAGCAC | |
| | | AGGTTTGGTGACCTT | |
| | | (SEQ ID NO: 199) | |
| TIGIT | miR-204 | AGGAGGGTGGGGGTGG | GGCTACAGTCTTTC |
| | | AGGCAAGCAGAGGACTT | TTCATGTGACTCGT |
| | | CCTGATCGCGTACCCAT | GGACTAACTCAGG |
| | | GGCTACAGTCTTTCTTCA | ACATTGAAGTAGTG |
| | | TGTGACTCGTGGACTAA | AGAATATATGAAG |
| | | CTCAGGACATTGAAGTA | GACTATTCAGAGTC |
| | | GTGAGAATATATGAAGG | C |
| | | ACTATTCAGAGTCCTGA | (SEQ ID NO: 369) |
| | | GTTCGTTCAATTGTCATC | |
| | | ACTGGCATCTTTTTTGAT | |
| | | CATTGCACCATCATCAAA | |
| | | TGCATTGGGATAACCAT | |
| | | GAC | |
| | | (SEQ ID NO: 200) | |
| TIGIT | miR-21 | TGTCTGCTTGTTTTGCCT | TGTCGGGTAACTCA |
| | | ACCATCGTGACATCTCCA | GGACATTGAAGTA |
| | | TGGCTGTACCACCTTGT | GTCTGTTGAATCTC |
| | | CGGGTAACTCAGGACAT | ATGGCTACTCCAAT |
| | | TGAAGTAGTCTGTTGAA | GCCTGAGTTGTCTG |
| | | TCTCATGGCTACTCCAAT | ACA |
| | | GCCTGAGTTGTCTGACA | (SEQ ID NO: 370) |
| | | TTTTGGTATCTTTCATCT | |
| | | GACCATCCATATCCAAT | |
| | | GTTCTCATTTAAACA | |
| | | (SEQ ID NO: 201) | |
| TIGIT | miR-494 | CAGTTCTGTTTTGATTTT | GATACTCGAAGGA |
| | | TTTTGTTTGTTTTTTGAT | ACTACTGTAATGTC |
| | | CAGTGCTAATCTTCGAT | TGACGTTTCTTTAT |
| | | ACTCGAAGGAACTACTG | TTATGATAACTCAG |
| | | TAATGTCTGACGTTTCTT | GACATTGAAGTAGT |
| | | TATTTATGATAACTCAG | TTTTTTAGTATC |
| | | GACATTGAAGTAGTTTT | (SEQ ID NO: 371) |
| | | TTTAGTATCAAATCCCAC | |
| | | CCTGGAGGCACTTCCTG | |
| | | TTCCTGATGCAGCCTTCA | |
| | | GGGAGG | |
| | | (SEQ ID NO: 202) | |
| TIGIT | miR-19 | CCAATAATTCAAGCCAA | GCAGTCCTCTGGAC |
| | | GCAAGTATATAGGTGTT | TACTTCAATGTTGA |
| | | TTAATAGTTTTTGTTTGC | GTTTACAAGAAGAA |
| | | AGTCCTCTGGACTACTTC | TGTAGTTAACTCAG |
| | | AATGTTGAGTTTACAAG | GACATTGAAGTAGT |
| | | AAGAATGTAGTTAACTC | CTGGTGGCCTGC |
| | | AGGACATTGAAGTAGTC | (SEQ ID NO: 372) |
| | | TGGTGGCCTGCTATTTC | |
| | | CTTCAAATGAATGATTTT | |
| | | TACTAATTTTGTGTACTT | |
| | | TTATTGTG | |
| | | (SEQ ID NO: 203) | |
| TIGIT | miR-1915 | CGGACCACGGTGTCCCC | TGAGAGGCCGCAG |
| | | TTCTCTCCAGCTGGGGG | ATCCACGTTACTCA |
| | | TCTCGGGTCCTGGCGCT | CCCTAGGTGCACCC |
| | | GAGAGGCCGCAGATCCA | GTGCTAGGCAGTG |
| | | CGTTACTCACCCTAGGT | AGCAAAGTGGATC |
| | | GCACCCGTGCTAGGCAG | GCGGCCCTAGCGA |
| | | TGAGCAAAGTGGATCGC | (SEQ ID NO: 373) |
| | | GGCCCTAGCGACCTGCG | |
| | | GCGGCGCCGGGAAAGC | |
| | | CCTGCCTCTGCAGCGGG | |
| | | TCCCAGGGGTC | |
| | | (SEQ ID NO: 204) | |
| TIGIT | miR-204 | AGGAGGGTGGGGGTGG | GGCTACAGTCTTTC |
| | | AGGCAAGCAGAGGACTT | TTCATGTGACTCGT |
| | | CCTGATCGCGTACCCAT | GGACAGATCCACGT |
| | | GGCTACAGTCTTTCTTCA | TACTCACCCTAGGA |
| | | TGTGACTCGTGGACAGA | GAATATATGAAGG |
| | | TCCACGTTACTCACCCTA | CTAGGTGAGAAAC |
| | | GGAGAATATATGAAGGC | GTGGATCGGTTCA |
| | | TAGGTGAGAAACGTGG | ATTGTCATCACTGG |
| | | ATCGGTTCAATTGTCAT | C |
| | | CACTGGCATCTTTTTTGA | (SEQ ID NO: 374) |
| | | TCATTGCACCATCATCAA | |
| | | ATGCATTGGGATAACCA | |
| | | TGAC | |
| | | (SEQ ID NO: 205) | |
| TIGIT | miR-206 | GATGCTACAAGTGGCCC | TGCTTCCCGAGGCT |
| | | ACTTCTGAGATGCGGGC | AGGGTGAGTAACG |
| | | TGCTTCTGGATGACACT | TGGCCTCTTATGGA |
| | | GCTTCCCGAGGCTAGGG | TTACTTTGCTAAGA |
| | | TGAGTAACGTGGCCTCT | TCCACGTTACTCAC |
| | | TATGGATTACTTTGCTA | CCTAGTTTCGGCAA |
| | | AGATCCACGTTACTCACC | GTG |
| | | CTAGTTTCGGCAAGTGC | (SEQ ID NO: 375) |
| | | CTCCTCGCTGGCCCCAG | |
| | | GGTACCACCCGGAGCAC | |
| | | AGGTTTGGTGACCTT | |
| | | (SEQ ID NO: 206) | |
| TIGIT | miR-21 | TGTCTGCTTGTTTTGCCT | TGTCGGGAGATCC |
| | | ACCATCGTGACATCTCCA | ACGTTACTCACCCT |
| | | TGGCTGTACCACCTTGT | AGCTGTTGAATCTC |
| | | CGGGAGATCCACGTTAC | ATGGTAGGGCGAG |
| | | TCACCCTAGCTGTTGAA | TACGTGGATCGTCT |
| | | TCTCATGGTAGGGCGAG | GACA |
| | | TACGTGGATCGTCTGAC | (SEQ ID NO: 376) |
| | | ATTTTGGTATCTTTCATC | |
| | | TGACCATCCATATCCAAT | |
| | | GTTCTCATTTAAACA | |
| | | (SEQ ID NO: 207) | |
| TIGIT | miR-22 | CATTTTCCCTCCCTTTCC | GGCTGAGCCGCAC |
| | | CTTAGGAGCCTGTTCCT | TAGGGTGAGTACG |
| | | CTCACGCCCTCACCTGG | TGGCATCTTATGTC |
| | | CTGAGCCGCACTAGGGT | CTGACCCAGCTAAG |
| | | GAGTACGTGGCATCTTA | ATCCACGTTACTCA |
| | | TGTCCTGACCCAGCTAA | CCCTAGTGCCCTCT |
| | | GATCCACGTTACTCACCC | GCC |
| | | TAGTGCCCTCTGCCCCT | (SEQ ID NO: 377) |
| | | GGCTTCGAGGAGGAGG | |
| | | AGGAGCTGCTTTCCCCA | |
| | | TCATCTGGAAGGTG | |
| | | (SEQ ID NO: 208) | |
| TIM3 | miR204 | AGGAGGGTGGGGGTGG | GGCTACAGTCTTTC |
| | | AGGCAAGCAGAGGACTT | TTCATGTGACTCGT |
| | | CCTGATCGCGTACCCAT | GGACCATTATGCCT |
| | | GGCTACAGTCTTTCTTCA | GGGATTTGGATCG |
| | | TGTGACTCGTGGACCAT | AGAATATATGAAG |
| | | TATGCCTGGGATTTGGA | GGATCAGATCGCA |
| | | TCGAGAATATATGAAGG | GGCATAATTGTTCA |
| | | GATCAGATCGCAGGCAT | ATTGTCATCACTGG |
| | | AATTGTTCAATTGTCATC | C |
| | | ACTGGCATCTTTTTTGAT | (SEQ ID NO: 378) |
| | | CATTGCACCATCATCAAA | |
| | | TGCATTGGGATAACCAT | |
| | | GAC | |
| | | (SEQ ID NO: 209) | |
| TIM3 | miR150 | AGGGACTGGGCCCACG | CTCCCCATGGCCCT |
| | | GGGAGGCAGCGTCCCC | GCATTATGCCTGGG |
| | | GAGGCAGCAGCGGCAG | ATTTGGATCCTGGG |
| | | CGGCGGCTCCTCTCCCC | CTCAGACCTCCAAA |
| | | ATGGCCCTGCATTATGC | TCCACACATAGTGC |
| | | CTGGGATTTGGATCCTG | AGGGACCTGGGGA |
| | | GGCTCAGACCTCCAAAT | C |
| | | CCACACATAGTGCAGGG | (SEQ ID NO: 379) |
| | | ACCTGGGGACCCCGGCA | |
| | | CCGGCAGGCCCCAAGG | |
| | | GGTGAGGTGAGCGGGC | |
| | | ATTGGGACCTCCCCTCCC | |
| | | TGTACTC | |
| | | (SEQ ID NO: 210) | |
| TIM3 | miR30a | CAGGTTAACCCAACAGA | GCGACCATTATGCC |
| | | AGGCTAAAGAAGGTATA | TGGGATTTGGATCC |
| | | TTGCTGTTGACAGTGAG | TGTGAAGCCACAG |
| | | CGACCATTATGCCTGGG | ATGGGGATCCAGA |
| | | ATTTGGATCCTGTGAAG | TCAGGCATAGTGG |
| | | CCACAGATGGGGATCCA | CTGC |
| | | GATCAGGCATAGTGGCT | (SEQ ID NO: 380) |
| | | GCCTACTGCCTCGGACT | |
| | | TCAAGGGGCTACTTTAG | |
| | | GAGCAATTATCTTGTTT | |
| | | A | |
| | | (SEQ ID NO: 211) | |
| TIM3 | miR206 | GATGCTACAAGTGGCCC | TGCTTCCCGAGGGA |
| | | ACTTCTGAGATGCGGGC | TCCGGATCCTAGGT |
| | | TGCTTCTGGATGACACT | ATCCATGTATGGAT |
| | | GCTTCCCGAGGGATCCG | TACTTTGCTACATT |
| | | GATCCTAGGTATCCATG | ATGCCTGGGATTTG |
| | | TATGGATTACTTTGCTAC | GATCTTTCGGCAAG |
| | | ATTATGCCTGGGATTTG | TG |
| | | GATCTTTCGGCAAGTGC | (SEQ ID NO: 381) |
| | | CTCCTCGCTGGCCCCAG | |
| | | GGTACCACCCGGAGCAC | |
| | | AGGTTTGGTGACCTT | |
| | | (SEQ ID NO: 212) | |
| TIM3 | miR16 | CTTCTGAAGAAAATATA | GTCAGCAGTGCCTC |
| | | TTTCTTTTTATTCATAGC | ATTATGCCTGGGAT |
| | | TCTTATGATAGCAATGT | TTGGATCTTAAGAT |
| | | CAGCAGTGCCTCATTAT | TCTAAAATTATCTTC |
| | | GCCTGGGATTTGGATCT | CGAATCACATGGCA |
| | | TAAGATTCTAAAATTATC | TAATGAAGTAAGG |
| | | TTCCGAATCACATGGCA | TTGAC |
| | | TAATGAAGTAAGGTTGA | (SEQ ID NO: 382) |
| | | CCATACTCTACAGTTGT | |
| | | GTTTTAATGTATATTAAT | |
| | | GTTACTAATGTGTTTT | |
| | | (SEQ ID NO: 213) | |
| TIM3 | miR17 | TTAGCAGGAAAAAAGAG | GTCAGAATAATGTC |
| | | AACATCACCTTGTAAAAC | ATTATGCCTGGGAT |
| | | TGAAGATTGTGACCAGT | TTGGATCGTGATAT |
| | | CAGAATAATGTCATTAT | GTGCATCGGCCAG |
| | | GCCTGGGATTTGGATCG | AACCAAGGCATAAG |
| | | TGATATGTGCATCGGCC | TAGCATTATGGTGA |
| | | AGAACCAAGGCATAAGT | C |
| | | AGCATTATGGTGACAGC | (SEQ ID NO: 383) |
| | | TGCCTCGGGAAGCCAAG | |
| | | TTGGGCTTTAAAGTGCA | |
| | | GGGCCTGCTGATGT | |
| | | (SEQ ID NO: 214) | |
| TIM3 | miR126 | CCGGGGTCCTGTCTGCA | CGCTGGCGACGGA |
| | | TCCAGCGCAGCATTCTG | CTCCAAATCCCGTG |
| | | GAAGACGCCACGCCTCC | CATAATCGCTGTGA |
| | | GCTGGCGACGGACTCCA | CACTTCAAACCATT |
| | | AATCCCGTGCATAATCG | ATGCCTGGGATTTG |
| | | CTGTGACACTTCAAACC | GATCCCGTCCACGG |
| | | ATTATGCCTGGGATTTG | CA |
| | | GATCCCGTCCACGGCAC | (SEQ ID NO: 384) |
| | | CGCATCGAAAACGCCGC | |
| | | TGAGACCTCAGCCTTGA | |
| | | CCTCCCTCAGCGTGG | |
| | | (SEQ ID NO: 215) | |
| TIM3 | miR122 | CAATGGTGGAATGTGG | CCTTAGCAGAGCTG |
| | | AGGTGAAGTTAACACCT | CATTATGCCTGGGA |
| | | TCGTGGCTACAGAGTTT | TTTGGATCTGTCTA |
| | | CCTTAGCAGAGCTGCAT | AACTATGATTCGAA |
| | | TATGCCTGGGATTTGGA | TCCAAGGCATATGG |
| | | TCTGTCTAAACTATGATT | TAGCTACTGCTAGG |
| | | CGAATCCAAGGCATATG | CAATCCTTCCCTCG |
| | | GTAGCTACTGCTAGGCA | ATAAATGTCTTGGC |
| | | ATCCTTCCCTCGATAAAT | (SEQ ID NO: 385) |
| | | GTCTTGGCATCGTTTGC | |
| | | TTTGAGCAAGAAGGTTC | |
| | | ATCTGATATCAGTCTTCT | |
| | | CAATCT | |
| | | (SEQ ID NO: 216) | |
| TIM3 | miR214 | ACAGGCTGATTGTATCT | GGCCTGGCTGGAC |
| | | GTCTATGAGCAAAGGAA | AGAGTTGTCATGTG |
| | | ACCTGAAGGAACCAAGG | ATGATGAAAGGAC |
| | | GCCTGGCTGGACAGAGT | AGTGAATTAGCAG |
| | | TGTCATGTGATGATGAA | AACATCCGCTCACC |
| | | AGGACAGTGAATTAGCA | TGTTAATTCACATC |
| | | GAACATCCGCTCACCTG | CCTTTCATCAGCAC |
| | | TTAATTCACATCCCTTTC | ATGACAACCCAGCC |
| | | ATCAGCACATGACAACC | T |
| | | CAGCCTGAATGACAACC | (SEQ ID NO: 386) |
| | | AGCCATTGAAAGAAAGC | |
| | | AGCCCTCACACCATAGC | |
| | | ATCTA | |
| | | (SEQ ID NO: 217) | |
| TIM3 | miR29b1 | GGGTTTATTGTAAGAGA | CTTCAGGACGATG |
| | | GCATTATGAAGAAAAAA | GAAGGGTTGTGAA |
| | | ATAGATCATAAAGCTTC | CGTTAGATTTAAAT |
| | | TTCAGGACGATGGAAG | AGTGATTGTCTAAT |
| | | GGTTGTGAACGTTAGAT | TCACATCCCTTTCAT |
| | | TTAAATAGTGATTGTCT | CAGTCTTGGGGG |
| | | AATTCACATCCCTTTCAT | (SEQ ID NO: 387) |
| | | CAGTCTTGGGGGAGACC | |
| | | AGCTGCGCTGCACTACC | |
| | | AACAGCAAAAGAAGTGA | |
| | | ATGGGACAGCT | |
| | | (SEQ ID NO: 218) | |
| TIM3 | miR204 | AGGAGGGTGGGGGTGG | GGCTACAGTCTTTC |
| | | AGGCAAGCAGAGGACTT | TTCATGTGACTCGT |
| | | CCTGATCGCGTACCCAT | GGACTAATTCACAT |
| | | GGCTACAGTCTTTCTTCA | CCCTTTCATCAGGA |
| | | TGTGACTCGTGGACTAA | GAATATATGAAGG |
| | | TTCACATCCCTTTCATCA | CTGAGGAAGAGGT |
| | | GGAGAATATATGAAGGC | GTGAATTCGTTCAA |
| | | TGAGGAAGAGGTGTGA | TTGTCATCACTGGC |
| | | ATTCGTTCAATTGTCATC | (SEQ ID NO: 388) |
| | | ACTGGCATCTTTTTTGAT | |
| | | CATTGCACCATCATCAAA | |
| | | TGCATTGGGATAACCAT | |
| | | GAC | |
| | | (SEQ ID NO: 219) | |
| TIM3 | miR133a1 | TTTACCAATGAAAAGCA | ACAATGCTTTGCTA |
| | | TTTAACTGTTTTGGATTC | GTGATGAATAGGC |
| | | CAAACTAGCAGCACTAC | TGCGAATTATCGCC |
| | | AATGCTTTGCTAGTGAT | TCTTCAATGGATAA |
| | | GAATAGGCTGCGAATTA | TTCACATCCCTTTCA |
| | | TCGCCTCTTCAATGGAT | TCAGTAGCTATGCA |
| | | AATTCACATCCCTTTCAT | TTGA |
| | | CAGTAGCTATGCATTGA | (SEQ ID NO: 389) |
| | | TTACTACGGGACAACCA | |
| | | ACGTTTTCATTTGTGAAT | |
| | | ATCAATTACTTGCCA | |
| | | (SEQ ID NO: 220) | |
| LAG3 | miR30a | CAGGTTAACCCAACAGA | GCGACTAGTCGTTG |
| | | AGGCTAAAGAAGGTATA | GGTAAAGTCGCCAC |
| | | TTGCTGTTGACAGTGAG | TGTGAAGCCACAG |
| | | CGACTAGTCGTTGGGTA | ATGGGTGGTGACT |
| | | AAGTCGCCACTGTGAAG | TCTCAACGATTAGC |
| | | CCACAGATGGGTGGTG | TGC |
| | | ACTTCTCAACGATTAGCT | (SEQ ID NO: 390) |
| | | GCCTACTGCCTCGGACT | |
| | | TCAAGGGGCTACTTTAG | |
| | | GAGCAATTATCTTGTTT | |
| | | A | |
| | | (SEQ ID NO: 221) | |
| LAG3 | miR206 | GATGCTACAAGTGGCCC | TGCTTCCCGAGGTG |
| | | ACTTCTGAGATGCGGGC | GCGATTTTACCCAA |
| | | TGCTTCTGGATGACACT | TGCTCTATATGGAT |
| | | GCTTCCCGAGGTGGCGA | TACTTTGCTATAGT |
| | | TTTTACCCAATGCTCTAT | CGTTGGGTAAAGT |
| | | ATGGATTACTTTGCTAT | CGCCATTTCGGCAA |
| | | AGTCGTTGGGTAAAGTC | GTG |
| | | GCCATTTCGGCAAGTGC | (SEQ ID NO: 391) |
| | | CTCCTCGCTGGCCCCAG | |
| | | GGTACCACCCGGAGCAC | |
| | | AGGTTTGGTGACCTT | |
| | | (SEQ ID NO: 222) | |
| LAG3 | miR204 | AGGAGGGTGGGGGTGG | GGCTACAGTCTTTC |
| | | AGGCAAGCAGAGGACTT | TTCATGTGACTCGT |
| | | CCTGATCGCGTACCCAT | GGACTAGTCGTTG |
| | | GGCTACAGTCTTTCTTCA | GGTAAAGTCGCCA |
| | | TGTGACTCGTGGACTAG | GAGAATATATGAA |
| | | TCGTTGGGTAAAGTCGC | GGTGGCACTTTCCT |
| | | CAGAGAATATATGAAGG | CAACGACTCGTTCA |
| | | TGGCACTTTCCTCAACG | ATTGTCATCACTGG |
| | | ACTCGTTCAATTGTCATC | C |
| | | ACTGGCATCTTTTTTGAT | (SEQ ID NO: 392) |
| | | CATTGCACCATCATCAAA | |
| | | TGCATTGGGATAACCAT | |
| | | GAC | |
| | | (SEQ ID NO: 223) | |
| LAG3 | miR150 | AGGGACTGGGCCCACG | CTCCCCATGGCCCT |
| | | GGGAGGCAGCGTCCCC | GTAGTCGTTGGGT |
| | | GAGGCAGCAGCGGCAG | AAAGTCGCCACTGG |
| | | CGGCGGCTCCTCTCCCC | GCTCAGACCGCGAC |
| | | ATGGCCCTGTAGTCGTT | TTACCACACGACTA |
| | | GGGTAAAGTCGCCACTG | CAGGGACCTGGGG |
| | | GGCTCAGACCGCGACTT | AC |
| | | ACCACACGACTACAGGG | (SEQ ID NO: 393) |
| | | ACCTGGGGACCCCGGCA | |
| | | CCGGCAGGCCCCAAGG | |
| | | GGTGAGGTGAGCGGGC | |
| | | ATTGGGACCTCCCCTCCC | |
| | | TGTACTC | |
| | | (SEQ ID NO: 224) | |
| LAG3 | miR17 | TTAGCAGGAAAAAAGAG | GTCAGAATAATGTG |
| | | AACATCACCTTGTAAAAC | TTGCTTTCCGCTAA |
| | | TGAAGATTGTGACCAGT | GTGGTGAGTGATA |
| | | CAGAATAATGTGTTGCT | TGTGCATCTCCCAC |
| | | TTCCGCTAAGTGGTGAG | TCAGAGGAAAGCA |
| | | TGATATGTGCATCTCCC | CTAGCATTATGGTG |
| | | ACTCAGAGGAAAGCACT | AC |
| | | AGCATTATGGTGACAGC | (SEQ ID NO: 394) |
| | | TGCCTCGGGAAGCCAAG | |
| | | TTGGGCTTTAAAGTGCA | |
| | | GGGCCTGCTGATGT | |
| | | (SEQ ID NO: 225) | |
| LAG3 | miR122 | CAATGGTGGAATGTGG | CCTTAGCAGAGCTG |
| | | AGGTGAAGTTAACACCT | GTTGCTTTCCGCTA |
| | | TCGTGGCTACAGAGTTT | AGTGGTGATGTCTA |
| | | CCTTAGCAGAGCTGGTT | AACTATTCACCACT |
| | | GCTTTCCGCTAAGTGGT | TAGAGGAAAGCCT |
| | | GATGTCTAAACTATTCAC | CTAGCTACTGCTAG |
| | | CACTTAGAGGAAAGCCT | GCAATCCTTCCCTC |
| | | CTAGCTACTGCTAGGCA | GATAAATGTCTTGG |
| | | ATCCTTCCCTCGATAAAT | C |
| | | GTCTTGGCATCGTTTGC | (SEQ ID NO: 395) |
| | | TTTGAGCAAGAAGGTTC | |
| | | ATCTGATATCAGTCTTCT | |
| | | CAATCT | |
| | | (SEQ ID NO: 226) | |
| LAG3 | miR126 | CCGGGGTCCTGTCTGCA | CGCTGGCGACGGG |
| | | TCCAGCGCAGCATTCTG | AACCACTTAGCGCG |
| | | GAAGACGCCACGCCTCC | AAGCAAGGCTGTG |
| | | GCTGGCGACGGGAACC | ACACTTCAAACGTT |
| | | ACTTAGCGCGAAGCAAG | GCTTTCCGCTAAGT |
| | | GCTGTGACACTTCAAAC | GGTGACCGTCCAC |
| | | GTTGCTTTCCGCTAAGT | GGCA |
| | | GGTGACCGTCCACGGCA | (SEQ ID NO: 396) |
| | | CCGCATCGAAAACGCCG | |
| | | CTGAGACCTCAGCCTTG | |
| | | ACCTCCCTCAGCGTGG | |
| | | (SEQ ID NO: 227) | |
| GITR | miR30a | CAGGTTAACCCAACAGA | GCGACTTTGCAGTG |
| | | AGGCTAAAGAAGGTATA | GCCTTCGTGGCCCC |
| | | TTGCTGTTGACAGTGAG | TGTGAAGCCACAG |
| | | CGACTTTGCAGTGGCCT | ATGGGGGGTCACG |
| | | TCGTGGCCCCTGTGAAG | AGCCACTGCGAAGC |
| | | CCACAGATGGGGGGTC | TGC |
| | | ACGAGCCACTGCGAAGC | (SEQ ID NO: 397) |
| | | TGCCTACTGCCTCGGAC | |
| | | TTCAAGGGGCTACTTTA | |
| | | GGAGCAATTATCTTGTT | |
| | | TA | |
| | | (SEQ ID NO: 228) | |
| GITR | miR206 | GATGCTACAAGTGGCCC | TGCTTCCCGAGGG |
| | | ACTTCTGAGATGCGGGC | GGCTATGAAGGCC |
| | | TGCTTCTGGATGACACT | ATTGAGAAATATG |
| | | GCTTCCCGAGGGGGCTA | GATTACTTTGCTAT |
| | | TGAAGGCCATTGAGAAA | TTGCAGTGGCCTTC |
| | | TATGGATTACTTTGCTAT | GTAGCCCTTTCGGC |
| | | TTGCAGTGGCCTTCGTA | AAGTG |
| | | GCCCTTTCGGCAAGTGC | (SEQ ID NO: 398) |
| | | CTCCTCGCTGGCCCCAG | |
| | | GGTACCACCCGGAGCAC | |
| | | AGGTTTGGTGACCTT | |
| | | (SEQ ID NO: 229) | |
| GITR | miR17 | TTAGCAGGAAAAAAGAG | GTCAGAATAATGTT |
| | | AACATCACCTTGTAAAAC | TTGCAGTGGCCTTC |
| | | TGAAGATTGTGACCAGT | GTGGCCCGTGATAT |
| | | CAGAATAATGTTTTGCA | GTGCATCGGTCACG |
| | | GTGGCCTTCGTGGCCCG | TAGACTACTGCACT |
| | | TGATATGTGCATCGGTC | CGCATTATGGTGAC |
| | | ACGTAGACTACTGCACT | (SEQ ID NO: 399) |
| | | CGCATTATGGTGACAGC | |
| | | TGCCTCGGGAAGCCAAG | |
| | | TTGGGCTTTAAAGTGCA | |
| | | GGGCCTGCTGATGT | |
| | | (SEQ ID NO: 230) | |
| GITR | miR122 | CAATGGTGGAATGTGG | CCTTAGCAGAGCTG |
| | | AGGTGAAGTTAACACCT | TTTGCAGTGGCCTT |
| | | TCGTGGCTACAGAGTTT | CGTGGCCCTGTCTA |
| | | CCTTAGCAGAGCTGTTT | AACTATGGGTCACG |
| | | GCAGTGGCCTTCGTGGC | AAGACCACTGCCCA |
| | | CCTGTCTAAACTATGGG | TAGCTACTGCTAGG |
| | | TCACGAAGACCACTGCC | CAATCCTTCCCTCG |
| | | CATAGCTACTGCTAGGC | ATAAATGTCTTGGC |
| | | AATCCTTCCCTCGATAAA | (SEQ ID NO: 400) |
| | | TGTCTTGGCATCGTTTG | |
| | | CTTTGAGCAAGAAGGTT | |
| | | CATCTGATATCAGTCTTC | |
| | | TCAATCT | |
| | | (SEQ ID NO: 231) | |
| GITR | miR150 | AGGGACTGGGCCCACG | CTCCCCATGGCCCT |
| | | GGGAGGCAGCGTCCCC | GAGCCTCCCGTCCT |
| | | GAGGCAGCAGCGGCAG | AAGACCCCACTGGG |
| | | CGGCGGCTCCTCTCCCC | CTCAGACAGGGTCT |
| | | ATGGCCCTGAGCCTCCC | TGGAAAGGAGGCT |
| | | GTCCTAAGACCCCACTG | CAGGGACCTGGGG |
| | | GGCTCAGACAGGGTCTT | AC |
| | | GGAAAGGAGGCTCAGG | (SEQ ID NO: 401) |
| | | GACCTGGGGACCCCGGC | |
| | | ACCGGCAGGCCCCAAGG | |
| | | GGTGAGGTGAGCGGGC | |
| | | ATTGGGACCTCCCCTCCC | |
| | | TGTACTC | |
| | | (SEQ ID NO: 232) | |
| GITR | miR29 | GGGTTTATTGTAAGAGA | CTTCAGGGTGGGT |
| | | GCATTATGAAGAAAAAA | CTTAGGTCGGGAG |
| | | ATAGATCATAAAGCTTC | CTGCTGATTTAAAT |
| | | TTCAGGGTGGGTCTTAG | AGTGATTGTCAGCC |
| | | GTCGGGAGCTGCTGATT | TCCCGTCCTAAGAC |
| | | TAAATAGTGATTGTCAG | CCCACCTTGGGGG |
| | | CCTCCCGTCCTAAGACCC | (SEQ ID NO: 402) |
| | | CACCTTGGGGGAGACCA | |
| | | GCTGCGCTGCACTACCA | |
| | | ACAGCAAAAGAAGTGAA | |
| | | TGGGACAGCT | |
| | | (SEQ ID NO: 233) | |
| GITR | miR181a1 | TCTCCCATCCCCTTCAGA | TGAGTTTTGAGGTT |
| | | TACTTACAGATACTGTA | GCTTCAGTGAGCCT |
| | | AAGTGAGTAGAATTCTG | CCCGTCCTAAGACC |
| | | AGTTTTGAGGTTGCTTC | CCACTTGGAATTAA |
| | | AGTGAGCCTCCCGTCCT | AATCAAGTGGGTCT |
| | | AAGACCCCACTTGGAAT | TGACGGGTGGCAC |
| | | TAAAATCAAGTGGGTCT | CCTATGGCTAACCA |
| | | TGACGGGTGGCACCCTA | TCATCTACTCCA |
| | | TGGCTAACCATCATCTAC | (SEQ ID NO: 403) |
| | | TCCATGGTGCTCAGAAT | |
| | | TCGCTGAAGACAGGAAA | |
| | | CCAAAGGTGGACACACC | |
| | | AGG | |
| | | (SEQ ID NO: 234) | |
| TIGIT | miR206 | GATGCTACAAGTGGCCC | TGCTTCCCGAGGCC |
| | | ACTTCTGAGATGCGGGC | AGGGTGAGTAACG |
| | | TGCTTCTGGATGACACT | TGGCCTCTTATGGA |
| | | GCTTCCCGAGGCCAGGG | TTACTTTGCTAAGA |
| | | TGAGTAACGTGGCCTCT | TCCACGTTACTCAC |
| | | TATGGATTACTTTGCTA | CCTGGTTTCGGCAA |
| | | AGATCCACGTTACTCACC | GTG |
| | | CTGGTTTCGGCAAGTGC | (SEQ ID NO: 404) |
| | | CTCCTCGCTGGCCCCAG | |
| | | GGTACCACCCGGAGCAC | |
| | | AGGTTTGGTGACCTT | |
| | | (SEQ ID NO: 235) | |
| TIGIT | miR30a | CAGGTTAACCCAACAGA | GCGACAGATCCACG |
| | | AGGCTAAAGAAGGTATA | TTACTCACCCAAGC |
| | | TTGCTGTTGACAGTGAG | TGTGAAGCCACAG |
| | | CGACAGATCCACGTTAC | ATGGGCTTGGGTG |
| | | TCACCCAAGCTGTGAAG | AGACGTGGATCTG |
| | | CCACAGATGGGCTTGGG | CTGC |
| | | TGAGACGTGGATCTGCT | (SEQ ID NO: 405) |
| | | GCCTACTGCCTCGGACT | |
| | | TCAAGGGGCTACTTTAG | |
| | | GAGCAATTATCTTGTTT | |
| | | A | |
| | | (SEQ ID NO: 236) | |
| TIGIT | miR133a1 | TTTACCAATGAAAAGCA | ACAATGCTTTGCTA |
| | | TTTAACTGTTTTGGATTC | GAGGGGTGTATAC |
| | | CAAACTAGCAGCACTAC | CGCGGATCTTCGCC |
| | | AATGCTTTGCTAGAGGG | TCTTCAATGGAAGA |
| | | GTGTATACCGCGGATCT | TCCACGTTACTCAC |
| | | TCGCCTCTTCAATGGAA | CCCTGTAGCTATGC |
| | | GATCCACGTTACTCACCC | ATTGA |
| | | CTGTAGCTATGCATTGA | (SEQ ID NO: 406) |
| | | TTACTACGGGACAACCA | |
| | | ACGTTTTCATTTGTGAAT | |
| | | ATCAATTACTTGCCA | |
| | | (SEQ ID NO: 237) | |
| TIGIT | miR122 | CAATGGTGGAATGTGG | CCTTAGCAGAGCTG |
| | | AGGTGAAGTTAACACCT | AGATCCACGTTACT |
| | | TCGTGGCTACAGAGTTT | CACCCTTGTGTCTA |
| | | CCTTAGCAGAGCTGAGA | AACTATCAAGGGTG |
| | | TCCACGTTACTCACCCTT | AGTCACGTGGAGA |
| | | GTGTCTAAACTATCAAG | TTAGCTACTGCTAG |
| | | GGTGAGTCACGTGGAG | GCAATCCTTCCCTC |
| | | ATTAGCTACTGCTAGGC | GATAAATGTCTTGG |
| | | AATCCTTCCCTCGATAAA | C |
| | | TGTCTTGGCATCGTTTG | (SEQ ID NO: 407) |
| | | CTTTGAGCAAGAAGGTT | |
| | | CATCTGATATCAGTCTTC | |
| | | TCAATCT | |
| | | (SEQ ID NO: 238) | |
| TIGIT | miR29b1 | GGGTTTATTGTAAGAGA | CTTCAGGAAAGATC |
| | | GCATTATGAAGAAAAAA | CACGTTACTCACCC |
| | | ATAGATCATAAAGCTTC | TTAGATTTAAATAG |
| | | TTCAGGAAAGATCCACG | TGATTGTCTAGGTG |
| | | TTACTCACCCTTAGATTT | AGTTACGTGGATCT |
| | | AAATAGTGATTGTCTAG | GTTCTTGGGGG |
| | | GTGAGTTACGTGGATCT | (SEQ ID NO: 408) |
| | | GTTCTTGGGGGAGACCA | |
| | | GCTGCGCTGCACTACCA | |
| | | ACAGCAAAAGAAGTGAA | |
| | | TGGGACAGCT | |
| | | (SEQ ID NO: 239) | |
| TIGIT | miR214 | ACAGGCTGATTGTATCT | GGCCTGGCTGGAC |
| | | GTCTATGAGCAAAGGAA | AGAGTTGTCATGTG |
| | | ACCTGAAGGAACCAAGG | TCAGATCCACGTTA |
| | | GCCTGGCTGGACAGAGT | CTCACCCTGCAGAA |
| | | TGTCATGTGTCAGATCC | CATCCGCTCACCTG |
| | | ACGTTACTCACCCTGCA | TAGGGTGAGACTC |
| | | GAACATCCGCTCACCTG | GTGGATCTGTCACA |
| | | TAGGGTGAGACTCGTG | TGACAACCCAGCCT |
| | | GATCTGTCACATGACAA | (SEQ ID NO: 409) |
| | | CCCAGCCTGAATGACAA | |
| | | CCAGCCATTGAAAGAAA | |
| | | GCAGCCCTCACACCATA | |
| | | GCATCTA | |
| | | (SEQ ID NO: 240) | |
| PD1 | miR30a | CAGGTTAACCCAACAGA | GCGACTTCAGGAAT |
| | | AGGCTAAAGAAGGTATA | GGGTTCCAAGGAG |
| | | TTGCTGTTGACAGTGAG | CTGTGAAGCCACAG |
| | | CGACTTCAGGAATGGGT | ATGGGCTCCTTGGA |
| | | TCCAAGGAGCTGTGAAG | CCATTCCTGAAGCT |
| | | CCACAGATGGGCTCCTT | GC |
| | | GGACCATTCCTGAAGCT | (SEQ ID NO: 410) |
| | | GCCTACTGCCTCGGACT | |
| | | TCAAGGGGCTACTTTAG | |
| | | GAGCAATTATCTTGTTT | |
| | | A | |
| | | (SEQ ID NO: 241) | |
| PD1 | miR412 | GTCTTGGAGGCTGGGG | CTGGGGTACGGGG |
| | | CACCTCGGGGAAGGAC | ATGGATTCAGGAAT |
| | | GCCGGCATCAGCACCAT | GGGTTCCAAGGAA |
| | | TCTGGGGTACGGGGAT | TTGTTTCTAATGTC |
| | | GGATTCAGGAATGGGTT | CTGAAGCCATTCAT |
| | | CCAAGGAATTGTTTCTA | GGAGCCGTCCGTAT |
| | | ATGTCCTGAAGCCATTC | CCGCTGCAG |
| | | ATGGAGCCGTCCGTATC | (SEQ ID NO: 411) |
| | | CGCTGCAGCCTGTGGG | |
| | | GCCTGCGGGCCGGGGA | |
| | | GCCGATCGCGCTTCAGC | |
| | | TCAGCGCCT | |
| | | (SEQ ID NO: 242) | |
| PD1 | miR17 | TTAGCAGGAAAAAAGAG | GTCAGAATAATGTT |
| | | AACATCACCTTGTAAAAC | TCAGGAATGGGTT |
| | | TGAAGATTGTGACCAGT | CCAAGGAAGTGAT |
| | | CAGAATAATGTTTCAGG | ATGTGCATCTTCTT |
| | | AATGGGTTCCAAGGAAG | GGTACACGTTCCTG |
| | | TGATATGTGCATCTTCTT | CTCACATTATGGTG |
| | | GGTACACGTTCCTGCTC | AC |
| | | ACATTATGGTGACAGCT | (SEQ ID NO: 412) |
| | | GCCTCGGGAAGCCAAGT | |
| | | TGGGCTTTAAAGTGCAG | |
| | | GGCCTGCTGATGT | |
| | | (SEQ ID NO: 243) | |
| PD1 | miR122 | CAATGGTGGAATGTGG | CCTTAGCAGAGCTG |
| | | AGGTGAAGTTAACACCT | TTCAGGAATGGGTT |
| | | TCGTGGCTACAGAGTTT | CCAAGGAGTGTCTA |
| | | CCTTAGCAGAGCTGTTC | AACTATCTCCTTGG |
| | | AGGAATGGGTTCCAAG | AACACATTCCTACA |
| | | GAGTGTCTAAACTATCT | TAGCTACTGCTAGG |
| | | CCTTGGAACACATTCCTA | CAATCCTTCCCTCG |
| | | CATAGCTACTGCTAGGC | ATAAATGTCTTGGC |
| | | AATCCTTCCCTCGATAAA | (SEQ ID NO: 413) |
| | | TGTCTTGGCATCGTTTG | |
| | | CTTTGAGCAAGAAGGTT | |
| | | CATCTGATATCAGTCTTC | |
| | | TCAATCT | |
| | | (SEQ ID NO: 244) | |
| PD1 | miR150 | AGGGACTGGGCCCACG | CTCCCCATGGCCCT |
| | | GGGAGGCAGCGTCCCC | GTATAATATAATAG |
| | | GAGGCAGCAGCGGCAG | AACCACAGGCTGG |
| | | CGGCGGCTCCTCTCCCC | GCTCAGACGTGTG |
| | | ATGGCCCTGTATAATAT | GTTTATCCTGTTGT |
| | | AATAGAACCACAGGCTG | ACAGGGACCTGGG |
| | | GGCTCAGACGTGTGGTT | GAC |
| | | TATCCTGTTGTACAGGG | (SEQ ID NO: 414) |
| | | ACCTGGGGACCCCGGCA | |
| | | CCGGCAGGCCCCAAGG | |
| | | GGTGAGGTGAGCGGGC | |
| | | ATTGGGACCTCCCCTCCC | |
| | | TGTACTC | |
| | | (SEQ ID NO: 245) | |
| PD1 | miR486 | TGTGGTGCTGGGGGCT | GCATATAATATAAT |
| | | TCAGCGGCCGGCTCTGA | AGAACCACAGGGC |
| | | TCTCCATCCTCCCTGGG | CCTTCATGCTGCCC |
| | | GCATATAATATAATAGA | AGCTTGTGGTTCTA |
| | | ACCACAGGGCCCTTCAT | TTATGTTATAAC |
| | | GCTGCCCAGCTTGTGGT | (SEQ ID NO: 415) |
| | | TCTATTATGTTATAACTC | |
| | | GGGGTGGGAGTCAGCA | |
| | | GGAGGTGAGGGGGCAT | |
| | | GGTGGCCCCAGTGCAGC | |
| | | (SEQ ID NO: 246) | |
| PD1 | miR206 | GATGCTACAAGTGGCCC | TGCTTCCCGAGGCG |
| | | ACTTCTGAGATGCGGGC | TGCTGAACTGGTAT |
| | | TGCTTCTGGATGACACT | CGAAATGTATGGAT |
| | | GCTTCCCGAGGCGTGCT | TACTTTGCTACATG |
| | | GAACTGGTATCGAAATG | CGGTACCAGTTTAG |
| | | TATGGATTACTTTGCTAC | CACGTTTCGGCAAG |
| | | ATGCGGTACCAGTTTAG | TG |
| | | CACGTTTCGGCAAGTGC | (SEQ ID NO: 416) |
| | | CTCCTCGCTGGCCCCAG | |
| | | GGTACCACCCGGAGCAC | |
| | | AGGTTTGGTGACCTT | |
| | | (SEQ ID NO: 247) | |
| PD1 | miR122 | CAATGGTGGAATGTGG | CCTTAGCAGAGCTG |
| | | AGGTGAAGTTAACACCT | CATGCGGTACCAGT |
| | | TCGTGGCTACAGAGTTT | TTAGCACGTGTCTA |
| | | CCTTAGCAGAGCTGCAT | AACTATCGTGCTAG |
| | | GCGGTACCAGTTTAGCA | ACTTGTACTGTCAG |
| | | CGTGTCTAAACTATCGT | TAGCTACTGCTAGG |
| | | GCTAGACTTGTACTGTC | CAATCCTTCCCTCG |
| | | AGTAGCTACTGCTAGGC | ATAAATGTCTTGGC |
| | | AATCCTTCCCTCGATAAA | (SEQ ID NO: 417) |
| | | TGTCTTGGCATCGTTTG | |
| | | CTTTGAGCAAGAAGGTT | |
| | | CATCTGATATCAGTCTTC | |
| | | TCAATCT | |
| | | (SEQ ID NO: 248) | |
| PD1 | miR30a | CAGGTTAACCCAACAGA | GCGACCATGCGGT |
| | | AGGCTAAAGAAGGTATA | ACCAGTTTAGCACG |
| | | TTGCTGTTGACAGTGAG | CTGTGAAGCCACAG |
| | | CGACCATGCGGTACCAG | ATGGGCGTGCTAG |
| | | TTTAGCACGCTGTGAAG | AGGTGTCGCATGG |
| | | CCACAGATGGGCGTGCT | CTGC |
| | | AGAGGTGTCGCATGGCT | (SEQ ID NO: 418) |
| | | GCCTACTGCCTCGGACT | |
| | | TCAAGGGGCTACTTTAG | |
| | | GAGCAATTATCTTGTTT | |
| | | A | |
| | | (SEQ ID NO: 249) | |
| CTLA4 | miR206 | GATGCTACAAGTGGCCC | TGCTTCCCGAGGCA |
| | | ACTTCTGAGATGCGGGC | GCGAGGGAGAAGA |
| | | TGCTTCTGGATGACACT | TTACCATTTATGGA |
| | | GCTTCCCGAGGCAGCGA | TTACTTTGCTAAAT |
| | | GGGAGAAGATTACCATT | ATAGTCTTCTCCCT |
| | | TATGGATTACTTTGCTA | CGCTGTTTCGGCAA |
| | | AATATAGTCTTCTCCCTC | GTG |
| | | GCTGTTTCGGCAAGTGC | (SEQ ID NO: 419) |
| | | CTCCTCGCTGGCCCCAG | |
| | | GGTACCACCCGGAGCAC | |
| | | AGGTTTGGTGACCTT | |
| | | (SEQ ID NO: 250) | |
| CTLA4 | miR26a1 | TGAAGCCACAGGAGCCA | GTGGCCTCGAATAT |
| | | AGAGCAGGAGGACCAA | AGTCTTCTCCCTCG |
| | | GGCCCTGGCGAAGGCC | CTTGTGCAGGTCCC |
| | | GTGGCCTCGAATATAGT | AATGGAGCGAGGG |
| | | CTTCTCCCTCGCTTGTGC | ATAGGACTATACTC |
| | | AGGTCCCAATGGAGCGA | GGGGACGC |
| | | GGGATAGGACTATACTC | (SEQ ID NO: 420) |
| | | GGGGACGCGGGCCTGG | |
| | | ACGCCGGCATCCGGGCT | |
| | | CAGGACCCCCCTCTCTG | |
| | | CCAGAGGC | |
| | | (SEQ ID NO: 251) | |
| CTLA4 | miR30a | CAGGTTAACCCAACAGA | GCGACAATATAGTC |
| | | AGGCTAAAGAAGGTATA | TTCTCCCTCGCTGC |
| | | TTGCTGTTGACAGTGAG | TGTGAAGCCACAG |
| | | CGACAATATAGTCTTCTC | ATGGGCAGTGAGG |
| | | CCTCGCTGCTGTGAAGC | GAAGACTATGTTGC |
| | | CACAGATGGGCAGTGA | TGC |
| | | GGGAAGACTATGTTGCT | (SEQ ID NO: 421) |
| | | GCCTACTGCCTCGGACT | |
| | | TCAAGGGGCTACTTTAG | |
| | | GAGCAATTATCTTGTTT | |
| | | A | |
| | | (SEQ ID NO: 252) | |
| PIK3IP1 | miR206 | GATGCTACAAGTGGCCC | TGCTTCCCGAGGCC |
| | | ACTTCTGAGATGCGGGC | GGTTGAACATTGGT |
| | | TGCTTCTGGATGACACT | TGCCTCATATGGAT |
| | | GCTTCCCGAGGCCGGTT | TACTTTGCTATGAA |
| | | GAACATTGGTTGCCTCA | CGACCAGTGTTTAA |
| | | TATGGATTACTTTGCTAT | CCGGTTTCGGCAAG |
| | | GAACGACCAGTGTTTAA | TG |
| | | CCGGTTTCGGCAAGTGC | (SEQ ID NO: 422) |
| | | CTCCTCGCTGGCCCCAG | |
| | | GGTACCACCCGGAGCAC | |
| | | AGGTTTGGTGACCTT | |
| | | (SEQ ID NO: 253) | |
| PIK3IP1 | miR126 | CCGGGGTCCTGTCTGCA | CGCTGGCGACGGG |
| | | TCCAGCGCAGCATTCTG | ACGGATGAACTTAG |
| | | GAAGACGCCACGCCTCC | AGGAGACGCTGTG |
| | | GCTGGCGACGGGACGG | ACACTTCAAACTTC |
| | | ATGAACTTAGAGGAGAC | TCCTTGGAGTTCAT |
| | | GCTGTGACACTTCAAAC | CCGCGCCGTCCACG |
| | | TTCTCCTTGGAGTTCATC | GCA |
| | | CGCGCCGTCCACGGCAC | (SEQ ID NO: 423) |
| | | CGCATCGAAAACGCCGC | |
| | | TGAGACCTCAGCCTTGA | |
| | | CCTCCCTCAGCGTGG | |
| | | (SEQ ID NO: 254) | |
| PIK3IP1 | miR30a | CAGGTTAACCCAACAGA | GCGACTTCTCCTTG |
| | | AGGCTAAAGAAGGTATA | GAGTTCATCCGCGC |
| | | TTGCTGTTGACAGTGAG | TGTGAAGCCACAG |
| | | CGACTTCTCCTTGGAGT | ATGGGCGCGGATG |
| | | TCATCCGCGCTGTGAAG | ATCCAAGGAGGAG |
| | | CCACAGATGGGCGCGG | CTGC |
| | | ATGATCCAAGGAGGAGC | (SEQ ID NO: 424) |
| | | TGCCTACTGCCTCGGAC | |
| | | TTCAAGGGGCTACTTTA | |
| | | GGAGCAATTATCTTGTT | |
| | | TA | |
| | | (SEQ ID NO: 255) | |
| TCRa3′UTR | miR150 | AGGGACTGGGCCCACG | CTCCCCATGGCCCT |
| | | GGGAGGCAGCGTCCCC | GATACACATCAGAA |
| | | GAGGCAGCAGCGGCAG | TCCTTACTGCTGGG |
| | | CGGCGGCTCCTCTCCCC | CTCAGACCGTAAGG |
| | | ATGGCCCTGATACACAT | ATCTCCTGTGTATC |
| | | CAGAATCCTTACTGCTG | AGGGACCTGGGGA |
| | | GGCTCAGACCGTAAGGA | C |
| | | TCTCCTGTGTATCAGGG | (SEQ ID NO: 425) |
| | | ACCTGGGGACCCCGGCA | |
| | | CCGGCAGGCCCCAAGG | |
| | | GGTGAGGCGAGCGGGC | |
| | | ATTGGGACCTCCCCTCCC | |
| | | TGTACTC | |
| | | (SEQ ID NO: 256) | |
| TCRa3′UTR | miR204 | AGGAGGGTGGGGGTGG | GGCTACAGTCTTTC |
| | | AGGCAAGCAGAGGACTT | TTCATGTGACTCGT |
| | | CCTGATCGCGTACCCAT | GGACATACACATCA |
| | | GGCTACAGTCTTTCTTCA | GAATCCTTACTTGA |
| | | TGTGACTCGTGGACATA | GAATATATGAAGG |
| | | CACATCAGAATCCTTACT | AGGTAGGATACTG |
| | | TGAGAATATATGAAGGA | ATGTGTACGTTCAA |
| | | GGTAGGATACTGATGTG | TTGTCATCACTGGC |
| | | TACGTTCAATTGTCATCA | (SEQ ID NO: 426) |
| | | CTGGCATCTTTTTTGATC | |
| | | ATTGCACCATCATCAAAT | |
| | | GCATTGGGATAACCATG | |
| | | AC | |
| | | (SEQ ID NO: 257) | |
| TCRa3′UTR | miR206 | GATGCTACAAGTGGCCC | TGCTTCCCGAGGCG |
| | | ACTTCTGAGATGCGGGC | GTAAGGATTCTGAT |
| | | TGCTTCTGGATGACACT | GTAATATTATGGAT |
| | | GCTTCCCGAGGCGGTAA | TACTTTGCTAATAC |
| | | GGATTCTGATGTAATAT | ACATCAGAATCCTT |
| | | TATGGATTACTTTGCTA | ACTGTTTCGGCAAG |
| | | ATACACATCAGAATCCTT | TG |
| | | ACTGTTTCGGCAAGTGC | (SEQ ID NO: 427) |
| | | CTCCTCGCTGGCCCCAG | |
| | | GGTACCACCCGGAGCAC | |
| | | AGGTTTGGTGACCTT | |
| | | (SEQ ID NO: 258) | |
| TCRa3′UTR | miR26a1 | TGAAGCCACAGGAGCCA | GTGGCCTCGATACA |
| | | AGAGCAGGAGGACCAA | CATCAGAATCCTTA |
| | | GGCCCTGGCGAAGGCC | CTTGTGCAGGTCCC |
| | | GTGGCCTCGATACACAT | AATGGAGTAAGGA |
| | | CAGAATCCTTACTTGTG | TCCTGATGTGTCTC |
| | | CAGGTCCCAATGGAGTA | GGGGACGC |
| | | AGGATCCTGATGTGTCT | (SEQ ID NO: 428) |
| | | CGGGGACGCGGGCCTG | |
| | | GACGCCGGCATCCGGGC | |
| | | TCAGGACCCCCCTCTCTG | |
| | | CCAGAGGC | |
| | | (SEQ ID NO: 259) | |
| TCRa3′UTR | miR150 | AGGGACTGGGCCCACG | CTCCCCATGGCCCT |
| | | GGGAGGCAGCGTCCCC | GTTGTTGAAGGCG |
| | | GAGGCAGCAGCGGCAG | TTTGCACATGCTGG |
| | | CGGCGGCTCCTCTCCCC | GCTCAGACCTGTGC |
| | | ATGGCCCTGTTGTTGAA | AATGCGATCAATAG |
| | | GGCGTTTGCACATGCTG | CAGGGACCTGGGG |
| | | GGCTCAGACCTGTGCAA | AC |
| | | TGCGATCAATAGCAGGG | (SEQ ID NO: 429) |
| | | ACCTGGGGACCCCGGCA | |
| | | CCGGCAGGCCCCAAGG | |
| | | GGTGAGGCGAGCGGGC | |
| | | ATTGGGACCTCCCCTCCC | |
| | | TGTACTC | |
| | | (SEQ ID NO: 260) | |
| TCRa3′UTR | miR16 | CTTCTGAAGAAAATATA | GTCAGCAGTGCCTT |
| | | TTTCTTTTTATTCATAGC | TGTTGAAGGCGTTT |
| | | TCTTATGATAGCAATGT | GCACATGTTAAGAT |
| | | CAGCAGTGCCTTTGTTG | TCTAAAATTATCTT |
| | | AAGGCGTTTGCACATGT | GTGCGGAAGCACT |
| | | TAAGATTCTAAAATTATC | TCAATAAAAGTAAG |
| | | TTGTGCGGAAGCACTTC | GTTGAC |
| | | AATAAAAGTAAGGTTGA | (SEQ ID NO: 430) |
| | | CCATACTCTACAGTTGT | |
| | | GTTTTAATGTATATTAAT | |
| | | GTTACTAATGTGTTTT | |
| | | (SEQ ID NO: 261) | |
| TCRa3′UTR | miR206 | GATGCTACAAGTGGCCC | TGCTTCCCGAGGCG |
| | | ACTTCTGAGATGCGGGC | TGTGTGAACGCCTT |
| | | TGCTTCTGGATGACACT | CACATAATATGGAT |
| | | GCTTCCCGAGGCGTGTG | TACTTTGCTATTGT |
| | | TGAACGCCTTCACATAA | TGAAGGCGTTTGC |
| | | TATGGATTACTTTGCTAT | ACATGTTTCGGCAA |
| | | TGTTGAAGGCGTTTGCA | GTG |
| | | CATGTTTCGGCAAGTGC | (SEQ ID NO: 431) |
| | | CTCCTCGCTGGCCCCAG | |
| | | GGTACCACCCGGAGCAC | |
| | | AGGTTTGGTGACCTT | |
| | | (SEQ ID NO: 262) | |
| TCRa3′UTR | miR26a1 | TGAAGCCACAGGAGCCA | GTGGCCTCGTTGTT |
| | | AGAGCAGGAGGACCAA | GAAGGCGTTTGCA |
| | | GGCCCTGGCGAAGGCC | CATTGTGCAGGTCC |
| | | GTGGCCTCGTTGTTGAA | CAATGGGTGTGCA |
| | | GGCGTTTGCACATTGTG | AAGGTCTTCAATCA |
| | | CAGGTCCCAATGGGTGT | CGGGGACGC |
| | | GCAAAGGTCTTCAATCA | (SEQ ID NO: 432) |
| | | CGGGGACGCGGGCCTG | |
| | | GACGCCGGCATCCGGGC | |
| | | TCAGGACCCCCCTCTCTG | |
| | | CCAGAGGC | |
| | | (SEQ ID NO: 263) | |
| CD70 | miR206 | GATGCTACAAGTGGCCC | TGCTTCCCGAGGTG |
| | | ACTTCTGAGATGCGGGC | TGCGTCCAGCGCTT |
| | | TGCTTCTGGATGACACT | CGAGACATATGGAT |
| | | GCTTCCCGAGGTGTGCG | TACTTTGCTATGTG |
| | | TCCAGCGCTTCGAGACA | CGAAGCGCTGGAT |
| | | TATGGATTACTTTGCTAT | GCACATTTCGGCAA |
| | | GTGCGAAGCGCTGGAT | GTG |
| | | GCACATTTCGGCAAGTG | (SEQ ID NO: 433) |
| | | CCTCCTCGCTGGCCCCA | |
| | | GGGTACCACCCGGAGCA | |
| | | CAGGTTTGGTGACCTT | |
| | | (SEQ ID NO: 323) | |
| CD70 | miR204 | AGGAGGGTGGGGGTGG | GGCTACAGTCTTTC |
| | | AGGCAAGCAGAGGACTT | TTCATGTGACTCGT |
| | | CCTGATCGCGTACCCAT | GGACTGTGCGAAG |
| | | GGCTACAGTCTTTCTTCA | CGCTGGATGCACA |
| | | TGTGACTCGTGGACTGT | GAGAATATATGAA |
| | | GCGAAGCGCTGGATGC | GGtgTGGTCCACCG |
| | | ACAGAGAATATATGAAG | CTTCGCACCGTTCA |
| | | GtgTGGTCCACCGCTTCG | ATTGTCATCACTGG |
| | | CACCGTTCAATTGTCATC | C |
| | | ACTGGCATCTTTTTTGAT | (SEQ ID NO: 434) |
| | | CATTGCACCATCATCAAA | |
| | | TGCATTGGGATAACCAT | |
| | | GAC | |
| | | (SEQ ID NO: 324) | |
| CD70 | miR22 | CATTTTCCCTCCCTTTCC | GGCTGAGCCGCAtg |
| | | CTTAGGAGCCTGTTCCT | TGCATCCAGCCTTC |
| | | CTCACGCCCTCACCTGG | GACACATATGTCCT |
| | | CTGAGCCGCAtgTGCATC | GACCCAGCTATGTG |
| | | CAGCCTTCGACACATAT | CGAAGCGCTGGAT |
| | | GTCCTGACCCAGCTATG | GCACATGCCCTCTG |
| | | TGCGAAGCGCTGGATGC | CC |
| | | ACATGCCCTCTGCCCCTG | (SEQ ID NO: 435) |
| | | GCTTCGAGGAGGAGGA | |
| | | GGAGCTGCTTTCCCCAT | |
| | | CATCTGGAAGGTG | |
| | | (SEQ ID NO: 325) | |
| CD70 | miR16 | CTTCTGAAGAAAATATA | GTCAGCAGTGCCTT |
| | | TTTCTTTTTATTCATAGC | GTGCGAAGCGCTG |
| | | TCTTATGATAGCAATGT | GATGCACATTAAGA |
| | | CAGCAGTGCCTTGTGCG | TTCTAAAATTATCTT |
| | | AAGCGCTGGATGCACAT | GCATCCACCGACTT |
| | | TAAGATTCTAAAATTATC | CGCACGAAGTAAG |
| | | TTGCATCCACCGACTTC | GTTGAC |
| | | GCACGAAGTAAGGTTGA | (SEQ ID NO: 436) |
| | | CCATACTCTACAGTIGT | |
| | | GTTTTAATGTATATTAAT | |
| | | GTTACTAATGTGTTTT | |
| | | (SEQ ID NO: 326) | |
| CD70 | miR21 | TGTCTGCTTGTTTTGCCT | TGTCGGGTGTGCG |
| | | ACCATCGTGACATCTCCA | AAGCGCTGGATGC |
| | | TGGCTGTACCACCTTGT | ACACTGTTGAATCT |
| | | CGGGTGTGCGAAGCGC | CATGGGTGCACCCA |
| | | TGGATGCACACTGTTGA | GCCTTCGCACGTCT |
| | | ATCTCATGGGTGCACCC | GACA |
| | | AGCCTTCGCACGTCTGA | (SEQ ID NO: 437) |
| | | CATTTTGGTATCTTTCAT | |
| | | CTGACCATCCATATCCAA | |
| | | TGTTCTCATTTAAACA | |
| | | (SEQ ID NO: 327) | |
| CD70 | miR206 | GATGCTACAAGTGGCCC | TGCTTCCCGAGGGC |
| | | ACTTCTGAGATGCGGGC | TGCAGCTGAATCAC |
| | | TGCTTCTGGATGACACT | ACTCGGATATGGAT |
| | | GCTTCCCGAGGGCTGCA | TACTTTGCTATCCT |
| | | GCTGAATCACACTCGGA | GTGTGATTCAGCTG |
| | | TATGGATTACTTTGCTAT | CAGCTTTCGGCAAG |
| | | CCTGTGTGATTCAGCTG | TG |
| | | CAGCTTTCGGCAAGTGC | (SEQ ID NO: 438) |
| | | CTCCTCGCTGGCCCCAG | |
| | | GGTACCACCCGGAGCAC | |
| | | AGGTTTGGTGACCTT | |
| | | (SEQ ID NO: 328) | |
| CD70 | miR204 | AGGAGGGTGGGGGTGG | GGCTACAGTCTTTC |
| | | AGGCAAGCAGAGGACTT | TTCATGTGACTCGT |
| | | CCTGATCGCGTACCCAT | GGACTCCTGTGTGA |
| | | GGCTACAGTCTTTCTTCA | TTCAGCTGCAGCGA |
| | | TGTGACTCGTGGACTCC | GAATATATGAAGG |
| | | TGTGTGATTCAGCTGCA | GCTGAGCTGCATCA |
| | | GCGAGAATATATGAAGG | CACAGGCGTTCAAT |
| | | GCTGAGCTGCATCACAC | TGTCATCACTGGC |
| | | AGGCGTTCAATTGTCAT | (SEQ ID NO: 439) |
| | | CACTGGCATCTTTTTTGA | |
| | | TCATTGCACCATCATCAA | |
| | | ATGCATTGGGATAACCA | |
| | | TGAC | |
| | | (SEQ ID NO: 329) | |
| CD70 | miR19 | CCAATAATTCAAGCCAA | GCAGTCCTCTGAGC |
| | | GCAAGTATATAGGTGTT | TGCAGCTGAATCAC |
| | | TTAATAGTTTTTGTTTGC | AGGTACAAGAAGA |
| | | AGTCCTCTGAGCTGCAG | ATGTAGTTCCTGTG |
| | | CTGAATCACAGGTACAA | TGATTCAGCTGCAG |
| | | GAAGAATGTAGTTCCTG | CtTGGTGGCCTGC |
| | | TGTGATTCAGCTGCAGC | (SEQ ID NO: 440) |
| | | TTGGTGGCCTGCTATTT | |
| | | CCTTCAAATGAATGATTT | |
| | | TTACTAATTTTGTGTACT | |
| | | TTTATTGTG | |
| | | (SEQ ID NO: 330) | |
| CD70 | miR494 | CAGTTCTGTTTTGATTTT | GATACTCGAAGGA |
| | | TTTTGTTTGTTTTTTGAT | GCTGCATATGAATC |
| | | CAGTGCTAATCTTCGAT | CACCAGGTCTTTAT |
| | | ACTCGAAGGAGCTGCAT | TTATGATCCTGTGT |
| | | ATGAATCCACCAGGTCT | GATTCAGCTGCAGC |
| | | TTATTTATGATCCTGTGT | TTTTTTAGTATC |
| | | GATTCAGCTGCAGCTTT | (SEQ ID NO: 441) |
| | | TTTAGTATCAAATCCCAC | |
| | | CCTGGAGGCACTTCCTG | |
| | | TTCCTGATGCAGCCTTCA | |
| | | GGGAGG | |
| | | (SEQ ID NO: 331) | |
| CD70 | miR150 | AGGGACTGGGCCCACG | CTCCCCATGGCCCT |
| | | GGGAGGCAGCGTCCCC | GTCCTGTGTGATTC |
| | | GAGGCAGCAGCGGCAG | AGCTGCAGCCTGG |
| | | CGGCGGCTCCTCTCCCC | GCTCAGACaTGCAG |
| | | ATGGCCCTGTCCTGTGT | CTAATGTCACAGGA |
| | | GATTCAGCTGCAGCCTG | CAGGGACCTGGGG |
| | | GGCTCAGACATGCAGCT | AC |
| | | AATGTCACAGGACAGGG | (SEQ ID NO: 442) |
| | | ACCTGGGGACCCCGGCA | |
| | | CCGGCAGGCCCCAAGG | |
| | | GGTGAGGTGAGCGGGC | |
| | | ATTGGGACCTCCCCTCCC | |
| | | TGTACTC | |
| | | (SEQ ID NO: 332) | |
| CD70 | miR206 | GATGCTACAAGTGGCCC | TGCTTCCCGAGGAT |
| | | ACTTCTGAGATGCGGGC | GGCATCTACATGGT |
| | | TGCTTCTGGATGACACT | ACTTCATTATGGAT |
| | | GCTTCCCGAGGATGGCA | TACTTTGCTAATGT |
| | | TCTACATGGTACTTCATT | GTACCATGTAGATG |
| | | ATGGATTACTTTGCTAA | CCATTTTCGGCAAG |
| | | TGTGTACCATGTAGATG | TG |
| | | CCATTTTCGGCAAGTGC | (SEQ ID NO: 443) |
| | | CTCCTCGCTGGCCCCAG | |
| | | GGTACCACCCGGAGCAC | |
| | | AGGTTTGGTGACCTT | |
| | | (SEQ ID NO: 333) | |
| CD70 | miR204 | AGGAGGGTGGGGGTGG | GGCTACAGTCTTTC |
| | | AGGCAAGCAGAGGACTT | TTCATGTGACTCGT |
| | | CCTGATCGCGTACCCAT | GGACATGTGTACCA |
| | | GGCTACAGTCTTTCTTCA | TGTAGATGCCATGA |
| | | TGTGACTCGTGGACATG | GAATATATGAAGG |
| | | TGTACCATGTAGATGCC | ATGGATCTAGATG |
| | | ATGAGAATATATGAAGG | GTACACACGTTCAA |
| | | ATGGATCTAGATGGTAC | TTGTCATCACTGGC |
| | | ACACGTTCAATTGTCATC | (SEQ ID NO: 444) |
| | | ACTGGCATCTTTTTTGAT | |
| | | CATTGCACCATCATCAAA | |
| | | TGCATTGGGATAACCAT | |
| | | GAC | |
| | | (SEQ ID NO: 334) | |
| CD70 | miR22 | CATTTTCCCTCCCTTTCC | GGCTGAGCCGCAA |
| | | CTTAGGAGCCTGTTCCT | TGGCATCTACAGGT |
| | | CTCACGCCCTCACCTGG | ACAACATTATGTCC |
| | | CTGAGCCGCAATGGCAT | TGACCCAGCTAATG |
| | | CTACAGGTACAACATTA | TGTACCATGTAGAT |
| | | TGTCCTGACCCAGCTAA | GCCATTGCCCTCTG |
| | | TGTGTACCATGTAGATG | CC |
| | | CCATTGCCCTCTGCCCCT | (SEQ ID NO: 445) |
| | | GGCTTCGAGGAGGAGG | |
| | | AGGAGCTGCTTTCCCCA | |
| | | TCATCTGGAAGGTG | |
| | | (SEQ ID NO: 335) | |
| CD70 | miR142 | GGAGTCAGGAGGCCTG | GACAGTGCAGTCAC |
| | | GGCAGCCTGAAGAGTAC | CATGTGTACCATGT |
| | | ACGCCGACGGACAGACA | AGATGCCAAACAGC |
| | | GACAGTGCAGTCACCAT | ACTGGAGGGAGGC |
| | | GTGTACCATGTAGATGC | ATCTACCTGGTACA |
| | | CAAACAGCACTGGAGGG | CATGATGAGTGTAC |
| | | AGGCATCTACCTGGTAC | TGTG |
| | | ACATGATGAGTGTACTG | (SEQ ID NO: 446) |
| | | TGGGCTTCGGAGATCAC | |
| | | GCCACTGCTGCCGCCCG | |
| | | CTGCCCGCCACCATCTTC | |
| | | (SEQ ID NO: 336) | |
| CD70 | miR26a1 | ATGAAGCCACAGGAGCC | GTGGCCTCGATGT |
| | | AAGAGCAGGAGGACCA | GTACCATGTAGATG |
| | | AGGCCCTGGCGAAGGC | CCATGTGCAGGTCC |
| | | CGTGGCCTCGATGTGTA | CAATGGTGGCATCT |
| | | CCATGTAGATGCCATGT | AGATGGTACACCTC |
| | | GCAGGTCCCAATGGTGG | GGGGACGC |
| | | CATCTAGATGGTACACC | (SEQ ID NO: 447) |
| | | TCGGGGACGCGGGCCT | |
| | | GGACGCCGGCATCCGG | |
| | | GCTCAGGACCCCCCTCT | |
| | | CTGCCAGAGGC | |
| | | (SEQ ID NO: 337) | |

Non-naturally occurring miRNA sequences
(two or more pri-miRNAs)

| miRNA | miRNA | |
|---|---|---|
| Target | backbone | DNA Sequence |
| PD1 + | miR204 + | AGGAGGGTGGGGGTGGAGGCAAGCAGAGGACTTCCTGATC |
| PD1 | miR206 | GCGTACCCATGGCTACAGTCTTTCTTCATGTGACTCGTGG |
| | | ACTTCAGGAATGGGTTCCAAGGATGAGAATATATGAAGGA |
| | | TCCTGGAAGCTATTCCTGACGTTCAATTGTCATCACTGGC |
| | | ATCTTTTTTGATCATTGCACCATCATCAAATGCATTGGGA |
| | | TAACCATGACGATGCTACAAGTGGCCCACTTCTGAGATGC |
| | | GGGCTGCTTCTGGATGACACTGCTTCCCGAGGCCTGTGGT |
| | | TCTGTTATATCCATATATGGATTACTTTGCTATATAATAT |
| | | AATAGAACCACAGGTTTCGGCAAGTGCCTCCTCGCTGGCC |
| | | CCAGGGTACCACCCGGAGCACAGGTTTGGTGACCTT |
| | | (SEQ ID NO: 267) |
| PD1 + | miR204 + | AGGAGGGTGGGGGTGGAGGCAAGCAGAGGACTTCCTGATC |
| TIGIT | miR150 | GCGTACCCATGGCTACAGTCTTTCTTCATGTGACTCGTGG |
| | | ACTTCAGGAATGGGTTCCAAGGATGAGAATATATGAAGGA |
| | | TCCTGGAAGCTATTCCTGACGTTCAATTGTCATCACTGGC |
| | | ATCTTTTTTGATCATTGCACCATCATCAAATGCATTGGGA |
| | | TAACCATGACAGGGACTGGGCCCACGGGGAGGCAGCGTCC |
| | | CCGAGGCAGCAGCGGCAGCGGCGGCTCCTCTCCCCATGGC |
| | | CCTGAGATCCACGTTACTCACCCGTGCTGGGCTCAGACCC |
| | | GGGTGATAATATGGATCTCAGGGACCTGGGGACCCCGGCA |
| | | CCGGCAGGCCCCAAGGGGTGAGGTGAGCGGGCATTGGGAC |
| | | CTCCCCTCCCTGTACTC |
| | | (SEQ ID NO: 268) |
| PD1 + | miR206 + | GATGCTACAAGTGGCCCACTTCTGAGATGCGGGCTGCTTC |
| TIGIT | miR150 | TGGATGACACTGCTTCCCGAGGCCTGTGGTTCTGTTATAT |
| | | CCATATATGGATTACTTTGCTATATAATATAATAGAACCA |
| | | CAGGTTTCGGCAAGTGCCTCCTCGCTGGCCCCAGGGTACC |
| | | ACCCGGAGCACAGGTTTGGTGACCTTAGGGACTGGGCCCA |
| | | CGGGGAGGCAGCGTCCCCGAGGCAGCAGCGGCAGCGGCGG |
| | | CTCCTCTCCCCATGGCCCTGAGATCCACGTTACTCACCCG |
| | | TGCTGGGCTCAGACCCGGGTGATAATATGGATCTCAGGGA |
| | | CCTGGGGACCCCGGCACCGGCAGGCCCCAAGGGGTGAGGT |
| | | GAGCGGGCATTGGGACCTCCCCTCCCTGTACTC |
| | | (SEQ ID NO: 269) |
| TIGIT + | miR17 + | TTAGCAGGAAAAAAGAGAACATCACCTTGTAAAACTGAAG |
| PD1 | miR204 | ATTGTGACCAGTCAGAATAATGTAGATCCACGTTACTCAC |
| | | CCTAGTGATATGTGCATCTAGGGTGTGTCATGTGGATGAA |
| | | GCATTATGGTGACAGCTGCCTCGGGAAGCCAAGTTGGGCT |
| | | TTAAAGTGCAGGGCCTGCTGATGTAGGAGGGTGGGGGTGG |
| | | AGGCAAGCAGAGGACTTCCTGATCGCGTACCCATGGCTAC |
| | | AGTCTTTCTTCATGTGACTCGTGGACTTCAGGAATGGGTT |
| | | CCAAGGATGAGAATATATGAAGGATCCTGGAAGCTATTCC |
| | | TGACGTTCAATTGTCATCACTGGCATCTTTTTTGATCATT |
| | | GCACCATCATCAAATGCATTGGGATAACCATGAC |
| | | (SEQ ID NO: 270) |
| TIGIT + | miR17 + | TTAGCAGGAAAAAAGAGAACATCACCTTGTAAAACTGAAG |
| PD1 | miR206 | ATTGTGACCAGTCAGAATAATGTAGATCCACGTTACTCAC |
| | | CCTAGTGATATGTGCATCTAGGGTGTGTCATGTGGATGAA |
| | | GCATTATGGTGACAGCTGCCTCGGGAAGCCAAGTTGGGCT |
| | | TTAAAGTGCAGGGCCTGCTGATGTGATGCTACAAGTGGCC |
| | | CACTTCTGAGATGCGGGCTGCTTCTGGATGACACTGCTTC |
| | | CCGAGGCCTGTGGTTCTGTTATATCCATATATGGATTACT |
| | | TTGCTATATAATATAATAGAACCACAGGTTTCGGCAAGTG |
| | | CCTCCTCGCTGGCCCCAGGGTACCACCCGGAGCACAGGTT |
| | | TGGTGACCTT |
| | | (SEQ ID NO: 271) |
| TIGIT + | miR204 + | AGGAGGGTGGGGGTGGAGGCAAGCAGAGGACTTCCTGATC |
| PD1 | miR206 | GCGTACCCATGGCTACAGTCTTTCTTCATGTGACTCGTGG |
| | | ACAGATCCACGTTACTCACCCCCTGAGAATATATGAAGGA |
| | | GGGGTGAGAAGCGTGGATCCGTTCAATTGTCATCACTGGC |
| | | ATCTTTTTTGATCATTGCACCATCATCAAATGCATTGGGA |
| | | TAACCATGACGATGCTACAAGTGGCCCACTTCTGAGATGC |
| | | GGGCTGCTTCTGGATGACACTGCTTCCCGAGGCCTGTGGT |
| | | TCTGTTATATCCATATATGGATTACTTTGCTATATAATAT |
| | | AATAGAACCACAGGTTTCGGCAAGTGCCTCCTCGCTGGCC |
| | | CCAGGGTACCACCCGGAGCACAGGTTTGGTGACCTT |
| | | (SEQ ID NO: 272) |
| TIGIT + | miR150 + | AGGGACTGGGCCCACGGGGAGGCAGCGTCCCCGAGGCAGC |
| PD1 | miR204 | AGCGGCAGCGGCGGCTCCTCTCCCCATGGCCCTGAGATCC |
| | | ACGTTACTCACCCGTGCTGGGCTCAGACCCGGGTGATAAT |
| | | ATGGATCTCAGGGACCTGGGGACCCCGGCACCGGCAGGCC |
| | | CCAAGGGGTGAGGTGAGCGGGCATTGGGACCTCCCCTCCC |
| | | TGTACTCAGGAGGGTGGGGGTGGAGGCAAGCAGAGGACTT |
| | | CCTGATCGCGTACCCATGGCTACAGTCTTTCTTCATGTGA |
| | | CTCGTGGACTTCAGGAATGGGTTCCAAGGATGAGAATATA |
| | | TGAAGGATCCTGGAAGCTATTCCTGACGTTCAATTGTCAT |
| | | CACTGGCATCTTTTTTGATCATTGCACCATCATCAAATGC |
| | | ATTGGGATAACCATGAC |
| | | (SEQ ID NO: 273) |
| TIGIT + | miR150 + | AGGGACTGGGCCCACGGGGAGGCAGCGTCCCCGAGGCAGC |
| PD1 | miR206 | AGCGGCAGCGGCGGCTCCTCTCCCCATGGCCCTGAGATCC |
| | | ACGTTACTCACCCGTGCTGGGCTCAGACCCGGGTGATAAT |
| | | ATGGATCTCAGGGACCTGGGGACCCCGGCACCGGCAGGCC |
| | | CCAAGGGGTGAGGTGAGCGGGCATTGGGACCTCCCCTCCC |
| | | TGTACTCGATGCTACAAGTGGCCCACTTCTGAGATGCGGG |
| | | CTGCTTCTGGATGACACTGCTTCCCGAGGCCTGTGGTTCT |
| | | GTTATATCCATATATGGATTACTTTGCTATATAATATAAT |
| | | AGAACCACAGGTTTCGGCAAGTGCCTCCTCGCTGGCCCCA |
| | | GGGTACCACCCGGAGCACAGGTTTGGTGACCTT |
| | | (SEQ ID NO: 274) |
| TIGIT + | miR17 + | TTAGCAGGAAAAAAGAGAACATCACCTTGTAAAACTGAAG |
| PD1 + | miR204 + | ATTGTGACCAGTCAGAATAATGTAGATCCACGTTACTCAC |
| PD1 | miR206 | CCTAGTGATATGTGCATCTAGGGTGTGTCATGTGGATGAA |
| | | GCATTATGGTGACAGCTGCCTCGGGAAGCCAAGTTGGGCT |
| | | TTAAAGTGCAGGGCCTGCTGATGTAGGAGGGTGGGGGTGG |
| | | AGGCAAGCAGAGGACTTCCTGATCGCGTACCCATGGCTAC |
| | | AGTCTTTCTTCATGTGACTCGTGGACTTCAGGAATGGGTT |
| | | CCAAGGATGAGAATATATGAAGGATCCTGGAAGCTATTCC |
| | | TGACGTTCAATTGTCATCACTGGCATCTTTTTTGATCATT |
| | | GCACCATCATCAAATGCATTGGGATAACCATGACGATGCT |
| | | ACAAGTGGCCCACTTCTGAGATGCGGGCTGCTTCTGGATG |
| | | ACACTGCTTCCCGAGGCCTGTGGTTCTGTTATATCCATAT |
| | | ATGGATTACTTTGCTATATAATATAATAGAACCACAGGTT |
| | | TCGGCAAGTGCCTCCTCGCTGGCCCCAGGGTACCACCCGG |
| | | AGCACAGGTTTGGTGACCTT |
| | | (SEQ ID NO: 275) |
| TIGIT + | miR17 + | TTAGGATGAGTTGAGATCCCAGTGATCTTCTCGCTAAGAG |
| PD1 + | miR204 + | TTTCCTGCCTGGGCAAGGAGGAAATTAGCAGGAAAAAAGA |
| PD1 | miR206 | GAACATCACCTTGTAAAACTGAAGATTGTGACCAGTCAGA |
| | extra | ATAATGTAGATCCACGTTACTCACCCTAGTGATATGTGCA |
| | spacing | TCTAGGGTGTGTCATGTGGATGAAGCATTATGGTGACAGC |
| | 1 | TGCCTCGGGAAGCCAAGTTGGGCTTTAAAGTGCAGGGCCT |
| | | GCTGATGTAGGAGGGTGGGGGTGGAGGCAAGCAGAGGACT |
| | | TCCTGATCGCGTACCCATGGCTACAGTCTTTCTTCATGTG |
| | | ACTCGTGGACTTCAGGAATGGGTTCCAAGGATGAGAATAT |
| | | ATGAAGGATCCTGGAAGCTATTCCTGACGTTCAATTGTCA |
| | | TCACTGGCATCTTTTTTGATCATTGCACCATCATCAAATG |
| | | CATTGGGATAACCATGACGATGCTACAAGTGGCCCACTTC |
| | | TGAGATGCGGGCTGCTTCTGGATGACACTGCTTCCCGAGG |
| | | CCTGTGGTTCTGTTATATCCATATATGGATTACTTTGCTA |
| | | TATAATATAATAGAACCACAGGTTTCGGCAAGTGCCTCCT |
| | | CGCTGGCCCCAGGGTACCACCCGGAGCACAGGTTTGGTGA |
| | | CCTTCTTCCTCATCAGGGCTTTGTGCCAGCAAATGACTCC |
| | | CTCACCAAGGAAGCAAGAGCCTCTGAATCCCATCTGGGCT |
| | | CTTCCTGAACACCCCTATCTCCCCCTCT |
| | | (SEQ ID NO: 276) |
| TIGIT + | miR17 + | TTAGGGATTATGCTGAATTTGTATGGTTTATAGTTGTTAG |
| PD1 + | miR204 + | AGTTTGAGGTGTTAATTCTAATTATCTATTTCAAATTTTA |
| PD1 | miR206 | GCAGGAAAAAAGAGAACATCACCTTGTAAAACTGAAGATT |
| | extra | GTGACCAGTCAGAATAATGTAGATCCACGTTACTCACCCT |
| | spacing | AGTGATATGTGCATCTAGGGTGTGTCATGTGGATGAAGCA |
| | 2 | TTATGGTGACAGCTGCCTCGGGAAGCCAAGTTGGGCTTTA |
| | | AAGTGCAGGGCCTGCTGATGTAGGAGGGTGGGGGTGGAGG |
| | | CAAGCAGAGGACTTCCTGATCGCGTACCCATGGCTACAGT |
| | | CTTTCTTCATGTGACTCGTGGACTTCAGGAATGGGTTCCA |
| | | AGGATGAGAATATATGAAGGATCCTGGAAGCTATTCCTGA |
| | | CGTTCAATTGTCATCACTGGCATCTTTTTTGATCATTGCA |
| | | CCATCATCAAATGCATTGGGATAACCATGACGATGCTACA |
| | | AGTGGCCCACTTCTGAGATGCGGGCTGCTTCTGGATGACA |
| | | CTGCTTCCCGAGGCCTGTGGTTCTGTTATATCCATATATG |
| | | GATTACTTTGCTATATAATATAATAGAACCACAGGTTTCG |
| | | GCAAGTGCCTCCTCGCTGGCCCCAGGGTACCACCCGGAGC |
| | | ACAGGTTTGGTGACCTTCTTCCTCATCAGGGCTTTGTGCC |
| | | AGCAAATGACTCCCTCACCAAGGAAGCAAGAGCCTCTGAA |
| | | TCCCATCTGGGCTCTTCCTGAACACCCCTATCTCCCCCTC |
| | | T (SEQ ID NO: 277) |
| PD1 + | miR204 + | AGGAGGGTGGGGGTGGAGGCAAGCAGAGGACTTCCTGATC |
| PD1 + | miR206 + | GCGTACCCATGGCTACAGTCTTTCTTCATGTGACTCGTGG |
| TIGIT | miR17 | ACTTCAGGAATGGGTTCCAAGGATGAGAATATATGAAGGA |
| | | TCCTGGAAGCTATTCCTGACGTTCAATTGTCATCACTGGC |
| | | ATCTTTTTTGATCATTGCACCATCATCAAATGCATTGGGA |
| | | TAACCATGACGATGCTACAAGTGGCCCACTTCTGAGATGC |
| | | GGGCTGCTTCTGGATGACACTGCTTCCCGAGGCCTGTGGT |
| | | TCTGTTATATCCATATATGGATTACTTTGCTATATAATAT |
| | | AATAGAACCACAGGTTTCGGCAAGTGCCTCCTCGCTGGCC |
| | | CCAGGGTACCACCCGGAGCACAGGTTTGGTGACCTTTTAG |
| | | CAGGAAAAAAGAGAACATCACCTTGTAAAACTGAAGATTG |
| | | TGACCAGTCAGAATAATGTAGATCCACGTTACTCACCCTA |
| | | GTGATATGTGCATCTAGGGTGTGTCATGTGGATGAAGCAT |
| | | TATGGTGACAGCTGCCTCGGGAAGCCAAGTTGGGCTTTAA |
| | | AGTGCAGGGCCTGCTGATGT |
| | | (SEQ ID NO: 278) |
| PD1 + | miR204 + | TTAGGATGAGTTGAGATCCCAGTGATCTTCTCGCTAAGAG |
| PD1 + | miR206 + | TTTCCTGCCTGGGCAAGGAGGAAAAGGAGGGTGGGGGTGG |
| TIGIT | miR17 | AGGCAAGCAGAGGACTTCCTGATCGCGTACCCATGGCTAC |
| | extra | AGTCTTTCTTCATGTGACTCGTGGACTTCAGGAATGGGTT |
| | spacing | CCAAGGATGAGAATATATGAAGGATCCTGGAAGCTATTCC |
| | 1 | TGACGTTCAATTGTCATCACTGGCATCTTTTTTGATCATT |
| | | GCACCATCATCAAATGCATTGGGATAACCATGACGATGCT |
| | | ACAAGTGGCCCACTTCTGAGATGCGGGCTGCTTCTGGATG |
| | | ACACTGCTTCCCGAGGCCTGTGGTTCTGTTATATCCATAT |
| | | ATGGATTACTTTGCTATATAATATAATAGAACCACAGGTT |
| | | TCGGCAAGTGCCTCCTCGCTGGCCCCAGGGTACCACCCGG |
| | | AGCACAGGTTTGGTGACCTTTTAGCAGGAAAAAAGAGAAC |
| | | ATCACCTTGTAAAACTGAAGATTGTGACCAGTCAGAATAA |
| | | TGTAGATCCACGTTACTCACCCTAGTGATATGTGCATCTA |
| | | GGGTGTGTCATGTGGATGAAGCATTATGGTGACAGCTGCC |
| | | TCGGGAAGCCAAGTTGGGCTTTAAAGTGCAGGGCCTGCTG |
| | | ATGTCTTCCTCATCAGGGCTTTGTGCCAGCAAATGACTCC |
| | | CTCACCAAGGAAGCAAGAGCCTCTGAATCCCATCTGGGCT |
| | | CTTCCTGAACACCCCTATCTCCCCCTCT |
| | | (SEQ ID NO: 279) |
| PD1 + | miR204 + | TGGAGAGGAGGGTGGGGGTGGAGGCAAGCAGAGGACCTCC |
| PD1 + | miR206 + | TGATCATGTACCCATAGGACAGGGTGATGGAAAGGAGGGT |
| TIGIT | miR17 | GGGGGTGGAGGCAAGCAGAGGACTTCCTGATCGCGTACCC |
| | extra | ATGGCTACAGTCTTTCTTCATGTGACTCGTGGACTTCAGG |
| | spacing | AATGGGTTCCAAGGATGAGAATATATGAAGGATCCTGGAA |
| | 2 | GCTATTCCTGACGTTCAATTGTCATCACTGGCATCTTTTT |
| | | TGATCATTGCACCATCATCAAATGCATTGGGATAACCATG |
| | | ACGATGCTACAAGTGGCCCACTTCTGAGATGCGGGCTGCT |
| | | TCTGGATGACACTGCTTCCCGAGGCCTGTGGTTCTGTTAT |
| | | ATCCATATATGGATTACTTTGCTATATAATATAATAGAAC |
| | | CACAGGTTTCGGCAAGTGCCTCCTCGCTGGCCCCAGGGTA |
| | | CCACCCGGAGCACAGGTTTGGTGACCTTTTAGCAGGAAAA |
| | | AAGAGAACATCACCTTGTAAAACTGAAGATTGTGACCAGT |
| | | CAGAATAATGTAGATCCACGTTACTCACCCTAGTGATATG |
| | | TGCATCTAGGGTGTGTCATGTGGATGAAGCATTATGGTGA |
| | | CAGCTGCCTCGGGAAGCCAAGTTGGGCTTTAAAGTGCAGG |
| | | GCCTGCTGATGTCTTCCTCATCAGGGCTTTGTGCCAGCAA |
| | | ATGACTCCCTCACCAAGGAAGCAAGAGCCTCTGAATCCCA |
| | | TCTGGGCTCTTCCTGAACACCCCTATCTCCCCCTCT |
| | | (SEQ ID NO: 280) |
| PD1 + | miR204 + | AGGAGGGTGGGGGTGGAGGCAAGCAGAGGACTTCCTGATC |
| CTLA4 | miR26a1 | GCGTACCCATGGCTACAGTCTTTCTTCATGTGACTCGTGG |
| | | ACTTCAGGAATGGGTTCCAAGGATGAGAATATATGAAGGA |
| | | TCCTGGAAGCTATTCCTGACGTTCAATTGTCATCACTGGC |
| | | ATCTTTTTTGATCATTGCACCATCATCAAATGCATTGGGA |
| | | TAACCATGACTGAAGCCACAGGAGCCAAGAGCAGGAGGAC |
| | | CAAGGCCCTGGCGAAGGCCGTGGCCTCGAATATAGTCTTC |
| | | TCCCTCGCTTGTGCAGGTCCCAATGGAGCGAGGGATAGGA |
| | | CTATACTCGGGGACGCGGGCCTGGACGCCGGCATCCGGGC |
| | | TCAGGACCCCCCTCTCTGCCAGAGGC |
| | | (SEQ ID NO: 281) |
| PD1 + | miR204 + | AGGAGGGTGGGGGTGGAGGCAAGCAGAGGACTTCCTGATC |
| PD1 | miR206 | GCGTACCCATGGCTACAGTCTTTCTTCATGTGACTCGTGG |
| | | ACTTCAGGAATGGGTTCCAAGGATGAGAATATATGAAGGA |
| | | TCCTGGAAGCTATTCCTGACGTTCAATTGTCATCACTGGC |
| | | ATCTTTTTTGATCATTGCACCATCATCAAATGCATTGGGA |
| | | TAACCATGACGATGCTACAAGTGGCCCACTTCTGAGATGC |
| | | GGGCTGCTTCTGGATGACACTGCTTCCCGAGGCCTGTGGT |
| | | TCTGTTATATCCATATATGGATTACTTTGCTATATAATAT |
| | | AATAGAACCACAGGTTTCGGCAAGTGCCTCCTCGCTGGCC |
| | | CCAGGGTACCACCCGGAGCACAGGTTTGGTGACCTT |
| | | (SEQ ID NO: 282) |
| PD1 + | miR206 + | GATGCTACAAGTGGCCCACTTCTGAGATGCGGGCTGCTTC |
| CTLA4 | miR26a1 | TGGATGACACTGCTTCCCGAGGCCTGTGGTTCTGTTATAT |
| | | CCATATATGGATTACTTTGCTATATAATATAATAGAACCA |
| | | CAGGTTTCGGCAAGTGCCTCCTCGCTGGCCCCAGGGTACC |
| | | ACCCGGAGCACAGGTTTGGTGACCTTTGAAGCCACAGGAG |
| | | CCAAGAGCAGGAGGACCAAGGCCCTGGCGAAGGCCGTGGC |
| | | CTCGAATATAGTCTTCTCCCTCGCTTGTGCAGGTCCCAAT |
| | | GGAGCGAGGGATAGGACTATACTCGGGGACGCGGGCCTGG |
| | | ACGCCGGCATCCGGGCTCAGGACCCCCCTCTCTGCCAGAG |
| | | GC (SEQ ID NO: 283) |
| PD1 + | miR206 + | GATGCTACAAGTGGCCCACTTCTGAGATGCGGGCTGCTTC |
| CTLA4 | miR204 | TGGATGACACTGCTTCCCGAGGCCTGTGGTTCTGTTATAT |
| | | CCATATATGGATTACTTTGCTATATAATATAATAGAACCA |
| | | CAGGTTTCGGCAAGTGCCTCCTCGCTGGCCCCAGGGTACC |
| | | ACCCGGAGCACAGGTTTGGTGACCTTAGGAGGGTGGGGGT |
| | | GGAGGCAAGCAGAGGACTTCCTGATCGCGTACCCATGGCT |
| | | ACAGTCTTTCTTCATGTGACTCGTGGACAATATAGTCTTC |
| | | TCCCTCGCTTGAGAATATATGAAGGAAGCAGGGACAGGAC |
| | | TATATTGTTCAATTGTCATCACTGGCATCTTTTTTGATCA |
| | | TTGCACCATCATCAAATGCATTGGGATAACCATGAC |
| | | (SEQ ID NO: 284) |
| TIGIT + | miR17 + | TTAGCAGGAAAAAAGAGAACATCACCTTGTAAAACTGAAG |
| CTLA4 | miR26a1 | ATTGTGACCAGTCAGAATAATGTAGATCCACGTTACTCAC |
| | | CCTAGTGATATGTGCATCTAGGGTGTGTCATGTGGATGAA |
| | | GCATTATGGTGACAGCTGCCTCGGGAAGCCAAGTTGGGCT |
| | | TTAAAGTGCAGGGCCTGCTGATGTTGAAGCCACAGGAGCC |
| | | AAGAGCAGGAGGACCAAGGCCCTGGCGAAGGCCGTGGCCT |
| | | CGAATATAGTCTTCTCCCTCGCTTGTGCAGGTCCCAATGG |
| | | AGCGAGGGATAGGACTATACTCGGGGACGCGGGCCTGGAC |
| | | GCCGGCATCCGGGCTCAGGACCCCCCTCTCTGCCAGAGGC |
| | | (SEQ ID NO: 285) |
| TIGIT + | miR17 + | TTAGCAGGAAAAAAGAGAACATCACCTTGTAAAACTGAAG |
| CTLA4 | miR204 | ATTGTGACCAGTCAGAATAATGTAGATCCACGTTACTCAC |
| | | CCTAGTGATATGTGCATCTAGGGTGTGTCATGTGGATGAA |
| | | GCATTATGGTGACAGCTGCCTCGGGAAGCCAAGTTGGGCT |
| | | TTAAAGTGCAGGGCCTGCTGATGTAGGAGGGTGGGGGTGG |
| | | AGGCAAGCAGAGGACTTCCTGATCGCGTACCCATGGCTAC |
| | | AGTCTTTCTTCATGTGACTCGTGGACAATATAGTCTTCTC |
| | | CCTCGCTTGAGAATATATGAAGGAAGCAGGGACAGGACTA |
| | | TATTGTTCAATTGTCATCACTGGCATCTTTTTTGATCATT |
| | | GCACCATCATCAAATGCATTGGGATAACCATGAC |
| | | (SEQ ID NO: 286) |
| TIGIT + | miR17 + | TTAGCAGGAAAAAAGAGAACATCACCTTGTAAAACTGAAG |
| PD1 | miR204 | ATTGTGACCAGTCAGAATAATGTAGATCCACGTTACTCAC |
| | | CCTAGTGATATGTGCATCTAGGGTGTGTCATGTGGATGAA |
| | | GCATTATGGTGACAGCTGCCTCGGGAAGCCAAGTTGGGCT |
| | | TTAAAGTGCAGGGCCTGCTGATGTAGGAGGGTGGGGGTGG |
| | | AGGCAAGCAGAGGACTTCCTGATCGCGTACCCATGGCTAC |
| | | AGTCTTTCTTCATGTGACTCGTGGACTTCAGGAATGGGTT |
| | | CCAAGGATGAGAATATATGAAGGATCCTGGAAGCTATTCC |
| | | TGACGTTCAATTGTCATCACTGGCATCTTTTTTGATCATT |
| | | GCACCATCATCAAATGCATTGGGATAACCATGAC |
| | | (SEQ ID NO: 287) |
| TIGIT + | miR17 + | TTAGCAGGAAAAAAGAGAACATCACCTTGTAAAACTGAAG |
| PD1 | 206 | ATTGTGACCAGTCAGAATAATGTAGATCCACGTTACTCAC |
| | | CCTAGTGATATGTGCATCTAGGGTGTGTCATGTGGATGAA |
| | | GCATTATGGTGACAGCTGCCTCGGGAAGCCAAGTTGGGCT |
| | | TTAAAGTGCAGGGCCTGCTGATGTGATGCTACAAGTGGCC |
| | | CACTTCTGAGATGCGGGCTGCTTCTGGATGACACTGCTTC |
| | | CCGAGGCCTGTGGTTCTGTTATATCCATATATGGATTACT |
| | | TTGCTATATAATATAATAGAACCACAGGTTTCGGCAAGTG |
| | | CCTCCTCGCTGGCCCCAGGGTACCACCCGGAGCACAGGTT |
| | | TGGTGACCTT |
| | | (SEQ ID NO: 288) |
| TIGIT + | miR204 + | AGGAGGGTGGGGGTGGAGGCAAGCAGAGGACTTCCTGATC |
| CTLA4 | miR26a1 | GCGTACCCATGGCTACAGTCTTTCTTCATGTGACTCGTGG |
| | | ACAGATCCACGTTACTCACCCCCTGAGAATATATGAAGGA |
| | | GGGGTGAGAAGCGTGGATCCGTTCAATTGTCATCACTGGC |
| | | ATCTTTTTTGATCATTGCACCATCATCAAATGCATTGGGA |
| | | TAACCATGACTGAAGCCACAGGAGCCAAGAGCAGGAGGAC |
| | | CAAGGCCCTGGCGAAGGCCGTGGCCTCGAATATAGTCTTC |
| | | TCCCTCGCTTGTGCAGGTCCCAATGGAGCGAGGGATAGGA |
| | | CTATACTCGGGGACGCGGGCCTGGACGCCGGCATCCGGGC |
| | | TCAGGACCCCCCTCTCTGCCAGAGGC |
| | | (SEQ ID NO: 289) |
| TIGIT + | miR204 + | AGGAGGGTGGGGGTGGAGGCAAGCAGAGGACTTCCTGATC |
| PD1 | miR206 | GCGTACCCATGGCTACAGTCTTTCTTCATGTGACTCGTGG |
| | | ACAGATCCACGTTACTCACCCCCTGAGAATATATGAAGGA |
| | | GGGGTGAGAAGCGTGGATCCGTTCAATTGTCATCACTGGC |
| | | ATCTTTTTTGATCATTGCACCATCATCAAATGCATTGGGA |
| | | TAACCATGACGATGCTACAAGTGGCCCACTTCTGAGATGC |
| | | GGGCTGCTTCTGGATGACACTGCTTCCCGAGGCCTGTGGT |
| | | TCTGTTATATCCATATATGGATTACTTTGCTATATAATAT |
| | | AATAGAACCACAGGTTTCGGCAAGTGCCTCCTCGCTGGCC |
| | | CCAGGGTACCACCCGGAGCACAGGTTTGGTGACCTT |
| | | (SEQ ID NO: 290) |
| PD1 + | miR204 + | AGGAGGGTGGGGGTGGAGGCAAGCAGAGGACTTCCTGATC |
| PD1 + | miR206 + | GCGTACCCATGGCTACAGTCTTTCTTCATGTGACTCGTGG |
| CD70 | miR16 | ACTTCAGGAATGGGTTCCAAGGATGAGAATATATGAAGGA |
| | | TCCTGGAAGCTATTCCTGACGTTCAATTGTCATCACTGGC |
| | | ATCTTTTTTGATCATTGCACCATCATCAAATGCATTGGGA |
| | | TAACCATGACGATGCTACAAGTGGCCCACTTCTGAGATGC |
| | | GGGCTGCTTCTGGATGACACTGCTTCCCGAGGCCTGTGGT |
| | | TCTGTTATATCCATATATGGATTACTTTGCTATATAATAT |
| | | AATAGAACCACAGGTTTCGGCAAGTGCCTCCTCGCTGGCC |
| | | CCAGGGTACCACCCGGAGCACAGGTTTGGTGACCTTCTTC |
| | | TGAAGAAAATATATTTCTTTTTATTCATAGCTCTTATGAT |
| | | AGCAATGTCAGCAGTGCCTIGTGCGAAGCGCTGGATGCAC |
| | | ATTAAGATTCTAAAATTATCTTGCATCCACCGACTTCGCA |
| | | CGAAGTAAGGTTGACCATACTCTACAGTTGTGTTTTAATG |
| | | TATATTAATGTTACTAATGTGTTTT |
| | | (SEQ ID NO: 448) |
| PD1 + | miR204 + | AGGAGGGTGGGGGTGGAGGCAAGCAGAGGACTTCCTGATC |
| CD70 + | miR206 + | GCGTACCCATGGCTACAGTCTTTCTTCATGTGACTCGTGG |
| CD70 | miR22 | ACTTCAGGAATGGGTTCCAAGGATGAGAATATATGAAGGA |
| | | TCCTGGAAGCTATTCCTGACGTTCAATTGTCATCACTGGC |
| | | ATCTTTTTTGATCATTGCACCATCATCAAATGCATTGGGA |
| | | TAACCATGACGATGCTACAAGTGGCCCACTTCTGAGATGC |
| | | GGGCTGCTTCTGGATGACACTGCTTCCCGAGGCCTGTGGT |
| | | TCTGTTATATCCATATATGGATTACTTTGCTATATAATAT |
| | | AATAGAACCACAGGTTTCGGCAAGTGCCTCCTCGCTGGCC |
| | | CCAGGGTACCACCCGGAGCACAGGTTTGGTGACCTTCATT |
| | | TTCCCTCCCTTTCCCTTAGGAGCCTGTTCCTCTCACGCCC |
| | | TCACCTGGCTGAGCCGCAATGGCATCTACAGGTACAACAT |
| | | TATGTCCTGACCCAGCTAATGTGTACCATGTAGATGCCAT |
| | | TGCCCTCTGCCCCTGGCTTCGAGGAGGAGGAGGAGCTGCT |
| | | TTCCCCATCATCTGGAAGGTG |
| | | (SEQ ID NO: 449) |
| PD1 + | miR16 + | CTTCTGAAGAAAATATATTTCTTTTTATTCATAGCTCTTA |
| CD70 | miR204 + | TGATAGCAATGTCAGCAGTGCCTTGTGCGAAGCGCTGGAT |
| | miR206 | GCACATTAAGATTCTAAAATTATCTTGCATCCACCGACTT |
| | | CGCACGAAGTAAGGTTGACCATACTCTACAGTTGTGTTTT |
| | | AATGTATATTAATGTTACTAATGTGTTTTAGGAGGGTGGG |
| | | GGTGGAGGCAAGCAGAGGACTTCCTGATCGCGTACCCATG |
| | | GCTACAGTCTTTCTTCATGTGACTCGTGGACTTCAGGAAT |
| | | GGGTTCCAAGGATGAGAATATATGAAGGATCCTGGAAGCT |
| | | ATTCCTGACGTTCAATTGTCATCACTGGCATCTTTTTTGA |
| | | TCATTGCACCATCATCAAATGCATTGGGATAACCATGACG |
| | | ATGCTACAAGTGGCCCACTTCTGAGATGCGGGCTGCTTCT |
| | | GGATGACACTGCTTCCCGAGGCCTGTGGTTCTGTTATATC |
| | | CATATATGGATTACTTTGCTATATAATATAATAGAACCAC |
| | | AGGTTTCGGCAAGTGCCTCCTCGCTGGCCCCAGGGTACCA |
| | | CCCGGAGCACAGGTTTGGTGACCTT |
| | | (SEQ ID NO: 450) |
| CD70 + | miR22 + | CATTTTCCCTCCCTTTCCCTTAGGAGCCTGTTCCTCTCAC |
| PD1 + | miR204 + | GCCCTCACCTGGCTGAGCCGCAATGGCATCTACAGGTACA |
| PD1 | miR206 | ACATTATGTCCTGACCCAGCTAATGTGTACCATGTAGATG |
| | | CCATTGCCCTCTGCCCCTGGCTTCGAGGAGGAGGAGGAGC |
| | | TGCTTTCCCCATCATCTGGAAGGTGAGGAGGGTGGGGGTG |
| | | GAGGCAAGCAGAGGACTTCCTGATCGCGTACCCATGGCTA |
| | | CAGTCTTTCTTCATGTGACTCGTGGACTTCAGGAATGGGT |
| | | TCCAAGGATGAGAATATATGAAGGATCCTGGAAGCTATTC |
| | | CTGACGTTCAATTGTCATCACTGGCATCTTTTTTGATCAT |
| | | TGCACCATCATCAAATGCATTGGGATAACCATGACGATGC |
| | | TACAAGTGGCCCACTTCTGAGATGCGGGCTGCTTCTGGAT |
| | | GACACTGCTTCCCGAGGCCTGTGGTTCTGTTATATCCATA |
| | | TATGGATTACTTTGCTATATAATATAATAGAACCACAGGT |
| | | TTCGGCAAGTGCCTCCTCGCTGGCCCCAGGGTACCACCCG |
| | | GAGCACAGGTTTGGTGACCTT |
| | | (SEQ ID NO: 451) |
| CD70 + | miR204 + | AGGAGGGTGGGGGTGGAGGCAAGCAGAGGACTTCCTGATC |
| CD70 | miR206 | GCGTACCCATGGCTACAGTCTTTCTTCATGTGACTCGTGG |
| | | ACATGTGTACCATGTAGATGCCATGAGAATATATGAAGGA |
| | | TGGATCTAGATGGTACACACGTTCAATTGTCATCACTGGC |
| | | ATCTTTTTTGATCATTGCACCATCATCAAATGCATTGGGA |
| | | TAACCATGACGATGCTACAAGTGGCCCACTTCTGAGATGC |
| | | GGGCTGCTTCTGGATGACACTGCTTCCCGAGGGCTGCAGC |
| | | TGAATCACACTCGGATATGGATTACTTTGCTATCCTGTGT |
| | | GATTCAGCTGCAGCTTTCGGCAAGTGCCTCCTCGCTGGCC |
| | | CCAGGGTACCACCCGGAGCACAGGTTTGGTGACCTT |
| | | (SEQ ID NO: 452) |
| CD70 + | miR26a1 + | ATGAAGCCACAGGAGCCAAGAGCAGGAGGACCAAGGCCCT |
| CD70 | miR206 | GGCGAAGGCCGTGGCCTCGATGTGTACCATGTAGATGCCA |
| | | TGTGCAGGTCCCAATGGTGGCATCTAGATGGTACACCTCG |
| | | GGGACGCGGGCCTGGACGCCGGCATCCGGGCTCAGGACCC |
| | | CCCTCTCTGCCAGAGGCGATGCTACAAGTGGCCCACTTCT |
| | | GAGATGCGGGCTGCTTCTGGATGACACTGCTTCCCGAGGG |
| | | CTGCAGCTGAATCACACTCGGATATGGATTACTTTGCTAT |
| | | CCTGTGTGATTCAGCTGCAGCTTTCGGCAAGTGCCTCCTC |
| | | GCTGGCCCCAGGGTACCACCCGGAGCACAGGTTTGGTGAC |
| | | CTT (SEQ ID NO: 453) |
| CD70 + | miR26a1 + | ATGAAGCCACAGGAGCCAAGAGCAGGAGGACCAAGGCCCT |
| CD70 | miR16 | GGCGAAGGCCGTGGCCTCGATGTGTACCATGTAGATGCCA |
| | | TGTGCAGGTCCCAATGGTGGCATCTAGATGGTACACCTCG |
| | | GGGACGCGGGCCTGGACGCCGGCATCCGGGCTCAGGACCC |
| | | CCCTCTCTGCCAGAGGCCTTCTGAAGAAAATATATTTCTT |
| | | TTTATTCATAGCTCTTATGATAGCAATGTCAGCAGTGCCT |
| | | TGTGCGAAGCGCTGGATGCACATTAAGATTCTAAAATTAT |
| | | CTTGCATCCACCGACTTCGCACGAAGTAAGGTTGACCATA |
| | | CTCTACAGTTGTGTTTTAATGTATATTAATGTTACTAATG |
| | | TGTTTT (SEQ ID NO: 454) |
| CD70 + | miR204 + | AGGAGGGTGGGGGTGGAGGCAAGCAGAGGACTTCCTGATC |
| CD70 | miR26a1 | GCGTACCCATGGCTACAGTCTTTCTTCATGTGACTCGTGG |
| | | ACTCCTGTGTGATTCAGCTGCAGCGAGAATATATGAAGGG |
| | | CTGAGCTGCATCACACAGGCGTTCAATTGTCATCACTGGC |
| | | ATCTTTTTTGATCATTGCACCATCATCAAATGCATTGGGA |
| | | TAACCATGACATGAAGCCACAGGAGCCAAGAGCAGGAGGA |
| | | CCAAGGCCCTGGCGAAGGCCGTGGCCTCGATGTGTACCAT |
| | | GTAGATGCCATGTGCAGGTCCCAATGGTGGCATCTAGATG |
| | | GTACACCTCGGGGACGCGGGCCTGGACGCCGGCATCCGGG |
| | | CTCAGGACCCCCCTCTCTGCCAGAGGC |
| | | (SEQ ID NO: 455) |
| CD70 + | miR16 + | CTTCTGAAGAAAATATATTTCTTTTTATTCATAGCTCTTA |
| CD70 | miR26a1 | TGATAGCAATGTCAGCAGTGCCTTGTGCGAAGCGCTGGAT |
| | | GCACATTAAGATTCTAAAATTATCTTGCATCCACCGACTT |
| | | CGCACGAAGTAAGGTTGACCATACTCTACAGTTGTGTTTT |
| | | AATGTATATTAATGTTACTAATGTGTTTTATGAAGCCACA |
| | | GGAGCCAAGAGCAGGAGGACCAAGGCCCTGGCGAAGGCCG |
| | | TGGCCTCGATGTGTACCATGTAGATGCCATGTGCAGGTCC |
| | | CAATGGTGGCATCTAGATGGTACACCTCGGGGACGCGGGC |
| | | CTGGACGCCGGCATCCGGGCTCAGGACCCCCCTCTCTGCC |
| | | AGAGGC (SEQ ID NO: 456) |
| CD70 + | miR16 + | CTTCTGAAGAAAATATATTTCTTTTTATTCATAGCTCTTA |
| CD70 | miR22 | TGATAGCAATGTCAGCAGTGCCTTGTGCGAAGCGCTGGAT |
| | | GCACATTAAGATTCTAAAATTATCTTGCATCCACCGACTT |
| | | CGCACGAAGTAAGGTTGACCATACTCTACAGTTGTGTTTT |
| | | AATGTATATTAATGTTACTAATGTGTTTTCATTTTCCCTC |
| | | CCTTTCCCTTAGGAGCCTGTTCCTCTCACGCCCTCACCTG |
| | | GCTGAGCCGCAATGGCATCTACAGGTACAACATTATGTCC |
| | | TGACCCAGCTAATGTGTACCATGTAGATGCCATTGCCCTC |
| | | TGCCCCTGGCTTCGAGGAGGAGGAGGAGCTGCTTTCCCCA |
| | | TCATCTGGAAGGTG |
| | | (SEQ ID NO: 457) |
| CD70 + | miR22 + | CTTCTGAAGAAAATATATTTTTTTTATTCATAGCTCTTAT |
| CD70 | miR16 | GATAGCAATGTCAGCAGTGCCTTGTGCGAAGCGCTGGATG |
| | | CACATTAAGATTCTAAAATTATCTTGCATCCACCGACTTC |
| | | GCACGAAGTAAGGTTGACCATACTCTACAGTTGTGTTTTA |
| | | ATGTATATTAATGTTACTAATGTGTTTTCATTTTCCCTCC |
| | | CTTTCCCTTAGGAGCCTGTTCCTCTCACGCCCTCACCTGG |
| | | CTGAGCCGCAATGGCATCTACAGGTACAACATTATGTCCT |
| | | GACCCAGCTAATGTGTACCATGTAGATGCCATTGCCCTCT |
| | | GCCCCTGGCTTCGAGGAGGAGGAGGAGCTGCTTTCCCCAT |
| | | CATCTGGAAGGTG |
| | | (SEQ ID NO: 458) |
| CD70 + | miR204 + | AGGAGGGTGGGGGTGGAGGCAAGCAGAGGACTTCCTGATC |
| CD70 | miR16 | GCGTACCCATGGCTACAGTCTTTCTTCATGTGACTCGTGG |
| | | ACTCCTGTGTGATTCAGCTGCAGCGAGAATATATGAAGGG |
| | | CTGAGCTGCATCACACAGGCGTTCAATTGTCATCACTGGC |
| | | ATCTTTTTTGATCATTGCACCATCATCAAATGCATTGGGA |
| | | TAACCATGACCTTCTGAAGAAAATATATTTCTTTTTATTC |
| | | ATAGCTCTTATGATAGCAATGTCAGCAGTGCCTTGTGCGA |
| | | AGCGCTGGATGCACATTAAGATTCTAAAATTATCTTGCAT |
| | | CCACCGACTTCGCACGAAGTAAGGTTGACCATACTCTACA |
| | | GTTGTGTTTTAATGTATATTAATGTTACTAATGTGTTTT |
| | | (SEQ ID NO: 459) |
| CD70 + | miR204 + | AGGAGGGTGGGGGTGGAGGCAAGCAGAGGACTTCCTGATC |
| CD70 | miR16 | GCGTACCCATGGCTACAGTCTTTCTTCATGTGACTCGTGG |
| | | ACATGTGTACCATGTAGATGCCATGAGAATATATGAAGGA |
| | | TGGATCTAGATGGTACACACGTTCAATTGTCATCACTGGC |
| | | ATCTTTTTTGATCATTGCACCATCATCAAATGCATTGGGA |
| | | TAACCATGACCTTCTGAAGAAAATATATTTCTTTTTATTC |
| | | ATAGCTCTTATGATAGCAATGTCAGCAGTGCCTTGTGCGA |
| | | AGCGCTGGATGCACATTAAGATTCTAAAATTATCTTGCAT |
| | | CCACCGACTTCGCACGAAGTAAGGTTGACCATACTCTACA |
| | | GTTGTGTTTTAATGTATATTAATGTTACTAATGTGTTTT |
| | | (SEQ ID NO: 460) |

Splice donor and splice acceptor
site sequences

| | |
|---|---|
| 5′ side of intron | AGGTAAGAGTCGATCG |
| (SEQ ID NO: 291) | (SEQ ID NO: 291) |
| 3′ side of intron | ACGCGTTACTAACTGG |
| (SEQ ID NO: 292) | TACCTCTTTTTTTTTT |
| | TTGATATCCTGCAGGC |
| | (SEQ ID NO: 292) |

Exemplary control sequences

| miRNA | SEQ ID | miRNA | |
|---|---|---|---|
| Target | NO | backbone | DNA sequence of Pri-miRNA |
| Scrambled | 582 | miR206, | GATGCTACAAGTGGCCCACTTCTGAGATGCGGGC |
| control 2 | | shorter | TGCTTCTGGATGACACTGCTTCCCGAGGCATTTC |
| | | arms | GCCCTATCTGCAAGTACTATGGATTACTTTGCTA |
| | | | GTGGTGTAGATAGGGTGAAATGTTTCGGCAAGTG |
| | | | CCTCCTCGCTGGCCCCAGGGTACCACCCGGAGCA |
| | | | CAGGTTTGGTGACCTT |
| neg. ctl | 583 | miR150 | AGGGACTGGGCCCACGGGGAGGCAGCGTCCCCGA |
| c. elegans | | | GGCAGCAGCGGCAGCGGCGGCTCCTCTCCCCATG |
| cel-miR-67 | | | GCCCTGTCACAACCTCCTAGAAAGAGTACTGGGC |
| | | | TCAGACCCTCTTTCAGGCCGTTGTGACAGGGACC |
| | | | TGGGGACCCCGGCACCGGCAGGCCCCAAGGGGTG |
| | | | AGGTGAGCGGGCATTGGGACCTCCCCTCCCTGTA |
| | | | CTC |
| neg. ctl | 584 | miR204 | AGGAGGGTGGGGGTGGAGGCAAGCAGAGGACTTC |
| c. elegans | | | CTGATCGCGTACCCATGGCTACAGTCTTTCTTCA |
| cel-miR-67 | | | TGTGACTCGTGGACTTGTACTACACAAAAGTACT |
| | | | GTGAGAATATATGAAGGACAGGCTTTAGTGTAGT |
| | | | ATGCGTTCAATTGTCATCACTGGCATCTTTTTTG |
| | | | ATCATTGCACCATCATCAAATGCATTGGGATAAC |
| | | | CATGAC |
| 210bp | 585 | | GGTAAGTCATGACTCCCTACAATGGACATGATCA |
| stuffer | | | TAGTAGGTACTATAAGGCACCTAGCTATACCCTC |
| | | | CTATAGAGAGTTTGAGTCTATTGTAGCAAGTCTT |
| | | | TCTTTTAGGGCCTAGGACTCTTCCCACTCTCTTC |
| | | | CATCGCCTAAGCTCTAACTCTTCCTTGTAGTAGA |
| | | | AATAAGTCACTTCTAAGGCTGGGACCCTCCTAGA |
| | | | CCCTAA |
| Scrambled | 586 | miR206 | TTAGGATGAGTTGAGATCCCAGTGATCTTCTCGC |
| control 1 | | | TAAGAGTTTCCTGCCTGGGCAAGGAGGAAAGATG |
| | | | CTACAAGTGGCCCACTTCTGAGATGCGGGCTGCT |
| | | | TCTGGATGACACTGCTTCCCGAGGCATTTCGCCC |
| | | | TATCTGCAAGTACTATGGATTACTTTGCTAGTGG |
| | | | TGTAGATAGGGTGAAATGTTTCGGCAAGTGCCTC |
| | | | CTCGCTGGCCCCAGGGTACCACCCGGAGCACAGG |
| | | | TTTGGTGACCTTCTTCCTCATCAGGGCTTTGTGC |
| | | | CAGCAAATGACTCCCTCACCAAGGAAGCAAGAGC |
| | | | CTCTGAATCCCATCTGGGCTCTTCCTGAACACCC |
| | | | CTATCTCCCCCTCT |

1. A ribonucleic acid comprising two non-natural pre-miRNA sequences, wherein each pre-miRNA sequence comprises a guide miRNA that inhibits the expression of an immune checkpoint protein.

2. The ribonucleic acid of claim 1, wherein the non-natural pre-miRNA sequences have less than about 50% sequence identity with each other.

3. The ribonucleic acid of claim 1, wherein the nucleic acid sequence of at least one non-natural pre-miRNA sequence has at least about 90% sequence identity with that of a naturally-occurring pre-miRNA sequence.

4. The ribonucleic acid of claim 1, wherein the two non-natural pre-miRNA sequences are separated from each other by at least 10 nucleotides.

5. The ribonucleic acid of claim 1, wherein each non-natural pre-miRNA sequence targets a different gene.

6. The ribonucleic acid of claim 1, wherein each non-natural pre-miRNA sequence targets different regions of the same gene.

7. The ribonucleic acid of claim 1, wherein each non-natural pre-miRNA comprises backbone sequences that are identical to the corresponding backbone segments of a naturally-occurring pre-miRNA.

8. The ribonucleic acid of claim 1, wherein each non-natural pre-miRNA comprises backbone sequences from miR16, miR17, miR19, miR21, miR22, miR26a1, miR29b1, miR30a, miR122, miR126, miR133a1, miR142, miR150, miR155, miR204, miR206, miR214, miR412, miR486, miR494, or miR1915.

9. The ribonucleic acid of claim 1, wherein each non-natural pre-miRNA comprises backbone sequences from miR16, miR17, miR21, miR22, miR26a1, miR142, miR150, miR204, or miR206.

10. The ribonucleic acid of claim 1, wherein each non-natural pre-miRNA comprises backbone sequences from miR16, miR21, miR22, miR204, or miR206.

11. The ribonucleic acid of claim 1, wherein each non-natural pre-miRNA comprises backbone sequences from miR204 or miR206.

12. The ribonucleic acid of claim 1, wherein the non-natural pre-miRNA comprises a mature miRNA sequence that is capable of binding to an mRNA and thereby interfering with the translation thereof and/or prompting its degradation.

13. The ribonucleic acid of claim 1, wherein the non-natural pre-miRNA comprises a mature miRNA sequence that is capable of binding to an mRNA under stringent hybridization conditions.

14. The ribonucleic acid of claim 1, wherein the immune checkpoint protein is CTLA4, CD70, PD-1, PD-L1, TIGIT, TIM3, LAG3, GITR, or PIK3IP1.

15. The ribonucleic acid of claim 1, wherein the immune checkpoint protein is CTLA4, CD70, PD-1, TIGIT, TIM3, LAG3, GITR, or PIK3IP1.

16. The ribonucleic acid of claim 1, wherein the immune checkpoint protein is CD70, PD-1, or TIGIT.

17. The ribonucleic acid of claim 1, wherein the immune checkpoint protein is PD-1.

18. A deoxyribonucleic acid encoding the ribonucleic acid of any one of claims 1-17.

19. The deoxyribonucleic acid of claim 18, further encoding a protein.

20. The deoxyribonucleic acid of claim 19, wherein the protein is a chimeric antigen receptor.

21. The deoxyribonucleic acid of claim 20, wherein the chimeric antigen receptor comprises an antigen-binding domain that binds an antigen that is overexpressed in a cancer.

22. The deoxyribonucleic acid of claim 21, wherein the chimeric antigen receptor comprises an antigen-binding domain that binds CD19, CD33, MUC-16, or ROR-1.

23. The deoxyribonucleic acid of claim 22, wherein the chimeric antigen receptor comprises an antigen-binding domain that binds ROR-1.

24. The deoxyribonucleic acid of claim 19, wherein the protein is a cytokine.

25. The deoxyribonucleic acid of claim 24, wherein the protein comprises IL-15, or a functional fragment or variant thereof, and IL-15Rα, or a functional fragment or variant thereof.

26. The deoxyribonucleic acid of claim 19, wherein the protein is a cell tag.

27. The deoxyribonucleic acid of claim 26, wherein the cell tag comprises a HER1 Domain III, or a functional fragment or variant thereof, and a truncated HER1 Domain IV, or a functional fragment or variant thereof.

28. The deoxyribonucleic acid of claim 27, wherein the cell tag further comprises a CD28 transmembrane domain or a functional fragment or variant thereof.

29. The deoxyribonucleic acid of claim 19, further encoding: (a) a chimeric antigen receptor; (b) a protein comprising IL-15, or a functional fragment or variant thereof, and IL-15Rα, or a functional fragment or variant thereof; and (c) a cell tag.

30. The deoxyribonucleic acid of claim 19, wherein the protein is an immune checkpoint inhibitor.

31. A vector comprising the ribonucleoic acid of any one of claims 1-17 or the deoxyribonucleic acid of any one of claims 18-30.

32. The vector of claim 31, wherein the vector is a plasmid, a nanoplasmid, a viral vector, an episomal vector, or a non-viral vector.

33. The vector of claim 32, wherein the vector is a Sleeping Beauty transposon.

34. The vector of claim 32, wherein the vector is a viral vector.

35. The vector of claim 34, wherein the vector is an adenoviral vector.

36. A method for modifying the expression of a gene in a cell, wherein the method comprises introducing the ribonucleic acid of any one of claims 1-17 or the deoxyribonucleic acid of any one of claims 18-30 to the cell.

37. A method for modifying the expression of a gene in a cell, wherein the method comprises transfecting the cell with the ribonucleic acid of any one of claims 1-17 or the deoxyribonucleic acid of any one of claims 18-30.

38. A method for modifying the expression of a gene in a cell, wherein the method comprises transfecting the cell with the vector of claim 31.

39. The method of claim 38, further comprising transfecting the cell with a vector encoding a transposase.

40. A method for producing a genetically-engineered cell, wherein the method comprises introducing the ribonucleic acid of any one of claims 1-17 or the deoxyribonucleic acid of any one of claims 18-30 to the cell.

41. A genetically-modified cell comprising the ribonucleic acid of any one of claims 1-17 or the deoxyribonucleic acid of any one of claims 18-30.

42. A genetically-modified cell produced by the method of claim 40.

43. A composition comprising the ribonucleic acid of any one of claims 1-17 or the deoxyribonucleic acid of any one of claims 18-30.

44. A composition comprising the vector of claim 31.

45. A composition comprising the cell of claim 41.

46. The composition of claim 43 for use in modifying the expression of a gene.

47. The composition of claim 43 for use in treating a disease or disorder in a subject.

48. A kit comprising the ribonucleic acid of any one of claims 1-17 or the deoxynucleic acid of any one of claims 18-30.

51. A kit comprising the cell of claim 41.

52. A method of treating a disease or disorder in a subject, comprising administering the ribonucleic acid of any one of claims 1-17 or the deoxynucleic acid of any one of claims 18-30 to the subject.

53. A method of treating a disease or disorder in a subject, comprising administering the cell of claim 41 to the subject.

54. The use of the ribonucleic acid of any one of claims 1-17 or the deoxyribonucleic acid of any one of claims 18-30 in the manufacture of a medicament for modifying the expression of a gene.

55. The use of the ribonucleic acid of any one of claims 1-17 or the deoxyribonucleic acid of any one of claims 18-30 in the manufacture of a medicament for the treatment of a disease or disorder in a subject.


SEQUENCE LISTING (713 sequences)

SEQ ID NO: 1 - DNA, 82 bp, unidentified
  source 1..82: mol_type = unassigned DNA
aggagggtgggggtggaggcaagcagaggacttcctgatcgcgtacccatggctacagtctttcttcatgtgactcgtggac

SEQ ID NO: 2 - DNA, 15 bp, unidentified
  source 1..15: mol_type = unassigned DNA
gagaatatatgaagg

SEQ ID NO: 3 - DNA, 70 bp, unidentified
  source 1..70: mol_type = unassigned DNA
gttcaattgtcatcactggcatcttttttgatcattgcaccatcatcaaatgcattgggataaccatgac

SEQ ID NO: 4 - DNA, 62 bp, unidentified
  source 1..62: mol_type = unassigned DNA
gatgctacaagtggcccacttctgagatgcgggctgcttctggatgacactgcttcccgagg

SEQ ID NO: 5 - DNA, 17 bp, unidentified
  source 1..17: mol_type = unassigned DNA
tatggattactttgcta

SEQ ID NO: 6 - DNA, 62 bp, unidentified
  source 1..62: mol_type = unassigned DNA
tttcggcaagtgcctcctcgctggccccagggtaccacccggagcacaggtttggtgacctt

SEQ ID NO: 7 - DNA, 63 bp, unidentified
  source 1..63: mol_type = unassigned DNA
ttagcaggaaaaaagagaacatcaccttgtaaaactgaagattgtgaccagtcagaataatgt

SEQ ID NO: 8 - DNA, 12 bp, unidentified
  source 1..12: mol_type = unassigned DNA
tgatatgtgcat

SEQ ID NO: 9 - DNA, 64 bp, unidentified
  source 1..64: mol_type = unassigned DNA
gcattatggtgacagctgcctcgggaagccaagttgggctttaaagtgcagggcctgctgatgt

SEQ ID NO: 10 - DNA, 74 bp, unidentified
  source 1..74: mol_type = unassigned DNA
agggactgggcccacggggaggcagcgtccccgaggcagcagcggcagcggcggctcctctccccatggccctg

SEQ ID NO: 11 - DNA, 10 bp, unidentified
  source 1..10: mol_type = unassigned DNA
ctgggctcag

SEQ ID NO: 12 - DNA, 79 bp, unidentified
  source 1..79: mol_type = unassigned DNA
cagggacctggggaccccggcaccggcaggccccaaggggtgaggtgagcgggcattgggacctcccctccctgtactc

SEQ ID NO: 13 - DNA, 71 bp, unidentified
  source 1..71: mol_type = unassigned DNA
agggactgggcccacggggaggcagcgtccccgaggcagcagcggcagcggcggctcctctccccatggcc

SEQ ID NO: 14 - DNA, 15 bp, unidentified
  source 1..15: mol_type = unassigned DNA
tgctgggctcagacc

SEQ ID NO: 15 - DNA, 76 bp, unidentified
  source 1..76: mol_type = unassigned DNA
ggacctggggaccccggcaccggcaggccccaaggggtgaggtgagcgggcattgggacctcccctccctgtactc

SEQ ID NO: 16 - DNA, 63 bp, unidentified
  source 1..63: mol_type = unassigned DNA
cttctgaagaaaatatatttctttttattcatagctcttatgatagcaatgtcagcagtgcct

SEQ ID NO: 17 - DNA, 20 bp, unidentified
  source 1..20: mol_type = unassigned DNA
ttaagattctaaaattatct

SEQ ID NO: 18 - DNA, 63 bp, unidentified
  source 1..63: mol_type = unassigned DNA
aagtaaggttgaccatactctacagttgtgttttaatgtatattaatgttactaatgtgtttt

SEQ ID NO: 19 - DNA, 55 bp, unidentified
  source 1..55: mol_type = unassigned DNA
caggttaacccaacagaaggctaaagaaggtatattgctgttgacagtgagcgac

SEQ ID NO: 20 - DNA, 19 bp, unidentified
  source 1..19: mol_type = unassigned DNA
ctgtgaagccacagatggg

SEQ ID NO: 21 - DNA, 53 bp, unidentified
  source 1..53: mol_type = unassigned DNA
tgcctactgcctcggacttcaaggggctactttaggagcaattatcttgttta

SEQ ID NO: 22 - DNA, 62 bp, unidentified
  source 1..62: mol_type = unassigned DNA
ccggggtcctgtctgcatccagcgcagcattctggaagacgccacgcctccgctggcgacgg

SEQ ID NO: 23 - DNA, 16 bp, unidentified
  source 1..16: mol_type = unassigned DNA
ctgtgacacttcaaac

SEQ ID NO: 24 - DNA, 62 bp, unidentified
  source 1..62: mol_type = unassigned DNA
ccgtccacggcaccgcatcgaaaacgccgctgagacctcagccttgacctccctcagcgtgg

SEQ ID NO: 25 - DNA, 64 bp, unidentified
  source 1..64: mol_type = unassigned DNA
caatggtggaatgtggaggtgaagttaacaccttcgtggctacagagtttccttagcagagctg

SEQ ID NO: 26 - DNA, 12 bp, unidentified
  source 1..12: mol_type = unassigned DNA
tgtctaaactat

SEQ ID NO: 27 - DNA, 92 bp, unidentified
  source 1..92: mol_type = unassigned DNA
tagctactgctaggcaatccttccctcgataaatgtcttggcatcgtttgctttgagcaagaaggttcatctgatatcagtcttctcaatct

SEQ ID NO: 28 - DNA, 77 bp, unidentified
  source 1..77: mol_type = unassigned DNA
acaggctgattgtatctgtctatgagcaaaggaaacctgaaggaaccaagggcctggctggacagagttgtcatgtg

SEQ ID NO: 29 - DNA, 21 bp, unidentified
  source 1..21: mol_type = unassigned DNA
gcagaacatccgctcacctgt

SEQ ID NO: 30 - DNA, 68 bp, unidentified
  source 1..68: mol_type = unassigned DNA
cacatgacaacccagcctgaatgacaaccagccattgaaagaaagcagccctcacaccatagcatcta

SEQ ID NO: 31 - DNA, 79 bp, unidentified
  source 1..79: mol_type = unassigned DNA
acaggctgattgtatctgtctatgagcaaaggaaacctgaaggaaccaagggcctggctggacagagttgtcatgtgtc

SEQ ID NO: 32 - DNA, 17 bp, unidentified
  source 1..17: mol_type = unassigned DNA
agaacatccgctcacct

SEQ ID NO: 33 - DNA, 68 bp, unidentified; identical to SEQ ID NO: 30
  source 1..68: mol_type = unassigned DNA

SEQ ID NO: 34 - DNA, 57 bp, unidentified
  source 1..57: mol_type = unassigned DNA
gggtttattgtaagagagcattatgaagaaaaaaatagatcataaagcttcttcagg

SEQ ID NO: 35 - DNA, 19 bp, unidentified
  source 1..19: mol_type = unassigned DNA
gatttaaatagtgattgtc

SEQ ID NO: 36 - DNA, 58 bp, unidentified
  source 1..58: mol_type = unassigned DNA
cttgggggagaccagctgcgctgcactaccaacagcaaaagaagtgaatgggacagct

SEQ ID NO: 37 - DNA, 59 bp, unidentified
  source 1..59: mol_type = unassigned DNA
gggtttattgtaagagagcattatgaagaaaaaaatagatcataaagcttcttcaggaa

SEQ ID NO: 38 - DNA, 15 bp, unidentified
  source 1..15: mol_type = unassigned DNA
tttaaatagtgattg

SEQ ID NO: 39 - DNA, 61 bp, unidentified
  source 1..61: mol_type = unassigned DNA
gttcttgggggagaccagctgcgctgcactaccaacagcaaaagaagtgaatgggacagct

SEQ ID NO: 40 - DNA, 64 bp, unidentified
  source 1..64: mol_type = unassigned DNA
tttaccaatgaaaagcatttaactgttttggattccaaactagcagcactacaatgctttgcta

SEQ ID NO: 41 - DNA, 16 bp, unidentified
  source 1..16: mol_type = unassigned DNA
tcgcctcttcaatgga

SEQ ID NO: 42 - DNA, 64 bp, unidentified
  source 1..64: mol_type = unassigned DNA
tagctatgcattgattactacgggacaaccaacgttttcatttgtgaatatcaattacttgcca

SEQ ID NO: 43 - DNA, 58 bp, unidentified
  source 1..58: mol_type = unassigned DNA
tgaagccacaggagccaagagcaggaggaccaaggccctggcgaaggccgtggcctcg

SEQ ID NO: 44 - DNA, 15 bp, unidentified
  source 1..15: mol_type = unassigned DNA
gtgcaggtcccaatg

SEQ ID NO: 45 - DNA, 59 bp, unidentified
  source 1..59: mol_type = unassigned DNA
cggggacgcgggcctggacgccggcatccgggctcaggacccccctctctgccagaggc

SEQ ID NO: 46 - DNA, 68 bp, unidentified
  source 1..68: mol_type = unassigned DNA
gtcttggaggctggggcacctcggggaaggacgccggcatcagcaccattctggggtacggggatgga

SEQ ID NO: 47 - skipped

SEQ ID NO: 48 - DNA, 70 bp, unidentified
  source 1..70: mol_type = unassigned DNA
gccgtccgtatccgctgcagcctgtggggcctgcgggccggggagccgatcgcgcttcagctcagcgcct

SEQ ID NO: 49 - DNA, 61 bp, unidentified
  source 1..61: mol_type = unassigned DNA
ccaataattcaagccaagcaagtatataggtgttttaatagtttttgtttgcagtcctctg

SEQ ID NO: 50 - DNA, 17 bp, unidentified
  source 1..17: mol_type = unassigned DNA
tacaagaagaatgtagt

SEQ ID NO: 51 - DNA, 61 bp, unidentified
  source 1..61: mol_type = unassigned DNA
tggtggcctgctatttccttcaaatgaatgatttttactaattttgtgtacttttattgtg

SEQ ID NO: 52 - DNA, 57 bp, unidentified
  source 1..57: mol_type = unassigned DNA
tgtctgcttgttttgcctaccatcgtgacatctccatggctgtaccaccttgtcggg

SEQ ID NO: 53 - DNA, 16 bp, unidentified
  source 1..16: mol_type = unassigned DNA
ctgttgaatctcatgg

SEQ ID NO: 54 - DNA, 57 bp, unidentified
  source 1..57: mol_type = unassigned DNA
tctgacattttggtatctttcatctgaccatccatatccaatgttctcatttaaaca

SEQ ID NO: 55 - DNA, 65 bp, unidentified
  source 1..65: mol_type = unassigned DNA
ggagtcaggaggcctgggcagcctgaagagtacacgccgacggacagacagacagtgcagtcacc

SEQ ID NO: 56 - DNA, 15 bp, unidentified
  source 1..15: mol_type = unassigned DNA
aacagcactggaggg

SEQ ID NO: 57 - DNA, 65 bp, unidentified
  source 1..65: mol_type = unassigned DNA
gatgagtgtactgtgggcttcggagatcacgccactgctgccgcccgctgcccgccaccatcttc

SEQ ID NO: 58 - DNA, 63 bp, unidentified
  source 1..63: mol_type = unassigned DNA
cagttctgttttgattttttttgtttgttttttgatcagtgctaatcttcgatactcgaagga

SEQ ID NO: 59 - DNA, 13 bp, unidentified
  source 1..13: mol_type = unassigned DNA
tctttatttatga

SEQ ID NO: 60 - DNA, 62 bp, unidentified
  source 1..62: mol_type = unassigned DNA
ttttttagtatcaaatcccaccctggaggcacttcctgttcctgatgcagccttcagggagg

SEQ ID NO: 61 - DNA, 61 bp, unidentified
  source 1..61: mol_type = unassigned DNA
cggaccacggtgtccccttctctccagctgggggtctcgggtcctggcgctgagaggccgc

SEQ ID NO: 62 - DNA, 11 bp, unidentified
  source 1..11: mol_type = unassigned DNA
gtgcacccgtg

SEQ ID NO: 63 - DNA, 63 bp, unidentified
  source 1..63: mol_type = unassigned DNA
gcggccctagcgacctgcggcggcgccgggaaagccctgcctctgcagcgggtcccaggggtc

SEQ ID NO: 64 - DNA, 22 bp, unidentified
  source 1..22: mol_type = unassigned DNA
aatatagtcttctccctcgctt

SEQ ID NO: 65 - DNA, 21 bp, unidentified
  source 1..21: mol_type = unassigned DNA
aagcagggacaggactatatt

SEQ ID NO: 66 - DNA, 22 bp, unidentified
  source 1..22: mol_type = unassigned DNA
aatatagtcttctccctcgctt

SEQ ID NO: 67 - DNA, 22 bp, unidentified
  source 1..22: mol_type = unassigned DNA
gagcgagggataggactatact

SEQ ID NO: 68 - DNA, 22 bp, unidentified
  source 1..22: mol_type = unassigned DNA
aatatagtcttctccctcgctg

SEQ ID NO: 69 - DNA, 20 bp, unidentified
  source 1..20: mol_type = unassigned DNA
cagtgagggaagactatgtt

SEQ ID NO: 70 - DNA, 22 bp, unidentified
  source 1..22: mol_type = unassigned DNA
aatatagtcttctccctcgctg

SEQ ID NO: 71 - DNA, 23 bp, unidentified
  source 1..23: mol_type = unassigned DNA
cagcgagggagaagattaccatt

SEQ ID NO: 72 - DNA, 22 bp, unidentified
  source 1..22: mol_type = unassigned DNA
ttcaggaatgggttccaaggat

SEQ ID NO: 73 - DNA, 21 bp, unidentified
  source 1..21: mol_type = unassigned DNA
atcctggaagctattcctgac

SEQ ID NO: 74 - DNA, 22 bp, unidentified
  source 1..22: mol_type = unassigned DNA
tataatataatagaaccacagg

SEQ ID NO: 75 - DNA, 23 bp, unidentified
  source 1..23: mol_type = unassigned DNA
cctgtggttctgttatatccata

SEQ ID NO: 76 - DNA, 22 bp, unidentified
  source 1..22: mol_type = unassigned DNA
ttcaggaatgggttccaaggag

SEQ ID NO: 77 - DNA, 20 bp, unidentified
  source 1..20: mol_type = unassigned DNA
ctccttggaccattcctgaa

SEQ ID NO: 78 - DNA, 24 bp, unidentified
  source 1..24: mol_type = unassigned DNA
ttcaggaatgggttccaaggaatt

SEQ ID NO: 79 - DNA, 23 bp, unidentified
  source 1..23: mol_type = unassigned DNA
aatgtcctgaagccattcatgga

SEQ ID NO: 80 - DNA, 22 bp, unidentified
  source 1..22: mol_type = unassigned DNA
ttcaggaatgggttccaaggag

SEQ ID NO: 81 - DNA, 22 bp, unidentified
  source 1..22: mol_type = unassigned DNA
ctccttggaacacattcctaca

SEQ ID NO: 82 - DNA, 23 bp, unidentified
  source 1..23: mol_type = unassigned DNA
ttcaggaatgggttccaaggaag

SEQ ID NO: 83 - DNA, 24 bp, unidentified
  source 1..24: mol_type = unassigned DNA
cttcttggtacacgttcctgctca

SEQ ID NO: 84 - skipped

SEQ ID NO: 85 - DNA, 22 bp, unidentified
  source 1..22: mol_type = unassigned DNA
acgtgtggtttatcctgttgta

SEQ ID NO: 86 - skipped

SEQ ID NO: 87 - DNA, 26 bp, unidentified
  source 1..26: mol_type = unassigned DNA
ccagcttgtggttctattatgttata

SEQ ID NO: 88 - DNA, 22 bp, unidentified
  source 1..22: mol_type = unassigned DNA
agatccacgttactcaccctag

SEQ ID NO: 89 - DNA, 23 bp, unidentified
  source 1..23: mol_type = unassigned DNA
ctagggtgtgtcatgtggatgaa

SEQ ID NO: 90 - DNA, 22 bp, unidentified
  source 1..22: mol_type = unassigned DNA
agatccacgttactcacccgtg

SEQ ID NO: 91 - DNA, 22 bp, unidentified
  source 1..22: mol_type = unassigned DNA
acccgggtgataatatggatct

SEQ ID NO: 92 - DNA, 22 bp, unidentified
  source 1..22: mol_type = unassigned DNA
agatccacgttactcaccccct

SEQ ID NO: 93 - DNA, 21 bp, unidentified
  source 1..21: mol_type = unassigned DNA
aggggtgagaagcgtggatcc

SEQ ID NO: 94 - DNA, 22 bp, unidentified
  source 1..22: mol_type = unassigned DNA
agatccacgttactcaccctta

SEQ ID NO: 95 - DNA, 23 bp, unidentified
  source 1..23: mol_type = unassigned DNA
taggtgagttacgtggatctgtt

SEQ ID NO: 96 - DNA, 22 bp, unidentified
  source 1..22: mol_type = unassigned DNA
agatccacgttactcaccctgc

SEQ ID NO: 97 - DNA, 22 bp, unidentified
  source 1..22: mol_type = unassigned DNA
gtagggtgagactcgtggatct

SEQ ID NO: 98 - DNA, 22 bp, unidentified
  source 1..22: mol_type = unassigned DNA
accacgatgactgctgtgcaga

SEQ ID NO: 99 - DNA, 23 bp, unidentified
  source 1..23: mol_type = unassigned DNA
tctgcgtagcagtcatcgccggt

SEQ ID NO: 100 - DNA, 22 bp, unidentified
  source 1..22: mol_type = unassigned DNA
accacgatgactgctgtgcaga

SEQ ID NO: 101 - DNA, 21 bp, unidentified
  source 1..21: mol_type = unassigned DNA
tctgatggccgtcatcgtggc

SEQ ID NO: 102 - DNA, 22 bp, unidentified
  source 1..22: mol_type = unassigned DNA
accacgatgactgctgtgcaga

SEQ ID NO: 103 - DNA, 22 bp, unidentified
  source 1..22: mol_type = unassigned DNA
tctgtacagcagcatcgatggt

SEQ ID NO: 104 - DNA, 22 bp, unidentified
  source 1..22: mol_type = unassigned DNA
accacgatgactgctgtgcaga

SEQ ID NO: 105 - DNA, 21 bp, unidentified
  source 1..21: mol_type = unassigned DNA
tgcgcagctgttcatcgtggt

SEQ ID NO: 106 - DNA, 22 bp, unidentified
  source 1..22: mol_type = unassigned DNA
accacgatgactgctgtgcaga

SEQ ID NO: 107 - DNA, 20 bp, unidentified
  source 1..20: mol_type = unassigned DNA
ctgcgaagcagcatcgtggc

SEQ ID NO: 108 - DNA, 22 bp, unidentified
  source 1..22: mol_type = unassigned DNA
accacgatgactgctgtgcaga

SEQ ID NO: 109 - DNA, 21 bp, unidentified
  source 1..21: mol_type = unassigned DNA
tctgcaacgcagtctcgctgg

SEQ ID NO: 110 - DNA, 22 bp, unidentified
  source 1..22: mol_type = unassigned DNA
accacgatgactgctgtgcaga

SEQ ID NO: 111 - DNA, 22 bp, unidentified
  source 1..22: mol_type = unassigned DNA
gatgcacagcaatcatcgtggt

SEQ ID NO: 112 - DNA, 23 bp, unidentified
  source 1..23: mol_type = unassigned DNA
accacgatgactgctgtgcagat

SEQ ID NO: 113 - DNA, 20 bp, unidentified
  source 1..20: mol_type = unassigned DNA
atctgcacagcagttcgtgg

SEQ ID NO: 114 - DNA, 22 bp, unidentified
  source 1..22: mol_type = unassigned DNA
accacgatgactgctgtgcaga

SEQ ID NO: 115 - DNA, 23 bp, unidentified
  source 1..23: mol_type = unassigned DNA
tctgccaatagctgtaatcgtgg

SEQ ID NO: 116 - DNA, 22 bp, unidentified
  source 1..22: mol_type = unassigned DNA
tatcgttcacggtcagcgactg

SEQ ID NO: 117 - DNA, 23 bp, unidentified
  source 1..23: mol_type = unassigned DNA
cagtcgttgactgtggacttata

SEQ ID NO: 118 - DNA, 22 bp, unidentified
  source 1..22: mol_type = unassigned DNA
tatcgttcacggtcagcgactg

SEQ ID NO: 119 - DNA, 21 bp, unidentified
  source 1..21: mol_type = unassigned DNA
cagtgctgaacgtgaacgatc

SEQ ID NO: 120 - DNA, 22 bp, unidentified
  source 1..22: mol_type = unassigned DNA
tatcgttcacggtcagcgactg

SEQ ID NO: 121 - DNA, 22 bp, unidentified
  source 1..22: mol_type = unassigned DNA
tagtcgctgacttgaacagata

SEQ ID NO: 122 - DNA, 21 bp, unidentified
  source 1..21: mol_type = unassigned DNA
tatcgttcacggtcagcgact

SEQ ID NO: 123 - DNA, 21 bp, unidentified
  source 1..21: mol_type = unassigned DNA
tgtcgctgacagtgaacgata

SEQ ID NO: 124 - DNA, 22 bp, unidentified
  source 1..22: mol_type = unassigned DNA
tatcgttcacggtcagcgactg

SEQ ID NO: 125 - DNA, 21 bp, unidentified
  source 1..21: mol_type = unassigned DNA
gtcgctgagcgctgaacgatg

SEQ ID NO: 126 - DNA, 22 bp, unidentified
  source 1..22: mol_type = unassigned DNA
taactcaggacattgaagtagt

SEQ ID NO: 127 - DNA, 23 bp, unidentified
  source 1..23: mol_type = unassigned DNA
actacttcaatgtcctgacctta

SEQ ID NO: 128 - DNA, 22 bp, unidentified
  source 1..22: mol_type = unassigned DNA
taactcaggacattgaagtagt

SEQ ID NO: 129 - DNA, 21 bp, unidentified
  source 1..21: mol_type = unassigned DNA
actattcagagtcctgagttc

SEQ ID NO: 130 - DNA, 22 bp, unidentified
  source 1..22: mol_type = unassigned DNA
taactcaggacattgaagtagt

SEQ ID NO: 131 - DNA, 20 bp, unidentified
  source 1..20: mol_type = unassigned DNA
ctactccaatgcctgagttg

SEQ ID NO: 132 - DNA, 22 bp, unidentified
  source 1..22: mol_type = unassigned DNA
taactcaggacattgaagtagt

SEQ ID NO: 133 - DNA, 21 bp, unidentified
  source 1..21: mol_type = unassigned DNA
actactgtaatgtctgacgtt

SEQ ID NO: 134 - DNA, 23 bp, unidentified
  source 1..23: mol_type = unassigned DNA
taactcaggacattgaagtagtc

SEQ ID NO: 135 - DNA, 20 bp, unidentified
  source 1..20: mol_type = unassigned DNA
gactacttcaatgttgagtt

SEQ ID NO: 136 - DNA, 22 bp, unidentified
  source 1..22: mol_type = unassigned DNA
agatccacgttactcaccctag

SEQ ID NO: 137 - DNA, 23 bp, unidentified
  source 1..23: mol_type = unassigned DNA
ctaggcagtgagcaaagtggatc

SEQ ID NO: 138 - DNA, 22 bp, unidentified
  source 1..22: mol_type = unassigned DNA
agatccacgttactcaccctag

SEQ ID NO: 139 - DNA, 21 bp, unidentified
  source 1..21: mol_type = unassigned DNA
ctaggtgagaaacgtggatcg

SEQ ID NO: 140 - DNA, 22 bp, unidentified
  source 1..22: mol_type = unassigned DNA
agatccacgttactcaccctag

SEQ ID NO: 141 - DNA, 23 bp, unidentified
  source 1..23: mol_type = unassigned DNA
ctagggtgagtaacgtggcctct

SEQ ID NO: 142 - DNA, 22 bp, unidentified
  source 1..22: mol_type = unassigned DNA
agatccacgttactcaccctag

SEQ ID NO: 143 - DNA, 20 bp, unidentified
  source 1..20: mol_type = unassigned DNA
tagggcgagtacgtggatcg

SEQ ID NO: 144 - DNA, 22 bp, unidentified
  source 1..22: mol_type = unassigned DNA
agatccacgttactcaccctag

SEQ ID NO: 145 - DNA, 22 bp, unidentified
  source 1..22: mol_type = unassigned DNA
ctagggtgagtacgtggcatct

SEQ ID NO: 146 - DNA, 22 bp, unidentified
  source 1..22: mol_type = unassigned DNA
cattatgcctgggatttggatc

SEQ ID NO: 147 - DNA, 21 bp, unidentified
  source 1..21: mol_type = unassigned DNA
gatcagatcgcaggcataatt

SEQ ID NO: 148 - DNA, 22 bp, unidentified
  source 1..22: mol_type = unassigned DNA
cattatgcctgggatttggatc

SEQ ID NO: 149 - DNA, 23 bp, unidentified
  source 1..23: mol_type = unassigned DNA
gatccggatcctaggtatccatg

SEQ ID NO: 150 - DNA, 23 bp, unidentified
  source 1..23: mol_type = unassigned DNA
cattatgcctgggatttggatcg

SEQ ID NO: 151 - DNA, 23 bp, unidentified
  source 1..23: mol_type = unassigned DNA
cggccagaaccaaggcataagta

SEQ ID NO: 152 - DNA, 22 bp, unidentified
  source 1..22: mol_type = unassigned DNA
cattatgcctgggatttggatc

SEQ ID NO: 153 - DNA, 23 bp, unidentified
  source 1..23: mol_type = unassigned DNA
actccaaatcccgtgcataatcg

SEQ ID NO: 154 - DNA, 22 bp, unidentified
  source 1..22: mol_type = unassigned DNA
cattatgcctgggatttggatc

SEQ ID NO: 155 - DNA, 22 bp, unidentified
  source 1..22: mol_type = unassigned DNA
gattcgaatccaaggcatatgg

SEQ ID NO: 156 - DNA, 22 bp, unidentified
  source 1..22: mol_type = unassigned DNA
taattcacatccctttcatcag

SEQ ID NO: 157 - DNA, 22 bp, unidentified
  source 1..22: mol_type = unassigned DNA
atgatgaaaggacagtgaatta

SEQ ID NO: 158 - DNA, 22 bp, unidentified
  source 1..22: mol_type = unassigned DNA
tagtcgttgggtaaagtcgcca

SEQ ID NO: 159 - DNA, 20 bp, unidentified
  source 1..20: mol_type = unassigned DNA
tggtgacttctcaacgatta

SEQ ID NO: 160 - DNA, 22 bp, unidentified
  source 1..22: mol_type = unassigned DNA
gttgctttccgctaagtggtga

SEQ ID NO: 161 - DNA, 22 bp, unidentified
  source 1..22: mol_type = unassigned DNA
tcaccacttagaggaaagcctc

SEQ ID NO: 162 - DNA, 22 bp, unidentified
  source 1..22: mol_type = unassigned DNA
tttgcagtggccttcgtggccc

SEQ ID NO: 163 - DNA, 23 bp, unidentified
  source 1..23: mol_type = unassigned DNA
gggctatgaaggccattgagaaa

SEQ ID NO: 164 - DNA, 23 bp, unidentified
  source 1..23: mol_type = unassigned DNA
agcctcccgtcctaagaccccac

SEQ ID NO: 165 - DNA, 24 bp, unidentified
  source 1..24: mol_type = unassigned DNA
gtgggtcttaggtcgggagctgct

SEQ ID NO: 166 - DNA, 22 bp, unidentified
  source 1..22: mol_type = unassigned DNA
tgaacgaccagtgtttaaccgg

SEQ ID NO: 167 - DNA, 23 bp, unidentified
  source 1..23: mol_type = unassigned DNA
ccggttgaacattggttgcctca

SEQ ID NO: 168 - DNA, 22 bp, unidentified
  source 1..22: mol_type = unassigned DNA
ttctccttggagttcatccgcg

SEQ ID NO: 169 - DNA, 18 bp, unidentified
  source 1..18: mol_type = unassigned DNA
atgaacttagaggagacg

SEQ ID NO: 170 - DNA, 22 bp, unidentified
  source 1..22: mol_type = unassigned DNA
ttctccttggagttcatccgcg

SEQ ID NO: 171 - DNA, 20 bp, unidentified
  source 1..20: mol_type = unassigned DNA
cgcggatgatccaaggagga

SEQ ID NO: 172-177 - skipped (6 entries)

SEQ ID NO: 178 - DNA, 210 bp, synthetic construct
  source 1..210: mol_type = other DNA
aggagggtgggggtggaggcaagcagaggacttcctgatcgcgtacccatggctacagtctttcttcatgtgactcgtggacaatatagtcttctccctcgcttgagaatatatgaaggaagcagggacaggactatattgttcaattgtcatcactggcatcttttttgatcattgcaccatcatcaaatgcattgggataaccatgac

SEQ ID NO: 179 - DNA, 210 bp, synthetic construct
  source 1..210: mol_type = other DNA
aggagggtgggggtggaggcaagcagaggacttcctgatcgcgtacccatggctacagtctttcttcatgtgactcgtggacttcaggaatgggttccaaggatgagaatatatgaaggatcctggaagctattcctgacgttcaattgtcatcactggcatcttttttgatcattgcaccatcatcaaatgcattgggataaccatgac

SEQ ID NO: 180 - DNA, 186 bp, synthetic construct
  source 1..186: mol_type = other DNA
gatgctacaagtggcccacttctgagatgcgggctgcttctggatgacactgcttcccgaggcctgtggttctgttatatccatatatggattactttgctatataatataatagaaccacaggtttcggcaagtgcctcctcgctggccccagggtaccacccggagcacaggtttggtgacctt

SEQ ID NO: 181 - DNA, 184 bp, synthetic construct
  source 1..184: mol_type = other DNA
ttagcaggaaaaaagagaacatcaccttgtaaaactgaagattgtgaccagtcagaataatgtagatccacgttactcaccctagtgatatgtgcatctagggtgtgtcatgtggatgaagcattatggtgacagctgcctcgggaagccaagttgggctttaaagtgcagggcctgctgatgt

SEQ ID NO: 182 - DNA, 205 bp, synthetic construct
  source 1..205: mol_type = other DNA
agggactgggcccacggaggcagcgtccccgaggcagcagcggcagcggcggctcctctccccatggccctgagatccacgttactcacccgtgctgggctcagacccgggtgataatatggatctcagggacctggggaccccggcaccggcaggccccaaggggtgaggtgagcgggcattgggacctcccctccctgtactc

SEQ ID NO: 183 - DNA, 210 bp, synthetic construct
  source 1..210: mol_type = other DNA
aggagggtgggggtggaggcaagcagaggacttcctgatcgcgtacccatggctacagtctttcttcatgtgactcgtggacagatccacgttactcaccccctgagaatatatgaaggaggggtgagaagcgtggatccgttcaattgtcatcactggcatcttttttgatcattgcaccatcatcaaatgcattgggataaccatgac

SEQ ID NO: 184 - DNA, 186 bp, synthetic construct
  source 1..186: mol_type = other DNA
gatgctacaagtggcccacttctgagatgcgggctgcttctggatgacactgcttcccgaggtctgcgtagcagtcatcgccggttatggattactttgctaaccacgatgactgctgtgcagatttcggcaagtgcctcctcgctggccccagggtaccacccggagcacaggtttggtgacctt

SEQ ID NO: 185 - DNA, 210 bp, synthetic construct
  source 1..210: mol_type = other DNA
aggagggtgggggtggaggcaagcagaggacttcctgatcgcgtacccatggctacagtctttcttcatgtgactcgtggacaccacgatgactgctgtgcagagagaatatatgaaggtctgatggccgtcatcgtggcgttcaattgtcatcactggcatcttttttgatcattgcaccatcatcaaatgcattgggataaccatgac

SEQ ID NO: 186 - DNA, 185 bp, synthetic construct
  source 1..185: mol_type = other DNA
cattttccctccctttcccttaggagcctgttcctctcacgccctcacctggctgagccgcatctgtacagcagcatcgatggttatgtcctgacccagctaaccacgatgactgctgtgcagatgccctctgcccctggcttcgaggaggaggaggagctgctttccccatcatctggaaggtg

SEQ ID NO: 187 - DNA, 188 bp, synthetic construct
  source 1..188: mol_type = other DNA
cttctgaagaaaatatatttctttttattcatagctcttatgatagcaatgtcagcagtgcctaccacgatgactgctgtgcagattaagattctaaaattatcttgcgcagctgttcatcgtggtagtaaggttgaccatactctacagttgtgttttaatgtatattaatgttactaatgtgtttt

SEQ ID NO: 188 - DNA, 189 bp, synthetic construct
  source 1..189: mol_type = other DNA
cttctgaagaaaatatatttctttttattcatagctcttatgatagcaatgtcagcagtgcctaccacgatgactgctgtgcagattaagattctaaaattatcttgcgcagctgttcatcgtggtaagtaaggttgaccatactctacagttgtgttttaatgtatattaatgttactaatgtgtttt

SEQ ID NO: 189 - DNA, 172 bp, synthetic construct
  source 1..172: mol_type = other DNA
tgtctgcttgttttgcctaccatcgtgacatctccatggctgtaccaccttgtcgggaccacgatgactgctgtgcagactgttgaatctcatggctgcgaagcagcatcgtggctctgacattttggtatctttcatctgaccatccatatccaatgttctcatttaaaca

SEQ ID NO: 190 - DNA, 181 bp, synthetic construct
  source 1..181: mol_type = other DNA
cagttctgttttgattttttttgtttgttttttgatcagtgctaatcttcgatactcgaaggatctgcaacgcagtctcgctggtctttatttatgaaccacgatgactgctgtgcagattttttagtatcaaatcccaccctggaggcacttcctgttcctgatgcagccttcagggagg

SEQ ID NO: 191 - DNA, 187 bp, synthetic construct
  source 1..187: mol_type = other DNA
ggagtcaggaggcctgggcagcctgaagagtacacgccgacggacagacagacagtgcagtcaccaccacgatgactgctgtgcagaacagcactggagggatgcacagcaatcatcgtggtgatgagtgtactgtgggcttcggagatcacgccactgctgccgcccgctgcccgccaccatcttc

SEQ ID NO: 192 - DNA, 182 bp, synthetic construct
  source 1..182: mol_type = other DNA
ccaataattcaagccaagcaagtatataggtgttttaatagtttttgtttgcagtcctctgatctgcacagcagttcgtggtacaagaagaatgtagtaccacgatgactgctgtgcagattggtggcctgctatttccttcaaatgaatgatttttactaattttgtgtacttttattgtg

SEQ ID NO: 193 - DNA, 180 bp, synthetic construct
  source 1..180: mol_type = other DNA
cggaccacggtgtccccttctctccagctgggggtctcgggtcctggcgctgagaggccgcaccacgatgactgctgtgcagagtgcacccgtgtctgccaatagctgtaatcgtgggcggccctagcgacctgcggcggcgccgggaaagccctgcctctgcagcgggtcccaggggtc

SEQ ID NO: 194 - DNA, 186 bp, synthetic construct
  source 1..186: mol_type = other DNA
gatgctacaagtggcccacttctgagatgcgggctgcttctggatgacactgcttcccgaggcagtcgttgactgtggacttatatatggattactttgctatatcgttcacggtcagcgactgtttcggcaagtgcctcctcgctggccccagggtaccacccggagcacaggtttggtgacctt

SEQ ID NO: 195 - DNA, 210 bp, synthetic construct
  source 1..210: mol_type = other DNA
aggagggtgggggtggaggcaagcagaggacttcctgatcgcgtacccatggctacagtctttcttcatgtgactcgtggactatcgttcacggtcagcgactggagaatatatgaaggcagtgctgaacgtgaacgatcgttcaattgtcatcactggcatcttttttgatcattgcaccatcatcaaatgcattgggataaccatgac

SEQ ID NO: 196 - DNA, 185 bp, synthetic construct
  source 1..185: mol_type = other DNA
cattttccctccctttcccttaggagcctgttcctctcacgccctcacctggctgagccgcatagtcgctgacttgaacagatatatgtcctgacccagctatatcgttcacggtcagcgactgtgccctctgcccctggcttcgaggaggaggaggagctgctttccccatcatctggaaggtg

SEQ ID NO: 197 - DNA, 187 bp, synthetic construct
  source 1..187: mol_type = other DNA
ggagtcaggaggcctgggcagcctgaagagtacacgccgacggacagacagacagtgcagtcacctatcgttcacggtcagcgactaacagcactggagggtgtcgctgacagtgaacgatagatgagtgtactgtgggcttcggagatcacgccactgctgccgcccgctgcccgccaccatcttc

SEQ ID NO: 198 - DNA, 189 bp, synthetic construct
  source 1..189: mol_type = other DNA
cttctgaagaaaatatatttctttttattcatagctcttatgatagcaatgtcagcagtgccttatcgttcacggtcagcgactgttaagattctaaaattatctgtcgctgagcgctgaacgatgaagtaaggttgaccatactctacagttgtgttttaatgtatattaatgttactaatgtgtttt

SEQ ID NO: 199 - DNA, 186 bp, synthetic construct
  source 1..186: mol_type = other DNA
gatgctacaagtggcccacttctgagatgcgggctgcttctggatgacactgcttcccgaggactacttcaatgtcctgaccttatatggattactttgctataactcaggacattgaagtagttttcggcaagtgcctcctcgctggccccagggtaccacccggagcacaggtttggtgacctt

SEQ ID NO: 200 - DNA, 210 bp, synthetic construct
  source 1..210: mol_type = other DNA
aggagggtgggggtggaggcaagcagaggacttcctgatcgcgtacccatggctacagtctttcttcatgtgactcgtggactaactcaggacattgaagtagtgagaatatatgaaggactattcagagtcctgagttcgttcaattgtcatcactggcatcttttttgatcattgcaccatcatcaaatgcattgggataaccatgac

SEQ ID NO: 201 - DNA, 172 bp, synthetic construct
  source 1..172: mol_type = other DNA
tgtctgcttgttttgcctaccatcgtgacatctccatggctgtaccaccttgtcgggtaactcaggacattgaagtagtctgttgaatctcatggctactccaatgcctgagttgtctgacattttggtatctttcatctgaccatccatatccaatgttctcatttaaaca

SEQ ID NO: 202 - DNA, 181 bp, synthetic construct
  source 1..181: mol_type = other DNA
cagttctgttttgattttttttgtttgttttttgatcagtgctaatcttcgatactcgaaggaactactgtaatgtctgacgtttctttatttatgataactcaggacattgaagtagtttttttagtatcaaatcccaccctggaggcacttcctgttcctgatgcagccttcagggagg

SEQ ID NO: 203 - DNA, 182 bp, synthetic construct
  source 1..182: mol_type = other DNA
ccaataattcaagccaagcaagtatataggtgttttaatagtttttgtttgcagtcctctggactacttcaatgttgagtttacaagaagaatgtagttaactcaggacattgaagtagtctggtggcctgctatttccttcaaatgaatgatttttactaattttgtgtacttttattgtg

SEQ ID NO: 204 - DNA, 180 bp, synthetic construct
  source 1..180: mol_type = other DNA
cggaccacggtgtccccttctctccagctgggggtctcgggtcctggcgctgagaggccgcagatccacgttactcaccctaggtgcacccgtgctaggcagtgagcaaagtggatcgcggccctagcgacctgcggcggcgccgggaaagccctgcctctgcagcgggtcccaggggtc

SEQ ID NO: 205 - DNA, 210 bp, synthetic construct
  source 1..210: mol_type = other DNA
aggagggtgggggtggaggcaagcagaggacttcctgatcgcgtacccatggctacagtctttcttcatgtgactcgtggacagatccacgttactcaccctaggagaatatatgaaggctaggtgagaaacgtggatcggttcaattgtcatcactggcatcttttttgatcattgcaccatcatcaaatgcattgggataaccatgac

SEQ ID NO: 206 - DNA, 186 bp, synthetic construct
  source 1..186: mol_type = other DNA
gatgctacaagtggcccacttctgagatgcgggctgcttctggatgacactgcttcccgaggctagggtgagtaacgtggcctcttatggattactttgctaagatccacgttactcaccctagtttcggcaagtgcctcctcgctggccccagggtaccacccggagcacaggtttggtgacctt

SEQ ID NO: 207 - DNA, 172 bp, synthetic construct
  source 1..172: mol_type = other DNA
tgtctgcttgttttgcctaccatcgtgacatctccatggctgtaccaccttgtcgggagatccacgttactcaccctagctgttgaatctcatggtagggcgagtacgtggatcgtctgacattttggtatctttcatctgaccatccatatccaatgttctcatttaaaca

SEQ ID NO: 208 - DNA, 185 bp, synthetic construct
  source 1..185: mol_type = other DNA
cattttccctccctttcccttaggagcctgttcctctcacgccctcacctggctgagccgcactagggtgagtacgtggcatcttatgtcctgacccagctaagatccacgttactcaccctagtgccctctgcccctggcttcgaggaggaggaggagctgctttccccatcatctggaaggtg

SEQ ID NO: 209 - DNA, 210 bp, synthetic construct
  source 1..210: mol_type = other DNA
aggagggtgggggtggaggcaagcagaggacttcctgatcgcgtacccatggctacagtctttcttcatgtgactcgtggaccattatgcctgggatttggatcgagaatatatgaagggatcagatcgcaggcataattgttcaattgtcatcactggcatcttttttgatcattgcaccatcatcaaatgcattgggataaccatgac

SEQ ID NO: 210 - DNA, 207 bp, synthetic construct
  source 1..207: mol_type = other DNA
agggactgggcccacggggaggcagcgtccccgaggcagcagcggcagcggcggctcctctccccatggccctgcattatgcctgggatttggatcctgggctcagacctccaaatccacacatagtgcagggacctggggaccccggcaccggcaggccccaaggggtgaggtgagcgggcattgggacctcccctccctgtactc

SEQ ID NO: 211 - DNA, 171 bp, synthetic construct
  source 1..171: mol_type = other DNA
caggttaacccaacagaaggctaaagaaggtatattgctgttgacagtgagcgaccattatgcctgggatttggatcctgtgaagccacagatggggatccagatcaggcatagtggctgcctactgcctcggacttcaaggggctactttaggagcaattatcttgttta

SEQ ID NO: 212 - DNA, 186 bp, synthetic construct
  source 1..186: mol_type = other DNA
gatgctacaagtggcccacttctgagatgcgggctgcttctggatgacactgcttcccgagggatccggatcctaggtatccatgtatggattactttgctacattatgcctgggatttggatctttcggcaagtgcctcctcgctggccccagggtaccacccggagcacaggtttggtgacctt

SEQ ID NO: 213 - DNA, 189 bp, synthetic construct
  source 1..189: mol_type = other DNA
cttctgaagaaaatatatttctttttattcatagctcttatgatagcaatgtcagcagtgcctcattatgcctgggatttggatcttaagattctaaaattatcttccgaatcacatggcataatgaagtaaggttgaccatactctacagttgtgttttaatgtatattaatgttactaatgtgtttt

SEQ ID NO: 214 - DNA, 185 bp, synthetic construct
  source 1..185: mol_type = other DNA
ttagcaggaaaaaagagaacatcaccttgtaaaactgaagattgtgaccagtcagaataatgtcattatgcctgggatttggatcgtgatatgtgcatcggccagaaccaaggcataagtagcattatggtgacagctgcctcgggaagccaagttgggctttaaagtgcagggcctgctgatgt

SEQ ID NO: 215 - DNA, 185 bp, synthetic construct
  source 1..185: mol_type = other DNA
ccggggtcctgtctgcatccagcgcagcattctggaagacgccacgcctccgctggcgacggactccaaatcccgtgcataatcgctgtgacacttcaaaccattatgcctgggatttggatcccgtccacggcaccgcatcgaaaacgccgctgagacctcagccttgacctccctcagcgtgg

SEQ ID NO: 216 - DNA, 212 bp, synthetic construct
  source 1..212: mol_type = other DNA
caatggtggaatgtggaggtgaagttaacaccttcgtggctacagagtttccttagcagagctgcattatgcctgggatttggatctgtctaaactatgattcgaatccaaggcatatggtagctactgctaggcaatccttccctcgataaatgtcttggcatcgtttgctttgagcaagaaggttcatctgatatcagtcttctcaatct

SEQ ID NO: 217 - DNA, 210 bp, synthetic construct
  source 1..210: mol_type = other DNA
acaggctgattgtatctgtctatgagcaaaggaaacctgaaggaaccaagggcctggctggacagagttgtcatgtgatgatgaaaggacagtgaattagcagaacatccgctcacctgttaattcacatccctttcatcagcacatgacaacccagcctgaatgacaaccagccattgaaagaaagcagccctcacaccatagcatcta

SEQ ID NO: 218 - DNA, 181 bp, synthetic construct
  source 1..181: mol_type = other DNA
gggtttattgtaagagagcattatgaagaaaaaaatagatcataaagcttcttcaggacgatggaagggttgtgaacgttagatttaaatagtgattgtctaattcacatccctttcatcagtcttgggggagaccagctgcgctgcactaccaacagcaaaagaagtgaatgggacagct

SEQ ID NO: 219 - DNA, 210 bp, synthetic construct
  source 1..210: mol_type = other DNA
aggagggtgggggtggaggcaagcagaggacttcctgatcgcgtacccatggctacagtctttcttcatgtgactcgtggactaattcacatccctttcatcaggagaatatatgaaggctgaggaagaggtgtgaattcgttcaattgtcatcactggcatcttttttgatcattgcaccatcatcaaatgcattgggataaccatgac

SEQ ID NO: 220 - DNA, 188 bp, synthetic construct
  source 1..188: mol_type = other DNA
tttaccaatgaaaagcatttaactgttttggattccaaactagcagcactacaatgctttgctagtgatgaataggctgcgaattatcgcctcttcaatggataattcacatccctttcatcagtagctatgcattgattactacgggacaaccaacgttttcatttgtgaatatcaattacttgcca

SEQ ID NO: 221 - DNA, 171 bp, synthetic construct
  source 1..171: mol_type = other DNA
caggttaacccaacagaaggctaaagaaggtatattgctgttgacagtgagcgactagtcgttgggtaaagtcgccactgtgaagccacagatgggtggtgacttctcaacgattagctgcctactgcctcggacttcaaggggctactttaggagcaattatcttgttta

SEQ ID NO: 222 - DNA, 186 bp, synthetic construct
  source 1..186: mol_type = other DNA
gatgctacaagtggcccacttctgagatgcgggctgcttctggatgacactgcttcccgaggtggcgattttacccaatgctctatatggattactttgctatagtcgttgggtaaagtcgccatttcggcaagtgcctcctcgctggccccagggtaccacccggagcacaggtttggtgacctt

SEQ ID NO: 223 - DNA, 210 bp, synthetic construct
  source 1..210: mol_type = other DNA
aggagggtgggggtggaggcaagcagaggacttcctgatcgcgtacccatggctacagtctttcttcatgtgactcgtggactagtcgttgggtaaagtcgccagagaatatatgaaggtggcactttcctcaacgactcgttcaattgtcatcactggcatcttttttgatcattgcaccatcatcaaatgcattgggataaccatgac

SEQ ID NO: 224 - DNA, 207 bp, synthetic construct
  source 1..207: mol_type = other DNA
agggactgggcccacggggaggcagcgtccccgaggcagcagcggcagcggcggctcctctccccatggccctgtagtcgttgggtaaagtcgccactgggctcagaccgcgacttaccacacgactacagggacctggggaccccggcaccggcaggccccaaggggtgaggtgagcgggcattgggacctcccctccctgtactc

SEQ ID NO: 225 - DNA, 185 bp, synthetic construct
  source 1..185: mol_type = other DNA
ttagcaggaaaaaagagaacatcaccttgtaaaactgaagattgtgaccagtcagaataatgtgttgctttccgctaagtggtgagtgatatgtgcatctcccactcagaggaaagcactagcattatggtgacagctgcctcgggaagccaagttgggctttaaagtgcagggcctgctgatgt

SEQ ID NO: 226 - DNA, 212 bp, synthetic construct
  source 1..212: mol_type = other DNA
caatggtggaatgtggaggtgaagttaacaccttcgtggctacagagtttccttagcagagctggttgctttccgctaagtggtgatgtctaaactattcaccacttagaggaaagcctctagctactgctaggcaatccttccctcgataaatgtcttggcatcgtttgctttgagcaagaaggttcatctgatatcagtcttctcaatct

SEQ ID NO: 227 - DNA, 185 bp, synthetic construct
  source 1..185: mol_type = other DNA
ccggggtcctgtctgcatccagcgcagcattctggaagacgccacgcctccgctggcgacgggaaccacttagcgcgaagcaaggctgtgacacttcaaacgttgctttccgctaagtggtgaccgtccacggcaccgcatcgaaaacgccgctgagacctcagccttgacctccctcagcgtgg

SEQ ID NO: 228 - DNA, 171 bp, synthetic construct
  source 1..171: mol_type = other DNA
caggttaacccaacagaaggctaaagaaggtatattgctgttgacagtgagcgactttgcagtggccttcgtggcccctgtgaagccacagatggggggtcacgagccactgcgaagctgcctactgcctcggacttcaaggggctactttaggagcaattatcttgttta

SEQ ID NO: 229 - DNA, 186 bp, synthetic construct
  source 1..186: mol_type = other DNA
gatgctacaagtggcccacttctgagatgcgggctgcttctggatgacactgcttcccgagggggctatgaaggccattgagaaatatggattactttgctatttgcagtggccttcgtagccctttcggcaagtgcctcctcgctggccccagggtaccacccggagcacaggtttggtgacctt

SEQ ID NO: 230 - DNA, 185 bp, synthetic construct
  source 1..185: mol_type = other DNA
ttagcaggaaaaaagagaacatcaccttgtaaaactgaagattgtgaccagtcagaataatgttttgcagtggccttcgtggcccgtgatatgtgcatcggtcacgtagactactgcactcgcattatggtgacagctgcctcgggaagccaagttgggctttaaagtgcagggcctgctgatgt

SEQ ID NO: 231 - DNA, 212 bp, synthetic construct
  source 1..212: mol_type = other DNA
caatggtggaatgtggaggtgaagttaacaccttcgtggctacagagtttccttagcagagctgtttgcagtggccttcgtggccctgtctaaactatgggtcacgaagaccactgcccatagctactgctaggcaatccttccctcgataaatgtcttggcatcgtttgctttgagcaagaaggttcatctgatatcagtcttctcaatct

SEQ ID NO: 232 - DNA, 207 bp, synthetic construct
  source 1..207: mol_type = other DNA
agggactgggcccacggggaggcagcgtccccgaggcagcagcggcagcggcggctcctctccccatggccctgagcctcccgtcctaagaccccactgggctcagacagggtcttggaaaggaggctcagggacctggggaccccggcaccggcaggccccaaggggtgaggtgagcgggcattgggacctcccctccctgtactc

SEQ ID NO: 233 - DNA, 181 bp, synthetic construct
  source 1..181: mol_type = other DNA
gggtttattgtaagagagcattatgaagaaaaaaatagatcataaagcttcttcagggtgggtcttaggtcgggagctgctgatttaaatagtgattgtcagcctcccgtcctaagaccccaccttgggggagaccagctgcgctgcactaccaacagcaaaagaagtgaatgggacagct

SEQ ID NO: 234 - DNA, 209 bp, synthetic construct
  source 1..209: mol_type = other DNA
tctcccatccccttcagatacttacagatactgtaaagtgagtagaattctgagttttgaggttgcttcagtgagcctcccgtcctaagaccccacttggaattaaaatcaagtgggtcttgacgggtggcaccctatggctaaccatcatctactccatggtgctcagaattcgctgaagacaggaaaccaaaggtggacacaccagg

SEQ ID NO: 235 - DNA, 186 bp, synthetic construct
  source 1..186: mol_type = other DNA
gatgctacaagtggcccacttctgagatgcgggctgcttctggatgacactgcttcccgaggccagggtgagtaacgtggcctcttatggattactttgctaagatccacgttactcaccctggtttcggcaagtgcctcctcgctggccccagggtaccacccggagcacaggtttggtgacctt

SEQ ID NO: 236 - DNA, 171 bp, synthetic construct
  source 1..171: mol_type = other DNA
caggttaacccaacagaaggctaaagaaggtatattgctgttgacagtgagcgacagatccacgttactcacccaagctgtgaagccacagatgggcttgggtgagacgtggatctgctgcctactgcctcggacttcaaggggctactttaggagcaattatcttgttta

SEQ ID NO: 237 - DNA, 188 bp, synthetic construct
  source 1..188: mol_type = other DNA
tttaccaatgaaaagcatttaactgttttggattccaaactagcagcactacaatgctttgctagaggggtgtataccgcggatcttcgcctcttcaatggaagatccacgttactcacccctgtagctatgcattgattactacgggacaaccaacgttttcatttgtgaatatcaattacttgcca

SEQ ID NO: 238 - DNA, 212 bp, synthetic construct
  source 1..212: mol_type = other DNA
caatggtggaatgtggaggtgaagttaacaccttcgtggctacagagtttccttagcagagctgagatccacgttactcacccttgtgtctaaactatcaagggtgagtcacgtggagattagctactgctaggcaatccttccctcgataaatgtcttggcatcgtttgctttgagcaagaaggttcatctgatatcagtcttctcaatct

SEQ ID NO: 239 - DNA, 181 bp, synthetic construct
  source 1..181: mol_type = other DNA
gggtttattgtaagagagcattatgaagaaaaaaatagatcataaagcttcttcaggaaagatccacgttactcacccttagatttaaatagtgattgtctaggtgagttacgtggatctgttcttgggggagaccagctgcgctgcactaccaacagcaaaagaagtgaatgggacagct

SEQ ID NO: 240 - DNA, 210 bp, synthetic construct
  source 1..210: mol_type = other DNA
acaggctgattgtatctgtctatgagcaaaggaaacctgaaggaaccaagggcctggctggacagagttgtcatgtgtcagatccacgttactcaccctgcagaacatccgctcacctgtagggtgagactcgtggatctgtcacatgacaacccagcctgaatgacaaccagccattgaaagaaagcagccctcacaccatagcatcta

SEQ ID NO: 241 - DNA, 171 bp, synthetic construct
  source 1..171: mol_type = other DNA
caggttaacccaacagaaggctaaagaaggtatattgctgttgacagtgagcgacttcaggaatgggttccaaggagctgtgaagccacagatgggctccttggaccattcctgaagctgcctactgcctcggacttcaaggggctactttaggagcaattatcttgttta

SEQ ID NO: 242 - DNA, 191 bp, synthetic construct
  source 1..191: mol_type = other DNA
gtcttggaggctggggcacctcggggaaggacgccggcatcagcaccattctggggtacggggatggattcaggaatgggttccaaggaattgtttctaatgtcctgaagccattcatggagccgtccgtatccgctgcagcctgtggggcctgcgggccggggagccgatcgcgcttcagctcagcgcct

SEQ ID NO: 243 - DNA, 185 bp, synthetic construct
  source 1..185: mol_type = other DNA
ttagcaggaaaaaagagaacatcaccttgtaaaactgaagattgtgaccagtcagaataatgtttcaggaatgggttccaaggaagtgatatgtgcatcttcttggtacacgttcctgctcacattatggtgacagctgcctcgggaagccaagttgggctttaaagtgcagggcctgctgatgt

SEQ ID NO: 244 - DNA, 212 bp, synthetic construct
  source 1..212: mol_type = other DNA
caatggtggaatgtggaggtgaagttaacaccttcgtggctacagagtttccttagcagagctgttcaggaatgggttccaaggagtgtctaaactatctccttggaacacattcctacatagctactgctaggcaatccttccctcgataaatgtcttggcatcgtttgctttgagcaagaaggttcatctgatatcagtcttctcaatct

SEQ ID NO: 245 - DNA, 207 bp, synthetic construct
  source 1..207: mol_type = other DNA
agggactgggcccacggggaggcagcgtccccgaggcagcagcggcagcggcggctcctctccccatggccctgtataatataatagaaccacaggctgggctcagacgtgtggtttatcctgttgtacagggacctggggaccccggcaccggcaggccccaaggggtgaggtgagcgggcattgggacctcccctccctgtactc

SEQ ID NO: 246 - DNA, 168 bp, synthetic construct
  source 1..168: mol_type = other DNA
tgtggtgctgggggcttcagcggccggctctgatctccatcctccctggggcatataatataatagaaccacagggcccttcatgctgcccagcttgtggttctattatgttataactcggggtgggagtcagcaggaggtgagggggcatggtggccccagtgcagc

SEQ ID NO: 247 - DNA, 186 bp, synthetic construct
  source 1..186: mol_type = other DNA
gatgctacaagtggcccacttctgagatgcgggctgcttctggatgacactgcttcccgaggcgtgctgaactggtatcgaaatgtatggattactttgctacatgcggtaccagtttagcacgtttcggcaagtgcctcctcgctggccccagggtaccacccggagcacaggtttggtgacctt

SEQ ID NO: 248 - DNA, 212 bp, synthetic construct
  source 1..212: mol_type = other DNA
caatggtggaatgtggaggtgaagttaacaccttcgtggctacagagtttccttagcagagctgcatgcggtaccagtttagcacgtgtctaaactatcgtgctagacttgtactgtcagtagctactgctaggcaatccttccctcgataaatgtcttggcatcgtttgctttgagcaagaaggttcatctgatatcagtcttctcaatct

SEQ ID NO: 249 - DNA, 171 bp, synthetic construct
  source 1..171: mol_type = other DNA
caggttaacccaacagaaggctaaagaaggtatattgctgttgacagtgagcgaccatgcggtaccagtttagcacgctgtgaagccacagatgggcgtgctagaggtgtcgcatggctgcctactgcctcggacttcaaggggctactttaggagcaattatcttgttta

SEQ ID NO: 250 - DNA, 186 bp, synthetic construct
  source 1..186: mol_type = other DNA
gatgctacaagtggcccacttctgagatgcgggctgcttctggatgacactgcttcccgaggcagcgagggagaagattaccatttatggattactttgctaaatatagtcttctccctcgctgtttcggcaagtgcctcctcgctggccccagggtaccacccggagcacaggtttggtgacctt

SEQ ID NO: 251 - DNA, 176 bp, synthetic construct
  source 1..176: mol_type = other DNA
tgaagccacaggagccaagagcaggaggaccaaggccctggcgaaggccgtggcctcgaatatagtcttctccctcgcttgtgcaggtcccaatggagcgagggataggactatactcggggacgcgggcctggacgccggcatccgggctcaggacccccctctctgccagaggc

SEQ ID NO: 252 - DNA, 171 bp, synthetic construct
  source 1..171: mol_type = other DNA
caggttaacccaacagaaggctaaagaaggtatattgctgttgacagtgagcgacaatatagtcttctccctcgctgctgtgaagccacagatgggcagtgagggaagactatgttgctgcctactgcctcggacttcaaggggctactttaggagcaattatcttgttta

SEQ ID NO: 253 - DNA, 186 bp, synthetic construct
  source 1..186: mol_type = other DNA
gatgctacaagtggcccacttctgagatgcgggctgcttctggatgacactgcttcccgaggccggttgaacattggttgcctcatatggattactttgctatgaacgaccagtgtttaaccggtttcggcaagtgcctcctcgctggccccagggtaccacccggagcacaggtttggtgacctt

SEQ ID NO: 254 - DNA, 185 bp, synthetic construct
  source 1..185: mol_type = other DNA
ccggggtcctgtctgcatccagcgcagcattctggaagacgccacgcctccgctggcgacgggacggatgaacttagaggagacgctgtgacacttcaaacttctccttggagttcatccgcgccgtccacggcaccgcatcgaaaacgccgctgagacctcagccttgacctccctcagcgtgg

SEQ ID NO: 255 - DNA, 171 bp, synthetic construct
  source 1..171: mol_type = other DNA
caggttaacccaacagaaggctaaagaaggtatattgctgttgacagtgagcgacttctccttggagttcatccgcgctgtgaagccacagatgggcgcggatgatccaaggaggagctgcctactgcctcggacttcaaggggctactttaggagcaattatcttgttta

SEQ ID NO: 256 - DNA, 207 bp, synthetic construct
  source 1..207: mol_type = other DNA
agggactgggcccacggggaggcagcgtccccgaggcagcagcggcagcggcggctcctctccccatggccctgatacacatcagaatccttactgctgggctcagaccgtaaggatctcctgtgtatcagggacctggggaccccggcaccggcaggccccaaggggtgaggcgagcgggcattgggacctcccctccctgtactc

SEQ ID NO: 257 - DNA, 210 bp, synthetic construct
  source 1..210: mol_type = other DNA
aggagggtgggggtggaggcaagcagaggacttcctgatcgcgtacccatggctacagtctttcttcatgtgactcgtggacatacacatcagaatccttacttgagaatatatgaaggaggtaggatactgatgtgtacgttcaattgtcatcactggcatcttttttgatcattgcaccatcatcaaatgcattgggataaccatgac

SEQ ID NO: 258 - DNA, 186 bp, synthetic construct
  source 1..186: mol_type = other DNA
gatgctacaagtggcccacttctgagatgcgggctgcttctggatgacactgcttcccgaggcggtaaggattctgatgtaatattatggattactttgctaatacacatcagaatccttactgtttcggcaagtgcctcctcgctggccccagggtaccacccggagcacaggtttggtgacctt

SEQ ID NO: 259 - DNA, 176 bp, synthetic construct
  source 1..176: mol_type = other DNA
tgaagccacaggagccaagagcaggaggaccaaggccctggcgaaggccgtggcctcgatacacatcagaatccttacttgtgcaggtcccaatggagtaaggatcctgatgtgtctcggggacgcgggcctggacgccggcatccgggctcaggacccccctctctgccagaggc

SEQ ID NO: 260 - DNA, 207 bp, synthetic construct
  source 1..207: mol_type = other DNA
agggactgggcccacggggaggcagcgtccccgaggcagcagcggcagcggcggctcctctccccatggccctgttgttgaaggcgtttgcacatgctgggctcagacctgtgcaatgcgatcaatagcagggacctggggaccccggcaccggcaggccccaaggggtgaggcgagcgggcattgggacctcccctccctgtactc

SEQ ID NO: 261 - DNA, 189 bp, synthetic construct
  source 1..189: mol_type = other DNA
cttctgaagaaaatatatttctttttattcatagctcttatgatagcaatgtcagcagtgcctttgttgaaggcgtttgcacatgttaagattctaaaattatcttgtgcggaagcacttcaataaaagtaaggttgaccatactctacagttgtgttttaatgtatattaatgttactaatgtgtttt

SEQ ID NO: 262 - DNA, 186 bp, synthetic construct
  source 1..186: mol_type = other DNA
gatgctacaagtggcccacttctgagatgcgggctgcttctggatgacactgcttcccgaggcgtgtgtgaacgccttcacataatatggattactttgctattgttgaaggcgtttgcacatgtttcggcaagtgcctcctcgctggccccagggtaccacccggagcacaggtttggtgacctt

SEQ ID NO: 263 - DNA, 176 bp, synthetic construct
  source 1..176: mol_type = other DNA
tgaagccacaggagccaagagcaggaggaccaaggccctggcgaaggccgtggcctcgttgttgaaggcgtttgcacattgtgcaggtcccaatgggtgtgcaaaggtcttcaatcacggggacgcgggcctggacgccggcatccgggctcaggacccccctctctgccagaggc

SEQ ID NO: 264-266 - skipped (3 entries)

SEQ ID NO: 267 - DNA, 396 bp, synthetic construct
  source 1..396: mol_type = other DNA
aggagggtgggggtggaggcaagcagaggacttcctgatcgcgtacccatggctacagtctttcttcatgtgactcgtggacttcaggaatgggttccaaggatgagaatatatgaaggatcctggaagctattcctgacgttcaattgtcatcactggcatcttttttgatcattgcaccatcatcaaatgcattgggataaccatgacgatgctacaagtggcccacttctgagatgcgggctgcttctggatgacactgcttcccgaggcctgtggttctgttatatccatatatggattactttgctatataatataatagaaccacaggtttcggcaagtgcctcctcgctggccccagggtaccacccggagcacaggtttggtgacctt

SEQ ID NO: 268 - DNA, 417 bp, synthetic construct
  source 1..417: mol_type = other DNA
aggagggtgggggtggaggcaagcagaggacttcctgatcgcgtacccatggctacagtctttcttcatgtgactcgtggacttcaggaatgggttccaaggatgagaatatatgaaggatcctggaagctattcctgacgttcaattgtcatcactggcatcttttttgatcattgcaccatcatcaaatgcattgggataaccatgacagggactgggcccacggggaggcagcgtccccgaggcagcagcggcagcggcggctcctctccccatggccctgagatccacgttactcacccgtgctgggctcagacccgggtgataatatggatctcagggacctggggaccccggcaccggcaggccccaaggggtgaggtgagcgggcattgggacctcccctccctgtactc

SEQ ID NO: 269 - DNA, 393 bp, synthetic construct
  source 1..393: mol_type = other DNA
gatgctacaagtggcccacttctgagatgcgggctgcttctggatgacactgcttcccgaggcctgtggttctgttatatccatatatggattactttgctatataatataatagaaccacaggtttcggcaagtgcctcctcgctggccccagggtaccacccggagcacaggtttggtgaccttagggactgggcccacggggaggcagcgtccccgaggcagcagcggcagcggcggctcctctccccatggccctgagatccacgttactcacccgtgctgggctcagacccgggtgataatatggatctcagggacctggggaccccggcaccggcaggccccaaggggtgaggtgagcgggcattgggacctcccctccctgtactc

SEQ ID NO: 270 - DNA, 394 bp, synthetic construct
  source 1..394: mol_type = other DNA
ttagcaggaaaaaagagaacatcaccttgtaaaactgaagattgtgaccagtcagaataatgtagatccacgttactcaccctagtgatatgtgcatctagggtgtgtcatgtggatgaagcattatggtgacagctgcctcgggaagccaagttgggctttaaagtgcagggcctgctgatgtaggagggtgggggtggaggcaagcagaggacttcctgatcgcgtacccatggctacagtctttcttcatgtgactcgtggacttcaggaatgggttccaaggatgagaatatatgaaggatcctggaagctattcctgacgttcaattgtcatcactggcatcttttttgatcattgcaccatcatcaaatgcattgggataaccatgac

SEQ ID NO: 271 - DNA, 370 bp, synthetic construct
  source 1..370: mol_type = other DNA
ttagcaggaaaaaagagaacatcaccttgtaaaactgaagattgtgaccagtcagaataatgtagatccacgttactcaccctagtgatatgtgcatctagggtgtgtcatgtggatgaagcattatggtgacagctgcctcgggaagccaagttgggctttaaagtgcagggcctgctgatgtgatgctacaagtggcccacttctgagatgcgggctgcttctggatgacactgcttcccgaggcctgtggttctgttatatccatatatggattactttgctatataatataatagaaccacaggtttcggcaagtgcctcctcgctggccccagggtaccacccggagcacaggtttggtgacctt

SEQ ID NO: 272 - DNA, 396 bp, synthetic construct
  source 1..396: mol_type = other DNA
aggagggtgggggtggaggcaagcagaggacttcctgatcgcgtacccatggctacagtctttcttcatgtgactcgtggacagatccacgttactcaccccctgagaatatatgaaggaggggtgagaagcgtggatccgttcaattgtcatcactggcatcttttttgatcattgcaccatcatcaaatgcattgggataaccatgacgatgctacaagtggcccacttctgagatgcgggctgcttctggatgacactgcttcccgaggcctgtggttctgttatatccatatatggattactttgctatataatataatagaaccacaggtttcggcaagtgcctcctcgctggccccagggtaccacccggagcacaggtttggtgacctt

SEQ ID NO: 273 - DNA, 417 bp, synthetic construct
  source 1..417: mol_type = other DNA
agggactgggcccacggggaggcagcgtccccgaggcagcagcggcagcggcggctcctctccccatggccctgagatccacgttactcacccgtgctgggctcagacccgggtgataatatggatctcagggacctggggaccccggcaccggcaggccccaaggggtgaggtgagcgggcattgggacctcccctccctgtactcaggagggtgggggtggaggcaagcagaggacttcctgatcgcgtacccatggctacagtctttcttcatgtgactcgtggacttcaggaatgggttccaaggatgagaatatatgaaggatcctggaagctattcctgacgttcaattgtcatcactggcatcttttttgatcattgcaccatcatcaaatgcattgggataaccatgac

SEQ ID NO: 274 - DNA, 393 bp, synthetic construct
  source 1..393: mol_type = other DNA
agggactgggcccacggggaggcagcgtccccgaggcagcagcggcagcggcggctcctctccccatggccctgagatccacgttactcacccgtgctgggctcagacccgggtgataatatggatctcagggacctggggaccccggcaccggcaggccccaaggggtgaggtgagcgggcattgggacctcccctccctgtactcgatgctacaagtggcccacttctgagatgcgggctgcttctggatgacactgcttcccgaggcctgtggttctgttatatccatatatggattactttgctatataatataatagaaccacaggtttcggcaagtgcctcctcgctggccccagggtaccacccggagcacaggtttggtgacctt

SEQ ID NO: 275 - DNA, 580 bp, synthetic construct
  source 1..580: mol_type = other DNA
ttagcaggaaaaaagagaacatcaccttgtaaaactgaagattgtgaccagtcagaataatgtagatccacgttactcaccctagtgatatgtgcatctagggtgtgtcatgtggatgaagcattatggtgacagctgcctcgggaagccaagttgggctttaaagtgcagggcctgctgatgtaggagggtgggggtggaggcaagcagaggacttcctgatcgcgtacccatggctacagtctttcttcatgtgactcgtggacttcaggaatgggttccaaggatgagaatatatgaaggatcctggaagctattcctgacgttcaattgtcatcactggcatcttttttgatcattgcaccatcatcaaatgcattgggataaccatgacgatgctacaagtggcccacttctgagatgcgggctgcttctggatgacactgcttcccgaggcctgtggttctgttatatccatatatggattactttgctatataatataatagaaccacaggtttcggcaagtgcctcctcgctggccccagggtaccacccggagcacaggtttggtgacctt

SEQ ID NO: 276 - DNA, 748 bp, synthetic construct
  source 1..748: mol_type = other DNA
ttaggatgagttgagatcccagtgatcttctcgctaagagtttcctgcctgggcaaggaggaaattagcaggaaaaaagagaacatcaccttgtaaaactgaagattgtgaccagtcagaataatgtagatccacgttactcaccctagtgatatgtgcatctagggtgtgtcatgtggatgaagcattatggtgacagctgcctcgggaagccaagttgggctttaaagtgcagggcctgctgatgtaggagggtgggggtggaggcaagcagaggacttcctgatcgcgtacccatggctacagtctttcttcatgtgactcgtggacttcaggaatgggttccaaggatgagaatatatgaaggatcctggaagctattcctgacgttcaattgtcatcactggcatcttttttgatcattgcaccatcatcaaatgcattgggataaccatgacgatgctacaagtggcccacttctgagatgcgggctgcttctggatgacactgcttcccgaggcctgtggttctgttatatccatatatggattactttgctatataatataatagaaccacaggtttcggcaagtgcctcctcgctggccccagggtaccacccggagcacaggtttggtgaccttcttcctcatcagggctttgtgccagcaaatgactccctcaccaaggaagcaagagcctctgaatcccatctgggctcttcctgaacacccctatctccccctct

SEQ ID NO: 277 - DNA, 761 bp, synthetic construct
  source 1..761: mol_type = other DNA
ttagggattatgctgaatttgtatggtttatagttgttagagtttgaggtgttaattctaattatctatttcaaattttagcaggaaaaaagagaacatcaccttgtaaaactgaagattgtgaccagtcagaataatgtagatccacgttactcaccctagtgatatgtgcatctagggtgtgtcatgtggatgaagcattatggtgacagctgcctcgggaagccaagttgggctttaaagtgcagggcctgctgatgtaggagggtgggggtggaggcaagcagaggacttcctgatcgcgtacccatggctacagtctttcttcatgtgactcgtggacttcaggaatgggttccaaggatgagaatatatgaaggatcctggaagctattcctgacgttcaattgtcatcactggcatcttttttgatcattgcaccatcatcaaatgcattgggataaccatgacgatgctacaagtggcccacttctgagatgcgggctgcttctggatgacactgcttcccgaggcctgtggttctgttatatccatatatggattactttgctatataatataatagaaccacaggtttcggcaagtgcctcctcgctggccccagggtaccacccggagcacaggtttggtgaccttcttcctcatcagggctttgtgccagcaaatgactccctcaccaaggaagcaagagcctctgaatcccatctgggctcttcctgaacacccctatctccccctct

SEQ ID NO: 278 - DNA, 580 bp, synthetic construct
  source 1..580: mol_type = other DNA
aggagggtgggggtggaggcaagcagaggacttcctgatcgcgtacccatggctacagtctttcttcatgtgactcgtggacttcaggaatgggttccaaggatgagaatatatgaaggatcctggaagctattcctgacgttcaattgtcatcactggcatcttttttgatcattgcaccatcatcaaatgcattgggataaccatgacgatgctacaagtggcccacttctgagatgcgggctgcttctggatgacactgcttcccgaggcctgtggttctgttatatccatatatggattactttgctatataatataatagaaccacaggtttcggcaagtgcctcctcgctggccccagggtaccacccggagcacaggtttggtgaccttttagcaggaaaaaagagaacatcaccttgtaaaactgaagattgtgaccagtcagaataatgtagatccacgttactcaccctagtgatatgtgcatctagggtgtgtcatgtggatgaagcattatggtgacagctgcctcgggaagccaagttgggctttaaagtgcagggcctgctgatgt

SEQ ID NO: 279 - DNA, 748 bp, synthetic construct
  source 1..748: mol_type = other DNA
ttaggatgagttgagatcccagtgatcttctcgctaagagtttcctgcctgggcaaggaggaaaaggagggtgggggtggaggcaagcagaggacttcctgatcgcgtacccatggctacagtctttcttcatgtgactcgtggacttcaggaatgggttccaaggatgagaatatatgaaggatcctggaagctattcctgacgttcaattgtcatcactggcatcttttttgatcattgcaccatcatcaaatgcattgggataaccatgacgatgctacaagtggcccacttctgagatgcgggctgcttctggatgacactgcttcccgaggcctgtggttctgttatatccatatatggattactttgctatataatataatagaaccacaggtttcggcaagtgcctcctcgctggccccagggtaccacccggagcacaggtttggtgaccttttagcaggaaaaaagagaacatcaccttgtaaaactgaagattgtgaccagtcagaataatgtagatccacgttactcaccctagtgatatgtgcatctagggtgtgtcatgtggatgaagcattatggtgacagctgcctcgggaagccaagttgggctttaaagtgcagggcctgctgatgtcttcctcatcagggctttgtgccagcaaatgactccctcaccaaggaagcaagagcctctgaatcccatctgggctcttcctgaacacccctatctccccctct

SEQ ID NO: 280 - DNA, 756 bp, synthetic construct
  source 1..756: mol_type = other DNA
tggagaggagggtgggggtggaggcaagcagaggacctcctgatcatgtacccataggacagggtgatggaaaggagggtgggggtggaggcaagcagaggacttcctgatcgcgtacccatggctacagtctttcttcatgtgactcgtggacttcaggaatgggttccaaggatgagaatatatgaaggatcctggaagctattcctgacgttcaattgtcatcactggcatcttttttgatcattgcaccatcatcaaatgcattgggataaccatgacgatgctacaagtggcccacttctgagatgcgggctgcttctggatgacactgcttcccgaggcctgtggttctgttatatccatatatggattactttgctatataatataatagaaccacaggtttcggcaagtgcctcctcgctggccccagggtaccacccggagcacaggtttggtgaccttttagcaggaaaaaagagaacatcaccttgtaaaactgaagattgtgaccagtcagaataatgtagatccacgttactcaccctagtgatatgtgcatctagggtgtgtcatgtggatgaagcattatggtgacagctgcctcgggaagccaagttgggctttaaagtgcagggcctgctgatgtcttcctcatcagggctttgtgccagcaaatgactccctcaccaaggaagcaagagcctctgaatcccatctgggctcttcctgaacacccctatctccccctct

SEQ ID NO: 281 - DNA, 386 bp, synthetic construct
  source 1..386: mol_type = other DNA
aggagggtgggggtggaggcaagcagaggacttcctgatcgcgtacccatggctacagtctttcttcatgtgactcgtggacttcaggaatgggttccaaggatgagaatatatgaaggatcctggaagctattcctgacgttcaattgtcatcactggcatcttttttgatcattgcaccatcatcaaatgcattgggataaccatgactgaagccacaggagccaagagcaggaggaccaaggccctggcgaaggccgtggcctcgaatatagtcttctccctcgcttgtgcaggtcccaatggagcgagggataggactatactcggggacgcgggcctggacgccggcatccgggctcaggacccccctctctgccagaggc

SEQ ID NO: 282 - DNA, 396 bp, synthetic construct; identical to SEQ ID NO: 267
  source 1..396: mol_type = other DNA

SEQ ID NO: 283 - DNA, 362 bp, synthetic construct
  source 1..362: mol_type = other DNA
gatgctacaagtggcccacttctgagatgcgggctgcttctggatgacactgcttcccgaggcctgtggttctgttatatccatatatggattactttgctatataatataatagaaccacaggtttcggcaagtgcctcctcgctggccccagggtaccacccggagcacaggtttggtgacctttgaagccacaggagccaagagcaggaggaccaaggccctggcgaaggccgtggcctcgaatatagtcttctccctcgcttgtgcaggtcccaatggagcgagggataggactatactcggggacgcgggcctggacgccggcatccgggctcaggacccccctctctgccagaggc

SEQ ID NO: 284 - DNA, 396 bp, synthetic construct
  source 1..396: mol_type = other DNA
gatgctacaagtggcccacttctgagatgcgggctgcttctggatgacactgcttcccgaggcctgtggttctgttatatccatatatggattactttgctatataatataatagaaccacaggtttcggcaagtgcctcctcgctggccccagggtaccacccggagcacaggtttggtgaccttaggagggtgggggtggaggcaagcagaggacttcctgatcgcgtacccatggctacagtctttcttcatgtgactcgtggacaatatagtcttctccctcgcttgagaatatatgaaggaagcagggacaggactatattgttcaattgtcatcactggcatcttttttgatcattgcaccatcatcaaatgcattgggataaccatgac

SEQ ID NO: 285 - DNA, 360 bp, synthetic construct
  source 1..360: mol_type = other DNA
ttagcaggaaaaaagagaacatcaccttgtaaaactgaagattgtgaccagtcagaataatgtagatccacgttactcaccctagtgatatgtgcatctagggtgtgtcatgtggatgaagcattatggtgacagctgcctcgggaagccaagttgggctttaaagtgcagggcctgctgatgttgaagccacaggagccaagagcaggaggaccaaggccctggcgaaggccgtggcctcgaatatagtcttctccctcgcttgtgcaggtcccaatggagcgagggataggactatactcggggacgcgggcctggacgccggcatccgggctcaggacccccctctctgccagaggc

SEQ ID NO: 286 - DNA, 394 bp, synthetic construct
  source 1..394: mol_type = other DNA
ttagcaggaaaaaagagaacatcaccttgtaaaactgaagattgtgaccagtcagaataatgtagatccacgttactcaccctagtgatatgtgcatctagggtgtgtcatgtggatgaagcattatggtgacagctgcctcgggaagccaagttgggctttaaagtgcagggcctgctgatgtaggagggtgggggtggaggcaagcagaggacttcctgatcgcgtacccatggctacagtctttcttcatgtgactcgtggacaatatagtcttctccctcgcttgagaatatatgaaggaagcagggacaggactatattgttcaattgtcatcactggcatcttttttgatcattgcaccatcatcaaatgcattgggataaccatgac

SEQ ID NO: 287 - DNA, 394 bp, synthetic construct; identical to SEQ ID NO: 270
  source 1..394: mol_type = other DNA

SEQ ID NO: 288 - DNA, 370 bp, synthetic construct; identical to SEQ ID NO: 271
  source 1..370: mol_type = other DNA

SEQ ID NO: 289 - DNA, 386 bp, synthetic construct
  source 1..386: mol_type = other DNA
aggagggtgggggtggaggcaagcagaggacttcctgatcgcgtacccatggctacagtctttcttcatgtgactcgtggacagatccacgttactcaccccctgagaatatatgaaggaggggtgagaagcgtggatccgttcaattgtcatcactggcatcttttttgatcattgcaccatcatcaaatgcattgggataaccatgactgaagccacaggagccaagagcaggaggaccaaggccctggcgaaggccgtggcctcgaatatagtcttctccctcgcttgtgcaggtcccaatggagcgagggataggactatactcggggacgcgggcctggacgccggcatccgggctcaggacccccctctctgccagaggc

SEQ ID NO: 290 - DNA, 396 bp, synthetic construct; identical to SEQ ID NO: 272
  source 1..396: mol_type = other DNA

SEQ ID NO: 291 - DNA, 16 bp, unidentified
  source 1..16: mol_type = unassigned DNA
aggtaagagtcgatcg

SEQ ID NO: 292 - DNA, 48 bp, unidentified
  source 1..48: mol_type = unassigned DNA
acgcgttactaactggtacctcttttttttttttgatatcctgcaggc

SEQ ID NO: 293 - DNA, 22 bp, unidentified
  source 1..22: mol_type = unassigned DNA
tgtgcgaagcgctggatgcaca

SEQ ID NO: 294 - DNA, 23 bp, unidentified
  source 1..23: mol_type = unassigned DNA
tgtgcgtccagcgcttcgagaca

SEQ ID NO: 295 - DNA, 22 bp, unidentified
  source 1..22: mol_type = unassigned DNA
tgtgcgaagcgctggatgcaca

SEQ ID NO: 296 - DNA, 21 bp, unidentified
  source 1..21: mol_type = unassigned DNA
tgtggtccaccgcttcgcacc

SEQ ID NO: 297 - DNA, 22 bp, unidentified
  source 1..22: mol_type = unassigned DNA
tgtgcgaagcgctggatgcaca

SEQ ID NO: 298 - DNA, 22 bp, unidentified
  source 1..22: mol_type = unassigned DNA
tgtgcatccagccttcgacaca

SEQ ID NO: 299 - DNA, 22 bp, unidentified
  source 1..22: mol_type = unassigned DNA
tgtgcgaagcgctggatgcaca

SEQ ID NO: 300 - DNA, 22 bp, unidentified
  source 1..22: mol_type = unassigned DNA
tgtgcgaagcgctggatgcaca

SEQ ID NO: 301 - DNA, 22 bp, unidentified
  source 1..22: mol_type = unassigned DNA
tgtgcgaagcgctggatgcaca

SEQ ID NO: 302 - DNA, 22 bp, unidentified
  source 1..22: mol_type = unassigned DNA
tgtgcgaagcgctggatgcaca

SEQ ID NO: 303 - DNA, 22 bp, unidentified
  source 1..22: mol_type = unassigned DNA
tcctgtgtgattcagctgcagc

SEQ ID NO: 304 - DNA, 23 bp, unidentified
  source 1..23: mol_type = unassigned DNA
gctgcagctgaatcacactcgga

SEQ ID NO: 305 - DNA, 22 bp, unidentified
  source 1..22: mol_type = unassigned DNA
tcctgtgtgattcagctgcagc

SEQ ID NO: 306 - DNA, 22 bp, unidentified
  source 1..22: mol_type = unassigned DNA
tcctgtgtgattcagctgcagc

SEQ ID NO: 307 - DNA, 23 bp, unidentified
  source 1..23: mol_type = unassigned DNA
tcctgtgtgattcagctgcagct

SEQ ID NO: 308 - DNA, 20 bp, unidentified
  source 1..20: mol_type = unassigned DNA
agctgcagctgaatcacagg

SEQ ID NO: 309 - DNA, 22 bp, unidentified
  source 1..22: mol_type = unassigned DNA
tcctgtgtgattcagctgcagc

SEQ ID NO: 310 - DNA, 21 bp, unidentified
  source 1..21: mol_type = unassigned DNA
gctgcatatgaatccaccagg

SEQ ID NO: 311 - DNA, 22 bp, unidentified
  source 1..22: mol_type = unassigned DNA
tcctgtgtgattcagctgcagc

SEQ ID NO: 312 - DNA, 22 bp, unidentified
  source 1..22: mol_type = unassigned DNA
tcctgtgtgattcagctgcagc

SEQ ID NO: 313 - DNA, 22 bp, unidentified
  source 1..22: mol_type = unassigned DNA
atgtgtaccatgtagatgccat

SEQ ID NO: 314 - DNA, 23 bp, unidentified
  source 1..23: mol_type = unassigned DNA
atggcatctacatggtacttcat

SEQ ID NO: 315 - DNA, 22 bp, unidentified
  source 1..22: mol_type = unassigned DNA
atgtgtaccatgtagatgccat

SEQ ID NO: 316 - DNA, 21 bp, unidentified
  source 1..21: mol_type = unassigned DNA
atggatctagatggtacacac

SEQ ID NO: 317 - DNA, 22 bp, unidentified
  source 1..22: mol_type = unassigned DNA
atgtgtaccatgtagatgccat

SEQ ID NO: 318 - DNA, 22 bp, unidentified
  source 1..22: mol_type = unassigned DNA
atggcatctacaggtacaacat

SEQ ID NO: 319 - DNA, 21 bp, unidentified
  source 1..21: mol_type = unassigned DNA
atgtgtaccatgtagatgcca

SEQ ID NO: 320 - DNA, 21 bp, unidentified
  source 1..21: mol_type = unassigned DNA
atgtgtaccatgtagatgcca

SEQ ID NO: 321 - DNA, 22 bp, unidentified
  source 1..22: mol_type = unassigned DNA
atgtgtaccatgtagatgccat

SEQ ID NO: 322 - DNA, 22 bp, unidentified
  source 1..22: mol_type = unassigned DNA
atgtgtaccatgtagatgccat

SEQ ID NO: 323 - DNA, 186 bp, synthetic construct
  source 1..186: mol_type = other DNA
gatgctacaagtggcccacttctgagatgcgggctgcttctggatgacactgcttcccgaggtgtgcgtccagcgcttcgagacatatggattactttgctatgtgcgaagcgctggatgcacatttcggcaagtgcctcctcgctggccccagggtaccacccggagcacaggtttggtgacctt

SEQ ID NO: 324 - DNA, 210 bp, synthetic construct
  source 1..210: mol_type = other DNA
aggagggtgggggtggaggcaagcagaggacttcctgatcgcgtacccatggctacagtctttcttcatgtgactcgtggactgtgcgaagcgctggatgcacagagaatatatgaaggtgtggtccaccgcttcgcaccgttcaattgtcatcactggcatcttttttgatcattgcaccatcatcaaatgcattgggataaccatgac

SEQ ID NO: 325 - DNA, 185 bp, synthetic construct
  source 1..185: mol_type = other DNA
cattttccctccctttcccttaggagcctgttcctctcacgccctcacctggctgagccgcatgtgcatccagccttcgacacatatgtcctgacccagctatgtgcgaagcgctggatgcacatgccctctgcccctggcttcgaggaggaggaggagctgctttccccatcatctggaaggtg

SEQ ID NO: 326 - DNA, 189 bp, synthetic construct
  source 1..189: mol_type = other DNA
cttctgaagaaaatatatttctttttattcatagctcttatgatagcaatgtcagcagtgccttgtgcgaagcgctggatgcacattaagattctaaaattatcttgcatccaccgacttcgcacgaagtaaggttgaccatactctacagttgtgttttaatgtatattaatgttactaatgtgtttt

SEQ ID NO: 327 - DNA, 172 bp, synthetic construct
  source 1..172: mol_type = other DNA
tgtctgcttgttttgcctaccatcgtgacatctccatggctgtaccaccttgtcgggtgtgcgaagcgctggatgcacactgttgaatctcatgggtgcacccagccttcgcacgtctgacattttggtatctttcatctgaccatccatatccaatgttctcatttaaaca

SEQ ID NO: 328 - DNA, 186 bp, synthetic construct
  source 1..186: mol_type = other DNA
gatgctacaagtggcccacttctgagatgcgggctgcttctggatgacactgcttcccgagggctgcagctgaatcacactcggatatggattactttgctatcctgtgtgattcagctgcagctttcggcaagtgcctcctcgctggccccagggtaccacccggagcacaggtttggtgacctt

SEQ ID NO: 329 - DNA, 210 bp, synthetic construct
  source 1..210: mol_type = other DNA
aggagggtgggggtggaggcaagcagaggacttcctgatcgcgtacccatggctacagtctttcttcatgtgactcgtggactcctgtgtgattcagctgcagcgagaatatatgaagggctgagctgcatcacacaggcgttcaattgtcatcactggcatcttttttgatcattgcaccatcatcaaatgcattgggataaccatgac

SEQ ID NO: 330 - DNA, 182 bp, synthetic construct
  source 1..182: mol_type = other DNA
ccaataattcaagccaagcaagtatataggtgttttaatagtttttgtttgcagtcctctgagctgcagctgaatcacaggtacaagaagaatgtagttcctgtgtgattcagctgcagcttggtggcctgctatttccttcaaatgaatgatttttactaattttgtgtacttttattgtg

SEQ ID NO: 331 - DNA, 181 bp, synthetic construct
  source 1..181: mol_type = other DNA
cagttctgttttgattttttttgtttgttttttgatcagtgctaatcttcgatactcgaaggagctgcatatgaatccaccaggtctttatttatgatcctgtgtgattcagctgcagcttttttagtatcaaatcccaccctggaggcacttcctgttcctgatgcagccttcagggagg

SEQ ID NO: 332 - DNA, 207 bp, synthetic construct
  source 1..207: mol_type = other DNA
agggactgggcccacggggaggcagcgtccccgaggcagcagcggcagcggcggctcctctccccatggccctgtcctgtgtgattcagctgcagcctgggctcagacatgcagctaatgtcacaggacagggacctggggaccccggcaccggcaggccccaaggggtgaggtgagcgggcattgggacctcccctccctgtactc

SEQ ID NO: 333 - DNA, 186 bp, synthetic construct
  source 1..186: mol_type = other DNA
gatgctacaagtggcccacttctgagatgcgggctgcttctggatgacactgcttcccgaggatggcatctacatggtacttcattatggattactttgctaatgtgtaccatgtagatgccattttcggcaagtgcctcctcgctggccccagggtaccacccggagcacaggtttggtgacctt

SEQ ID NO: 334 - DNA, 210 bp, synthetic construct
  source 1..210: mol_type = other DNA
aggagggtgggggtggaggcaagcagaggacttcctgatcgcgtacccatggctacagtctttcttcatgtgactcgtggacatgtgtaccatgtagatgccatgagaatatatgaaggatggatctagatggtacacacgttcaattgtcatcactggcatcttttttgatcattgcaccatcatcaaatgcattgggataaccatgac

SEQ ID NO: 335 - DNA, 185 bp, synthetic construct
  source 1..185: mol_type = other DNA
cattttccctccctttcccttaggagcctgttcctctcacgccctcacctggctgagccgcaatggcatctacaggtacaacattatgtcctgacccagctaatgtgtaccatgtagatgccattgccctctgcccctggcttcgaggaggaggaggagctgctttccccatcatctggaaggtg

SEQ ID NO: 336 - DNA, 187 bp, synthetic construct
  source 1..187: mol_type = other DNA
ggagtcaggaggcctgggcagcctgaagagtacacgccgacggacagacagacagtgcagtcaccatgtgtaccatgtagatgccaaacagcactggagggaggcatctacctggtacacatgatgagtgtactgtgggcttcggagatcacgccactgctgccgcccgctgcccgccaccatcttc

SEQ ID NO: 337 - DNA, 177 bp, synthetic construct
  source 1..177: mol_type = other DNA
atgaagccacaggagccaagagcaggaggaccaaggccctggcgaaggccgtggcctcgatgtgtaccatgtagatgccatgtgcaggtcccaatggtggcatctagatggtacacctcggggacgcgggcctggacgccggcatccgggctcaggacccccctctctgccagaggc

SEQ ID NO: 338 - DNA, 62 bp, unidentified
  source 1..62: mol_type = unassigned DNA
cattttccctccctttcccttaggagcctgttcctctcacgccctcacctggctgagccgca

SEQ ID NO: 339 - DNA, 18 bp, unidentified
  source 1..18: mol_type = unassigned DNA
tatgtcctgacccagcta

SEQ ID NO: 340 - DNA, 61 bp, unidentified
  source 1..61: mol_type = unassigned DNA
tgccctctgcccctggcttcgaggaggaggaggagctgctttccccatcatctggaaggtg

SEQ ID NO: 341 - DNA, 59 bp, unidentified
  source 1..59: mol_type = unassigned DNA
atgaagccacaggagccaagagcaggaggaccaaggccctggcgaaggccgtggcctcg

SEQ ID NO: 342 - DNA, 15 bp, unidentified
  source 1..15: mol_type = unassigned DNA
gtgcaggtcccaatg

SEQ ID NO: 343 - DNA, 59 bp, unidentified; identical to SEQ ID NO: 45
  source 1..59: mol_type = unassigned DNA

SEQ ID NO: 344 - DNA, 53 bp, unidentified
  source 1..53: mol_type = unassigned DNA
tgtggtgctgggggcttcagcggccggctctgatctccatcctccctggggca

SEQ ID NO: 345 - DNA, 13 bp, unidentified
  source 1..13: mol_type = unassigned DNA
cccttcatgctgc

SEQ ID NO: 346 - DNA, 53 bp, unidentified
  source 1..53: mol_type = unassigned DNA
actcggggtgggagtcagcaggaggtgagggggcatggtggccccagtgcagc

SEQ ID NO: 347 - DNA, 83 bp, synthetic construct
  source 1..83: mol_type = other DNA
ggctacagtctttcttcatgtgactcgtggacaatatagtcttctccctcgcttgagaatatatgaaggaagcagggacagga

SEQ ID NO: 348 - DNA, 83 bp, synthetic construct
  source 1..83: mol_type = other DNA
ggctacagtctttcttcatgtgactcgtggacttcaggaatgggttccaaggatgagaatatatgaaggatcctggaagctat

SEQ ID NO: 349 - DNA, 84 bp, synthetic construct
  source 1..84: mol_type = other DNA
tgcttcccgaggcctgtggttctgttatatccatatatggattactttgctatataatataatagaaccacaggtttcggcaag

SEQ ID NO: 350 - DNA, 83 bp, synthetic construct
  source 1..83: mol_type = other DNA
gtcagaataatgtagatccacgttactcaccctagtgatatgtgcatctagggtgtgtcatgtggatgaagcattatggtgac

SEQ ID NO: 351 - DNA, 84 bp, synthetic construct
  source 1..84: mol_type = other DNA
ctccccatggccctgagatccacgttactcacccgtgctgggctcagacccgggtgataatatggatctcagggacctggggac

SEQ ID NO: 352 - DNA, 83 bp, synthetic construct
  source 1..83: mol_type = other DNA
ggctacagtctttcttcatgtgactcgtggacagatccacgttactcaccccctgagaatatatgaaggaggggtgagaagcg

SEQ ID NO: 353 - DNA, 83 bp, synthetic construct
  source 1..83: mol_type = other DNA
tgcttcccgaggtctgcgtagcagtcatcgccggttatggattactttgctaaccacgatgactgctgtgcagatttcggcaa

SEQ ID NO: 354 - DNA, 83 bp, synthetic construct
  source 1..83: mol_type = other DNA
ggctacagtctttcttcatgtgactcgtggacaccacgatgactgctgtgcagagagaatatatgaaggtctgatggccgtca

SEQ ID NO: 355 - DNA, 83 bp, synthetic construct
  source 1..83: mol_type = other DNA
ggctgagccgcatctgtacagcagcatcgatggttatgtcctgacccagctaaccacgatgactgctgtgcagatgccctctg

SEQ ID NO: 356 - DNA, 83 bp, synthetic construct
  source 1..83: mol_type = other DNA
gtcagcagtgcctaccacgatgactgctgtgcagattaagattctaaaattatcttgcgcagctgttcatcgtggtagtaagg

SEQ ID NO: 357 - DNA, 83 bp, synthetic construct
  source 1..83: mol_type = other DNA
gtcagcagtgcctaccacgatgactgctgtgcagattaagattctaaaattatcttgcgcagctgttcatcgtggtaagtaag

SEQ ID NO: 358 - DNA, 72 bp, synthetic construct
  source 1..72: mol_type = other DNA
tgtcgggaccacgatgactgctgtgcagactgttgaatctcatggctgcgaagcagcatcgtggctctgaca

SEQ ID NO: 359 - DNA, 81 bp, synthetic construct
  source 1..81: mol_type = other DNA
gatactcgaaggatctgcaacgcagtctcgctggtctttatttatgaaccacgatgactgctgtgcagattttttagtatc

SEQ ID NO: 360 - DNA, 82 bp, synthetic construct
  source 1..82: mol_type = other DNA
gacagtgcagtcaccaccacgatgactgctgtgcagaacagcactggagggatgcacagcaatcatcgtggtgatgagtgta

SEQ ID NO: 361 - DNA, 82 bp, synthetic construct
  source 1..82: mol_type = other DNA
gcagtcctctgatctgcacagcagttcgtggtacaagaagaatgtagtaccacgatgactgctgtgcagattggtggcctgc

SEQ ID NO: 362 - DNA, 80 bp, synthetic construct
  source 1..80: mol_type = other DNA
tgagaggccgcaccacgatgactgctgtgcagagtgcacccgtgtctgccaatagctgtaatcgtgggcggccctagcga

SEQ ID NO: 363 - DNA, 83 bp, synthetic construct
  source 1..83: mol_type = other DNA
tgcttcccgaggcagtcgttgactgtggacttatatatggattactttgctatatcgttcacggtcagcgactgtttcggcaa

SEQ ID NO: 364 - DNA, 81 bp, synthetic construct
  source 1..81: mol_type = other DNA
ggctacagtctttcttcatgtgactcgtggactatcgttcacggtcagcgactggagaatatatgaaggcagtgctgaacg

SEQ ID NO: 365 - DNA, 83 bp, synthetic construct
  source 1..83: mol_type = other DNA
ggctgagccgcatagtcgctgacttgaacagatatatgtcctgacccagctatatcgttcacggtcagcgactgtgccctctg

SEQ ID NO: 366 - DNA, 82 bp, synthetic construct
  source 1..82: mol_type = other DNA
gacagtgcagtcacctatcgttcacggtcagcgactaacagcactggagggtgtcgctgacagtgaacgatagatgagtgta

SEQ ID NO: 367 - DNA, 83 bp, synthetic construct
  source 1..83: mol_type = other DNA
gtcagcagtgccttatcgttcacggtcagcgactgttaagattctaaaattatctgtcgctgagcgctgaacgatgaagtaag

SEQ ID NO: 368 - DNA, 84 bp, synthetic construct
  source 1..84: mol_type = other DNA
tgcttcccgaggactacttcaatgtcctgaccttatatggattactttgctataactcaggacattgaagtagttttcggcaag

SEQ ID NO: 369 - DNA, 83 bp, synthetic construct
  source 1..83: mol_type = other DNA
ggctacagtctttcttcatgtgactcgtggactaactcaggacattgaagtagtgagaatatatgaaggactattcagagtcc

SEQ ID NO: 370 - DNA, 72 bp, synthetic construct
  source 1..72: mol_type = other DNA
tgtcgggtaactcaggacattgaagtagtctgttgaatctcatggctactccaatgcctgagttgtctgaca

SEQ ID NO: 371 - DNA, 81 bp, synthetic construct
  source 1..81: mol_type = other DNA
gatactcgaaggaactactgtaatgtctgacgtttctttatttatgataactcaggacattgaagtagtttttttagtatc

SEQ ID NO: 372 - DNA, 82 bp, synthetic construct
  source 1..82: mol_type = other DNA
gcagtcctctggactacttcaatgttgagtttacaagaagaatgtagttaactcaggacattgaagtagtctggtggcctgc

SEQ ID NO: 373 - DNA, 80 bp, synthetic construct
  source 1..80: mol_type = other DNA
tgagaggccgcagatccacgttactcaccctaggtgcacccgtgctaggcagtgagcaaagtggatcgcggccctagcga

SEQ ID NO: 374 - DNA, 110 bp, synthetic construct
  source 1..110: mol_type = other DNA
ggctacagtctttcttcatgtgactcgtggacagatccacgttactcaccctaggagaatatatgaaggctaggtgagaaacgtggatcggttcaattgtcatcactggc

SEQ ID NO: 375 - DNA, 86 bp, synthetic construct
  source 1..86: mol_type = other DNA
tgcttcccgaggctagggtgagtaacgtggcctcttatggattactttgctaagatccacgttactcaccctagtttcggcaagtg

SEQ ID NO: 376 - DNA, 72 bp, synthetic construct
  source 1..72: mol_type = other DNA
tgtcgggagatccacgttactcaccctagctgttgaatctcatggtagggcgagtacgtggatcgtctgaca

SEQ ID NO: 377 - DNA, 85 bp, synthetic construct
  source 1..85: mol_type = other DNA
ggctgagccgcactagggtgagtacgtggcatcttatgtcctgacccagctaagatccacgttactcaccctagtgccctctgcc

SEQ ID NO: 378 - DNA, 110 bp, synthetic construct
  source 1..110: mol_type = other DNA
ggctacagtctttcttcatgtgactcgtggaccattatgcctgggatttggatcgagaatatatgaagggatcagatcgcaggcataattgttcaattgtcatcactggc

SEQ ID NO: 379 - DNA, 84 bp, synthetic construct
  source 1..84: mol_type = other DNA
ctccccatggccctgcattatgcctgggatttggatcctgggctcagacctccaaatccacacatagtgcagggacctggggac

SEQ ID NO: 380 - DNA, 71 bp, synthetic construct
  source 1..71: mol_type = other DNA
gcgaccattatgcctgggatttggatcctgtgaagccacagatggggatccagatcaggcatagtggctgc

SEQ ID NO: 381 - DNA, 86 bp, synthetic construct
  source 1..86: mol_type = other DNA
tgcttcccgagggatccggatcctaggtatccatgtatggattactttgctacattatgcctgggatttggatctttcggcaagtg

SEQ ID NO: 382 - DNA, 89 bp, synthetic construct
  source 1..89: mol_type = other DNA
gtcagcagtgcctcattatgcctgggatttggatcttaagattctaaaattatcttccgaatcacatggcataatgaagtaaggttgac

SEQ ID NO: 383 - DNA, 84 bp, synthetic construct
  source 1..84: mol_type = other DNA
gtcagaataatgtcattatgcctgggatttggatcgtgatatgtgcatcggccagaaccaaggcataagtagcattatggtgac

SEQ ID NO: 384 - DNA, 85 bp, synthetic construct
  source 1..85: mol_type = other DNA
cgctggcgacggactccaaatcccgtgcataatcgctgtgacacttcaaaccattatgcctgggatttggatcccgtccacggca

SEQ ID NO: 385 - DNA, 112 bp, synthetic construct
  source 1..112: mol_type = other DNA
ccttagcagagctgcattatgcctgggatttggatctgtctaaactatgattcgaatccaaggcatatggtagctactgctaggcaatccttccctcgataaatgtcttggc

SEQ ID NO: 386 - DNA, 110 bp, synthetic construct
  source 1..110: mol_type = other DNA
ggcctggctggacagagttgtcatgtgatgatgaaaggacagtgaattagcagaacatccgctcacctgttaattcacatccctttcatcagcacatgacaacccagcct

SEQ ID NO: 387 - DNA, 81 bp, synthetic construct
  source 1..81: mol_type = other DNA
cttcaggacgatggaagggttgtgaacgttagatttaaatagtgattgtctaattcacatccctttcatcagtcttggggg

SEQ ID NO: 388 - DNA, 110 bp, synthetic construct
  source 1..110: mol_type = other DNA
ggctacagtctttcttcatgtgactcgtggactaattcacatccctttcatcaggagaatatatgaaggctgaggaagaggtgtgaattcgttcaattgtcatcactggc

SEQ ID NO: 389 - DNA, 88 bp, synthetic construct
  source 1..88: mol_type = other DNA
acaatgctttgctagtgatgaataggctgcgaattatcgcctcttcaatggataattcacatccctttcatcagtagctatgcattga

SEQ ID NO: 390 - DNA, 71 bp, synthetic construct
  source 1..71: mol_type = other DNA
gcgactagtcgttgggtaaagtcgccactgtgaagccacagatgggtggtgacttctcaacgattagctgc

SEQ ID NO: 391 - DNA, 86 bp, synthetic construct
  source 1..86: mol_type = other DNA
tgcttcccgaggtggcgattttacccaatgctctatatggattactttgctatagtcgttgggtaaagtcgccatttcggcaagtg

SEQ ID NO: 392 - DNA, 110 bp, synthetic construct
  source 1..110: mol_type = other DNA
ggctacagtctttcttcatgtgactcgtggactagtcgttgggtaaagtcgccagagaatatatgaaggtggcactttcctcaacgactcgttcaattgtcatcactggc

SEQ ID NO: 393 - DNA, 84 bp, synthetic construct
  source 1..84: mol_type = other DNA
ctccccatggccctgtagtcgttgggtaaagtcgccactgggctcagaccgcgacttaccacacgactacagggacctggggac

SEQ ID NO: 394 - DNA, 84 bp, synthetic construct
  source 1..84: mol_type = other DNA
gtcagaataatgtgttgctttccgctaagtggtgagtgatatgtgcatctcccactcagaggaaagcactagcattatggtgac

SEQ ID NO: 395 - DNA, 112 bp, synthetic construct
  source 1..112: mol_type = other DNA
ccttagcagagctggttgctttccgctaagtggtgatgtctaaactattcaccacttagaggaaagcctctagctactgctaggcaatccttccctcgataaatgtcttggc

SEQ ID NO: 396 - DNA, 85 bp, synthetic construct
  source 1..85: mol_type = other DNA
cgctggcgacgggaaccacttagcgcgaagcaaggctgtgacacttcaaacgttgctttccgctaagtggtgaccgtccacggca

SEQ ID NO: 397 - DNA, 71 bp, synthetic construct
  source 1..71: mol_type = other DNA
gcgactttgcagtggccttcgtggcccctgtgaagccacagatggggggtcacgagccactgcgaagctgc

SEQ ID NO: 398 - DNA, 86 bp, synthetic construct
  source 1..86: mol_type = other DNA
tgcttcccgagggggctatgaaggccattgagaaatatggattactttgctatttgcagtggccttcgtagccctttcggcaagtg

SEQ ID NO: 399 - DNA, 84 bp, synthetic construct
  source 1..84: mol_type = other DNA
gtcagaataatgttttgcagtggccttcgtggcccgtgatatgtgcatcggtcacgtagactactgcactcgcattatggtgac

SEQ ID NO: 400 - DNA, 112 bp, synthetic construct
  source 1..112: mol_type = other DNA
ccttagcagagctgtttgcagtggccttcgtggccctgtctaaactatgggtcacgaagaccactgcccatagctactgctaggcaatccttccctcgataaatgtcttggc

SEQ ID NO: 401 - DNA, 84 bp, synthetic construct
  source 1..84: mol_type = other DNA
ctccccatggccctgagcctcccgtcctaagaccccactgggctcagacagggtcttggaaaggaggctcagggacctggggac

SEQ ID NO: 402 - DNA, 81 bp, synthetic construct
  source 1..81: mol_type = other DNA
cttcagggtgggtcttaggtcgggagctgctgatttaaatagtgattgtcagcctcccgtcctaagaccccaccttggggg

SEQ ID NO: 403 - DNA, 109 bp, synthetic construct
  source 1..109: mol_type = other DNA
tgagttttgaggttgcttcagtgagcctcccgtcctaagaccccacttggaattaaaatcaagtgggtcttgacgggtggcaccctatggctaaccatcatctactcca

SEQ ID NO: 404 - DNA, 86 bp, synthetic construct
  source 1..86: mol_type = other DNA
tgcttcccgaggccagggtgagtaacgtggcctcttatggattactttgctaagatccacgttactcaccctggtttcggcaagtg

SEQ ID NO: 405 - DNA, 71 bp, synthetic construct
  source 1..71: mol_type = other DNA
gcgacagatccacgttactcacccaagctgtgaagccacagatgggcttgggtgagacgtggatctgctgc

SEQ ID NO: 406 - DNA, 88 bp, synthetic construct
  source 1..88: mol_type = other DNA
acaatgctttgctagaggggtgtataccgcggatcttcgcctcttcaatggaagatccacgttactcacccctgtagctatgcattga

SEQ ID NO: 407 - DNA, 112 bp, synthetic construct
  source 1..112: mol_type = other DNA
ccttagcagagctgagatccacgttactcacccttgtgtctaaactatcaagggtgagtcacgtggagattagctactgctaggcaatccttccctcgataaatgtcttggc

SEQ ID NO: 408 - DNA, 81 bp, synthetic construct
  source 1..81: mol_type = other DNA
cttcaggaaagatccacgttactcacccttagatttaaatagtgattgtctaggtgagttacgtggatctgttcttggggg

SEQ ID NO: 409 - DNA, 110 bp, synthetic construct
  source 1..110: mol_type = other DNA
ggcctggctggacagagttgtcatgtgtcagatccacgttactcaccctgcagaacatccgctcacctgtagggtgagactcgtggatctgtcacatgacaacccagcct

SEQ ID NO: 410 - DNA, 71 bp, synthetic construct
  source 1..71: mol_type = other DNA
gcgacttcaggaatgggttccaaggagctgtgaagccacagatgggctccttggaccattcctgaagctgc

SEQ ID NO: 411 - DNA, 91 bp, synthetic construct
  source 1..91: mol_type = other DNA
ctggggtacggggatggattcaggaatgggttccaaggaattgtttctaatgtcctgaagccattcatggagccgtccgtatccgctgcag

SEQ ID NO: 412 - DNA, 84 bp, synthetic construct
  source 1..84: mol_type = other DNA
gtcagaataatgtttcaggaatgggttccaaggaagtgatatgtgcatcttcttggtacacgttcctgctcacattatggtgac

SEQ ID NO: 413 - DNA, 112 bp, synthetic construct
  source 1..112: mol_type = other DNA
ccttagcagagctgttcaggaatgggttccaaggagtgtctaaactatctccttggaacacattcctacatagctactgctaggcaatccttccctcgataaatgtcttggc

SEQ ID NO: 414 - DNA, 84 bp, synthetic construct
  source 1..84: mol_type = other DNA
ctccccatggccctgtataatataatagaaccacaggctgggctcagacgtgtggtttatcctgttgtacagggacctggggac

SEQ ID NO: 415 - DNA, 67 bp, synthetic construct
  source 1..67: mol_type = other DNA
gcatataatataatagaaccacagggcccttcatgctgcccagcttgtggttctattatgttataac

SEQ ID NO: 416 - DNA, 86 bp, synthetic construct
  source 1..86: mol_type = other DNA
tgcttcccgaggcgtgctgaactggtatcgaaatgtatggattactttgctacatgcggtaccagtttagcacgtttcggcaagtg

SEQ ID NO: 417 - DNA, 112 bp, synthetic construct
  source 1..112: mol_type = other DNA
ccttagcagagctgcatgcggtaccagtttagcacgtgtctaaactatcgtgctagacttgtactgtcagtagctactgctaggcaatccttccctcgataaatgtcttggc

SEQ ID NO: 418 - DNA, 71 bp, synthetic construct
  source 1..71: mol_type = other DNA
gcgaccatgcggtaccagtttagcacgctgtgaagccacagatgggcgtgctagaggtgtcgcatggctgc

SEQ ID NO: 419 - DNA, 86 bp, synthetic construct
  source 1..86: mol_type = other DNA
tgcttcccgaggcagcgagggagaagattaccatttatggattactttgctaaatatagtcttctccctcgctgtttcggcaagtg

SEQ ID NO: 420 - DNA, 77 bp, synthetic construct
  source 1..77: mol_type = other DNA
gtggcctcgaatatagtcttctccctcgcttgtgcaggtcccaatggagcgagggataggactatactcggggacgc

SEQ ID NO: 421 - DNA, 71 bp, synthetic construct
  source 1..71: mol_type = other DNA
gcgacaatatagtcttctccctcgctgctgtgaagccacagatgggcagtgagggaagactatgttgctgc

SEQ ID NO: 422 - DNA, 86 bp, synthetic construct
  source 1..86: mol_type = other DNA
tgcttcccgaggccggttgaacattggttgcctcatatggattactttgctatgaacgaccagtgtttaaccggtttcggcaagtg

SEQ ID NO: 423 - DNA, 85 bp, synthetic construct
  source 1..85: mol_type = other DNA
cgctggcgacgggacggatgaacttagaggagacgctgtgacacttcaaacttctccttggagttcatccgcgccgtccacggca

SEQ ID NO: 424 - DNA, 71 bp, synthetic construct
  source 1..71: mol_type = other DNA
gcgacttctccttggagttcatccgcgctgtgaagccacagatgggcgcggatgatccaaggaggagctgc

SEQ ID NO: 425 - DNA, 84 bp, synthetic construct
  source 1..84: mol_type = other DNA
ctccccatggccctgatacacatcagaatccttactgctgggctcagaccgtaaggatctcctgtgtatcagggacctggggac

SEQ ID NO: 426 - DNA, 110 bp, synthetic construct
  source 1..110: mol_type = other DNA
ggctacagtctttcttcatgtgactcgtggacatacacatcagaatccttacttgagaatatatgaaggaggtaggatactgatgtgtacgttcaattgtcatcactggc

SEQ ID NO: 427 - DNA, 86 bp, synthetic construct
  source 1..86: mol_type = other DNA
tgcttcccgaggcggtaaggattctgatgtaatattatggattactttgctaatacacatcagaatccttactgtttcggcaagtg

SEQ ID NO: 428 - DNA, 77 bp, synthetic construct
  source 1..77: mol_type = other DNA
gtggcctcgatacacatcagaatccttacttgtgcaggtcccaatggagtaaggatcctgatgtgtctcggggacgc

SEQ ID NO: 429 - DNA, 84 bp, synthetic construct
  source 1..84: mol_type = other DNA
ctccccatggccctgttgttgaaggcgtttgcacatgctgggctcagacctgtgcaatgcgatcaatagcagggacctggggac

SEQ ID NO: 430 - DNA, 89 bp, synthetic construct
  source 1..89: mol_type = other DNA
gtcagcagtgcctttgttgaaggcgtttgcacatgttaagattctaaaattatcttgtgcggaagcacttcaataaaagtaaggttgac

SEQ ID NO: 431 - DNA, 86 bp, synthetic construct
  source 1..86: mol_type = other DNA
tgcttcccgaggcgtgtgtgaacgccttcacataatatggattactttgctattgttgaaggcgtttgcacatgtttcggcaagtg

SEQ ID NO: 432 - DNA, 77 bp, synthetic construct
  source 1..77: mol_type = other DNA
gtggcctcgttgttgaaggcgtttgcacattgtgcaggtcccaatgggtgtgcaaaggtcttcaatcacggggacgc

SEQ ID NO: 433 - DNA, 86 bp, synthetic construct
  source 1..86: mol_type = other DNA
tgcttcccgaggtgtgcgtccagcgcttcgagacatatggattactttgctatgtgcgaagcgctggatgcacatttcggcaagtg

SEQ ID NO: 434 - DNA, 110 bp, synthetic construct
  source 1..110: mol_type = other DNA
ggctacagtctttcttcatgtgactcgtggactgtgcgaagcgctggatgcacagagaatatatgaaggtgtggtccaccgcttcgcaccgttcaattgtcatcactggc

SEQ ID NO: 435 - DNA, 85 bp, synthetic construct
  source 1..85: mol_type = other DNA
ggctgagccgcatgtgcatccagccttcgacacatatgtcctgacccagctatgtgcgaagcgctggatgcacatgccctctgcc

SEQ ID NO: 436 - DNA, 89 bp, synthetic construct
  source 1..89: mol_type = other DNA
gtcagcagtgccttgtgcgaagcgctggatgcacattaagattctaaaattatcttgcatccaccgacttcgcacgaagtaaggttgac

SEQ ID NO: 437 - DNA, 72 bp, synthetic construct
  source 1..72: mol_type = other DNA
tgtcgggtgtgcgaagcgctggatgcacactgttgaatctcatgggtgcacccagccttcgcacgtctgaca

SEQ ID NO: 438 - DNA, 86 bp, synthetic construct
  source 1..86: mol_type = other DNA
tgcttcccgagggctgcagctgaatcacactcggatatggattactttgctatcctgtgtgattcagctgcagctttcggcaagtg

SEQ ID NO: 439 - DNA, 110 bp, synthetic construct
  source 1..110: mol_type = other DNA
ggctacagtctttcttcatgtgactcgtggactcctgtgtgattcagctgcagcgagaatatatgaagggctgagctgcatcacacaggcgttcaattgtcatcactggc

SEQ ID NO: 440 - DNA, 82 bp, synthetic construct
  source 1..82: mol_type = other DNA
gcagtcctctgagctgcagctgaatcacaggtacaagaagaatgtagttcctgtgtgattcagctgcagcttggtggcctgc

SEQ ID NO: 441 - DNA, 81 bp, synthetic construct
  source 1..81: mol_type = other DNA
gatactcgaaggagctgcatatgaatccaccaggtctttatttatgatcctgtgtgattcagctgcagcttttttagtatc

SEQ ID NO: 442 - DNA, 84 bp, synthetic construct
  source 1..84: mol_type = other DNA
ctccccatggccctgtcctgtgtgattcagctgcagcctgggctcagacatgcagctaatgtcacaggacagggacctggggac

SEQ ID NO: 443 - DNA, 86 bp, synthetic construct
  source 1..86: mol_type = other DNA
tgcttcccgaggatggcatctacatggtacttcattatggattactttgctaatgtgtaccatgtagatgccattttcggcaagtg

SEQ ID NO: 444 - DNA, 110 bp, synthetic construct
  source 1..110: mol_type = other DNA
ggctacagtctttcttcatgtgactcgtggacatgtgtaccatgtagatgccatgagaatatatgaaggatggatctagatggtacacacgttcaattgtcatcactggc

SEQ ID NO: 445 - DNA, 85 bp, synthetic construct
  source 1..85: mol_type = other DNA
ggctgagccgcaatggcatctacaggtacaacattatgtcctgacccagctaatgtgtaccatgtagatgccattgccctctgcc

SEQ ID NO: 446 - DNA, 87 bp, synthetic construct
  source 1..87: mol_type = other DNA
gacagtgcagtcaccatgtgtaccatgtagatgccaaacagcactggagggaggcatctacctggtacacatgatgagtgtactgtg

SEQ ID NO: 447 - DNA, 77 bp, synthetic construct
  source 1..77: mol_type = other DNA
gtggcctcgatgtgtaccatgtagatgccatgtgcaggtcccaatggtggcatctagatggtacacctcggggacgc

SEQ ID NO: 448 - DNA, 585 bp, synthetic construct
  source 1..585: mol_type = other DNA
aggagggtgggggtggaggcaagcagaggacttcctgatcgcgtacccatggctacagtctttcttcatgtgactcgtggacttcaggaatgggttccaaggatgagaatatatgaaggatcctggaagctattcctgacgttcaattgtcatcactggcatcttttttgatcattgcaccatcatcaaatgcattgggataaccatgacgatgctacaagtggcccacttctgagatgcgggctgcttctggatgacactgcttcccgaggcctgtggttctgttatatccatatatggattactttgctatataatataatagaaccacaggtttcggcaagtgcctcctcgctggccccagggtaccacccggagcacaggtttggtgaccttcttctgaagaaaatatatttctttttattcatagctcttatgatagcaatgtcagcagtgccttgtgcgaagcgctggatgcacattaagattctaaaattatcttgcatccaccgacttcgcacgaagtaaggttgaccatactctacagttgtgttttaatgtatattaatgttactaatgtgtttt

SEQ ID NO: 449 - DNA, 581 bp, synthetic construct
  source 1..581: mol_type = other DNA
aggagggtgggggtggaggcaagcagaggacttcctgatcgcgtacccatggctacagtctttcttcatgtgactcgtggacttcaggaatgggttccaaggatgagaatatatgaaggatcctggaagctattcctgacgttcaattgtcatcactggcatcttttttgatcattgcaccatcatcaaatgcattgggataaccatgacgatgctacaagtggcccacttctgagatgcgggctgcttctggatgacactgcttcccgaggcctgtggttctgttatatccatatatggattactttgctatataatataatagaaccacaggtttcggcaagtgcctcctcgctggccccagggtaccacccggagcacaggtttggtgaccttcattttccctccctttcccttaggagcctgttcctctcacgccctcacctggctgagccgcaatggcatctacaggtacaacattatgtcctgacccagctaatgtgtaccatgtagatgccattgccctctgcccctggcttcgaggaggaggaggagctgctttccccatcatctggaaggtg

SEQ ID NO: 450 - DNA, 585 bp, synthetic construct
  source 1..585: mol_type = other DNA
cttctgaagaaaatatatttctttttattcatagctcttatgatagcaatgtcagcagtgccttgtgcgaagcgctggatgcacattaagattctaaaattatcttgcatccaccgacttcgcacgaagtaaggttgaccatactctacagttgtgttttaatgtatattaatgttactaatgtgttttaggagggtgggggtggaggcaagcagaggacttcctgatcgcgtacccatggctacagtctttcttcatgtgactcgtggacttcaggaatgggttccaaggatgagaatatatgaaggatcctggaagctattcctgacgttcaattgtcatcactggcatcttttttgatcattgcaccatcatcaaatgcattgggataaccatgacgatgctacaagtggcccacttctgagatgcgggctgcttctggatgacactgcttcccgaggcctgtggttctgttatatccatatatggattactttgctatataatataatagaaccacaggtttcggcaagtgcctcctcgctggccccagggtaccacccggagcacaggtttggtgacctt

SEQ ID NO: 451 - DNA, 581 bp, synthetic construct
  source 1..581: mol_type = other DNA
cattttccctccctttcccttaggagcctgttcctctcacgccctcacctggctgagccgcaatggcatctacaggtacaacattatgtcctgacccagctaatgtgtaccatgtagatgccattgccctctgcccctggcttcgaggaggaggaggagctgctttccccatcatctggaaggtgaggagggtgggggtggaggcaagcagaggacttcctgatcgcgtacccatggctacagtctttcttcatgtgactcgtggacttcaggaatgggttccaaggatgagaatatatgaaggatcctggaagctattcctgacgttcaattgtcatcactggcatcttttttgatcattgcaccatcatcaaatgcattgggataaccatgacgatgctacaagtggcccacttctgagatgcgggctgcttctggatgacactgcttcccgaggcctgtggttctgttatatccatatatggattactttgctatataatataatagaaccacaggtttcggcaagtgcctcctcgctggccccagggtaccacccggagcacaggtttggtgacctt

SEQ ID NO: 452 - DNA, 396 bp, synthetic construct
  source 1..396: mol_type = other DNA
aggagggtgggggtggaggcaagcagaggacttcctgatcgcgtacccatggctacagtctttcttcatgtgactcgtggacatgtgtaccatgtagatgccatgagaatatatgaaggatggatctagatggtacacacgttcaattgtcatcactggcatcttttttgatcattgcaccatcatcaaatgcattgggataaccatgacgatgctacaagtggcccacttctgagatgcgggctgcttctggatgacactgcttcccgagggctgcagctgaatcacactcggatatggattactttgctatcctgtgtgattcagctgcagctttcggcaagtgcctcctcgctggccccagggtaccacccggagcacaggtttggtgacctt

SEQ ID NO: 453 - DNA, 363 bp, synthetic construct
  source 1..363: mol_type = other DNA
atgaagccacaggagccaagagcaggaggaccaaggccctggcgaaggccgtggcctcgatgtgtaccatgtagatgccatgtgcaggtcccaatggtggcatctagatggtacacctcggggacgcgggcctggacgccggcatccgggctcaggacccccctctctgccagaggcgatgctacaagtggcccacttctgagatgcgggctgcttctggatgacactgcttcccgagggctgcagctgaatcacactcggatatggattactttgctatcctgtgtgattcagctgcagctttcggcaagtgcctcctcgctggccccagggtaccacccggagcacaggtttggtgacctt

SEQ ID NO: 454 - DNA, 366 bp, synthetic construct
  source 1..366: mol_type = other DNA
atgaagccacaggagccaagagcaggaggaccaaggccctggcgaaggccgtggcctcgatgtgtaccatgtagatgccatgtgcaggtcccaatggtggcatctagatggtacacctcggggacgcgggcctggacgccggcatccgggctcaggacccccctctctgccagaggccttctgaagaaaatatatttctttttattcatagctcttatgatagcaatgtcagcagtgccttgtgcgaagcgctggatgcacattaagattctaaaattatcttgcatccaccgacttcgcacgaagtaaggttgaccatactctacagttgtgttttaatgtatattaatgttactaatgtgtttt

SEQ ID NO: 455 - DNA, 387 bp, synthetic construct
  source 1..387: mol_type = other DNA
aggagggtgggggtggaggcaagcagaggacttcctgatcgcgtacccatggctacagtctttcttcatgtgactcgtggactcctgtgtgattcagctgcagcgagaatatatgaagggctgagctgcatcacacaggcgttcaattgtcatcactggcatcttttttgatcattgcaccatcatcaaatgcattgggataaccatgacatgaagccacaggagccaagagcaggaggaccaaggccctggcgaaggccgtggcctcgatgtgtaccatgtagatgccatgtgcaggtcccaatggtggcatctagatggtacacctcggggacgcgggcctggacgccggcatccgggctcaggacccccctctctgccagaggc

SEQ ID NO: 456 - DNA, 366 bp, synthetic construct
  source 1..366: mol_type = other DNA
cttctgaagaaaatatatttctttttattcatagctcttatgatagcaatgtcagcagtgccttgtgcgaagcgctggatgcacattaagattctaaaattatcttgcatccaccgacttcgcacgaagtaaggttgaccatactctacagttgtgttttaatgtatattaatgttactaatgtgttttatgaagccacaggagccaagagcaggaggaccaaggccctggcgaaggccgtggcctcgatgtgtaccatgtagatgccatgtgcaggtcccaatggtggcatctagatggtacacctcggggacgcgggcctggacgccggcatccgggctcaggacccccctctctgccagaggc

SEQ ID NO: 457 - DNA, 374 bp, synthetic construct
  source 1..374: mol_type = other DNA
cttctgaagaaaatatatttctttttattcatagctcttatgatagcaatgtcagcagtgccttgtgcgaagcgctggatgcacattaagattctaaaattatcttgcatccaccgacttcgcacgaagtaaggttgaccatactctacagttgtgttttaatgtatattaatgttactaatgtgttttcattttccctccctttcccttaggagcctgttcctctcacgccctcacctggctgagccgcaatggcatctacaggtacaacattatgtcctgacccagctaatgtgtaccatgtagatgccattgccctctgcccctggcttcgaggaggaggaggagctgctttccccatcatctggaaggtg

SEQ ID NO: 458 - DNA, 374 bp, synthetic construct; identical to SEQ ID NO: 457
  source 1..374: mol_type = other DNA

SEQ ID NO: 459 - DNA, 399 bp, synthetic construct
  source 1..399: mol_type = other DNA
aggagggtgggggtggaggcaagcagaggacttcctgatcgcgtacccatggctacagtctttcttcatgtgactcgtggactcctgtgtgattcagctgcagcgagaatatatgaagggctgagctgcatcacacaggcgttcaattgtcatcactggcatcttttttgatcattgcaccatcatcaaatgcattgggataaccatgaccttctgaagaaaatatatttctttttattcatagctcttatgatagcaatgtcagcagtgccttgtgcgaagcgctggatgcacattaagattctaaaattatcttgcatccaccgacttcgcacgaagtaaggttgaccatactctacagttgtgttttaatgtatattaatgttactaatgtgtttt

SEQ ID NO: 460 - DNA, 399 bp, synthetic construct
  source 1..399: mol_type = other DNA
aggagggtgggggtggaggcaagcagaggacttcctgatcgcgtacccatggctacagtctttcttcatgtgactcgtggacatgtgtaccatgtagatgccatgagaatatatgaaggatggatctagatggtacacacgttcaattgtcatcactggcatcttttttgatcattgcaccatcatcaaatgcattgggataaccatgaccttctgaagaaaatatatttctttttattcatagctcttatgatagcaatgtcagcagtgccttgtgcgaagcgctggatgcacattaagattctaaaattatcttgcatccaccgacttcgcacgaagtaaggttgaccatactctacagttgtgttttaatgtatattaatgttactaatgtgtttt

SEQ ID NO: 461-581 - skipped (121 entries)

SEQ ID NO: 582 - DNA, 186 bp, synthetic construct
  source 1..186: mol_type = other DNA
gatgctacaagtggcccacttctgagatgcgggctgcttctggatgacactgcttcccgaggcatttcgccctatctgcaagtactatggattactttgctagtggtgtagatagggtgaaatgtttcggcaagtgcctcctcgctggccccagggtaccacccggagcacaggtttggtgacctt

SEQ ID NO: 583 - DNA, 207 bp, synthetic construct
  source 1..207: mol_type = other DNA
agggactgggcccacggggaggcagcgtccccgaggcagcagcggcagcggcggctcctctccccatggccctgtcacaacctcctagaaagagtactgggctcagaccctctttcaggccgttgtgacagggacctggggaccccggcaccggcaggccccaaggggtgaggtgagcgggcattgggacctcccctccctgtactc

SEQ ID NO: 584 - DNA, 210 bp, synthetic construct
  source 1..210: mol_type = other DNA
aggagggtgggggtggaggcaagcagaggacttcctgatcgcgtacccatggctacagtctttcttcatgtgactcgtggacttgtactacacaaaagtactgtgagaatatatgaaggacaggctttagtgtagtatgcgttcaattgtcatcactggcatcttttttgatcattgcaccatcatcaaatgcattgggataaccatgac

SEQ ID NO: 585 - DNA, 210 bp, unidentified
  source 1..210: mol_type = unassigned DNA
ggtaagtcatgactccctacaatggacatgatcatagtaggtactataaggcacctagctataccctcctatagagagtttgagtctattgtagcaagtctttcttttagggcctaggactcttcccactctcttccatcgcctaagctctaactcttccttgtagtagaaataagtcacttctaaggctgggaccctcctagaccctaa

SEQ ID NO: 586 - DNA, 354 bp, synthetic construct
  source 1..354: mol_type = other DNA
ttaggatgagttgagatcccagtgatcttctcgctaagagtttcctgcctgggcaaggaggaaagatgctacaagtggcccacttctgagatgcgggctgcttctggatgacactgcttcccgaggcatttcgccctatctgcaagtactatggattactttgctagtggtgtagatagggtgaaatgtttcggcaagtgcctcctcgctggccccagggtaccacccggagcacaggtttggtgaccttcttcctcatcagggctttgtgccagcaaatgactccctcaccaaggaagcaagagcctctgaatcccatctgggctcttcctgaacacccctatctccccctct

SEQ ID NO: 587-703 - skipped (117 entries)

SEQ ID NO: 704 - DNA, 21 bp, unidentified
  source 1..21: mol_type = unassigned DNA
ttcaggaatgggttccaagga

SEQ ID NO: 705 - DNA, 23 bp, unidentified
  source 1..23: mol_type = unassigned DNA
ttcaggaatgggttccaaggatg

SEQ ID NO: 706 - DNA, 21 bp, unidentified
  source 1..21: mol_type = unassigned DNA
tcctggaagctattcctgacg

SEQ ID NO: 707 - DNA, 22 bp, unidentified
  source 1..22: mol_type = unassigned DNA
tcctggaagctattcctgacgt

SEQ ID NO: 708 - DNA, 23 bp, unidentified
  source 1..23: mol_type = unassigned DNA
tcctggaagctattcctgacgtt

SEQ ID NO: 709 - DNA, 21 bp, unidentified
  source 1..21: mol_type = unassigned DNA
tataatataatagaaccacag

SEQ ID NO: 710 - DNA, 23 bp, unidentified
  source 1..23: mol_type = unassigned DNA
tataatataatagaaccacagga

SEQ ID NO: 711 - DNA, 21 bp, unidentified
  source 1..21: mol_type = unassigned DNA
tgtggttctgttatatccata

SEQ ID NO: 712 - DNA, 22 bp, unidentified
  source 1..22: mol_type = unassigned DNA
tgtggttctgttatatccatat

SEQ ID NO: 713 - DNA, 23 bp, unidentified
  source 1..23: mol_type = unassigned DNA
tgtggttctgttatatccatata